SEQUENCE LISTING (94 sequences)

SEQ ID NO: 1 - protein, 107 aa, Artificial Sequence
  Description of Artificial Sequence: Synthetic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 2 - DNA, 324 bp, Artificial Sequence
  Description of Artificial Sequence: Synthetic polynucleotide
atgggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaa

SEQ ID NO: 3 - protein, 8 aa, Artificial Sequence
  Description of Artificial Sequence: Synthetic peptide
SGGGSGVD

SEQ ID NO: 4 - DNA, 18 bp, Artificial Sequence
  Description of Artificial Sequence: Synthetic oligonucleotide
agcggaggaggatccgga

SEQ ID NO: 5 - protein, 287 aa, Homo sapiens
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAP

SEQ ID NO: 6 - DNA, 864 bp, Homo sapiens
gtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggcc

SEQ ID NO: 7 - protein, 18 aa, Thosea asigna virus
EGRGSLLTCGDVEENPGP

SEQ ID NO: 8 - DNA, 54 bp, Thosea asigna virus
gaaggccgagggagcctgctgacatgtggcgatgtggaggaaaacccaggacca

SEQ ID NO: 9 - protein, 21 aa, Artificial Sequence
  Description of Artificial Sequence: Synthetic peptide
MEFGLSWLFLVAILKGVQCSR

SEQ ID NO: 10 - DNA, 63 bp, Artificial Sequence
  Description of Artificial Sequence: Synthetic oligonucleotide
atggagtttggactttcttggttgtttttggtggcaattctgaagggtgtccagtgtagcagg

SEQ ID NO: 11 - protein, 107 aa, Artificial Sequence
  Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 12 - DNA, 321 bp, Artificial Sequence
  Description of Artificial Sequence: Synthetic polynucleotide
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 13 - protein, 8 aa, Artificial Sequence
  Description of Artificial Sequence: Synthetic peptide
GGGSGGGG

SEQ ID NO: 14 - DNA, 24 bp, Artificial Sequence
  Description of Artificial Sequence: Synthetic oligonucleotide
ggcggaggaagcggaggtgggggc

SEQ ID NO: 15 - protein, 120 aa, Artificial Sequence
  Description of Artificial Sequence: Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 16 - DNA, 360 bp, Artificial Sequence
  Description of Artificial Sequence: Synthetic polynucleotide
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 17 - protein, 16 aa, Homo sapiens
ELPTQGTFSNVSTNVS

SEQ ID NO: 18 - DNA, 48 bp, Homo sapiens
gaacttcctactcaggggactttctcaaacgttagcacaaacgtaagt

SEQ ID NO: 19 - protein, 42 aa, Homo sapiens
PAPRPPTPAPTIASQPLSLRPEACRPAAGGAVHTRGLDFACD

SEQ ID NO: 20 - DNA, 126 bp, Homo sapiens
cccgccccaagaccccccacacctgcgccgaccattgcttctcaacccctgagtttgagacccgaggcctgccggccagctgccggcggggccgtgcatacaagaggactcgatttcgcttgcgac

SEQ ID NO: 21 - protein, 37 aa, Homo sapiens
IYIWAPLAGTCGVLLLSLVITLYCNHRNRRRVCKCPR

SEQ ID NO: 22 - DNA, 111 bp, Homo sapiens
atctatatctgggcacctctcgctggcacctgtggagtccttctgctcagcctggttattactctgtactgtaatcaccggaatcgccgccgcgtttgtaagtgtcccagg

SEQ ID NO: 23 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 336 bp, Artificial Sequence
  Description of Artificial Sequence: Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgt

SEQ ID NO: 25 - protein, 19 aa, Porcine teschovirus-1
ATNFSLLKQAGDVEENPGP

SEQ ID NO: 26 - DNA, 57 bp, Porcine teschovirus-1
gcaacgaatttttccctgctgaaacaggcaggggacgtagaggaaaatcctggtcct

SEQ ID NO: 27 - protein, 172 aa, Artificial Sequence
  Description of Artificial Sequence: Synthetic polypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 28 - DNA, 516 bp, Artificial Sequence
  Description of Artificial Sequence: Synthetic polynucleotide
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatc

SEQ ID NO: 29 - protein, 62 aa, Artificial Sequence
  Description of Artificial Sequence: Synthetic polypeptide
KKVAKKPTNKAPHPKQEPQEINFPDDLPGSNTAAPVQETLHGCQPVTQEDGKESRISVQERQ

SEQ ID NO: 30 - DNA, 186 bp, Artificial Sequence
  Description of Artificial Sequence: Synthetic polynucleotide
aaaaaggtggccaagaagccaaccaataaggccccccaccccaagcaggagccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 31 - protein, 6 aa, Artificial Sequence
  Description of Artificial Sequence: Synthetic peptide
MLEMLE

SEQ ID NO: 32 - DNA, 18 bp, Artificial Sequence
  Description of Artificial Sequence: Synthetic oligonucleotide
atgctcgagatgctggag

SEQ ID NO: 33 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 34 - DNA, 327 bp, Homo sapiens
atgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatga

SEQ ID NO: 35 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 36 - DNA, 891 bp, Homo sapiens
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctga

SEQ ID NO: 37 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 38 - DNA, 834 bp, Homo sapiens
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 39 - protein, 194 aa, Homo sapiens
MKWKALFTAAILQAQLPITASSLPHPTQQSPEKKVLGPGGCTCRHNRFCNEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 585 bp, Homo sapiens
atgaagtggaaggcgcttttcaccgcggccatcctgcaggcacagttgccgattacagcctccagcctcccccacccaactcagcagagccctgagaagaaagtcctgggtcccggaggctgcacctgcagacacaacagattctgcaatgaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 41 - protein, 416 aa, Homo sapiens [sequence omitted]

SEQ ID NO: 42 - DNA, 1251 bp, Homo sapiens
atggacgaagcggatcggcggctcctgcggcggtgccggctgcggctggtggaagagctgcaggtggaccagctctgggacgccctgctgagcagcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgccgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggaggaaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataa

SEQ ID NO: 43 - protein, 3 aa, Artificial Sequence
  Description of Artificial Sequence: Synthetic peptide
MLE

SEQ ID NO: 44 - DNA, 9 bp, Artificial Sequence
  Description of Artificial Sequence: Synthetic oligonucleotide
atgctcgag

SEQ ID NO: 45 - protein, 119 aa, Artificial Sequence
  Description of Artificial Sequence: Synthetic polypeptide
EVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSS

SEQ ID NO: 46 - DNA, 357 bp, Artificial Sequence
  Description of Artificial Sequence: Synthetic polynucleotide
gaagtccaattgcaacagtcaggccccgaattgaaaaagcccggcgaaacagtgaagatatcttgtaaagcctccggttacccttttacgaactatggaatgaactgggtcaaacaagcccctggacagggattgaagtggatgggatggatcaatacatcaacaggcgagtctaccttcgcagatgatttcaaaggtcgctttgacttctcactggagaccagtgcaaataccgcctaccttcagattaacaatcttaaaagcgaggatatggcaacctacttttgcgcaagatgggaagtttatcacgggtacgtgccatactggggacaaggaacgacagtgacagttagtagc

SEQ ID NO: 47 - protein, 109 aa, Artificial Sequence
  Description of Artificial Sequence: Synthetic polypeptide
DIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKAL

SEQ ID NO: 48 - DNA, 327 bp, Artificial Sequence
  Description of Artificial Sequence: Synthetic polynucleotide
gacatccaattgacacaatcacacaaatttctctcaacttctgtaggagacagagtgagcataacctgcaaagcatcccaggacgtgtacaatgctgtggcttggtaccaacagaagcctggacaatccccaaaattgctgatttattctgcctctagtaggtacactggggtaccttctcggtttacgggctctgggtccggaccagatttcacgttcacaatcagttccgttcaagctgaagacctcgctgtttatttttgccagcagcacttccgaaccccttttacttttggctcaggcactaagttggaaatcaaggctttg

SEQ ID NO: 49 - DNA, 45 bp, Artificial Sequence
  Description of Artificial Sequence: Synthetic oligonucleotide
ggcggtggaggctccggtggaggcggctctggaggaggaggttca

SEQ ID NO: 50 - skipped

SEQ ID NO: 51 - protein, 14 aa, Unknown
  Description of Unknown: Myristoylation domain sequence
MGSSKSKPKDPSQR

SEQ ID NO: 52 - DNA, 42 bp, Unknown
  Description of Unknown: Myristoylation domain sequence
atggggagtagcaagagcaagcctaaggaccccagccagcgc

SEQ ID NO: 53 - protein, 106 aa, Artificial Sequence
  Description of Artificial Sequence: Synthetic polypeptide
DIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIK

SEQ ID NO: 54 - DNA, 318 bp, Artificial Sequence
  Description of Artificial Sequence: Synthetic polynucleotide
gacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaa

SEQ ID NO: 55 - protein, 112 aa, Artificial Sequence
  Description of Artificial Sequence: Synthetic polypeptide
EVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 56 - DNA, 336 bp, Artificial Sequence
  Description of Artificial Sequence: Synthetic polynucleotide
gaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttcc

SEQ ID NO: 57 - protein, 106 aa, Artificial Sequence
  Description of Artificial Sequence: Synthetic polypeptide
DIQLTQSPSSLSASVGDRVTITCSASSSVRFIHWYQQKPGKAPKRLIYDTSKLASGVPSRFSGSGSGTSYTLTISSLQPEDFATYYCQQWSSSPFTFGQGTKVEIK

SEQ ID NO: 58 - DNA, 318 bp, Artificial Sequence
  Description of Artificial Sequence: Synthetic polynucleotide
gacatccagctgacacaaagtcccagtagcctgtcagccagtgtcggcgatagggtgacaattacatgctccgcaagtagtagcgtcagattcatacactggtaccagcagaagcctgggaaggccccaaagaggcttatctacgataccagtaaactcgcctctggagttcctagccggttttctggatctggcagcggaactagctacaccctcacaatctccagtctgcaaccagaggactttgcaacctactactgccagcaatggagcagctcccctttcacctttgggcagggtactaaggtggagatcaag

SEQ ID NO: 59 - protein, 112 aa, Artificial Sequence
  Description of Artificial Sequence: Synthetic polypeptide
EVQLVESGGGLVQPGGSLRLSCAASGFNIKDYYIHWVRQAPGKGLEWIGWIDPENGDTEFVPKFQGKATMSADTSKNTAYLQMNSLRAEDTAVYYCKTGGFWGQGTLVTVSS

SEQ ID NO: 60 - DNA, 336 bp, Artificial Sequence
  Description of Artificial Sequence: Synthetic polynucleotide
gaggtgcagcttgtagagagcgggggaggcctcgtacagccagggggctctctgcgcctgtcatgtgcagcttcaggattcaatataaaggactattacattcactgggtacggcaagctcccggtaagggcctggaatggatcggttggatcgaccctgaaaacggagatacagaatttgtgcccaagttccagggaaaggctaccatgtctgccgatacttctaagaatacagcataccttcagatgaattctctccgcgccgaggacacagccgtgtattattgtaaaacgggagggttctggggtcagggtacccttgtgactgtgtcttcc

SEQ ID NO: 61 - protein, 288 aa, Artificial Sequence
  Description of Artificial Sequence: Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLEAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 62 - DNA, 864 bp, Artificial Sequence
  Description of Artificial Sequence: Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 63 - protein, 282 aa, Artificial Sequence
  Description of Artificial Sequence: Synthetic polypeptide
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFQFLRKKLFFKTS

SEQ ID NO: 64 - DNA, 846 bp, Artificial Sequence
  Description of Artificial Sequence: Synthetic polynucleotide
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttcagttcctccggaaaaaacttttctttaaaacatca

SEQ ID NO: 65 - protein, 282 aa, Artificial Sequence
  Description of Artificial Sequence: Synthetic polypeptide
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFQFLRKKLFFKTS

SEQ ID NO: 66 - DNA, 846 bp, Artificial Sequence
  Description of Artificial Sequence: Synthetic polynucleotide
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttcagttcctccggaaaaaacttttctttaaaacatca

SEQ ID NO: 67 - protein, 282 aa, Artificial Sequence
  Description of Artificial Sequence: Synthetic polypeptide
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 68 - DNA, 846 bp, Artificial Sequence
  Description of Artificial Sequence: Synthetic polynucleotide
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 69 - protein, 333 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 70 - DNA, 1002 bp, Homo sapiens
atgccccctcctagactgctgtttttcctgctctttctcaccccaatggaagttagacctgaggaaccactggtcgttaaagtggaagaaggtgataatgctgtcctccaatgccttaaagggaccagcgacggaccaacgcagcaactgacttggagccgggagtcccctctcaagccgtttctcaagctgtcacttggcctgccaggtcttggtattcacatgcgcccccttgccatttggctcttcatattcaatgtgtctcaacaaatgggtggattctacctttgccagcccggccccccttctgagaaagcttggcagcctggatggaccgtcaatgttgaaggctccggtgagctgtttagatggaatgtgagcgaccttggcggactcggttgcggactgaaaaataggagctctgaaggaccctcttctccctccggtaagttgatgtcacctaagctgtacgtgtgggccaaggaccgccccgaaatctgggagggcgagcctccatgcctgccgcctcgcgattcactgaaccagtctctgtcccaggatctcactatggcgcccggatctactctttggctgtcttgcggcgttcccccagatagcgtgtcaagaggacctctgagctggacccacgtacaccctaagggccctaagagcttgttgagcctggaactgaaggacgacagacccgcacgcgatatgtgggtaatggagaccggccttctgctccctcgcgctaccgcacaggatgcagggaaatactactgtcatagagggaatctgactatgagctttcatctcgaaattacagcacggcccgttctttggcattggctcctccggactggaggctggaaggtgtctgccgtaacactcgcttacttgattttttgcctgtgtagcctggttgggatcctgcatcttcagcgagcccttgtattgcgccgaaaaagaaaacgaatgactgaccctacacgacgattctga

SEQ ID NO: 71 - protein, 63 aa, Artificial Sequence
  Description of Artificial Sequence: Synthetic polypeptide
VAAILGLGLVLGLLGPLAILLALYLLRRDQRLPPDAHKPPGGGSFRTPIQEEQADAHSTLAKI

SEQ ID NO: 72 - DNA, 186 bp, Artificial Sequence
  Description of Artificial Sequence: Synthetic polynucleotide
gttgccgccatcctgggcctgggcctggtgctggggctgctgggccccctggccatcctgctggccctgtacctgctccgggaccagaggctgccccccgatgcccacaagccccctgggggaggcagtttccggacccccatccaagaggagcaggccgacgcccactccaccctggccaagatc

SEQ ID NO: 73 - protein, 45 aa, Artificial Sequence
  Description of Artificial Sequence: Synthetic polypeptide
SVVKRGRKKLLYIFKQPFMRPVQTTQEEDGCSCRFPEEEEGGCEL

SEQ ID NO: 74 - DNA, 135 bp, Artificial Sequence
  Description of Artificial Sequence: Synthetic polynucleotide
agtgtagttaaaagaggaagaaaaaagttgctgtatatatttaaacaaccatttatgagaccagtgcaaaccacccaagaagaagacggatgttcatgcagattcccagaagaagaagaaggaggatgtgaattg

SEQ ID NO: 75 - protein, 68 aa, Artificial Sequence
  Description of Artificial Sequence: Synthetic polypeptide
FWVLVVVGGVLACYSLLVTVAFIIFWVRSKRSRLLHSDYMNMTPRRPGPTRKHYQPYAPPRDFAAYRS

SEQ ID NO: 76 - DNA, 204 bp, Artificial Sequence
  Description of Artificial Sequence: Synthetic polynucleotide
ttctgggtactggttgtagtcggtggcgtacttgcttgttattctcttcttgttaccgtagccttcattatattctgggtccgatcaaagcgctcaagactcctccattccgattatatgaacatgacacctcgccgacctggtcctacacgcaaacattatcaaccctacgcacccccccgagacttcgctgcttatcgatcc

SEQ ID NO: 77 - protein, 107 aa, Artificial Sequence; identical to SEQ ID NO: 1
  Description of Artificial Sequence: Synthetic polypeptide

SEQ ID NO: 78 - DNA, 321 bp, Artificial Sequence
  Description of Artificial Sequence: Synthetic polynucleotide
ggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 79 - protein, 106 aa, Artificial Sequence
  Description of Artificial Sequence: Synthetic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKL

SEQ ID NO: 80 - DNA, 318 bp, Artificial Sequence
  Description of Artificial Sequence: Synthetic polynucleotide
ggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctc

SEQ ID NO: 81 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 82 - DNA, 321 bp, Homo sapiens
ggcgtgcaagtggaaactataagcccgggagacggccgcacatttcccaagagaggccagacctgcgtggtgcactatacaggaatgctggaggacgggaagaaattcgatagctcccgggatcgaaataagcctttcaaattcatgctgggcaagcaagaagtcatcagaggctgggaagaaggcgtcgcccagatgtccgtgggtcagcgcgccaagctgacaattagtccagattacgcctatggcgcaacaggccatcccggcatcatccccccacatgccacactcgtctttgatgtcgagctcctgaaactggag

SEQ ID NO: 83 - protein, 296 aa, Homo sapiens; identical to SEQ ID NO: 35

SEQ ID NO: 84 - DNA, 888 bp, Homo sapiens
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccc

SEQ ID NO: 85 - protein, 35 aa, Artificial Sequence
  Description of Artificial Sequence: Synthetic polypeptide
TKKKYSSSVHDPNGEYMFMRAVNTAKKSRLTDVTL

SEQ ID NO: 86 - DNA, 105 bp, Artificial Sequence
  Description of Artificial Sequence: Synthetic polynucleotide
acaaaaaagaagtattcatccagtgtgcacgaccctaacggtgaatacatgttcatgagagcagtgaacacagccaaaaaatctagactcacagatgtgacccta

SEQ ID NO: 87 - protein, 48 aa, Artificial Sequence
  Description of Artificial Sequence: Synthetic polypeptide
QRRKYRSNKGESPVEPAEPCRYSCPREEEGSTIPIQEDYRKPEPACSP

SEQ ID NO: 88 - skipped

SEQ ID NO: 89 - protein, 383 aa, Artificial Sequence
  Description of Artificial Sequence: Synthetic polypeptide
CYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 90 - protein, 5 aa, Artificial Sequence
  Description of Artificial Sequence: Synthetic peptide
SGGGS

SEQ ID NO: 91 - protein, 5 aa, Artificial Sequence
  Description of Artificial Sequence: Synthetic peptide
GGGGS

SEQ ID NO: 92 - protein, 5 aa, Artificial Sequence
  Description of Artificial Sequence: Synthetic peptide
GSGPR

SEQ ID NO: 93 - protein, 16 aa, Homo sapiens
MGSNKSKPKDASQRRR

SEQ ID NO: 94 - protein, 5 aa, Artificial Sequence
  Description of Artificial Sequence: Synthetic peptide
  MOD_RES (4)..(4): Any amino acid
MGCXC

(19) United States
(12) Patent Application Publication
(10) Pub. No.: US 2020/0347148 A1
(43) Pub. Date: Nov. 5, 2020

(54) METHODS FOR ENHANCING AND MAINTAINING CAR-T CELL EFFICACY

(71) Applicant: Bellicum Pharmaceuticals, Inc., Frisco, TX (US)

(72) Inventors: Aaron Edward Foster, Houston, TX (US); David Michael Spencer, Frisco, TX (US)

(21) Appl. No.: 16/770,019

(22) PCT Filed: Dec. 7, 2018
(86) PCT No.: PCT/US2018/064568

§ 371 Date: Jun. 4, 2020

Related U.S. Application Data
(60) Provisional application No. 62/596,744, filed on Dec. 8, 2017.

(51) Int. Cl. C07K 16/32; C12N 5/0783; C07K 14/705; A61K 39/00; A61P 35/00; A61K 39/395; C07K 14/725

(52) U.S. Cl. CPC C07K 16/32; C12N 5/0636; C07K 14/70578; A61K 39/001102; A61P 35/00; A61K 39/3955; A61K 2039/5158; C07K 14/7051; C07K 2317/24; C07K 2319/03; C07K 2319/33; C07K 2319/02; C07K 14/70517

(57) ABSTRACT

The technology relates generally to the field of immunology and relates in part to compositions and methods for activating T cells and other cells resulting in an immune response against a target antigen. The technology also relates to compositions and methods for enhancing and maintaining chimeric antigen receptor-expressing T cells, while reducing cytotoxic effects of CAR-T cell therapies

### RELATED APPLICATIONS

Priority is claimed to U.S. Provisional Patent Application Ser. No. 62/596,744, filed Dec. 8, 2018, by Aaron Edward Foster and David Michael Spencer, entitled “Methods for Enhancing and Maintaining CAR-T cell Efficacy” which is referred to and incorporated by reference thereof, in its entirety.

### FIELD

The technology relates generally to the field of immunology and relates in part to compositions and methods for activating T cells and other cells resulting in an immune response against a target antigen. The technology also relates to compositions and methods for enhancing and maintaining chimeric antigen receptor-expressing T cells, while reducing cytotoxic effects of CAR-T cell therapies

### BACKGROUND

T cell activation is an important step in the protective immunity against pathogenic microorganisms (e.g., viruses, bacteria, and parasites), foreign proteins, and harmful chemicals in the environment, and also as immunity against cancer and other hyperproliferative diseases. T cells express receptors on their surfaces (i.e., T cell receptors) that recognize antigens presented on the surface of cells. During a normal immune response, binding of these antigens to the T cell receptor, in the context of MHC antigen presentation, initiates intracellular changes leading to T cell activation.

Chimeric antigen receptors (CARs) are artificial receptors designed to convey antigen specificity to T cells without the requirement for MHC antigen presentation. Chimeric antigen receptor-expressing T cells may be used in various therapies, including cancer therapies. For example, adoptive transfer of T cells expressing CARs is an effective therapy for the treatment of certain hematological malignancies. In these patients, antitumor activity is associated with robust CAR-T cell expansion post-infusion that is often associated with toxicity (i.e., severe cytokine-release syndrome and neurotoxicity), while patients with poor CAR-T proliferation and persistence show reduced rates of durable remissions. Thus, successful adoptive CAR T cell therapies requires CAR-T expansion and durable persistence following infusion while balancing CAR-T potency with safety.

### SUMMARY

Provided herein are modified cell populations and methods for enhancing and maintaining chimeric antigen receptor-expressing T cells, while reducing cytotoxic effects of CAR-T cell therapies. In some embodiments, a modified cell population is provided comprising modified T cells, wherein the modified T cells comprise a polynucleotide that encodes a chimeric antigen receptor, wherein the chimeric antigen receptor comprises: a transmembrane region; a T cell activation molecule; and an antigen recognition moiety wherein the ratio of CD8⁺ to CD4⁺ T cells in the modified cell population is 3:2 or greater. In some embodiments of the present application, the chimeric antigen receptor comprises a transmembrane region; a costimulatory polypeptide cytoplasmic signaling region, a truncated MyD88 polypeptide region lacking the TIR domain, a truncated MyD88 polypeptide region lacking the TIR domain and a costimulatory polypeptide cytoplasmic signaling region, or a truncated MyD88 polypeptide region lacking the TIR domain and a CD40 cytoplasmic polypeptide region lacking the CD40 extracellular domain; a T cell activation molecule; and an antigen recognition moiety. In some embodiments, the modified T cells comprise a second polynucleotide that encodes an inducible chimeric pro-apoptotic polypeptide. In some embodiments, the modified T cells comprise a second polynucleotide that encodes a chimeric signaling polypeptide, wherein the chimeric signaling polypeptide comprises: a costimulatory polypeptide cytoplasmic signaling region; a truncated MyD88 polypeptide region lacking the TIR domain; a truncated MyD88 polypeptide region lacking the TIR domain and a costimulatory polypeptide cytoplasmic signaling region; or a truncated MyD88 polypeptide region lacking the TIR domain and a CD40 cytoplasmic polypeptide region lacking the CD40 extracellular domain. In some embodiments, the chimeric signaling polypeptide comprises a membrane targeting region. In some embodiments, the costimulatory polypeptide cytoplasmic signaling region is a signaling region that activates the signaling pathways activated by MyD88, CD40 and/or MyD88-CD40 fusion chimeric polypeptide.

In some embodiments, the modified cell population comprises modified T cells, comprising a nucleic acid comprising a promoter operably linked to a first polynucleotide encoding the chimeric antigen receptor; and a second polynucleotide encoding a chimeric signaling polypeptide, wherein the chimeric signaling polypeptide comprises a costimulatory polypeptide cytoplasmic signaling region; a truncated MyD88 polypeptide region lacking the TIR domain; a truncated MyD88 polypeptide region lacking the TIR domain and a costimulatory polypeptide cytoplasmic signaling region; or a truncated MyD88 polypeptide region lacking the TIR domain and a CD40 cytoplasmic polypeptide region lacking the CD40 extracellular domain. In some embodiments, the nucleic acid comprises, in 5′ to 3′ order, the first polynucleotide and the second polynucleotide. In some embodiments, the first polynucleotide encodes, in 5′ to 3′ order, an antigen recognition moiety, a transmembrane region, and a T cell activation molecule, and the second polynucleotide is 3′ of the polynucleotide sequence encoding the T cell activation molecule. In some embodiments, the nucleic acid comprises a third polynucleotide that encodes a linker polypeptide between the first and the second polynucleotides. In some embodiments, the linker polypeptide comprises a 2A polypeptide. In some embodiments, the nucleic acid comprises a fourth polynucleotide encoding an inducible chimeric pro-apoptotic polypeptide. In some embodiments, the costimulatory polypeptide cytoplasmic signaling region is selected from the group consisting of CD27, CD28, 4-1BB, OX40, ICOS, RANK, TRANCE, and DAP10, or a signaling region that activates the signaling pathways activated by MyD88, CD40, CD27, CD28, 4-1BB, OX40, ICOS, RANK, TRANCE, and DAP10. In some embodiments, the chimeric antigen receptor comprises two costimulatory polypeptide cytoplasmic signaling regions selected from the group consisting of CD27, CD28, 4-1BB, OX40, ICOS, RANK, TRANCE, and DAP10, or a signaling region that activates the signaling pathways activated by CD27, CD28, 4-1BB, OX40, ICOS, RANK, TRANCE, and DAP10, or a signaling region that activates the signaling pathways activated by MyD88, CD40, CD27, CD28, 4-1BB, OX40, ICOS, RANK, TRANCE, and DAP10. In some embodiments, the chimeric signaling polypeptide comprises two costimulatory polypeptide cytoplasmic signaling regions selected from the group consisting of CD27, CD28, 4-1BB, OX40, ICOS, RANK, TRANCE, and DAP10, or a signaling region that activates the signaling pathways activated by MyD88, CD40, CD27, CD28, 4-1BB, OX40, ICOS, RANK, TRANCE, and DAP10.

Provided in some embodiments, are modified cell populations of the present application, wherein 80% or more of the modified cells are CD8⁺ T cells.

Provided in some embodiments are methods for stimulating a cell mediated immune response to a target cell or tissue in a subject, comprising administering a modified cell population of the present application. Provided in some embodiments are methods for treating a subject having a disease or condition associated with an elevated expression of a target antigen, comprising administering to the subject an effective amount of a modified cell population of the present application. Provided in some embodiments are methods for reducing the size of a tumor in a subject, comprising administering a modified cell population of the present application to the subject, wherein the antigen recognition moiety binds to an antigen on the tumor. Provided in some embodiments are methods for preparing a modified cell population of the present application, comprising contacting T cells with a nucleic acid that comprises a polynucleotide that encodes the chimeric antigen receptor with a cell population under conditions in which the nucleic acid is incorporated into the cells, and enriching the T cells to obtain a modified cell population wherein the ratio of CD8⁺ to CD4⁺ T cells in the cell population is 3:2 or greater. In some embodiments, the methods comprise the step of administering the modified cell population to a subject.

In some embodiments, the invention provides for combination therapies comprising the modified cell population described herein with cytokines or chemokines neutralizing agent, e.g. a neutralizing antibody. In some embodiments, the invention provides for combination therapies comprising the modified cell population described herein and a TNFα neutralizing agent, e.g., an anti-TNFα antibody.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIGS. 1A and 1B provide schematics comparing a conventional 1st generation CAR with an enhanced CAR including the signaling domains from MC, expressed in cis with the CD3 intracellular domain. These bicistronic vectors also express iC9 in the first position of the retroviral vector. FIGS. 1C and 1D: CD3⁺ CD34⁺ expression using flow cytometry was used to measure transduction efficiency and CAR mean fluorescence intensity (MFI). Fig. E: Potency of non-transduced (NT) T cells or T cells modified with either iC9-CD19.ζ or iC9-CD19.MC.ζ were assess in 7-day coculture assays with CD19⁺ Raji-EGPFluc tumor cells at a 1:1 effector to target (E:T) ratio. Tumor and T cell frequency (%) were analyzed by flow cytometry and IL-2 production assess by ELISA after 48 hours of the start of the coculture. FIGS. 1F and 1G: Immune deficient NSG mice were engrafted with CD19⁺ Raji-EGFPluc tumor cells on day 0 via tail vein injection and subsequently treated with NT, iC9-CD19.ζ or iC9-CD19.MC.ζ-modified T cells on day 4 post-tumor injection. Mice were assessed by bioluminescence imaging (BLI) on an approximately weekly basis to determine tumor growth and CAR-T cell activity. FIG. 1H: Analysis of tumor BLI was assessed on day 14 post-T cell injection. ** represents P-value <0.01; *** represents P-value <0.005.

FIG. 2A provides a schematic representation of an example of a construct that may be used to express a chimeric antigen receptor targeting CD19, a MyD88/CD40 chimeric costimulatory molecule, and an inducible chimeric iCaspase-9 safety switch polypeptide. FIG. 2B provides flow cytometry data, demonstrating that while transduction efficiency was unaffected, CAR levels were diminished by the inclusion of the MyD88 signaling domain. FIG. 2C provides a graph of the percentage of CD3⁺ CD34⁺ cells, and FIG. 2D provides a graph of CD34 MFI of cells transduced with the vectors depicted in FIG. 2A. *** represents a P-value of <0.005.

FIG. 3A provides a schematic representation of an example of a construct that may be used to express a chimeric antigen receptor targeting CD19, a MyD88/CD40 chimeric costimulatory molecule, and a inducible chimeric iCaspase-9 safety switch polypeptide. FIG. 3B: Non-transduced (NT) and T cells transduced with each vector were compared for transduction efficiency and CAR MFI. Dotted line labeled “CD3 (MFI)” indicates the approximate lower limit of CD3 expression on NT and iC9-CD19.ζ T cells. FIG. 3C: NT and iC9-CD19.ζ-MC-modified T cells were assessed for basal cytokine production after 48 hours. FIG. 3D A Western blot analysis was performed on NT, iMC-CD19.ζ and iC9-CD19.ζ-MC using an anti-MyD88, anti-Casp-9 and b-Actin antibodies demonstrating fusion of CAR-MC and high levels of iCasp-9 expression. FIG. 3E: Long-term cultures were established to assess the contribution of basal activation to CAR-T survival and proliferation with or without exogenous cytokine support (100 U/ml IL-2), showing that CAR-MC basal activity is sufficient to drive T cell expansion in the presence of IL-2. FIG. 3F provides a graph of the percentage of CD3⁺ CD34⁺ before and after treatment of modified T cells with rimiducid. (Left: no rimiducid (square); Right: plus 10 nM rimiducid (circle)). FIG. 3G provides a graph of IL-2 production in modified cells that express the chimeric MyD88/CD40 costimulatory molecule, and control cells. (From left to right: non-transduced cells (square); iC9-CD19.ζ day 14 (triangle); iC9-CD19.ζ-MC day 14 (upside down triangle); iC9-CD19ζ-MC day 100 (circle)); FIG. 3H provides a graph of PD-1 expression in modified cells that express the chimeric MyD88/CD40 costimulatory molecule, and control cells. (From left to right: iC9-CD19.ζ day 8 (square); iC9-CD19.ζ-MC day 8 (triangle); iC9-CD19.ζ-MC day 100 (circle)).

FIG. 4A: NSG mice engrafted with CD19⁺ Raji-EGFPluc tumor cells were treated with 5×10⁶ non-transduced (NT) or 1.25×10⁶ or 5×10⁶ iC9-CD19.ζ-MC-modified T cells via i.v. injection after 7 days. FIG. 4B: Tumor growth was assessed by bioluminescence imaging (BLI) on a weekly basis for 70 days post-tumor challenge. FIG. 4C: Weight of control (NT) and CAR-T-treated animals was measured to assess CAR-related toxicities. Mice exhibited a >20% reduction in weight on days 6 and 13 after receiving 5×10⁶ and 1×10⁶ iC9-CD19.ζ-MC-modified T cells, respectively. At this time, a single injection of 5 mg/kg rimiducid was administered i.p. which promptly resolved toxicity. FIG. 4D: Serum cytokine levels were assessed in naive (untreated), NT and CAR-treated before and 24 hours after rimiducid injection showing high levels of hIFN-γ and hIL-6 prior to drug administration, and returning to background levels following activation of the iC9 safety switch. FIG. 4E and FIG. 4F: Naive mice and mice that received CAR-T cells and rimiducid were subsequently rechallenged with Raji-EGFPluc tumor cells demonstrating that residual iC9-CD19.ζ-MC-modified can effectively control tumor outgrowth. FIG. 4G: 25 days post-tumor rechallenge, mice were sacrificed and the splenocytes were analyzed for the presence of CAR-T cells (CD3⁺CD34⁺) by flow cytometry and compared to the original product for frequency and FIG. 4H: CAR expression (mean fluorescence intensity; MFI) In FIG. 4H “pre-infusion” indicates pre-rimiducid administration. *** represents a P-value <0.005.

FIG. 5A and FIG. 5B: NSG mice were engrafted with CD123⁺ THP-1-EGFPluc tumor cells and subsequently treated with 2.5×10⁶ non-transduced (NT) or iC9-CD123.ζ-MC-modified T cells. Tumor growth was evaluated on a weekly basis using BLI measurements (FIG. 5B) and 100-day survival (FIG. 5C) were assessed showing robust and long-term anti-tumor activity from T cells expressing constitutively active MC compared to iC9-CD19.ζ-modified T cells. FIG. 5D: Similar to CD19-targeted, MC-enhanced CARs, iC9-CD123.ζ-MC-expressing T cells showed similar toxicity in NSG animals, but that weight loss could be resolved by administration of rimiducid without affecting anti-tumor activity.

FIG. 6A: NSG mice were engrafted with non-modified CD19⁺ Raji tumor cells and subsequently treated with 5×10⁶ T cells transduced with iC9-CD19.ζ-MC and EGFPluc retroviral vectors on day 7 post-tumor injection. CAR-T cell levels were assessed by BLI before and 24 and 48 hours after i.p. injection of rimiducid (0.00005, 0.0005, 0.005, 0.05, 0.5 and 5 mg/kg). CAR-T cell BLI (FIG. 6B) and serum cytokine levels of IFN-γ, IL-6, IL-13 and TNF-α at 24 hours post-rimiducid treatment (FIG. 6C) were measured. **, ***, and **** represent a P-value of <0.01, 0.005 and 0.001, respectively.

FIG. 7A: Additional vectors were designed to better understand the contribution of CAR-MC basal effects on anti-tumor activity and cytokine-related toxicities in animal models. iC9-CD19.ζ (i) and iC9-CD19.ζ-MC (ii) were compared with constructs bearing high efficiency 2A cleavage peptides (GSG-2A) (iii) or with MC moved to the first position to eliminate CAR-MC fusion pairing (iiii). In addition, a vector was constructed with a myristoylated MC domain to enhance basal activity by tethering the signaling domain to the cell membrane (iv). FIG. 7B: Basal activity of CAR-modified T cells was assessed by measuring IFN-γ and IL-6 in the absence of antigen.

FIG. 7C: To measure CAR-T expansion, T cells were co-transduced with a CAR vector and EGFPluc and subsequently administered to CD19⁺ Raji-bearing mice, Figs. D and E: CAR-T expansion was measured on days 0 (post-T cell injection), 12 and 19. FIG. 7F: Toxicity from MC-based CAR-T cells was assessed by measuring weight loss. Groups exhibiting >10% weight loss were treated with a single injection of rimiducid at 0.5 mg/kg. FIG. 7G: Serum levels of cytokines and chemokines was assessed on day 7 post-CAR-T cell injection. Changes in cytokine/chemokine levels are represented as fold-change from pre-CAR-T infusion samples.

FIG. 8A: Additional CD19-specific CAR constructs containing iC9 were developed using the CD28 and 4-1BB endodomains. Mice were engrafted with CD19⁺ Raji-EGFPluc tumor cells and subsequently treated with non-transduced (NT) or CAR-modified T cells 7 days post-tumor engraftment. FIG. 8B and FIG. 8C: Tumor growth was measured by bioluminescent imaging on a weekly basis. FIG. 8D: Mice treated with iC9-CD19.ζ-MC-modified T cells were treated with 5 mg/kg rimiducid on day 12 (red arrow) to resolve acute CAR-related weight loss.

FIG. 9A: NSG mice engrafted with CD19⁺ Raji-EGFPluc tumor cells were treated with 5×10⁶ non-transduced (NT) or iC9-CD19.ζ-MC-modified T cells. Mice receiving CAR-T cells were subsequently treated by twice weekly i.p. Injections of neutralizing antibodies to hIFN-γ, hIL-6 or hTNF-α, or a control non-specific isotype antibody after >15% weight loss was observed (day 15). As a control, one group was given 5 mg/kg rimiducid to resolve toxicity. FIG. 9B: Tumor growth was measured by bioluminescent imaging (BLI), and CAR-dependent toxicity by measuring weight loss. FIG. 9C: Serum concentration of hTNF-α was measured on days −7, 7 and 14 post-administration of neutralizing antibody cycle.

FIG. 10A: Transduced T cells forming bulk populations containing both CD4⁺ (high cytokine producers) and CD8⁺ (low cytokine production) were purified for either CD4 or CD8 expression using MACS columns. FIG. 10B: CAR expression of non-transduced (NT), unselected or CD4 and CD8-selected CAR-T cells. FIG. 10C: Purity of unselected and selected CAR-T cells.

FIG. 11A: Non-transduced (NT), unselected (CD3⁺), CD4 and CD8-selected iC9-CD19.ζ-MC-modified T cells were cultured with CD19⁺ Raji tumor cells and measured for IL-6 and TNF-α secretion after 48 hours. FIGS. 11B and 11C: NT, non-selected, CD4 and CD8-selected CAR-T cells were infused into CD19⁺ Raji-EGFPluc cells and tumor growth was measured by bioluminescence imaging. Mice exhibiting severe toxicity post-CAR-T infusion were sacrificed. Rimiducid to activate iC9 as not administered to any animals. FIG. 11D: Mice bearing CD19⁺ Raji-EGFPluc tumors were treated with 6.3×10⁵, 1.3×10⁶, 2.5×10⁶ or 5×10⁶ CD8-selected iC9-CD19.ζ-MC-modified T cells on day 4 and measured for BLI and weight loss. None of the groups received rimiducid to control CAR-related toxicity. FIG. 11E: Representative bioluminescence images of mice receiving 5×10⁶ CD8-selected iC9-CD19.ζ-MC-modified T cells. Arrows denote late resolution of intracranial tumors. ** and **** represent P-value of <0.01 and 0.001, respectively.

FIG. 12 provides a graph of basal cytokine production in transduced and iC9-CD19.ζ-MC-transduced cells. For each cytokine, left to right, the bars represent non-transduced CD3⁺ cells, non-transduced CD4⁺ cells, non-transduced CD8⁺ cells, CD3⁺ transduced cells, CD4⁺ transduced cells, and CD8⁺ transduced cells.

FIG. 13A is a graph of IL-6 concentration from non-transduced (NT) and transduced selected cells; FIG. 13B is a graph of IL-13 concentration from non-transduced (NT) and transduced selected cells; FIG. 13C is a graph of TNF-α concentration from non-transduced (NT) and transduced selected cells.

FIG. 14A provides a graph of bioluminescence of tumor-bearing mice following administration of non-transduced or increasing doses of transduced CAR-T cells (lines on right side of graph, top to bottom: NT, 0.625, 1.25, 2.5, and 5×10⁶ transduced cells). FIG. 14B provides a graph of mouse weight following administration of non-transduced or increasing doses of transduced CAR-T cells (lines on right side of graph, top to bottom: 0.625, 2.5 or 5, 1.25×10⁶ transduced cells; day 15, top to bottom: NT, 1.25, 2.5, 0.625, and 5×10⁶ transduced cells).

FIG. 15A provides a FACs analysis of non-transduced T cells; FIG. 15B provides a FACs analysis of transduced CAR-T cells 5 days following transduction, to measure CAR-expression using the CD34 epitope.

FIG. 16A provides FACs analyses of CD4-selected iC9-Her2.ζ-MC transduced T cells; FIG. 16B provides FACs analyses of CD8-selected iC9-Her2.ζ-MC transduced CAR-T cells.

FIG. 17A provides a graph of tumor size measured by calipers in tumor-bearing mice following administration of non-transduced T cells; FIG. 17B provides a graph of tumor size following administration of transduced non-selected CAR-T cells; FIG. 17C provides a graph of tumor size following administration of transduced CD4-selected CAR-T cells; FIG. 17D provides a graph of tumor size following administration of transduced CD8-selected CAR-T cells.

FIG. 18A provides a graph of tumor size measured by bioluminescence in tumor-bearing mice following administration of non-transduced T cells; FIG. 18B provides a graph of tumor size following administration of transduced non-selected CAR-T cells; FIG. 18C provides a graph of tumor size following administration of transduced CD4-selected CAR-T cells; FIG. 18D provides a graph of tumor size following administration of transduced CD8-selected CAR-T cells.

FIG. 19A provides a graph of weight change in tumor-bearing mice following administration of non-transduced T cells; FIG. 19B provides a graph of weight change following administration of transduced non-selected CAR-T cells; FIG. 19C provides a graph of weight change following administration of transduced CD4-selected CAR-T cells; FIG. 19D provides a graph of weight change following administration of transduced CD8-selected CAR-T cells.

FIG. 20 provides a graph of mouse survival following administration of non-transduced or transduced CAR-T cells (right side of graph, lines top to bottom: non-selected, CD8-selected, CD4-selected); line touching x axis at day 20 is NT.

FIG. 21A provides a graph of CAR-expression in non-transduced, non-selected transduced, CD4-selected transduced, and CD8-selected transduced CAR-T cells; FIG. 21B provides a graph of CD4 purity in non-transduced, non-selected transduced, CD4-selected transduced, and CD8-selected transduced CAR-T cells; FIG. 21C provides a graph of CD8 purity in non-transduced, non-selected transduced, CD4-selected transduced, and CD8-selected transduced CAR-T cells.

FIG. 22A provides photographs of bioluminescence in tumor-bearing mice following administration of non-transduced, non-selected transduced, CD4-selected transduced, and CD8-selected transduced CAR-T cells. FIG. 22B provides a graph of percent survival of the treated mice (lines, left to right, parallel to y-axis: CD4-selected, non-selected, non-transduced, CD8-selected).

FIG. 23A is a graph of weight change following administration of non-transduced cells to tumor bearing mice; FIG. 23B is a graph of weight change following administration of non-selected transduced CAR-T cells to tumor bearing mice; FIG. 23C is a graph of weight change following administration of CD4-selected CAR-T cells to tumor bearing mice; FIG. 23D is a graph of weight change following administration of CD8-selected CAR-T cells to tumor bearing mice.

FIG. 24A is a graph of tumor size following administration of non-transduced cells to tumor bearing mice; FIG. 24B is a graph of tumor size following administration of non-selected transduced CAR-T cells to tumor bearing mice; FIG. 24C is a graph of tumor size following administration of CD4-selected CAR-T cells to tumor bearing mice; FIG. 24D is a graph of tumor size following administration of CD8-selected CAR-T cells to tumor bearing mice.

FIG. 25A provides the results of FACs sorting of iC9-CD19.ζ and iC9-CD19.ζ-MC-modified T cells following long-term culture. FIG. 25B provides a graph of T cell subset distribution of iC9-CD19.ζ and iC9-CD19.ζ-MC-modified T cells following long-term culture.

FIGS. 26A and 26B provide schematics comparing a constitutive MC-CAR polypeptide co-expressed with an inducible Casp-9 polypeptide, and an inducible MC polypeptide co-expressed with a first generation CAR polypeptide. FIGS. 26C and 26D provide an outline of an assay and a graph comparing the results of administration of modified T cells expressing the polypeptides of FIGS. 26A and 26B, using the CD19+ Raji tumor model.

### DETAILED DESCRIPTION

Immunotherapy strategies for treating cancer involve enlisting a patient's immune system to attack and kill tumor cells. One type of immunotherapy is adoptive cell transfer in which a subject's immune cells are collected and modified ex vivo to provide for specific and targeted tumor cell killing when the modified cells are returned to the body. A particular adoptive cell transfer method uses CAR-modified T cells and holds great promise for the treatment of a variety of malignancies. In this therapy, T cells are extracted from a patient's blood and genetically engineered to express chimeric antigen receptors (CARs) on the cell surface.

As mentioned above, antitumor activity of CAR-T cells is associated with robust CAR-T cell expansion post-infusion that is often associated with toxicity (i.e., severe cytokine-release syndrome and neurotoxicity), while patients with poor CAR-T proliferation and persistence show reduced rates of durable remissions. In the Examples presented herein, it is demonstrated that signaling from costimulatory molecules, e.g., MyD88 and CD40 (MC), can enhance CAR-T survival, proliferative capacity and antitumor activity. Importantly, also shown in the Examples section, cytokine-related toxicity from these highly active CAR-T cells can be controlled using inducible caspase-9 (iC9) to safely maximize tumor killing.

Without intending to be limited to any theory, the studies described in the Examples show that the toxicity of CAR-T cells that produced high levels of cytokines (i.e., IFN-γ, TNF-α and IL-6) could be resolved with the use of rimiducid. In addition, rimiducid could be titrated to “partially” eliminate CAR-T cells preserving long-term antitumor efficacy. In addition, upon finding that CAR-T secreted cytokines were responsible for cachexia, the selection of CD8⁺ effector T cells resulted in lower levels of toxicity with increased antitumor effects in a CD4⁺ helper-independent manner. The results were consistent across experiments using CAR-T cells with different antigenic targets.

In one aspect the invention described herein relates to compositions and methods for enhancing and maintaining chimeric antigen receptor-expressing T cells, while reducing cytotoxic effects of CAR-T cell therapies.

In some embodiments, the invention provides compositions and methods comprising a CAR-T cell population. In some embodiments, the CAR-T cell population is selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34.

In some embodiments, the invention provides compositions and methods comprising a CAR-T cell population comprising a costimulatory polypeptide. The costimulatory polypeptide can be inducible or constitutively activated. The costimulatory polypeptide can comprise one or more costimulatory signaling regions such as CD27, ICOS, RANK, TRANCE, CD28, 4-1BB, OX40, DAP10, MyD88, or CD40. The costimulatory polypeptide can comprise one or more costimulatory signaling regions that activate the signaling pathways activated by CD27, ICOS, RANK, TRANCE, CD28, 4-1BB, OX40, DAP10, MyD88, or CD40. In some embodiments, the CAR-T cell population comprising the costimulatory polypeptide is selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34.

In some embodiments, the invention provides compositions and methods comprising a CAR-T cell population comprising a costimulatory polypeptide comprising MyD88 and/or CD40, or any suitable cytoplasmic signaling regions that activates the MyD88 and/or CD40 signaling pathways. The costimulatory polypeptide can be inducible or constitutively activated. In some embodiments, the CAR-T cell population comprising the costimulatory polypeptide is selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34.

In some embodiments, the invention provides compositions and methods comprising a CAR-T cell population comprising an inducible pro-apoptotic polypeptide. In some embodiments, the CAR-T cell population comprising the pro-apoptotic polypeptide is selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34.

In some embodiments, the invention provides compositions and methods comprising a CAR-T cell population comprising a costimulatory polypeptide and an inducible pro-apoptotic polypeptide. The costimulatory polypeptide can be inducible or constitutively activated. In some embodiments, the CAR-T cell population comprising the pro-apoptotic polypeptide and the costimulatory polypeptide is selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34.

### T Cells

T cells (also referred to as T lymphocytes) belong to a group of white blood cells referred to as lymphocytes. Lymphocytes generally are involved in cell-mediated immunity. The “T” in “T cells” refers to cells derived from or whose maturation is influenced by the thymus. T cells can be distinguished from other lymphocytes types such as B cells and Natural Killer (NK) cells by the presence of cell surface proteins known as T cell receptors. The term “activated T cells” as used herein, refers to T cells that have been stimulated to produce an immune response (e.g., clonal expansion of activated T cells) by recognition of an antigenic determinant, such as, for example, presented in the context of a Class II major histo-compatibility (MHC) marker. T cells are activated by the presence of an antigenic determinant, cytokines and/or lymphokines and cluster of differentiation cell surface proteins (e.g., CD3, CD4, CD8, the like and combinations thereof). Cells that express a cluster of differential protein often are said to be “positive” for expression of that protein on the surface of T cells (e.g., cells positive for CD3, CD4, or CD8 expression are referred to as CD3⁺, CD4⁺, or CD8⁺). CD3 and CD4 proteins are cell surface receptors or co-receptors that may be directly and/or indirectly involved in signal transduction in T cells.

T cells express receptors on their surfaces (i.e., T cell receptors) that recognize antigens presented on the surface of cells. During a normal immune response, binding of these antigens to the T cell receptor, in the context of MHC antigen presentation, initiates intracellular changes leading to T cell activation. Chimeric antigen receptors (CARs) are artificial receptors designed to convey antigen specificity to T cells without the requirement for MHC antigen presentation. They include an antigen-specific component, a transmembrane component, and an intracellular component selected to activate the T cell and provide specific immunity. Chimeric antigen receptor-expressing T cells may be used in various therapies, including cancer therapies.

By “chimeric antigen receptor” or “CAR” is meant, for example, a chimeric polypeptide that comprises a polypeptide sequence that recognizes a target antigen (an antigen-recognition domain, antigen recognition region, antigen recognition moiety, or antigen binding region) linked to a transmembrane polypeptide and intracellular domain polypeptide selected to activate the T cell and provide specific immunity. An antigen recognition domain may be any polypeptide or fragment thereof, such as, for example, an antibody fragment variable domain, either naturally-derived, or synthetic, which binds to an antigen. Examples of antigen recognition moieties include, but are not limited to, polypeptides derived from antibodies, such as, for example, single chain variable fragments (scFv), Fab, Fab′, F(ab′)2, and Fv fragments; polypeptides derived from T Cell receptors, such as, for example, TCR variable domains; polypeptides derived from Pattern Recognition Receptors, and any ligand or receptor fragment that binds to the extracellular cognate protein.

By “T cell activation molecule” is meant a polypeptide that, when incorporated into a T cell expressing a chimeric antigen receptor, enhances activation of the T cell. Examples include, but are not limited to, ITAM-containing, Signal 1 conferring molecules such as, for example, CD3 polypeptide, and Fc receptor gamma, such as, for example, Fc epsilon receptor gamma (FcεR1γ) subunit (Haynes, N. M., et al. J. Immunol. 166:182-7 (2001)). J. Immunology). The intracellular domain comprises at least one polypeptide which causes activation of the T cell, such as, for example, but not limited to, CD3 zeta.

In some embodiments, the basic components of a chimeric antigen receptor (CAR) include the following. The variable heavy (VH) and light (VL) chains for a tumor-specific monoclonal antibody are fused in-frame with the CD3 ζ chain (ζ) from the T cell receptor complex. The VH and VL are generally connected together using a flexible glycine-serine linker, and then attached to the transmembrane domain by a spacer (e.g., CD8a stalk or CH₂CH₃) to extend the scFv away from the cell surface so that it can interact with tumor antigens.

The term “chimeric antigen receptor” may also refer to chimeric receptors that are not derived from antibodies, but are chimeric T cell receptors. These chimeric T cell receptors may comprise a polypeptide sequence that recognizes a target antigen, where the recognition sequence may be, for example, but not limited to, the recognition sequence derived from a T cell receptor or an scFv. The intracellular domain polypeptides are those that act to activate the T cell. Chimeric T cell receptors are discussed in, for example, Gross, G., and Eshhar, Z., FASEB Journal 6:3370-3378 (1992), and Zhang, Y., et al., PLOS Pathogens 6:1-13 (2010).

In some embodiments, the invention provides compositions and methods comprising a CAR-T cell population. In some embodiments, the CAR-T cell population is selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34.

In some embodiments, the CAR-T cell population include CD4+ and CD8+ T cells. In some embodiments the CAR-T cell population is enriched to comprise at least 20%, 30%, 40%, 50%, 60%, 70%, 80%, 90%, 95, or 99% CD8+ T cells. In some embodiments the CAR-T cell population is enriched to comprise at least 80% CD8+ T cells. In some embodiments the CAR-T cell population is enriched to comprise at least 90% CD8+ T cells. Thus, in some embodiments, there are more genetically-modified CD8+ T cells than genetically-modified CD4+ T cells in the composition i.e. the ratio of CD4+ cells to CD8+ cells is less than 1 e.g. less than 0.9, less than 0.8, less than 0.7, less than 0.6, or less than 0.5.

### Costimulation

In some embodiments, the invention provides compositions and methods comprising a CAR-T cell population comprising a costimulatory polypeptide.

While CARs were first designed with a single signaling domain, for example, CD3ζ, also known as “first generation CARs” (see, e.g., Becker et al. (1989) Cell 58:911-921; Goverman et al. (1990) Cell 60:929-939; Gross et al. (1989) Proc Natl Acad Sci U.S.A. 86:10024-10028; Kuwana et al. (1987) Biochem Biophys Res Commun 149:960-968), clinical trials evaluating the feasibility of CAR immunotherapy showed limited clinical benefit (see, e.g., Till et al. (2012) Blood 119:3040-3050; Pule et al. (2008) Nat Med 14:1264-1270; Jensen et al. (2010) Biol Blood Marrow Transplant 16:1245-1256; Park et al. (2007) Mol Ther 15:825-833). The limited clinical benefit has been primarily attributed to the incomplete activation of T cells following tumor recognition, which leads to limited persistence and expansion of the cells in vivo (see, e.g., Ramos et al. (2011) Expert Opin Biol Ther 11:855-873).

To address this deficiency, CARs have been engineered to include another stimulating domain, often derived from the cytoplasmic portion of T cell costimulating molecules, including CD28, 4-1BB, OX40, ICOS and DAP10 (see, e.g., Carpenito et al. (2009) Proc Natl Acad Sci U.S.A. 106:3360-3365; Finney et al. (1998) J Immunol 161:2791-2797; Hombach et al. J Immunol 167:6123-6131; Maher et al. (2002) Nat Biotechnol 20:70-75; Imai et al. (2004) Leukemia 18:676-684; Wang et al. (2007) Hum Gene Ther 18:712-725; Zhao et al. (2009) J Immunol 183:5563-5574; Milone et al. (2009) Mol Ther 17:1453-1464; Yvon et al. (2009) Clin Cancer Res 15:5852-5860), which allow CAR-T cells to receive appropriate costimulation upon engagement of the target antigen. The most commonly used costimulating molecules include CD28 and 4-1BB, which, following tumor recognition, can initiate a signaling cascade resulting in NF-κB activation, which promotes both T cell proliferation and cell survival. Clinical trials conducted with anti-CD19 CARs having CD28 or 4-1BB signaling domains for the treatment of refractory acute lymphoblastic leukemia (ALL) have demonstrated significant T cell persistence, expansion and serial tumor killing following adoptive transfer (Kalos et al. (2011) Sci Transl Med 3:95ra73; Porter et al. (2011) N Engl J Med 365:725-733; Brentjens et al. (2013) Sci Transl Med 5:177ra38). Third generation CAR-T cells append CD28-modified CARs with additional signaling molecules from tumor necrosis factor (TNF)-family proteins, such as OX40 and 4-1BB (Finney H M, et al. J Immunol 172:104-13, 2004; Guedan S, et al., Blood, 2014).

Some second and third-generation CAR-T cells have been implicated in patient deaths, due to cytokine storm and tumor lysis syndrome caused by highly activated T cells. In one aspect, the invention described herein relates to compositions and methods comprising CAR-T cell comprising costimulatory polypeptides for enhancing and maintaining chimeric antigen receptor-expressing T cells, while reducing cytotoxic effects of CAR-T cell therapies.

The costimulatory polypeptide of the present invention can be inducible or constitutively activated. The costimulatory polypeptide can comprise one or more costimulatory signaling regions such as CD27, ICOS, RANK, TRANCE, CD28, 4-1BB, OX40, DAP10, MyD88, or CD40 or, for example, the cytoplasmic regions thereof. The costimulatory polypeptide can comprise one or more suitable costimulatory signaling regions that activate the signaling pathways activated by CD27, ICOS, RANK, TRANCE, CD28, 4-1BB, OX40, DAP10, MyD88, or CD40. Costimulating polypeptides include any molecule or polypeptide that activates the NF-κB pathway, Akt pathway, and/or p38 pathway of tumor necrosis factor receptor (TNFR) family (i.e., CD40, RANK/TRANCE-R, OX40, 4-1BB) and CD28 family members (CD28, ICOS). More than one costimulating polypeptide or costimulating polypeptide cytoplasmic region may be expressed in the modified T cells discussed herein.

In some embodiments, the CAR-T cell population comprising the costimulatory polypeptide is selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34.

In some embodiments, the CAR-T cell population comprising the costimulatory polypeptide include CD4+ and CD8+ T cells. In some embodiments the CAR-T cell population comprising the costimulatory polypeptide is enriched to comprise at least 20%, 30%, 40%, 50%, 60%, 70%, 80%, 90%, 95, or 99% CD8+ T cells. In some embodiments the CAR-T cell population comprising the costimulatory polypeptide is enriched to comprise at least 80% CD8+ T cells. In some embodiments the CAR-T cell population comprising the costimulatory polypeptide is enriched to comprise at least 90% CD8+ T cells. Thus, in some embodiments, there are more genetically-modified CD8+ T cells than genetically-modified CD4+ T cells in the composition i.e. the ratio of CD4+ cells to CD8+ cells is less than 1 e.g. less than 0.9, less than 0.8, less than 0.7, less than 0.6, or less than 0.5.

### Costimulation Provided by MyD88 and CD40

In some embodiments, the CAR T cell population describe herein comprise a costimulatory polypeptide. The costimulatory polypeptide can comprise one or more costimulatory signaling regions that activate the signaling pathways activated by CD27, ICOS, RANK, TRANCE, CD28, 4-1BB, OX40, DAP10, MyD88, or CD40

One of the principal functions of second generation CARs is the ability to produce IL-2 that supports T cell survival and growth through activation of the nuclear factor of activated T cells (NFAT) transcription factor by CD3ζ (signal 1) and NF-κB (signal 2) by CD28 or 4-1BB.32. Other molecules that similarly activate NF-κB may also be paired with the CD3ζ chain within a CAR molecule. One approach employs a T cell costimulating molecule that was originally developed as an adjuvant for a dendritic cell (DC) vaccine (Narayanan et al. (2011) J Clin Invest 121:1524-1534; Kemnade et al. (2012) Mol Ther 20(7):1462-1471). For full activation or licensing of DCs, Toll-like receptor (TLR) signaling is usually involved. In TLR signaling, the cytoplasmic TLR/IL-1 domains (referred to as TIR domains) of TLRs dimerize which leads to recruitment and association of cytosolic adaptor proteins such as, for example, the myeloid differentiation primary response protein (MyD88; see SEQ ID NO: 35 or SEQ ID NO: 83 for full length amino acid sequence and SEQ ID NO: 36 or SEQ ID NO: 84 for a nucleotide sequence encoding it).

In some embodiments, the CAR T cell population describe herein comprise a costimulatory polypeptide comprising one or more costimulatory signaling regions that activate the signaling pathways activated by MyD88, CD40 and/or MyD88-CD40 fusion chimeric polypeptide.

MyD88 is an universal adaptor molecule for TLRs and a critical signaling component of the innate immune system, triggering an alert for foreign invaders, priming immune cell recruitment and activation. MyD88 is a cytosolic adapter protein that plays a central role in the innate and adaptive immune response. This protein functions as an essential signal transducer in the interleukin-1 and Toll-like receptor signaling pathways. These pathways regulate that activation of numerous proinflammatory genes. The encoded protein consists of an N-terminal death domain and a C-terminal Toll-interleukin1 receptor domain. MyD88 TIR domain is able to heterodimerize with TLRs and homodimerize with other MyD88 proteins. This in turn results in recruitment and activation of IRAK family kinases through interaction of the death domains (DD) at the amino terminus of MyD88 and IRAK kinases which thereby initiates a signaling pathway that leads to activation of JNK, p38 MAPK (mitogen-activated protein kinase) and NF-κB, a transcription factor that induces expression of cytokine- and chemokine-encoding genes (as well as other genes). MyD88 acts acts via IRAK1, IRAK2, IRF7 and TRAF6, leading to NF-kappa-B activation, cytokine secretion and the inflammatory response. It also Activates IRF1 resulting in its rapid migration into the nucleus to mediate an efficient induction of IFN-beta, NOS2/INOS, and IL12A genes. MyD88-mediated signaling in intestinal epithelial cells is crucial for maintenance of gut homeostasis and controls the expression of the antimicrobial lectin REG3G in the small intestine. TLR signaling also upregulates expression of CD40, a member of the tumor necrosis factor receptor (TNFR) family, which interacts with CD40 ligand (CD154 or CD40L) on antigen-primed CD4⁺ T cells.

CD40 is an important part of the adaptive immune response, aiding to activate APCs through engagement with its cognate CD40L, in turn polarizing a stronger CTL response. The CD40/CD154 signaling system is an important component in T cell function and B cell-T cell interactions. CD40 signaling proceeds through formation of CD40 homodimers and interactions with TNFR-associated factors (TRAFs), carried out by recruitment of TRAFs to the cytoplasmic domain of CD40, which leads to T cell activation involving several secondary signals such as the NF-κB, JNK and AKT pathways.

Apart from survival and growth advantages, MyD88 or MyD88-CD40 fusion chimeric polypeptide-based costimulation may also provide additional functions to CAR-modified T cells. MyD88 signaling is critical for both Th1 and Th17 responses and acts via IL-1 to render CD4⁺ T cells refractory to regulatory T cell (Treg)-driven inhibition (see, e.g., Schenten et al. (2014) Immunity 40:78-90). In addition, CD40 signaling in CD8⁺ T cells via Ras, PI3K and protein kinase C, results in NF-κB-dependent induction of cytotoxic mediators granzyme and perforin that lyse CD4⁺ CD25⁺ Treg cells (Martin et al. (2010) J Immunol 184:5510-5518). Thus, MyD88 and CD40 co-activation may render CAR-T cells resistant to the immunosuppressive effects of Treg cells, a function that could be critically important in the treatment of solid tumors and other types of cancers.

MyD88 and CD40 together in immune cells, including T cells, can act downstream on transcription factors to upregulate proinflammtory cytokines, Type I IFNs, and promote proliferation and survival. Along with signaling input from CD3ζ from a CAR, MyD88/CD40 makes for a potent and pleotropic costimulatory molecule. In some embodiments, the invention provides for CAR T cells comprising a costimulatory polypeptide comprising one or more costimulatory signaling regions that activate the signaling pathways activated by MyD88, CD40 and/or MyD88-CD40 fusion chimeric polypeptide. Examples of suitable costimulatory signaling regions include, but are not limited to, IRAK-4, IRAK-1, TRAF6, TRAF2, TRAF3, TRAF5, Act, JAK3, or any functional fragments thereof.

One approach to costimulation of CAR-T cells is to express a fusion protein (referred to as MC) of the signaling elements of MyD88. Survival and growth of such cells can be enhanced through activation of the NFAT transcription factor by CD3ζ, which is part of the chimeric antigen receptor (signal 1), and NF-κB (signal 2) by MyD88 and CD40. The activation of CAR-T cells expressing MC is observed with a cytoplasmic MyD88/CD40 chimeric fusion protein, lacking a membrane targeting region, and with a chimeric fusion protein comprising MyD88/CD40 and a membrane targeting region, such as, for example, a myristoylation region. CAR-T cells may co-express an inducible chimeric signaling polypeptide comprising a multimeric ligand binding region, such as, for example, FKBP12v36, and a MyD88 polypeptide or truncated MyD8 polypeptide, or a MyD88-CD40 or truncated MyD88-CD40 polypeptide (iMC). Cells that express both iMC and a first generation CAR allowed complete T cell activation that required both iMC and tumor recognition through the CAR, resulting in IL-2 production, CD25 receptor upregulation and T cell expansion, and the therapeutic efficacy was controlled by AP1903 in vivo. In some embodiments, the inducible chimeric signaling polypeptide comprises two costimulatory polypeptide cytoplasmic signaling regions, such as, for example, 4-1BB and CD28, or one, or two or more costimulatory polypeptide cytoplasmic signaling regions selected from the group consisting of CD27, ICOS, RANK, TRANCE, CD28, 4-1BB, OX40, DAP10, rather than the MyD88, truncated MyD88, MyD88-CD40, or truncated MyD88-CD40 polypeptides. In some embodiments, CAR-T cells comprise a nucleic acid that encodes a first polynucleotide encoding the inducible chimeric signaling polypeptide and a second polynucleotide encoding the CAR. In some embodiments, the first polynucleotide is positioned 5′ of the second polynucleotide. In some embodiments, the first polynucleotide is positioned 3′ of the second polynucleotide. In some embodiments, a third polynucleotide encoding a linker polypeptide is positioned between the first and second polynucleotides. In some embodiments, the linker polypeptide is a 2A polypeptide, which may separate the polypeptides encoded by the first and second polynucleotides during, or after translation.

By MyD88, or MyD88 polypeptide, is meant the polypeptide product of the myeloid differentiation primary response gene 88, for example, but not limited to the human version, cited as ncbi Gene ID 4615. One example of a MyD88 polypeptide is presented as SEQ ID NO: 83. Another example of a MyD88 polypeptide is presented as SEQ ID NO: 35. By “truncated,” is meant that the protein is not full length and may lack, for example, a domain. For example, a truncated MyD88 is not full length and may, for example, be missing the TIR domain. In some embodiments, the truncated MyD88 polypeptide is encoded by the nucleic acid sequence of SEQ ID NO: 28, and comprises the amino acid sequence of SEQ ID NO: 27. By a nucleic acid sequence coding for “truncated MyD88” is meant the nucleic acid sequence coding for the truncated MyD88 peptide, the term may also refer to the nucleic acid sequence including the portion coding for any amino acids added as an artifact of cloning, including any amino acids coded for by the linkers. It is understood that where a method or construct refers to a truncated MyD88 polypeptide, the method may also be used, or the construct designed to refer to another MyD88 polypeptide, such as a full length MyD88 polypeptide. Where a method or construct refers to a full length MyD88 polypeptide, the method may also be used, or the construct designed to refer to a truncated MyD88 polypeptide. Functionally equivalent” or “a functional fragment” of a MyD88 polypeptide refers, for example, to a truncated MyD88 polypeptide whether lacking the TIR domain or not that is capable of amplifying the cell-mediated tumor killing response when expressed in cells, for example, T cells, NK cells, or NK-T cells, such as, for example, the T cell-mediated, NK cell-mediated, or NK-T cell-mediated response, by, for example, activating the NFκB pathway. Truncated MyD88 polypeptides may, for example, comprise amino acid residues 1-172 of the full length MyD88 amino acid sequence, for example, residues 1-172 of SEQ ID NO: 35 or SEQ ID NO: 83. In some embodiments, Truncated MyD88 polypeptides may, for example, comprise amino acid residues 1-151 or 1-155 of the full length MyD88 amino acid sequence, for example, residues 1-151 or 1-155 of SEQ ID NO: 35 or SEQ ID NO: 83. In some embodiments, truncated MyD88 polypeptides may, for example, comprise amino acid residues 1-152, 153, 154, 155, 156, 157, 158, 159, 160, 161, 162, 163, 164, 165, 166, 167, 168, 169, 170, or 171 of the full length MyD88 amino acid sequence; an example of a full length MyD88 amino acid sequence is provided as SEQ ID NO: 35 or SEQ ID NO: 83. In some embodiments, the truncated MyD88 amino acid sequence does not include contiguous amino acid residues 173-296 of the full length MyD88 amino acid sequence. In some embodiments, the truncated MyD88 amino acid sequence does not include contiguous amino acid residues 152-296 of the full length MyD88 amino acid sequence. In some embodiments, the truncated MyD88 amino acid sequence does not include contiguous amino acid residues 156-296 of the full length MyD88 amino acid sequence. In some embodiments, the truncated MyD88 amino acid sequence does not include contiguous amino acid residues 152, 153, 154, 155, 156, 157, 158, 159, 160, 161, 162, 163, 164, 165, 166, 167, 168, 169, 170, 171, or 172-296 of the full length MyD88 amino acid sequence. By “full length MyD88 amino acid sequence” is meant a full length MyD88 amino acid sequence that corresponds to, for example, SEQ ID NO: 35 or SEQ ID NO: 83. In the embodiments provided herein, a cytoplasmic CD40 polypeptide lacking the extracellular domain, may be located either upstream or downstream from the MyD88 or truncated MyD88 polypeptide portion.

The term “chimeric signaling polypeptide” is interchangeable with “chimeric costimulating molecule,” “chimeric costimulating polypeptide.”

Further, the chimeric costimulating molecule, MyD88/CD40 (MC), in the absence of a multimeric ligand-binding region, provided costimulation of CAR-T cells when provided as part of a bi-cistronic (comprising a polynucleotide encoding the CAR, and a polynucleotide encoding the MC polypeptide), and when provided as part of a tri-cistronic (comprising a polynucleotide encoding the CAR, a polynucleotide encoding the MC polypeptide, and a polynucleotide encoding an inducible chimeric pro-apoptotic polypeptide). This costimulation was detected where the constitutive MC polypeptide was positioned 3′ of the CAR-encoding polynucleotide, for example, 3′ of the portion of the CAR-nucleotide encoding the CD3ζ region; this costimulation was detected in CAR-T cells transfected or transduced with an expression vector comprising, or not comprising, a polynucleotide encoding a 2A sequence between the CD3-encoding polynucleotide sequence and the MC-encoding polynucleotide sequence.

The terms “chimeric,” “fusion” and “chimeric fusion” are used interchangeably herein with reference to a polypeptide containing two or more proteins (or a portion(s) of one or more of the two or more proteins) that have been joined to create a chimeric polypeptide. The two or more proteins (or portions thereof) may be directly joined to each other, wherein a terminal amino acid residue of one protein (or portion thereof) is directly bonded to a terminal amino acid residue of another protein (or portion thereof), or may be joined through one or more intervening elements (e.g., one or more amino acids that are not part of either protein, such as a linker or adapter, or a non-amino acid polymer). For example, a polypeptide that is produced from nucleic acid encoding a fusion of a multimerizing protein (or portion thereof) and another protein (e.g., a DNA-binding protein, transcription activation protein, pro-apoptotic protein or protein component of an immune cell activation pathway), or portion thereof, may be referred to as a chimeric, fusion or chimeric fusion polypeptide.

In some embodiments, the cell populations provided herein comprise CAR-T cells designed to provide constitutively active therapy. In some embodiments, the CAR-T cells comprise a nucleic acid comprising a first polynucleotide encoding the CAR, and a second polynucleotide encoding a chimeric signaling polypeptide. In some embodiments, the second polynucleotide is positioned 5′ of the first polynucleotide. In some embodiments, the second polynucleotide is positioned 3′ of the first polynucleotide. In some embodiments, a third polynucleotide encoding a linker polypeptide is positioned between the first and second polynucleotides. Where the third polynucleotide is positioned 3′ of the first polynucleotide and 5′ of the second polynucleotide, the linker polypeptide, may remain intact following translation, or may separate the polypeptides encoded by the first and second polynucleotides during, or after translation. In some embodiments, the linker polypeptide is a 2A polypeptide, which may separate the polypeptides encoded by the first and second polynucleotides during, or after translation. High level costimulation is provided constitutively through an alternate mechanism in which a leaky 2A cotranslational sequence, for example one derived from porcine teschovirus-1 (P2A), is used to separate the CAR from the chimeric signaling polypeptide. Where the 2A separation is incomplete, for example from a leaky 2A sequence, most of the expressed chimeric signaling polypeptide molecules are separated from the chimeric antigen receptor polypeptide and may remain cytosolic, and some portion or the chimeric signaling polypeptide molecules remain attached, or linked, to the CAR.

By “constitutively active” is meant that the chimeric stimulating molecule's T cell activation activity, as demonstrated herein, is active in the absence of an inducer. Constitutively active chimeric stimulating molecules in the present application do not comprise a multimeric ligand binding region, or a functional multimeric ligand binding region, and are not inducible by AP1903, AP20187, or other CID.

In some embodiments, the chimeric signaling polypeptide comprises a truncated MyD88 polypeptide and a CD40 polypeptide lacking the extracellular domain, or two costimulatory polypeptide cytoplasmic signaling regions. In some embodiments, the chimeric signaling polypeptide comprises two costimulatory polypeptide cytoplasmic signaling regions, such as, for example, 4-1BB and CD28, or one, or two or more costimulatory polypeptide cytoplasmic signaling regions selected from the group consisting of CD27, ICOS, RANK, TRANCE, CD28, 4-1BB, OX40, DAP10. In some embodiments, the chimeric signaling polypeptide comprises a MyD88 polypeptide or a truncated MyD88 polypeptide and a costimulatory polypeptide cytoplasmic signaling region selected from the group consisting of CD27, ICOS, RANK, TRANCE, CD28, 4-1BB, OX40, DAP10.

Also provided in some embodiments, are cell populations provided herein that comprise an inducible safety switch, to stop, or reduce the level of, the therapy when needed. In some embodiments, immune cells, such as CAR-T cells, express a chimeric antigen receptor, and a chimeric signaling polypeptide comprising, for example, a truncated MyD88 polypeptide and a CD40 polypeptide lacking the extracellular domain, or two costimulatory polypeptide cytoplasmic signaling regions

Costimulation in T cells that express chimeric antigen receptors by MyD88 and CD40 polypeptides, and by chimeric signaling polypeptides comprising costimulatory polypeptide cytoplasmic signaling regions is discussed in U.S. patent application Ser. No. 14/842,710, filed Sep. 1, 2015, published as US2016-0058857-A1 on Mar. 3, 2016, entitled “Costimulation of Chimeric Antigen Receptors by MyD88 and CD40 Polypeptides,” and to in U.S. Provisional Patent Application Ser. No. 62/503,565, filed May 9, 2017, entitled “Methods to Augment or Alter Signal Transduction.”

Non-limiting examples of chimeric polypeptides useful for inducing cell activation, and related methods for inducing CAR-T cell activation including, for example, expression constructs, methods for constructing vectors, and assays for activity or function, may also be found in the following patents and patent applications, each of which is incorporated by reference herein in its entirety for all purposes. U.S. patent application Ser. No. 14/210,034, filed Mar. 13, 2014, entitled METHODS FOR CONTROLLING T CELL PROLIFERATION, published Sep. 25, 2014 as US2014-0286987-A1; International Patent Application No. PCT/US2014/026734, filed Mar. 13, 2014, published Sep. 25, 2014 as WO2014/151960, by Spencer et al.; U.S. patent application Ser. No. 14/622,018, filed Feb. 13, 2014, entitled METHODS FOR ACTIVATING T CELLS USING AN INDUCIBLE CHIMERIC POLYPEPTIDE, published Feb. 18, 2016 as US2016-0046700-A1; International Patent Application No. PCT/US2015/015829, filed Feb. 13, 2015, published Aug. 20, 2015 as WO2015/123527; U.S. patent application Ser. No. 10/781,384, filed Feb. 18, 2004, entitled INDUCED ACTIVATION OF DENDRITIC CELLS, published Oct. 21, 2004 as US2004-0209836-A1, issued Jun. 29, 2008 as U.S. Pat. No. 7,404,950, by Spencer et al.; International Patent Application No. PCT/US2004/004757, filed Feb. 18, 2004, published Mar. 24, 2005 as WO2004/073641A3; U.S. patent application Ser. No. 12/445,939, filed Oct. 26, 2010, entitled METHODS AND COMPOSITIONS FOR GENERATING AN IMMUNE RESPONSE BY INDUCING CD40 AND PATTERN RECOGNITION RECEPTORS AND ADAPTORS THEREOF, published Feb. 10, 2011 as US2011-0033388-A1, issued Apr. 8, 2014 as U.S. Pat. No. 8,691,210, by Spencer et al.; International Patent Application No. PCT/US2007/081963, filed Oct. 19, 2007, published Apr. 24, 2008 as WO2008/049113; U.S. patent application Ser. No. 13/763,591, filed Feb. 8, 2013, entitled METHODS AND COMPOSITIONS FOR GENERATING AN IMMUNE RESPONSE BY INDUCING CD40 AND PATTERN RECOGNITION RECEPTOR ADAPTERS, published Mar. 27, 2014 as US2014-0087468-A1, issued Apr. 19, 2016 as U.S. Pat. No. 9,315,559, by Spencer et al.; International Patent Application No. PCT/US2009/057738, filed Sep. 21, 2009, published Mar. 25, 2010 as WO201033949; U.S. patent application Ser. No. 13/087,329, filed Apr. 14, 2011, entitled METHODS FOR TREATING SOLID TUMORS, published Nov. 24, 2011 as US2011-0287038-A1, by Slawin et al.; International Patent Application No. PCT/US2011/032572, filed Apr. 14, 2011, published Oct. 20, 2011 as WO2011/130566, by Slawin et al; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/047957, published as WO2016/036746 on Mar. 10, 2016, entitled COSTIMULATION OF CHIMERIC ANTIGEN RECEPTORS BY MYD88 AND CD40 POLYPEPTIDES; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; International Patent Application No. PCT/US2016/066371, filed Dec. 13, 2016, published Jun. 22, 2017 as WO2017/106185, by Bayle et al.; International Patent Application No. PCT/US2018/031689, filed May 8, 2018, entitled METHODS TO AUGMENT OR ALTER SIGNAL TRANSDUCTION, published Nov. 15, 2018 as WO2018/208849, by Bayle et al., each of which is incorporated by reference herein in its entirety, including all text, tables and drawings, for all purposes.

### Safety Switches

Genetically-modified T cells of the invention may express a safety switch, also known as an inducible suicide gene or suicide switch, which can be used to eradicate the T cells in vivo if desired e.g. if GVHD develops. In some examples, T cells that express a chimeric antigen receptor are provided to the patient that trigger an adverse event, such as off-target toxicity. In some therapeutic instances, a patient might experience a negative symptom during therapy using chimeric antigen receptor-modified cells. In some cases these therapies have led to side effects due, in part, to non-specific attacks on healthy tissue. In some examples, the therapeutic T cells may no longer be needed, or the therapy is intended for a specified amount of time, for example, the therapeutic T cells may work to decrease the tumor cell, or tumor size, and may no longer be needed. Therefore, in some embodiments are provided nucleic acids, cells, and methods wherein the modified T cell also expresses an inducible Caspase-9 polypeptide. If there is a need, for example, to reduce the number of chimeric antigen receptor modified T cells, an inducible ligand may be administered to the patient, thereby inducing apoptosis of the modified T cells.

These switches respond to a trigger, such as a pharmacological agent, which is supplied when it is desired to eradicate the T cells, and which leads to cell death (e.g. by triggering necrosis or apoptosis). These agents can lead to expression of a toxic gene product, but a more rapid response can be obtained if the genetically-modified T cells already express a protein which is switched into a toxic form in response to the agent.

In some embodiments, a safety switch is based on a pro-apoptotic protein that can be triggered by administering a chemical inducer of dimerization to a subject. If the pro-apoptotic protein is fused to a polypeptide sequence which binds to the chemical inducer of dimerization, delivery of this chemical inducer can bring two pro-apoptotic proteins into proximity such that they trigger apoptosis. For instance, Caspase-9 can be fused to a modified human FK-binding protein which can be induced to dimerize in response to the pharmacological agent rimiducid (AP1903). The use of a safety switch based on a human pro-apoptotic protein, such as, for example, Caspase-9 minimizes the risk that cells expressing the switch will be recognized as foreign by a human subject's immune system. Delivery of rimiducid to a subject can therefore trigger apoptosis of T cells which express the caspase-9 switch.

Caspase-9 switches are described in Di Stasi et al. (2011) supra; see also Yagyu et al. (2015) Mol Ther 23(9):1475-85; Rossigloni et al. (2018) Cancer Gene Ther doi.org/10.1038/s41417-018-0034-1; Jones et al. (2014) Front Pharmacol doi.org/10.3389/fphar.2014.00254; U.S. Pat. No. 9,434,935, issued Sep. 16, 2016, entitled Modified Caspase Polypeptides and Uses Thereof; U.S. Pat. No. 9,913,882, issued Mar. 13, 2018, entitled Methods for Inducing Partial Apoptosis Using Caspase Polypeptides; U.S. Pat. No. 9,393,292, issued Jul. 19, 2016, entitled Methods for Inducing Selective Apoptosis; and patent application US2015/0328292, published Nov. 19, 2015, entitled Caspase Polypeptides Having Modified Activity and Uses Thereof. Suicide switches may also be based on Fas or on HSV thymidine kinase.

Examples of ligand inducers fo the switches include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology: From Small Molecules to Systems Biology and Drug Design (Schreiber, s., et al., eds., Wiley, 2007)

The ligand binding regions incorporated in the safety switches may comprise the FKBP12v36 modified FKBP12 polypeptide, or other suitable FKBP12 variant polypeptides, including variant polypeptides that bind to AP1903, or other synthetic homodimerizers such as, for example, AP20187 or AP2015. Variants may include, for example, an FKBP region that has an amino acid substitution at position 36 selected from the group consisting of valine, leucine, isoleuceine and alanine (Clackson T, et al., Proc Natl Acad Sci USA. 1998, 95:10437-10442). AP1903, also known as rimiducid, (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-di methoxyphenyl)propylidene]] ester, [2S-[1(R*),2R*[S*[S*[1(R*),2R*]]]]]-(9CI) CAS Registry Number: 195514-63-7; Molecular Formula: C78H98N4O20 Molecular Weight: 1411.65), is a synthetic molecule that has proven safe in healthy volunteers (Iuliucci J D, et al., J Clin Pharmacol. 2001, 41:870-879).

Provided in some embodiments are safety switches such as, for example, the safety switches discussed in Di Stasi et al. (2011) supra, which consists of the sequence of the human FK506-binding protein (FKBP12) (GenBank AH002 818) with an F36V mutation, connected through a SGGGS linker to a modified human caspase 9 (CASP9) which lacks its endogenous caspase activation and recruitment domain. The F36V mutation increases the binding affinity of FKBP12 to synthetic homodimerizers AP20187 and AP1903 (rimiducid).

The safety switch may comprise a modified Caspase-9 polypeptide having modified activity, such as, for example, reduced basal activity in the absence of the homodimerizer ligand. Modified Caspase-9 polypeptides are discussed in, for example, U.S. Pat. No. 9,913,882 and US-2015-0328292, supra, and may include, for example, amino acid substitutions at position 330 (e.g., D330E or D330!) or, for example, amino acid substitutions at position 450 (e.g., N405Q), or combinations thereof, including, for example, D330E-N405Q and D330A-N405Q.

An effective amount of the pharmaceutical composition, such as the dimerizing or multimerizing ligand presented herein, would be the amount that achieves this selected result of inducing apoptosis in the Caspase-9-expressing cells T cells, such that over 60%, 70%, 80%, 85%, 90%, 95%, or 97%, or that under 80%, 70%, 60%, 50%, 40%, 30%, 20%, or 10% of the therapeutic cells are killed. The term is also synonymous with “sufficient amount.” Any appropriate assay may be used to determine the percent of therapeutic cells that are killed. An assay may include the steps of obtaining a first sample from a subject before administration of the dimerizing or multimerizing ligand, and obtaining a second sample from the subject after administration of the dimerizing or multimerizing ligand, and comparing the number or concentration of therapeutic cells in the first and second samples to determine the percent of therapeutic cells that are killed. One can empirically determine the effective amount of a particular composition presented herein without necessitating undue experimentation.

Non-limiting examples of chimeric polypeptides useful for inducing cell death or apoptosis, and related methods for inducing cell death or apoptosis, including expression constructs, methods for constructing vectors, assays for activity or function, and multimerization of the chimeric polypeptides by contacting cells that express inducible chimeric polypeptides with a multimeric compound, or a pharmaceutically acceptable salt thereof, that binds to the multimerizing region of the chimeric polypeptides both ex vivo and in vivo, administration of expression vectors, cells, or multimeric compounds described herein, or pharmaceutically acceptable salts thereof, to subjects, and administration of multimeric compounds described herein, or pharmaceutically acceptable salts thereof, to subjects who have been administered cells that express the inducible chimeric polypeptides, may also be found in the following patents and patent applications, each of which is incorporated by reference herein in its entirety for all purposes. U.S. patent application Ser. No. 13/112,739, filed May 20, 2011, entitled METHODS FOR INDUCING SELECTIVE APOPTOSIS, published Nov. 24, 2011, as US2011-0286980-A1, issued Jul. 28, 2015 as U.S. Pat. No. 9,089,520; U.S. patent application Ser. No. 13/792,135, filed Mar. 10, 2013, entitled MODIFIED CASPASE POLYPEPTIDES AND USES THEREOF, published Sep. 11, 2014 as US2014-0255360-A1, issued Sep. 6, 2016 as U.S. Pat. No. 9,434,935, by Spencer et al.; International Patent Application No. PCT/US2014/022004, filed Mar. 7, 2014, published Oct. 9, 2014 as WO2014/16438; U.S. patent application Ser. No. 14/296,404, filed Jun. 4, 2014, entitled METHODS FOR INDUCING PARTIAL APOPTOSIS USING CASPASE POLYPEPTIDES, published Jun. 2, 2016 as US2016-0151465-A1, by Slawin et al; International Application No. PCT/US2014/040964 filed Jun. 4, 2014, published as WO2014/197638 on Feb. 5, 2015, by Slawin et al.; U.S. patent application Ser. No. 14/640,553, filed Mar. 6, 2015, entitled CASPASE POLYPEPTIDES HAVING MODIFIED ACTIVITY AND USES THEREOF, published Nov. 19, 2015 as US2015-0328292-A1; International Patent Application No. PCT/US2015/019186, filed Mar. 6, 2015, published Sep. 11, 2015 as WO2015/134877, by Spencer et al.; U.S. patent application Ser. No. 14/968,737, filed Dec. 14, 2015, entitled METHODS FOR CONTROLLED ELIMINATION OF THERAPEUTIC CELLS, published Jun. 16, 2016 as US2016-0166613-A1, by Spencer et al.; International Patent Application No. PCT/US2015/065629 filed Dec. 14, 2015, published Jun. 23, 2016 as WO2016/100236, by Spencer et al.; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; and International Patent Application No. PCT/US2016/066371, filed Dec. 13, 2016, published Jun. 22, 2017 as WO2017/106185, by Bayle et al., each of which is incorporated by reference herein in its entirety, including all text, tables and drawings, for all purposes. Multimeric compounds described herein, or pharmaceutically acceptable salts thereof, may be used essentially as discussed in examples provided in these publications, and other examples provided herein.

As used herein, the term “pharmaceutically or pharmacologically acceptable” refers to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells presented herein, its use in therapeutic compositions is contemplated. Supplementary active ingredients also can be incorporated into the compositions. In some embodiments, the subject is a mammal.

By “kill” or “killing” as in a percent of cells killed, is meant the death of a cell through apoptosis, as measured using any method known for measuring apoptosis. The term may also refer to cell ablation.

### Enriched T Cell Populations

In some embodiments, enriched cell populations are provided, where the enriched cell population has been selected to comprise specified ratios or percentages of one or more cell type. By “cell population” or “modified cell population” is meant a group of cells, such as more than two cells. The cell population may be homogenous, comprising the same type of cell, or each comprising the same marker, or it may be heterogeneous. In some examples, the cell population is derived from a sample obtained from a subject and comprises cells prepared from, for example, bone marrow, umbilical cord blood, peripheral blood, or any tissue. In some examples, the cell population has been contacted with a nucleic acid, wherein the nucleic acid comprises a heterologous polynucleotide, such as, for example, a polynucleotide that encodes a chimeric antigen receptor, an inducible chimeric pro-apoptotic polypeptide, or a costimulatory polypeptide, such as, for example, a chimeric MyD88 or truncated MyD88 and CD40 polypeptide. The terms cell population and modified cell population also refer to progeny of the original cells that have been contacted with the nucleic acid that comprises the heterologous polynucleotide. A cell population may be selected, or enriched, or purified, to comprise at least 20%, 30%, 40%, 50%, 60%, 70%, 80%, 90%, 95, or 99%, for example, of a cell type that expresses a certain marker, receptor, or cell surface glycoprotein, such as, for example, CD8, CD4, CD3, CD34. Without intending to be limited to any theory, in some embodiments, enriching the T cell populations to obtain increased ratios of CD8⁺ to CD4⁺ T cells may reduce the level of CAR-T cell associated cytokine-release syndrome and neurotoxicity.

The efficacy of chimeric antigen receptor-modified T cells (CAR-T) is dependent on their in vivo expansion following adoptive transfer. Additional genetic augmentations to improve CAR-T expansion may improve therapeutic efficacy but risk increasing CAR-T toxicity. CAR-T cells, CAR-T cells that express costimulating polypeptides, and CAR-T cells that express MyD88, or MyD88-CD40 chimeric proteins either constitutively or under the control of an inducible multimerizing region, are effective at eliminating tumors but may induce acute cytokine-related toxicity. The potential for cytotoxicity may reduce the dosage of CAR-T cells that may be administered to a subject. The Examples section shows that the toxicity may be avoided or reduced by enriching the CAR-T cells prior to administration, to provide a modified cell population with an increased concentration of CD8⁺ T cells.

The T cells can be derived from any healthy donor. The donor will generally be an adult (at least 18 years old) but children are also suitable as T cell donors (e.g. see Styczynski 2018, Transfus Apher Sci 57(3):323-330). An example of a suitable process for obtaining T cells from a donor is described in Di Stasi et al. (2011) N Engl J Med 365:1673-83. In general terms, T cells are obtained from a donor, subjected to genetic modification and selection, and can then be administered to recipient subjects. A useful source of T cells is the donor's peripheral blood. Peripheral blood samples will generally be subjected to leukapheresis to provide a sample enriched for white blood cells. This enriched sample (also known as a leukopak) can be composed of a variety of blood cells including monocytes, lymphocytes, platelets, plasma, and red cells. A leukopak typically contains a higher concentration of cells as compared to venipuncture or buffy coat products.

### CD8+ Enriched T Cell Populations

The selection, enrichment, or purification of a cell type in the modified cell population may be achieved by any suitable method. In some embodiments, the proportions of CD8⁺ and CD4⁺ T cells may be determined by flow cytometry. In some examples, a MACs column may be used. In some examples, the modified cell population is frozen and defrosted before administration to the subject, and the viable cells are tested for the percentage or ratio of a certain cell type before administration to the subject. T cells were separated into purified CD4⁺ and CD8⁺ T cells by magnetic selection (MACS columns), following transduction or transfection.

The composition may include CD4+ and CD8+ T cells, and ideally the population of genetically-modified CD3+ T cells within the composition includes CD4+ cells and CD8+ cells. Whereas the ratio of CD4+ cells to CD8+ cells in a leukopak is typically above 2, in some embodiments the ratio of genetically-modified CD4+ cells to genetically-modified CD8+ cells in a composition of the invention is less than 2 e.g. less than 1.5. In some embodiments, there are more genetically-modified CD8+ T cells than genetically-modified CD4+ T cells in the composition i.e. the ratio of CD4+ cells to CD8+ cells is less than 1 e.g. less than 0.9, less than 0.8, less than 0.7, less than 0.6, or less than 0.5. Thus, the overall procedure starting from donor cells and producing genetically-modified T cells is designed to enrich for CD8+ cells T cells relative to CD4+ T cells. In some embodiments, 60% or more of the genetically-modified T cells are CD8+ T cells, and in some embodiments, 65% or more of the genetically-modified T cells are CD8+ T cells. Within the population of genetically-modified CD3⁺ T cells, in some embodiments, the percent of CD8+ T cells is between 55-75%, for example, from 63-73%, from 60-70%, or from 65-71%. In some embodiments, a cell population is provided that is selected, or enriched, or purified, to comprise a ratio of one cell type to another, such as, for example, a ratio of CD8⁺ to CD4⁺ T cells of, for example, 3:2, 7:3, 4:1, 9:1, 19:1, or 39:1 or more. In some embodiments, the modified cell population is selected, or enriched, or purified, to comprise at least 20%, 30%, 40%, 50%, 60%, 70%, 75%, 80%, 85%, 90%, 95, 96, 97, 98, or 99%, CD8⁺ T cells. In some embodiments, the ratio of CD8⁺ to CD4⁺ T cells is 4 to 1, or 9:1 or greater.

In some embodiments, for a population of genetically-modified CD3+ T cells comprising a costimulatory polypeptide as described herein, the percent of CD8+ T cells is between 55-75%, for example, from 63-73%, from 60-70%, or from 65-71%. In some embodiments, the ratio of CD8⁺ to CD4⁺ T cells is 3:2, 7:3, 4:1, 9:1, 19:1, or 39:1 or more. In some embodiments, the modified cell population is selected, or enriched, or purified, to comprise at least 20%, 30%, 40%, 50%, 60%, 70%, 75%, 80%, 85%, 90%, 95, 96, 97, 98, or 99%, CD8⁺ T cells. In some embodiments, the ratio of CD8+ to CD4+ T cells is 4 to 1, or 9:1 or greater. The costimulatory polypeptide can comprise one or more costimulatory signaling regions such as CD27, ICOS, RANK, TRANCE, CD28, 4-1BB, OX40, DAP10, MyD88, or CD40. The costimulatory polypeptide can comprise one or more costimulatory signaling regions that activate the signaling pathways activated by CD27, ICOS, RANK, TRANCE, CD28, 4-1BB, OX40, DAP10, MyD88, or CD40.

In some embodiments, the invention provides compositions and methods comprising a CAR-T cell population comprising a costimulatory polypeptide comprising MyD88 and/or CD40, or any suitable cytoplasmic signaling regions that activates the MyD88 and/or CD40 signaling pathways where at least 80%, 85%, 90%, 95, 96, 97, 98, or 99%, CD8⁺ T cells. The costimulatory polypeptide can be inducible or constitutively activated. In some embodiments, the modified cell population is at least 80% CD8⁺ T cells. In some embodiments, the modified cell population is at least 90% CD8⁺ T cells.

In some embodiments, the invention provides compositions and methods comprising a CAR-T cell population comprising an inducible pro-apoptotic polypeptide where at least 80%, 85%, 90%, 95, 96, 97, 98, or 99%, CD8⁺ T cells. In some embodiments, the modified cell population is at least 80% CD8⁺ T cells. In some embodiments, the modified cell population is at least 90% CD8⁺ T cells.

In some embodiments, the invention provides compositions and methods comprising a CAR-T cell population comprising a costimulatory polypeptide and an inducible pro-apoptotic polypeptide where at least 80%, 85%, 90%, 95, 96, 97, 98, or 99%, CD8⁺ T cells. In some embodiments, the modified cell population is at least 80% CD8⁺ T cells. In some embodiments, the modified cell population is at least 90% CD8⁺ T cells. The costimulatory polypeptide can be inducible or constitutively activated. In some embodiments the costimulatory polypeptide comprises MyD88 and/or CD40, or any suitable cytoplasmic signaling regions that activates the MyD88 and/or CD40 signaling pathways.

### Engineering Expression Constructs

Expression constructs that express the present chimeric antigen receptors, chimeric signaling polypeptides, and inducible safety switches are provided herein.

As used herein, the term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

As used herein, the term “polypeptide” is defined as a chain of amino acid residues, usually having a defined sequence. As used herein the term polypeptide may be interchangeable with the term “proteins”.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct. As used herein with reference to a disease, disorder or condition, the terms “treatment”, “treat”, “treated”, or “treating” refer to prophylaxis and/or therapy.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra.

In certain examples, a polynucleotide coding for the chimeric antigen receptor, is included in the same vector, such as, for example, a viral or plasmid vector, as a polynucleotide coding for a second polypeptide. This second polypeptide may be, for example, a chimeric signaling polypeptide, an inducible caspase polypeptide, as discussed herein, or a marker polypeptide. In these examples, the construct may be designed with one promoter operably linked to a nucleic acid comprising a polynucleotide coding for the two polypeptides, linked by a 2A polypeptide. In this example, the first and second polypeptides are separated during translation, resulting in two polypeptides, or, in examples including a leaky 2A, either one, or two polypeptides. In other examples, the two polypeptides may be expressed separately from the same vector, where each nucleic acid comprising a polynucleotide coding for one of the polypeptides is operably linked to a separate promoter. In yet other examples, one promoter may be operably linked to the two polynucleotides, directing the production of two separate RNA transcripts, and thus two polypeptides; in one example, the promoter may be bi-directional, and the coding regions may be in opposite directions 5′-3′. Therefore, the expression constructs discussed herein may comprise at least one, or at least two promoters.

In some embodiments, a nucleic acid construct is contained within a viral vector. In certain embodiments, the viral vector is a retroviral vector. In certain embodiments, the viral vector is an adenoviral vector or a lentiviral vector. It is understood that in some embodiments, a cell is contacted with the viral vector ex vivo, and in some embodiments, the cell is contacted with the viral vector in vivo. Thus, an expression construct may be inserted into a vector, for example a viral vector or plasmid. The steps of the methods provided may be performed using any suitable method; these methods include, without limitation, methods of transducing, transforming, or otherwise providing nucleic acid to the cell, described herein.

As used herein, the term “gene” is defined as a functional protein-, polypeptide-, or peptide-encoding unit. As will be understood, this functional term includes genomic sequences, cDNA sequences, and smaller engineered gene segments that express, or are adapted to express, proteins, polypeptides, domains, peptides, fusion proteins and/or mutants.

As used herein, the term “polynucleotide” is defined as a chain of nucleotides. Furthermore, nucleic acids are polymers of nucleotides. Thus, nucleic acids and polynucleotides as used herein are interchangeable. Nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

“Function-conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Amino acids other than those indicated as conserved may differ in a protein or enzyme so that the percent protein or amino acid sequence similarity between any two proteins of similar function may vary and can be, for example, at least 70%, at least 80%, at least 90%, and at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the settings may be selected that result in the highest sequence similarity.

As used herein, the term “promoter” is defined as a DNA sequence recognized by the synthetic machinery of the cell, or introduced synthetic machinery, required to initiate the specific transcription of a gene. In some embodiments, the promoter is a developmentally regulated promoter. As used herein, the term “under transcriptional control,” “operably linked,” or “operatively linked” is defined as the promoter is in the correct location and orientation in relation to the nucleic acid to control RNA polymerase initiation and expression of the gene. In some examples, one or more polypeptides are said to be “operatively linked.” In general, the term “operably linked” is meant to indicate that the promoter sequence is functionally linked to a second sequence, wherein the promoter sequence initiates and mediates transcription of the DNA corresponding to the second sequence.

The particular promoter employed to control the expression of a polynucleotide sequence of interest is not believed to be important, so long as it is capable of directing the expression of the polynucleotide in the targeted cell. Thus, where a human cell is targeted the polynucleotide sequence-coding region may, for example, be placed adjacent to and under the control of a promoter that is capable of being expressed in a human cell. Generally speaking, such a promoter might include either a human or viral promoter. Promoters may be selected that are appropriate for the vector used to express the CARs and other polypeptides provided herein.

In various embodiments, where, for example, the expression vector is a retrovirus, an example of an appropriate promoter is the Murine Moloney leukemia virus promoter. In other embodiments, the promoter may be, for example, may be the(CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Promoters, and other regulatory elements, are selected such that they are functional in the desired cells or tissue. In addition, this list of promoters should not be construed to be exhaustive or limiting; other promoters that are used in conjunction with the promoters and methods disclosed herein.

The nucleic acids discussed herein may comprise one or more polynucleotides. In some embodiments, one or more polynucleotides may be described as being positioned, or “is” “5′” or or “3′” of another polynucleotide, or positioned in “5′ to 3′ order”. The reference 5′ to 3′ in these contexts is understood to refer to the direction of the coding regions of the polynucleotides in the nucleic acid, for example, where a first polynucleotide is positioned 5′ of a second polynucleotide and connected with a third polynucleotide encoding a non-cleave able linker polypeptide, the translation product would result in the polypeptide encoded by the first polynucleotide positioned at the amino terminal end of a larger polypeptide comprising the translation products of the first, third, and second polynucleotides.

In yet other examples, two polypeptides, such as, for example, the chimeric stimulating molecule or a MyD88/CD40 chimeric antigen receptor polypeptide, and a second polypeptide, may be expressed in a cell using two separate vectors. The cells may be co-transfected or co-transformed with the vectors, or the vectors may be introduced to the cells at different times.

The polypeptides may vary in their order, from the amino terminus to the carboxy terminus. For example, in the chimeric stimulating molecule, the order of the MyD88 polypeptide, CD40 polypeptide, and any additional polypeptide, may vary. In the chimeric antigen receptor, the order of the MyD88 polypeptide, CD40 polypeptide, and any additional polypeptide, such as, for example, the CD3 ζ polypeptide may vary. The order of the various domains may be assayed using methods such as, for example, those discussed herein, to obtain the optimal expression and activity.

In some embodiments, where an expression construct encodes a MyD88 polypeptide, the polypeptide may be a portion of the full-length MyD88 polypeptide. By MyD88, or MyD88 polypeptide, is meant the polypeptide product of the myeloid differentiation primary response gene 88, for example, but not limited to the human version, cited as NCBI Gene ID 4615. In some embodiments, an expression construct encodes a portion of the MyD88 polypeptide lacking the TIR domain. In some embodiments, the expression construct encodes a portion of the MyD88 polypeptide containing the DD (death domain) or the DD and intermediary domains. By “truncated,” is meant that the protein is not full length and may lack, for example, a domain. For example, a truncated MyD88 is not full length and may, for example, be missing the TIR domain. In some embodiments, the truncated MyD88 polypeptide has an amino acid sequence of SEQ ID NO: 27, or a functionally equivalent fragment thereof. In some embodiments, the truncated MyD88 polypeptide is encoded by the nucleotide sequences of SEQ ID NO: 28, or a functionally equivalent fragment thereof. A functionally equivalent portion of the MyD88 polypeptide has substantially the same ability to stimulate intracellular signaling as the polypeptide of SEQ ID NO: 27, with at least 50%, 60%, 70%, 80%, 90%, or 95% of the activity of the polypeptide of SEQ ID NO: 27. In some embodiments, the expression construct encodes a portion of a MyD88 polypeptide lacking the TIR domain such as the polypeptide encoded by the MyD88 polypeptide-encoding nucleotide sequence of pM006, pM007, or pM009. By a nucleic acid sequence coding for “truncated MyD88” is meant the nucleic acid sequence coding for a truncated MyD88 polypeptide, the term may also refer to the nucleic acid sequence including the portion coding for any amino acids added as an artifact of cloning, including any amino acids coded for by the linkers.

It is understood that where a method or construct refers to a truncated MyD88 polypeptide, the method may also be used, or the construct designed to refer to another MyD88 polypeptide, such as a full length MyD88 polypeptide. Where a method or construct refers to a full length MyD88 polypeptide, the method may also be used, or the construct designed to refer to a truncated MyD88 polypeptide. In the methods herein, in which a chimeric polypeptide comprises a MyD88 polypeptide (or portion thereof) and a CD40 polypeptide (or portion thereof), the MyD88 polypeptide of the chimeric polypeptide may be located either upstream or downstream from the CD40 polypeptide. In certain embodiments, the MyD88 polypeptide (or portion thereof) is located upstream of the CD40 polypeptide (or portion thereof). As used herein, the term “functionally equivalent,” as it relates to MyD88, or a portion thereof, for example, refers to a MyD88 polypeptide that stimulates a cell-signaling response or a nucleic acid encoding such a MyD88 polypeptide. “Functionally equivalent” refers, for example, to a MyD88 polypeptide that is lacking a TIR domain but is capable of stimulating a cell-signaling response.

In certain embodiments, a modified cell populations comprise a nucleic acid molecule that comprises a promoter operably linked to a first polynucleotide encoding a chimeric stimulating molecule, wherein the chimeric stimulating molecule comprises (i) a MyD88 polypeptide or a truncated MyD88 polypeptide lacking the TIR domain; and (ii) a CD40 cytoplasmic polypeptide region lacking the CD40 extracellular domain, and wherein the chimeric stimulating molecule does not include a membrane targeting region; and

b) a second polynucleotide encoding a T cell receptor, a T cell receptor-based chimeric antigen receptor, or a chimeric antigen receptor; and
c) a third polynucleotide encoding a chimeric Caspase-9 polypeptide comprising a multimeric ligand binding region and a Caspase-9 polypeptide. It is understood that the order of the polynucleotides may vary, and may be tested to determine the suitability of the construct for any particular method, thus, the nucleic acid may include the polynucleotides in the varying orders, which also take into account a variation in the order of the MyD88 polypeptide or truncated MyD88 polypeptide-encoding sequence and the CD40 cytoplasmic polypeptide region-encoding sequence in the first polynucleotide. Thus, the first polynucleotide may encode a polypeptide having and order of MyD88/CD40, truncatedMyD88/CD40, CD40/MyD88, or CD40/truncated MyD88. And, the nucleic acid may include the first through third polynucleotides in any of the following orders, where 1, 2, 3, indicate a first, second, or third order of the polynucleotides in the nucleic acid from the 5′ to 3′ direction. It is understood that other polynucleotides, such as those that code for a 2A polypeptide, for example, may be present between the three listed polynucleotides; this Table is meant to designate the order of the first through third polynucleotides:

TABLE 1

| First polynucleotide | | |
|---|---|---|
| encoding a Chimeric | Second polynucleotide | Third |
| stimulating molecule | encoding a T cell | polynucleotide |
| comprising MyD88 or | receptor, a T cell | encoding a |
| truncated MyD88 and | receptor-based chimeric | chimeric |
| CD40 cytoplasmic | antigen receptor, or | caspse-9 |
| polypeptide region. | a chimeric antigen receptor. | polypeptide. |
| 1 | 2 | 3 |
| 1 | 3 | 2 |
| 2 | 1 | 3 |
| 3 | 1 | 2 |
| 2 | 3 | 1 |
| 3 | 2 | 1 |

Similarly, the nucleic acids may include only two of the polynucleotides, coding for two of the polypeptides provided in the table above. In some examples, a cell is transfected or transduced with a nucleic acid comprising the three polynucleotides included in Table 1 above. In other examples, a cell is transfected or transduced with a nucleic acid that encodes two of the polynucleotides, coding for two of the polypeptides, as provided, for example, in Table 2.

TABLE 2

| First polynucleotide | | |
|---|---|---|
| encoding a Chimeric | Second polynucleotide | Third |
| stimulating molecule | encoding a T cell | polynucleotide |
| comprising MyD88 or | receptor, a T cell | encoding a |
| truncated MyD88 and | receptor-based chimeric | chimeric |
| CD40 cytoplasmic | antigen receptor, or | caspse-9 |
| polypeptide region. | a chimeric antigen receptor. | polypeptide. |
| 1 | 2 | |
| 1 | | 2 |
| 2 | 1 | |
| | 1 | 2 |
| 2 | | 1 |
| | 2 | 1 |

In some embodiments, the cell is transfected or transduced with the nucleic acid that encodes two of the polynucleotides, and the cell also comprises a nucleic acid comprising a polynucleotide coding for the third polypeptide. For example, a cell may comprise a nucleic acid comprising the first and second polynucleotides, and the cell may also comprise a nucleic acid comprising a polynucleotide coding for a chimeric Caspase-9 polypeptide. Also, a cell may comprise a nucleic acid comprising the first and third polynucleotides, and the cell may also comprise a nucleic acid comprising a polynucleotide coding for a T cell receptor, a T cell receptor-based chimeric antigen receptor, or a chimeric antigen receptor.

The steps of the methods provided may be performed using any suitable method; these methods include, without limitation, methods of transducing, transforming, or otherwise providing nucleic acid to the cell, presented herein. In some embodiments, the truncated MyD88 peptide is encoded by the nucleotide sequence of SEQ ID NO: 28 (with or without DNA linkers or has the amino acid sequence of SEQ ID NO: 27). In some embodiments, the CD40 cytoplasmic polypeptide region is encoded by a polynucleotide sequence in SEQ ID NO: 30.

Vectors

It is understood that the vectors provided herein may be modified using methods known in the art to vary the position or order of the regions, to substitute one region for another. For example, a vector comprising a polynucleotide encoding a chimeric signaling polypeptide comprising truncated MC may be substituted with a polynucleotide encoding chimeric signaling polypeptide comprising one, or two or more co-stimulatory polypeptide cytoplasmic signaling regions such as, for example, those selected from the group consisting of CD27, CD28, 4-1BB, OX40, ICOS, RANK, TRANCE, and DAP10. The polynucleotide encoding the CAR may also be modified so that the scFv region may be substituted with one having the same, or different target specificity; the transmembrane region may be substituted with a different transmembrane region; a stalk polypeptide may be added. Polynucleotides encoding marker polypeptides may be included within or separate from one of the polypeptides; polynucleotides encoding additional polypeptides coding for safety switches may be added, polynucleotides coding for linker polypeptides, or non-coding polynucleotides or spacers may be added, or the order of the polynucleotides 5′ to 3′ may be changed.

The vectors provided in the present application may be modified as discussed herein, for example, to substitute polynucleotides coding for regions of the chimeric antigen receptor, for example, the CD19-specific scFV, or other scFvs provided, with a scFv directed against other target antigens, such as, for example, CD33, NKG2D, PSMA, PSCA, MUC1, CD19, ROR1, Mesothelin, GD2, CD123, MUC16, Her2/Neu, CD20, CD30, PRAME, NY-ESO-1, and EGFRvIII. The vector may also be modified with appropriate substitutions of each polypeptide region, as discussed herein. The vector may be modified to remove the inducible caspase-9 safety switch (1), to position the inducible caspase-9 safety switch to a position 3′ of the MyD88-CD40 polypeptide (**), to substitute the inducible caspase-9 safety switch with a different inducible caspase polypeptide-based switch, or to substitute the inducible caspase-9 safety switch with a different polypeptide safety switch.

The vectors provided herein may be modified to substitute the MyD88-CD40 (MC) portions with one, or two or more co-stimulatory polypeptide cytoplasmic signaling regions such as, for example, those selected from the group consisting of CD27, CD28, 4-1BB, OX40, ICOS, RANK, TRANCE, and DAP10. Co-stimulating polypeptides may comprise, but are not limited to, the amino acid sequences provided herein, and may include functional conservative mutations, including deletions or truncations, and may comprise amino acid sequences that are 70%, 75%, 80%, 85%, 90%, 95% or 100% identical to the amino acid sequences provided herein.

The vectors provided herein may be modified to substitute a polynucleotide coding for a linker sequence, where the linker polypeptide is not a 2A polypeptide, between the CAR polypeptide and the MC polypeptide or other co-stimulatory polypeptide. For example, nucleic acids provided herein may comprise, a polynucleotide coding for a MC polypeptide, or a co-stimulatory polypeptide signaling region 3′ of a polynucleotide coding for the CD3ζ portion of the CAR, where the two polynucleotides are separated by a polynucleotide coding for a 2A linker, or, where the two polynucleotides are not separated by a polynucleotide coding for a 2A linker. In some embodiments, the two polynucleotides may be separated by a polynucleotide coding for a linker polypeptide having, for example, about 5 to 20 amino acids, or, for example, about 6 to 10 amino acids, where the linker polypeptide does not comprise a 2A polypeptide sequence.

Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as Herpes Simplex Virus thymidine kinase (tk) are employed. Immunologic surface markers containing the extracellular, non-signaling domains or various proteins (e.g. CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as GFP, EGFP, β-gal or chloramphenicol acetyltransferase (CAT). In certain embodiments, the marker protein, such as, for example, CD19 is used for selection of the cells for transfusion, such as, for example, in immunomagnetic selection. As discussed herein, a CD19 marker is distinguished from an anti-CD19 antibody, or, for example, a scFv, TCR, or other antigen recognition moiety that binds to CD19.

In certain embodiments, the marker polypeptide is linked to the inducible chimeric stimulating molecule. For example, the marker polypeptide may be linked to the inducible chimeric stimulating molecule via a polypeptide sequence, such as, for example, a cleavable 2A-like sequence.

The CAR-T cells provided herein may express a cell surface transgene marker, present on an expression vector that expresses the CAR, or, in some embodiments, present on an expression vector that encodes a protein other than the CAR, such as, for example a pro-apoptotic polypeptide safety switch, such as i-Casp9, that is co-expressed with the CAR.

In one embodiment, the cell surface transgene marker is a truncated CD19 (ΔCD19) polypeptide (Di Stasi et al. (2011) supra, that comprises a human CD19 truncated at amino acid 333 to remove most of the intracytoplasmic domain. The extracellular CD19 domain can still be recognised (e.g. in flow cytometry, FACS or MACS) but the potential to trigger intracellular signalling is minimised. CD19 is normally expressed by B cells, rather than by T cells, so selection of CD19+ T cells permits the genetically-modified T cells to be separated from unmodified donor T cells.

In some embodiments, a polypeptide may be included in the polypeptide, for example, the CAR encoded by the expression vector to aid in sorting cells. In some embodiments, the expression vectors used to express the chimeric antigen receptors or chimeric stimulating molecules provided herein further comprise a polynucleotide that encodes the 16 amino acid CD34 minimal epitope. In some embodiments, such as certain embodiments provided in the examples herein, the CD34 minimal epitope is incorporated at the amino terminal position of the CD8 stalk.

Linker Polypeptides

Linker polypeptides include, for example, cleavable and non-cleavable linker polypeptides. Non-cleavable polypeptides may include, for example, any polypeptide that may be operably linked between the MyD88-CD40 chimeric polypeptide, the MyD88 polypeptide, the CD40 polypeptide, or the costimulatory polypeptide cytoplasmic signaling region and the CD3ζ portion of the chimeric antigen receptor. Linker polypeptides include those for example, consisting of about 2 to about 30 amino acids, (e.g., furin cleavage site, (GGGGS)ₙ). In some embodiments, the linker polypeptide consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the linker polypeptide consists of about 18 to 22 amino acids. In some embodiments, the linker polypeptide consists of 20 amino acids. In some embodiments, cleavable linkers include linkers that are cleaved by an enzyme exogenous to the modified cells in the population, for example, an enzyme encoded by a polynucleotide that is introduced into the cells by transfection or transduction, either at the same time or a different time as the polynucleotide that encodes the linker. In some embodiments, cleavable linkers include linkers that are cleaved by an enzyme endogenous to the modified cells in the population, including, for example, enzymes that are naturally expressed in the cell, and enzymes encoded by polynucleotides native to the cell, such as, for example, lysozyme.

2A Peptide Bond-Skipping Sequences

2A-like sequences, or “peptide bond-skipping” 2A sequences, are derived from, for example, many different viruses, including, for example, from Thosea asigna. These sequences are sometimes also known as “peptide skipping sequences.” When this type of sequence is placed within a cistron, between two polypeptides that are intended to be separated, the ribosome appears to skip a peptide bond, in the case of Thosea asigna sequence; the bond between the Gly and Pro amino acids at the carboxy terminal “P-G-P” is omitted. This may, leave two to three polypeptides, for example, an inducible chimeric pro-apoptotic polypeptide and a chimeric antigen receptor, or, for example, a marker polypeptide and an inducible chimeric pro-apoptotic polypeptide. When this sequence is used, the polypeptide that is encoded 5′ of the 2A sequence may end up with additional amino acids at the carboxy terminus, including the Gly residue and any upstream residues in the 2A sequence. The peptide that is encoded 3′ of the 2A sequence may end up with additional amino acids at the amino terminus, including the Pro residue and any downstream residues following the 2A sequence. In some embodiments, the cleavable linker is a 2A polypeptide derived from porcine teschovirus-1 (P2A). In some embodiments, the 2A cotranslational sequence is a 2A-like sequence. In some embodiments, the 2A cotranslational sequence is T2A (thosea asigna virus 2A), F2A (foot and mouth disease virus 2A), P2A (porcine teschovirus-1 2A), BmCPV 2A (cytoplasmic polyhedrosis virus 2A) BmIFV 2A (flacherie virus of B. mori 2A), or E2A (equine rhinitis A virus 2A). In some embodiments, the 2A cotranslational sequence is T2A-GSG, F2A-GSG, P2A-GSG, or E2A-GSG. In some embodiments, the 2A cotranslational sequence is selected from the group consisting of T2A, P2A and F2A. By “cleavable linker” is meant that the linker is cleaved by any means, including, for example, non-enzymatic means, such as peptide skipping, or enzymatic means. (Donnelly, M L 2001, J. Gen. Virol. 82:1013-25).

The 2A-like sequences are sometimes “leaky” in that some of the polypeptides are not separated during translation, and instead, remain as one long polypeptide following translation. One theory as to the cause of the leaky linker, is that the short 2A sequence occasionally may not fold into the required structure that promotes ribosome skipping (a “2A fold”). In these instances, ribosomes may not miss the proline peptide bond, which then results in a fusion protein. To reduce the level of leakiness, and thus reduce the number of fusion proteins that form, a GSG (or similar) linker may be added to the amino terminal side of the 2A polypeptide; the GSG linker blocks secondary structures of newly-translated polypeptides from spontaneously folding and disrupting the ‘2A fold’.

In certain embodiments, a 2A linker includes the amino acid sequence of SEQ ID NO: 25. In certain embodiments, the 2A linker further includes a GSG amino acid sequence at the amino terminus of the polypeptide, in other embodiments, the 2A linker includes a GSGPR amino acid sequence at the amino terminus of the polypeptide. Thus, by a “2A” sequence, the term may refer to a 2A sequence in an example described herein or may also refer to a 2A sequence as listed herein further comprising a GSG or GSGPR sequence at the amino terminus of the linker.

In some embodiments, the linker, for example, the 2A linker, is cleaved in about 10, 20, 30, 40, 50, 60, 70, 75, 80, 85, 90, 95, 98, or 99% of the chimeric antigen receptors, that is, the chimeric antigen receptor portion is separated from the chimeric MyD88 and CD40, the MyD88 polypeptide, the CD40 polypeptide, or the costimulatory polypeptide cytoplasmic signaling region, such as, CD28, OX40, 4-1BB or the like. In other embodiments the 2A linker is cleaved in about 75, 80, 85, 90, 95, 98, or 99% of the chimeric antigen receptors. In some embodiments, the 2A linker is cleaved in about 80-99% of the chimeric antigen receptors. In some embodiments, the 2A linker is cleaved in about 90% of the chimeric antigen receptors. In some embodiments, a constitutive active chimeric antigen receptor polypeptide is present in the modified cells, where the 2A linker is not cleaved, that is, the chimeric antigen receptor portion is linked to the chimeric MyD88 and CD40, the MyD88 polypeptide, the CD40 polypeptide, or the costimulatory polypeptide cytoplasmic signaling region, such as, CD28, OX40, 4-1BB or the like, representing about 1, 2, 5, 10, 15, 20, 25, 30, 40, 50, 60, 70, 80, or 90% of the chimeric antigen receptor polypeptide. In other embodiments the 2A linker is not cleaved in about 5, 10, 15, 20, or 25% of the chimeric antigen receptors. In some embodiments, the 2A linker is not cleaved in about 5-20% of the chimeric antigen receptors. In some embodiments, the 2A linker is not cleaved in about 10% of the chimeric antigen receptors.

Membrane-Targeting

A membrane-targeting sequence provides for transport of the chimeric protein to the cell surface membrane, where the same or other sequences can encode binding of the chimeric protein to the cell surface membrane. Molecules in association with cell membranes contain certain regions that facilitate the membrane association, and such regions can be incorporated into a chimeric protein molecule to generate membrane-targeted molecules. For example, some proteins contain sequences at the N-terminus or C-terminus that are acylated, and these acyl moieties facilitate membrane association. Such sequences are recognized by acyltransferases and often conform to a particular sequence motif. Certain acylation motifs are capable of being modified with a single acyl moiety (often followed by several positively charged residues (e.g. human c-Src: M-G-S-N-K-S-K-P-K-D-A-S-Q-R-R-R) to improve association with anionic lipid head groups) and others are capable of being modified with multiple acyl moieties. For example the N-terminal sequence of the protein tyrosine kinase Src can comprise a single myristoyl moiety. Dual acylation regions are located within the N-terminal regions of certain protein kinases, such as a subset of Src family members (e.g., Yes, Fyn, Lck) and G-protein alpha subunits. Such dual acylation regions often are located within the first eighteen amino acids of such proteins, and conform to the sequence motif Met-Gly-Cys-Xaa-Cys, where the Met is cleaved, the Gly is N-acylated and one of the Cys residues is S-acylated. The Gly often is myristoylated and a Cys can be palmitoylated. Acylation regions conforming to the sequence motif Cys-Ala-Ala-Xaa (so called “CAAX boxes”), which can modified with C15 or C10 isoprenyl moieties, from the C-terminus of G-protein gamma subunits and other proteins (e.g., World Wide Web address ebi.ac.uk/interpro/DisplaylproEntry?ac=IPR001230) also can be utilized. These and other acylation motifs include, for example, those discussed in Gauthier-Campbell et al., Molecular Biology of the Cell 15: 2205-2217 (2004); Glabati et al., Biochem. J. 303: 697-700 (1994) and Zlakine et al., J. Cell Science 110: 673-679 (1997), and can be incorporated in chimeric molecules to induce membrane localization. In some embodiments, a chimeric polypeptide comprising a costimulatory polypeptide cytoplasmic signaling region provided herein comprises a membrane-targeting region, and optionally, a multimeric ligand binding region, in some embodiments, chimeric MyD88, chimeric truncated MyD88, chimeric MyD88-CD40, or chimeric truncated MyD88-CD40, polypeptides provided herein, comprise a membrane-targeting region, and optionally, a multimeric ligand binding region. In some embodiments, the membrane-targeting region comprises a myristoylation region. In some embodiments, the membrane-targeting region is selected from the group consisting of myristoylation-targeting sequence, palmitoylation-targeting sequence, prenylation sequences (i.e., farnesylation, geranyl-geranylation, CAAX Box), protein-protein interaction motifs or transmembrane sequences (utilizing signal peptides) from receptors. Examples include those discussed in, for example, ten Klooster J P et al, Biology of the Cell (2007) 99, 1-12, Vincent, S., et al., Nature Biotechnology 21:936-40, 1098 (2003).

Where a polypeptide does not include a membrane-targeting region, or lacks a membrane-targeting region, such as certain chimeric polypeptides provided herein, the polypeptide does not include a region that provides for transport of the chimeric protein to a cell membrane. The polypeptide may, for example, not include a sequence that transports the polypeptide to the cell surface membrane, or the polypeptide may, for example, include a dysfunctional membrane-targeting region, that does not transport the polypeptide to the cell surface membrane, for example, a myristoylation region that includes a proline that disrupts the function of the myristoylation-targeting region. (see, for example, Resh, M. D., Biochim. Biophys. Acta. 1451:1-16 (1999)). Polypeptides that are not transported to the membrane are considered to be cytoplasmic polypeptides.

### Chimeric Antigen Receptors

Antigen Recognition Moieties

An “antigen recognition moiety” may be any polypeptide or fragment thereof, such as, for example, an antibody fragment variable domain, either naturally derived, or synthetic, which binds to an antigen. Examples of antigen recognition moieties include, but are not limited to, polypeptides derived from antibodies, such as, for example, single chain variable fragments (scFv), Fab, Fab′, F(ab′)₂, and Fv fragments; polypeptides derived from T Cell receptors, such as, for example, TCR variable domains; secreted factors (e.g., cytokines, growth factors) that can be artificially fused to signaling domains (e.g., “zytokines”), and any ligand or receptor fragment (e.g., CD27, NKG2D) that binds to the extracellular cognate protein. Combinatorial libraries could also be used to identify peptides binding with high affinity to tumor-associated targets. Moreover, “universal” CARs can be made by fusing aviden to the signaling domains in combination with biotinylated tumor-targeting antibodies (Urbanska (12) Ca Res) or by using Fc gamma receptor/CD16 to bind to IgG-targeted tumors (Kudo K (13) Ca Res).

Transmembrane Regions

A chimeric protein herein may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFβ, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

In some embodiments, the transmembrane domain is fused to the extracellular domain of the CAR. In one embodiment, the transmembrane domain that naturally is associated with one of the domains in the CAR is used. In other embodiments, a transmembrane domain that is not naturally associated with one of the domains in the CAR is used. In some instances, the transmembrane domain can be selected or modified by amino acid substitution (e.g., typically charged to a hydrophobic residue) to avoid binding of such domains to the transmembrane domains of the same or different surface membrane proteins to minimize interactions with other members of the receptor complex.

Transmembrane domains may, for example, be derived from the alpha, beta, or zeta chain of the T cell receptor, CD3-ε, CD3 ζ, CD4, CD5, CD8, CD8α, CD9, CD16, CD22, CD28, CD33, CD38, CD64, CD80, CD86, CD134, CD137, or CD154. Or, in some examples, the transmembrane domain may be synthesized de novo, comprising mostly hydrophobic residues, such as, for example, leucine and valine. In certain embodiments a short polypeptide linker may form the linkage between the transmembrane domain and the intracellular domain of the chimeric antigen receptor. The chimeric antigen receptors may further comprise a stalk, that is, an extracellular region of amino acids between the extracellular domain and the transmembrane domain. For example, the stalk may be a sequence of amino acids naturally associated with the selected transmembrane domain. In some embodiments, the chimeric antigen receptor comprises a CD8 transmembrane domain, in certain embodiments, the chimeric antigen receptor comprises a CD8 transmembrane domain, and additional amino acids on the extracellular portion of the transmembrane domain, in certain embodiments, the chimeric antigen receptor comprises a CD8 transmembrane domain and a CD8 stalk. The chimeric antigen receptor may further comprise a region of amino acids between the transmembrane domain and the cytoplasmic domain, which are naturally associated with the polypeptide from which the transmembrane domain is derived.

Target Antigens

Chimeric antigen receptors bind to target antigens. When assaying T cell activation in vitro or ex vivo, target antigens may be obtained or isolated from various sources. The target antigen, as used herein, is an antigen or immunological epitope on the antigen, which is crucial in immune recognition and ultimate elimination or control of the disease-causing agent or disease state in a mammal. The immune recognition may be cellular and/or humoral. In the case of intracellular pathogens and cancer, immune recognition may, for example, be a T lymphocyte response.

The target antigen may be derived or isolated from, for example, a pathogenic microorganism such as viruses including HIV, (Korber et al, eds HIV Molecular Immunology Database, Los Alamos National Laboratory, Los Alamos, N. Mex. 1977) influenza, Herpes simplex, human papilloma virus (U.S. Pat. No. 5,719,054), Hepatitis B (U.S. Pat. No. 5,780,036), Hepatitis C (U.S. Pat. No. 5,709,995), EBV, Cytomegalovirus (CMV) and the like. Target antigen may be derived or isolated from pathogenic bacteria such as, for example, from Chlamydia (U.S. Pat. No. 5,869,608), Mycobacteria, Legionella, Meningiococcus, Group A Streptococcus, Salmonella, Listeria, Hemophilus influenzae (U.S. Pat. No. 5,955,596) and the like). Target antigen may be derived or isolated from, for example, pathogenic yeast including Aspergillus, invasive Candida (U.S. Pat. No. 5,645,992), Nocardia, Histoplasmosis, Cryptosporidia and the like. Target antigen may be derived or isolated from, for example, a pathogenic protozoan and pathogenic parasites including but not limited to Pneumocystis carinii, Trypanosoma, Leishmania (U.S. Pat. No. 5,965,242), Plasmodium (U.S. Pat. No. 5,589,343) and Toxoplasma gondii.

The term “antigen” as used herein is defined as a molecule that provokes an immune response. This immune response may involve either antibody production, or the activation of specific immunologically competent cells, or both. An antigen can be derived from organisms, subunits of proteins/antigens, killed or inactivated whole cells or lysates. Therefore, any macromolecules, including virtually all proteins or peptides, can serve as antigens. Furthermore, antigens can be derived from recombinant or genomic DNA, including, for example, any DNA that contains nucleotide sequences or partial nucleotide sequences of a pathogenic genome or a gene or a fragment of a gene for a protein that elicits an immune response results in synthesis of an antigen.

Target antigen includes an antigen associated with a preneoplastic or hyperplastic state. Target antigen may also be associated with, or causative of cancer. Such target antigen may be, for example, tumor specific antigen, tumor associated antigen (TAA) or tissue specific antigen, epitope thereof, and epitope agonist thereof. Such target antigens include but are not limited to carcinoembryonic antigen (CEA) and epitopes thereof such as CAP-1, CAP-1-6D and the like (GenBank Accession No. M29540), MART-1 (Kawakarni et al, J. Exp. Med. 180:347-352, 1994), MAGE-1 (U.S. Pat. No. 5,750,395), MAGE-3, GAGE (U.S. Pat. No. 5,648,226), GP-100 (Kawakami et al Proc. Nat'l Acad. Sci. USA 91:6458-6462, 1992), MUC-1, MUC-2, point mutated ras oncogene, normal and point mutated p53 oncogenes (Hollstein et al Nucleic Acids Res. 22:3551-3555, 1994), PSMA (Israeli et al Cancer Res. 53:227-230, 1993), tyrosinase (Kwon et al PNAS 84:7473-7477, 1987) TRP-1 (gp75) (Cohen et al Nucleic Acid Res. 18:2807-2808, 1990; U.S. Pat. No. 5,840,839), NY-ESO-1 (Chen et al PNAS 94: 1914-1918, 1997), TRP-2 (Jackson et al EMBOJ, 11:527-535, 1992), TAG72, KSA, CA-125, CD-123, PSA, HER-2/neu/c-erb/B2, (U.S. Pat. No. 5,550,214), BRC-I, BRC-II, bcr-abl, pax3-fkhr, ews-fli-1, modifications of TAAs and tissue specific antigen, splice variants of TAAs, epitope agonists, and the like. Other TAAs may be identified, isolated and cloned by methods known in the art such as those disclosed in U.S. Pat. No. 4,514,506. Target antigen may also include one or more growth factors and splice variants of each. A tumor antigen is any antigen such as, for example, a peptide or polypeptide, that triggers an immune response in a host against a tumor. The tumor antigen may be a tumor-associated antigen, which is associated with a neoplastic tumor cell.

### Methods of Gene Transfer/Genetic Modification of T Cells

In order to mediate the effect of the transgene expression in a cell, it will be necessary to transfer the expression constructs into a cell. Such transfer may employ viral or non-viral methods of gene transfer. This section provides a discussion of methods and compositions of gene transfer.

A transformed cell comprising an expression vector is generated by introducing into the cell the expression vector. Suitable methods for polynucleotide delivery for transformation of an organelle, a cell, a tissue or an organism for use with the current methods include virtually any method by which a polynucleotide (e.g., DNA) can be introduced into an organelle, a cell, a tissue or an organism.

The terms “cell,” “cell line,” and “cell culture” as used herein may be used interchangeably. All of these terms also include their progeny, which are any and all subsequent generations. It is understood that all progeny may not be identical due to deliberate or inadvertent mutations. As used herein, the term “ex vivo” refers to “outside” the body. The terms “ex vivo” and “in vitro” can be used interchangeably herein.

The term “transfection” and “transduction” are interchangeable and refer to the process by which an exogenous nucleic acid sequence is introduced into a eukaryotic host cell. Transfection (or transduction) can be achieved by any one of a number of means including electroporation, microinjection, gene gun delivery, retroviral infection, lipofection, superfection and the like.

Any appropriate method may be used to transfect or transform the cells, for example, the T cells, or to administer the nucleotide sequences or compositions of the present methods. Certain non-limiting examples are presented herein. In some embodiments, the virsl vector is an SFG-based viral vector, as discussed in Tey et al. (2007) Biol Blood Marrow Transpl 13:913-24 and by Di Stasi et al. (2011) N Engl J Med 365:1673-83 (2011).

T cells that are genetically modified as disclosed herein are useful for administering to subjects who can benefit from donor lymphocyte administration. These subjects will typically be humans, so the invention will typically be performed using human T cells.

The modified cells may be obtained from a donor, or may be cells obtained from the patient, for example, the cells may be autologous, syngeneic, or allogeneic. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. By “therapeutic cell” is meant a cell used for cell therapy, that is, a cell administered to a subject to treat or prevent a condition or disease.

By “obtained or prepared” as, for example, in the case of cells, is meant that the cells or cell culture are isolated, purified, or partially purified from the source, where the source may be, for example, umbilical cord blood, bone marrow, or peripheral blood. The terms may also apply to the case where the original source, or a cell culture, has been cultured and the cells have replicated, and where the progeny cells are now derived from the original source.

Peripheral blood: The term “peripheral blood” as used herein, refers to cellular components of blood (e.g., red blood cells, white blood cells and platelets), which are obtained or prepared from the circulating pool of blood and not sequestered within the lymphatic system, spleen, liver or bone marrow.

Umbilical cord blood: Umbilical cord blood is distinct from peripheral blood and blood sequestered within the lymphatic system, spleen, liver or bone marrow. The terms “umbilical cord blood”, “umbilical blood” or “cord blood”, which can be used interchangeably, refers to blood that remains in the placenta and in the attached umbilical cord after child birth. Cord blood often contains stem cells including hematopoietic cells.

The term “allogeneic” as used herein, refers to HLA or MHC loci that are antigenically distinct between the host and donor cells. Thus, cells or tissue transferred from the same species can be antigenically distinct. Syngeneic mice can differ at one or more loci (congenics) and allogeneic mice can have the same background. The term “autologous” means a cell, nucleic acid, protein, polypeptide, or the like derived from the same individual to which it is later administered. The modified cells of the present methods may, for example, be autologous cells, such as, for example, autologous T cells.

Donor T cells are generally cultured (usually under activating conditions e.g. using anti-CD3 and/or anti-CD28 antibodies, optionally with IL-2) prior to being genetically modified. This step provides higher yields of T cells at the end of the modification process.

The sample may be subjected to allodepletion in some embodiments, or may not be subjected to allodepletion. In examples provided herein, the samples are not subject to allodepletion, and are thus alloreplete, as discussed in Zhou et al. (2015) Blood 125:4103-13. These populations provide a more robust T cell repertoire for providing the therapeutic advantages of the donor cells.

The T cells can be transduced using a viral vector encoding polynucleotides of the present application. Suitable transduction techniques may involve fibronectin fragment CH-296. As an alternative to transduction using a viral vector, cells can be transfected with any suitable method known in the art such as with DNA encoding the suicide switch of interest and a cell surface transgene marker of interest e.g. using calcium phosphate, cationic polymers (such as PEI), magnetic beads, electroporation and commercial lipid-based reagents such as Lipofectamine™ and Fugene™. One result of the transduction/transfection step is that various donor T cells will now be genetically-modified T cells which can express the suicide switch of interest.

In some embodiments, the viral vector used for transduction is the retroviral vector disclosed by Tey et al. (2007) Biol Blood Marrow Transpl 13:913-24 and by Di Stasi et al. (2011) supra. This vector is based on Gibbon ape leukemia virus (Gal-V) pseudotyped retrovirus encoding an iCasp9 suicide switch and a ΔCD19 cell surface transgene marker (see further below). It can be produced in the PG13 packaging cell line, as discussed by Tey et al. (2007) supra. Other viral vectors encoding the desired proteins can also be used. In some embodiments, retroviral vectors that can provide a high copy number of proviral integrants per cell are used for transduction.

After transduction/transfection, cells can be separated from transduction/transfection materials and cultured again, to permit the genetically-modified T cells to expand. T cells can be expanded so that a desired minimum number of genetically-modified T cells is achieved.

Genetically-modified T cells can then be selected from the population of cells which has been obtained. The suicide switch will usually not be suitable for positive selection of desired T cells, so in some embodiments, the genetically-modified T cells should express a cell surface transgene marker of interest. Cells which express this surface marker can be selected e.g. using immunomagnetic techniques. For instance, paramagnetic beads conjugated to monoclonal antibodies which recognise the cell surface transgene marker of interest can be used, for example, using a CliniMACS system (available from Miltenyi Biotec).

In an alternative procedure, genetically-modified T cells are selected after a step of transduction, are cultured, and are then fed. Thus the order of transduction, feeding, and selection can be varied.

The result of these procedures is a composition containing donor T cells which have been genetically modified and which can thus express, e.g. the costimulatory polypeptide and/or the suicide switch of interest (and, typically, the cell surface transgene marker of interest). These genetically-modified T cells can be administered to a recipient, but they will usually be cryopreserved (optionally after further expansion) before being administered.

### Methods of Treatment

The term “terms “patient” or “subject” are interchangeable, and, as used herein include, but are not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate. The subject may be, for example, human, for example, a patient suffering from an infectious disease, and/or a subject that is immunocompromised, or is suffering from a hyperproliferative disease.

Modified cell populations provided herein may be used in methods for treating human subjects in need thereof, and may be used to prepare medicaments for treating such subjects. The cells will usually be delivered to the recipient subject by infusion. A typical dose of T cells for the subject is between 10⁵-10⁷ cells/kg. Pediatric patients will generally receive a dose of around 10⁶ cells/kg, whereas adult patients will receive a higher dose e.g. 3×10⁶ cells/kg.

The recipient may undergo myeloablative conditioning prior to receiving the modified cell population comprising genetically-modified T cells. Thus the recipient's own α/β T cells (and B cells) can be depleted prior to receiving the genetically-modified T cells. Similarly, haematopoietic (stem) cells which are administered to a recipient may be depleted for α/β cells. In contrast, genetically-modified donor T cells administered to the recipient are generally not depleted for α/β cells.

The recipient can be a child e.g. a child aged from 0-16 years old, or from 0-10 years old. In some embodiments, the recipient is an adult.

Subjects receiving the genetically-modified T cells may also receive other tissue from an allogeneic donor e.g. they can receive haematopoietic cells and/or haematopoietic stem cells (e.g. CD34⁺ cells). This allograft tissue and the genetically-modified T cells are ideally derived from the same donor, such that they will be genetically matched. In some embodiments, the donor and the recipient are a matched unrelated donor, or a suitable family member. For instance, the donor may be the recipient's parent or child. Where a subject is identified as being in need of genetically-modified T cells, therefore, a suitable donor can be identified as a T cell donor.

Where modified cell populations provided herein, for example, modified cell populations comprising modified T cells, are used in conjunction with haematopoietic cells and/or haematopoietic stem cells, the modified cell populations may, in some examples, be administered at a later timepoint e.g. between 20-100 days later.

If the recipient develops complications after receiving the genetically-modified T cells (e.g. they develop GVHD) then the suicide switch can be triggered e.g. by administering rimiducid to the recipient. The minimum dose of the inducible ligand (e.g., rimiducid) required to eliminate the modified cells, where the modified cells comprise an inducible chimeric pro-apoptotic polypeptide, will depend on the number of genetically-modified T cells which are present in the recipient. Doses above this minimum can be administered but, in accordance with normal pharmaceutical principles, excessive dosing should be avoided. In some embodiments, the suicide switch can be triggered with rimiducid, e.g., a dose of 0.4 mg/kg can eliminate cells which were infused at a dose of 1.5×10⁷ cells/kg. In general terms, a rimiducid dose between 0.1-5 mg/kg is administered, and usually 0.1-2 mg/kg or 0.1-1 mg/kg will suffice, and, in some embodiments, the dose is 0.4 mg/kg. A series of multiple doses of rimiducid can be administered e.g. if it is found that a first dose does not eliminate all genetically-modified T cells then a second dose can be administered, etc.

In some embodiments, a first dose of the inducing ligand (e.g. rimiducid) is administered which kills the most sensitive cells, and then a second dose (which is higher than the first dose) is administered which kills cells which are less sensitive. Further doses (escalating where necessary) can be administered if required.

The present methods also encompass methods of treatment or prevention of a disease caused by pathogenic microorganisms and/or a hyperproliferative disease.

Diseases that may be treated or prevented include diseases caused by viruses, bacteria, yeast, parasites, protozoa, cancer cells and the like. The pharmaceutical composition (transduced T cells, expression vector, expression construct, etc.) may be used as a generalized immune enhancer (T cell activating composition or system) and as such has utility in treating diseases. Exemplary diseases that can be treated and/or prevented include, but are not limited, to infections of viral etiology such as HIV, influenza, Herpes, viral hepatitis, Epstein Bar, polio, viral encephalitis, measles, chicken pox, Papilloma virus etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

Preneoplastic or hyperplastic states which may be treated or prevented using the pharmaceutical composition (transduced T cells, expression vector, expression construct, etc.) include but are not limited to preneoplastic or hyperplastic states such as colon polyps, Crohn's disease, ulcerative colitis, breast lesions and the like.

Cancers, including solid tumors, which may be treated using the pharmaceutical composition include, but are not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemias, uterine cancer, breast cancer, prostate cancer, ovarian cancer, pancreatic cancer, colon cancer, multiple myeloma, neuroblastoma, NPC, bladder cancer, cervical cancer and the like.

Solid tumors from any tissue or organ may be treated using the present methods, including, for example, for example, solid tumors present in, for example, lungs, bone, liver, prostate, or brain, and also, for example, in breast, ovary, bowel, testes, colon, pancreas, kidney, bladder, neuroendocrine system, soft tissue, boney mass, and lymphatic system. Other solid tumors that may be treated include, for example, glioblastoma, and malignant myeloma.

The recipient may have a hematological cancer (such as a treatment-refractory hematological cancer) or an inherited blood disorder. For instance, the recipient may have acute lymphoblastic leukemia (ALL), acute myeloid leukemia (AML), severe combined immune-deficiency (SCID), Wiskott-Aldrich syndrome (WA), Fanconi Anemia, chronic myelogenous leukemia (CML), non-Hodgkin lymphoma (NHL), Hodgkin lymphoma (HL), or multiple myeloma.

The term “cancer” as used herein is defined as a hyperproliferation of cells whose unique trait—loss of normal controls—results in unregulated growth, lack of differentiation, local tissue invasion, and metastasis. Examples include but are not limited to, melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

The term “hyperproliferative disease” is defined as a disease that results from a hyperproliferation of cells. Other hyperproliferative diseases, including solid tumors, that may be treated using the T cell and other therapeutic cell activation system presented herein include, but are not limited to rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

As used herein, the terms “treatment”, “treat”, “treated”, or “treating” refer to prophylaxis and/or therapy. When used with respect to a solid tumor, such as a cancerous solid tumor, for example, the term refers to prevention by prophylactic treatment, which increases the subject's resistance to solid tumors or cancer. In some examples, the subject may be treated to prevent cancer, where the cancer is familial, or is genetically associated. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, for example, reduce or eliminate the infection or prevent it from becoming worse.

The methods provided herein may be used, for example, to treat a disease, disorder, or condition wherein there is an elevated expression of a tumor antigen.

The administration of the pharmaceutical composition (expression construct, expression vector, fused protein, transduced cells, and activated T cells, transduced and loaded T cells) may be for either “prophylactic” or “therapeutic” purpose. When provided prophylactically, the pharmaceutical composition is provided in advance of any symptom. The prophylactic administration of modified cell populations serves to prevent or ameliorate any subsequent infection or disease. When provided therapeutically, the modified cell population is provided at or after the onset of a symptom of infection or disease. Thus the compositions presented herein may be provided either prior to the anticipated exposure to a disease-causing agent or disease state or after the initiation of the infection or disease. Thus provided herein are methods for prophylactic treatment of solid tumors such as those found in cancer, or for example, but not limited to, prostate cancer, using the modified cell populations discussed herein. For example, methods are provided of prophylactically preventing or reducing the size of a tumor in a subject comprising administering a the modified cell populations discussed herein, whereby the modified cell population is administered in an amount effect to prevent or reduce the size of a tumor in a subject.

An effective amount of the pharmaceutical composition would be the amount that achieves this selected result of enhancing the immune response, and such an amount could be determined. For example, an effective amount of for treating an immune system deficiency could be that amount necessary to cause activation of the immune system, resulting in the development of an antigen specific immune response upon exposure to antigen. The term is also synonymous with “sufficient amount.” In other examples, an effective amount could be that amount necessary for reducing tumor size or the number of tumors, or for reducing the growth rate of tumors, or the rate of proliferation of tumors. In other examples, an effective amount could be that amount necessary for reducing the amount or concentration of target antigen in a subject, measured by comparing the amount or concentration of target antigen in samples obtained before, during, and/or after administration of the modified cell populations provided herein.

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation. Thus, for example, in one embodiment, the transduced T cells or other cells are administered to a subject in an amount effective to, for example, induce an immune response, or, for example, to reduce the size of a tumor or reduce the amount of tumor vasculature.

In some embodiments, multiple doses of modified cells are administered to the subject, with an escalation of dosage levels among the multiple doses. In some embodiments, the escalation of dosage levels increases the level of CAR-T cell activity, and therefore increases the therapeutic effect, such as, for example, the reduction in the amount or concentration of target cells, such as, for example, tumor cells.

In some embodiments, personalized treatment is provided wherein the stage or level of the disease or condition is determined before administration of the modified cells, before the administration of an additional dose of the modified cells, or in determining method and dosage involved in the administration of the modified cells. These methods may be used in any of the methods of the present application. Where these methods of assessing the patient before administering the modified cells are discussed in the context of, for example, the treatment of a subject with a solid tumor, it is understood that these methods may be similarly applied to the treatment of other conditions and diseases. Thus, for example, in some embodiments of the present application, the method comprises administering the modified cells of the present application to a subject, and further comprises determining the appropriate dose of modified cells to achieve the effective level of reduction of tumor size. The amount of cells may be determined, for example, based on the subject's clinical condition, weight, and/or gender or other relevant physical characteristic. By controlling the amount of modified cells administered to the subject, the likelihood of adverse events such as, for example, a cytokine storm may be reduced.

The term “dosage” is meant to include both the amount of the dose and the frequency of administration, such as, for example, the timing of the next dose. The term “dosage level” refers to the amount of the modified cell population administered in relation to the body weight of the subject.

In some examples, the term dosage may refer to the dosage of the ligand inducer. For example, to induce the chimeric Caspase-9 polypeptide, the term “dosage level” refers to the amount of the multimeric ligand administered in relation to the body weight of the subject. Thus increasing the dosage level would mean increasing the amount of the ligand administered relative to the subject's weight. In addition, increasing the concentration of the dose administered, such as, for example, when the multimeric ligand is administered using a continuous infusion pump would mean that the concentration administered (and thus the amount administered) per minute, or second, is increased.

Methods as presented herein include without limitation the delivery of an effective amount of a modified cell population, a nucleic acid, or an expression construct encoding the same. An “effective amount” of the modified cell population, nucleic acid, or expression construct, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers, or other disease symptoms such as tumor size or tumor antigen expression, to evaluate the effectiveness of treatment and to control toxicity.

If needed, the method may further include additional leukaphereses to obtain more cells to be used in treatment.

### Optimized and Personalized Therapeutic Treatment

The dosage and administration schedule of the modified cells may be optimized by determining the level of the disease or condition to be treated. For example, the size of any remaining solid tumor, or the level of targeted cells such as, for example, tumor cells or CD19-expressing B cells, which remain in the patient, may be determined.

In some examples, about 1×10⁴, 5×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸ 4×10⁸ 5×10⁸, 6×10⁸, 7×10⁸, 8×10⁸, 9×10⁸, or 1×10⁹ modified cells, or cells from the modified cell population, per kg subject body weight are administered to the subject. In some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells. Thus, in some embodiments, about 1×10⁴, 5×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸ 4×10⁸ 5×10⁸, 6×10⁸, 7×10⁸, 8×10⁸, 9×10⁸, or 1×10⁹ modified cells, or cells from the modified cell population, per kg subject body weight are administered to the subject, where the modified cell population comprise at 60%, 70%, 75%, 80%, 85%, 90%, 95, 96, 97, 98, or 99%, CD8⁺ T cells. In some embodiments, the ratio of CD8⁺ to CD4⁺ T cells is 3:2, 4 to 1, or 9:1 or greater.

For example, determining that a patient has clinically relevant levels of tumor cells, or a solid tumor, after initial therapy, provides an indication to a clinician that it may be necessary to administer the modified cell population. In another example, determining that a patient has a reduced level of tumor cells or reduced tumor size after treatment with the modified cell population may indicate to the clinician that no additional dose of the modified cells is needed. Similarly, after treatment with the modified cells, determining that the patient continues to exhibit disease or condition symptoms, or suffers a relapse of symptoms may indicate to the clinician that it may be necessary to administer at least one additional dose of modified cells.

Thus, for example, in certain embodiments, the methods comprise determining the presence or absence of a tumor size increase and/or increase in the number of tumor cells in a subject relative to the tumor size and/or the number of tumor cells following administration of a first, or a previous dose of modified cells, and administering an additional dose of the modified cells acid to the subject in the event the presence of a tumor size increase and/or increase in the number of tumor cells is determined. The methods also comprise, for example, determining the presence or absence of an increase in a non-solid tumor cell, such as, for example, CD19-expressing B cells in the subject relative to the level of CD19-expressing B cells following a first, or a previous administration of the modified cell population, and administering an additional dose of the modified cells to the subject in the event the presence of an increase in CD19-expressing B cells in the subject is determined. In these embodiments, for example, the patient is initially treated with the therapeutic cells according to the methods provided herein. Following the initial treatment, the size of the tumor, the number of tumor cells, or the number of CD19-expressing B cells, for example, may decrease relative to the time prior to the initial treatment. At a certain time after this initial treatment, the patient is again tested, or the patient may be continually monitored for disease symptoms. If it is determined that the size of the tumor, the number of tumor cells, or the number of CD19-expressing B cells, for example, is increased relative to the time just after the initial treatment, then an additional dose of the modified cell population may be administered.

By “reducing tumor size” or “inhibiting tumor growth” of a solid tumor is meant a response to treatment, or stabilization of disease, according to standard guidelines, such as, for example, the Response Evaluation Criteria in Solid Tumors (RECIST) criteria. For example, this may include a reduction in the diameter of a solid tumor of about 5%, 10%, 20%, 30%, 40%, 50%, 60%, 70%, 80%, 90%, or 100%, or the reduction in the number of tumors, circulating tumor cells, or tumor markers, of about 5%, 10%, 20%, 30%, 40%, 50%, 60%, 70%, 80%, 90%, or 100%. The size of tumors may be analyzed by any method, including, for example, CT scan, MRI, for example, CT-MRI, chest X-ray (for tumors of the lung), or molecular imaging, for example, PET scan, such as, for example, a PET scan after administering an iodine 123-labelled PSA, for example, PSMA ligand, such as, for example, where the inhibitor is TROFEX™/MIP-1072/1095, or molecular imaging, for example, SPECT, or a PET scan using PSA, for example, PSMA antibody, such as, for example, capromad pendetide (Prostascint), a 111-iridium labeled PSMA antibody.

By “reducing, slowing, or inhibiting tumor vascularization” is meant a reduction in tumor vascularization of about 5%, 10%, 20%, 30%, 40%, 50%, 60%, 70%, 80%, 90%, or 100%, or a reduction in the appearance of new vasculature of about 5%, 10%, 20%, 30%, 40%, 50%, 60%, 70%, 80%, 90%, or 100%, when compared to the amount of tumor vascularization before treatment. The reduction may refer to one tumor, or may be a sum or an average of the vascularization in more than one tumor. Methods of measuring tumor vascularization include, for example, CAT scan, MRI, for example, CT-MRI, or molecular imaging, for example, SPECT, or a PET scan, such as, for example, a PET scan after administering an iodine 123-labelled PSA, for example, PSMA ligand, such as, for example, where the inhibitor is TROFEX™/MIP-1072/1095, or a PET scan using PSA, for example, PSMA antibody, such as, for example, capromad pendetide (Prostascint), a 111-iridium labeled PSMA antibody.

A tumor is classified, or named as part of an organ, such as a prostate cancer tumor when, for example, the tumor is present in the prostate gland, or has derived from or metastasized from a tumor in the prostate gland, or produces PSA. A tumor has metastasized from a tumor in the prostate gland, when, for example, it is determined that the tumor has chromosomal breakpoints that are the same as, or similar to, a tumor in the prostate gland of the subject.

In other embodiments, following administration of the modified cell population, wherein the modified cells express an inducible chimeric pro-apoptotic polypeptide, such a, for example, the inducible Caspase-9 polypeptide, in the event of a need to reduce the number of modified cells or in vivo modified cells, the multimeric ligand may be administered to the patient. In these embodiments, the methods comprise determining the presence or absence of a negative symptom or condition, such as, for example, cytokine storm, neurotoxicity, cytotoxicity, Graft vs Host Disease, or off target toxicity, and administering a dose of the multimeric ligand. The methods may further comprise monitoring the symptom or condition and administering an additional dose of the multimeric ligand in the event the symptom or condition persists. This monitoring and treatment schedule may continue while the therapeutic cells that express chimeric antigen receptors or chimeric stimulating molecules remain in the patient. In some embodiments, the number of modified cells comprising the chimeric Caspase-9 polypeptide is reduced by 50, 60, 70, 80, 90, 95, or 99% or more following administration of the multimeric ligand to the subject.

An indication of adjusting or maintaining a subsequent drug dose, such as, for example, a subsequent dose of the modified cells or nucleic acid, and/or the subsequent drug dosage, can be provided in any convenient manner. An indication may be provided in tabular form (e.g., in a physical or electronic medium) in some embodiments. For example, the size of the tumor cell, or the number or level of tumor cells in a sample may be provided in a table, and a clinician may compare the symptoms with a list or table of stages of the disease. The clinician then can identify from the table an indication for subsequent drug dose. In certain embodiments, an indication can be presented (e.g., displayed) by a computer, after the symptoms are provided to the computer (e.g., entered into memory on the computer). For example, this information can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

Treatment for solid tumor cancers, including, for example, prostate cancer, may be optimized by determining the concentration of a biomarker associated with the tumor, during the course of treatment. Because patients may have different responses to the course of treatment, the response to treatment may be monitored by following biomarker concentrations or levels in various body fluids or tissues. The determination of the concentration, level, or amount of a biomarker polypeptide may include detection of the full length polypeptide, or a fragment or variant thereof. The fragment or variant may be sufficient to be detected by, for example, immunological methods, mass spectrometry, nucleic acid hybridization, and the like. Optimizing treatment for individual patients may help to avoid side effects as a result of overdosing, may help to determine when the treatment is ineffective and to change the course of treatment, or may help to determine when doses may be increased, or to determine the timing of treatment.

For example, it has been determined that amount or concentration of certain biomarkers changes during the course of treatment of solid tumors. Predetermined target levels of such biomarkers, or biomarker thresholds may be identified in normal subject, are provided, which allow a clinician to determine whether a subsequent dose of a drug administered to a subject in need thereof, such as a subject with a solid tumor, such as, for example, a prostate tumor, may be increased, decreased or maintained. A clinician can make such a determination based on whether the presence, absence or amount of a biomarker is below, above or about the same as a biomarker threshold, respectively, in certain embodiments.

Cytokines are a large and diverse family of polypeptide regulators produced widely throughout the body by cells of diverse origin. The presence or the level of a cytokine may be used as a biomarker. The term “cytokine” is a general description of a large family of proteins and glycoproteins. Other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on cells that secrete them (autocrine action), on nearby cells (paracrine action), or in some instances on distant cells (endocrine action). The treatment of a subject with the modified cell populations of the present application, or optionally, subsequent administration of a drug such as, for example, rimiducid, to induce apoptosis and eliminate the cells may be monitored by detecting the level of cytokines associated with toxicity in the subject. Examples of cytokines include, without limitation, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18 and the like), interferons (e.g., IFN-β, IFN-γ and the like), tumor necrosis factors (e.g., TNF-α, TNF-β and the like), lymphokines, monokines and chemokines; growth factors (e.g., transforming growth factors (e.g., TGF-α, TGF-β and the like)); colony-stimulating factors (e.g. GM-CSF, granulocyte colony-stimulating factor (G-CSF) etc.); and the like.

Detection may be performed using any suitable method, including, without limitation, mass spectrometry (e.g., matrix-assisted laser desorption ionization mass spectrometry (MALDI-MS), electrospray mass spectrometry (ES-MS)), electrophoresis (e.g., capillary electrophoresis), high performance liquid chromatography (HPLC), nucleic acid affinity (e.g., hybridization), amplification and detection (e.g., real-time or reverse-transcriptase polymerase chain reaction (RT-PCR)), and antibody assays (e.g., antibody array, enzyme-linked immunosorbant assay (ELISA)).

A sample can be obtained from a subject at any suitable time of collection after the modified cell population or a drug is delivered to the subject. For example, a sample may be collected within about one hour after a drug is delivered to a subject (e.g., within about 5, 10, 15, 20, 25, 30, 35, 40, 45, 55 or 60 minutes of delivering a drug), within about one day after a drug is delivered to a subject (e.g., within about 2, 3, 4, 5, 6, 7, 8, 9, 10, 11, 12, 13, 14, 15, 16, 17, 18, 19, 20, 21, 22, 23 or 24 hours of delivering a drug) or within about two weeks after a drug is delivered to a subject (e.g., within about 2, 3, 4, 5, 6, 7, 8, 9, 10, 11, 12, 13 or 14 days of delivering the drug). A collection may be made on a specified schedule including hourly, daily, semi-weekly, weekly, bi-weekly, monthly, bi-monthly, quarterly, and yearly, and the like, for example. If a drug is administered continuously over a time period (e.g., infusion), the delay may be determined from the first moment of drug is introduced to the subject, from the time the drug administration ceases, or a point in-between (e.g., administration time frame midpoint or other point). Administration of a modified cell population to a subject is understood to be interchangeable with the phrase administration of modified cells, or modified T cells, for example. That is, a group of modified cells, in plural, is understood to also refer to a modified cell population, in discussions of administration or preparation of modified cells.

### Formulations and Routes for Administration to Patients

Where clinical applications are contemplated, it will be necessary to prepare pharmaceutical compositions—expression constructs, expression vectors, fused proteins, transduced cells, activated T cells, transduced and loaded T cells—in a form appropriate for the intended application. Generally, this will entail preparing compositions that are essentially free of pyrogens, as well as other impurities that could be harmful to humans or animals.

The multimeric ligand, such as, for example, AP1903 (rimiducid), may be delivered, for example at doses of about 0.01 to 1 mg/kg subject weight, of about 0.05 to 0.5 mg/kg subject weight, 0.1 to 2 mg/kg subject weight, of about 0.05 to 1.0 mg/kg subject weight, of about 0.1 to 5 mg/kg subject weight, of about 0.2 to 4 mg/kg subject weight, of about 0.3 to 3 mg/kg subject weight, of about 0.3 to 2 mg/kg subject weight, or about 0.3 to 1 mg/kg subject weight, for example, about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject weight. In some embodiments, the ligand is provided at 0.4 mg/kg per dose, for example at a concentration of 5 mg/mL. Vials or other containers may be provided containing the ligand at, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml. A suitable process for activating the inducible caspase-9 safety switch is provided in, for example, Di Stasi et al. (2011) N Engl J Med 365:1673-83, and in U.S. patent application Ser. No. 13/112,739, filed May 20, 2011, published Nov. 24, 2011, as US2011-0286980, issued Jul. 28, 2015, as U.S. Pat. No. 9,089,520.

### Combination Therapies

In order to increase the effectiveness of the expression vectors presented herein, it may be desirable to combine these compositions and methods with an agent effective in the treatment of the disease.

In certain embodiments, anti-cancer agents may be used in combination with the present methods. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

In some embodiments antibiotics can be used in combination with the pharmaceutical composition to treat and/or prevent an infectious disease. Such antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof.

More generally, such an agent would be provided in a combined amount with the expression vector effective to kill or inhibit proliferation of a cancer cell and/or microorganism. This process may involve contacting the cell(s) with an agent(s) and the pharmaceutical composition at the same time or within a period of time wherein separate administration of the pharmaceutical composition and an agent to a cell, tissue or organism produces a desired therapeutic benefit. This may be achieved by contacting the cell, tissue or organism with a single composition or pharmacological formulation that includes both the pharmaceutical composition and one or more agents, or by contacting the cell with two or more distinct compositions or formulations, wherein one composition includes the pharmaceutical composition and the other includes one or more agents.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing. In some embodiments, the chemotherapeutic agent is selected from the group consisting of carboplatin, estramustine phosphate (Emcyt), and thalidomide. In some embodiments, the chemotherapeutic agent is a taxane. The taxane may be, for example, selected from the group consisting of docetaxel (Taxotere), paclitaxel, and cabazitaxel. In some embodiments, the taxane is docetaxel. In some embodiments, the chemotherapeutic agent is administered at the same time or within one week after the administration of the modified cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the modified cell or nucleic acid. In some embodiments, the chemotherapeutic agent is administered at least 1 month before administering the cell or nucleic acid.

The administration of the pharmaceutical composition may precede, be concurrent with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

In some embodiments, the chemotherapeutic agent may be a lymphodepleting chemotherapeutic. In other examples, the chemotherapeutic agent may be Taxotere (docetaxel), or another taxane, such as, for example, cabazitaxel. The chemotherapeutic may be administered before, during, or after treatment with the cells and inducer. For example, the chemotherapeutic may be administered about 1 year, 11, 10, 9, 8, 7, 6, 5, or 4 months, or 18, 17, 16, 15, 14, 13, 12, 11, 10, 9, 8, 7, 6, 5, 4, 3, 2, weeks or 1 week prior to administering the first dose of activated nucleic acid. Or, for example, the chemotherapeutic may be administered about 1 week or 2, 3, 4, 5, 6, 7, 8, 9, 10, 11, 12, 13, 14, 15, 16, 17, or 18 weeks or 4, 5, 6, 7, 8, 9, 10, or 11 months or 1 year after administering the first dose of cells or inducer.

Administration of a chemotherapeutic agent may comprise the administration of more than one chemotherapeutic agent. For example, cisplatin may be administered in addition to Taxotere or other taxane, such as, for example, cabazitaxel.

In some embodiments, the invention provides for combination therapies comprising the modified cell population described herein with cytokines or chemokines neutralizing agent, e.g. a neutralizing antibody. In some embodiments, the invention provides for combination therapies comprising the modified cell population described herein and a TNFα neutralizing agent, e.g., an anti-TNFα antibody.

### EXAMPLES

### Example 1: MyD88/CD40 Enhanced CAR-T Cells Maintain Therapeutic Efficacy Following Resolution of Cytokine-Related Toxicity Using Inducible Caspase-9

### Abstract

Successful adoptive chimeric antigen receptor (CAR) T cell therapies against hematological malignancies requires CAR-T expansion and durable persistence following infusion. Balancing increased CAR-T potency with safety, including severe cytokine release syndrome (sCRS) and neurotoxicity, warrants inclusion of safety mechanisms to control in vivo CAR-T activity. Here, we describe a novel CAR-T cell platform that utilizes expression of the toll-like receptor (TLR) adaptor molecule MyD88 and tumor-necrosis factor family member, CD40, (MC), tethered to the CAR molecule through an intentionally inefficient 2A linker system, providing a constitutive signal that drives CAR-T survival, proliferation and anti-tumor activity against CD19⁺ and CD123⁺ hematological cancers. Robust activity of MC-enhanced CAR-T cells was associated with cachexia in animal models that corresponded with high levels of human cytokine production. However, toxicity could be mitigated by using inducible caspase-9 (iC9) to reduce serum cytokines, by administration of neutralizing antibody against TNF-α, or by selecting “low” cytokine producing CD8⁺ T cells without loss of anti-tumor activity. Interestingly, high basal activity was essential for in vivo CAR-T expansion. This study shows that co-opting novel signaling elements (i.e., MyD88 and CD40) and development of a unique CAR-T architecture can drive T cell proliferation in vivo to enhance CAR-T therapies.

Expression Constructs

### Plasmid Construction

The pB001 tricistronic SFG-based retroviral vector is an example of a vector that was used in some examples to prepare a modified CD19 CAR-T cell population, expressing a CD19-specific chimeric antigen receptor, a constitutively-active MyD88-CD40 chimeric polypeptide, and an iC9 safety switch.

Plasmid pB001 contains, in the 5′ to 3′ direction, nucleic acid encoding:
(1) an MLEMLE linker (SEQ ID NO: 31, encoded by SEQ ID NO: 32), a mutant human FKBP12 protein (FKBP12(F36V) also known as FKBP12v36, F_{V36}, FKBP_{V}, or F_{V}; SEQ ID NO: 1 encoded by SEQ ID NO: 2) in which the phenylalanine at amino acid position 36 (or 37 if the initial methionine of the protein is counted) is substituted by a valine which is fused, through an 8-amino acid linker (SEQ ID NO: 3 encoded by SEQ ID NO: 4) to a portion of human caspase 9 polypeptide (Δcaspase9 which contains amino acids 135-416 of caspase 9; SEQ ID NO: 5 encoded by SEQ ID NO: 6) (the entire fusion protein is termed iC9),
(2) a T2A polypeptide (SEQ ID NO: 7 encoded by SEQ ID NO: 8),
(3) a membrane signal peptide (SEQ ID NO: 9 encoded by SEQ ID NO: 10) fused to light (SEQ ID NO: 11 encoded by SEQ ID NO: 12) and heavy chain (SEQ ID NO: 15 encoded by SEQ ID NO: 16) variable regions of anti-CD19 monoclonal antibody FMC63 (with an intervening 8-amino acid flexible glycine-serine linker, i.e., flex peptide (SEQ ID NO: 13 encoded by SEQ ID NO: 14) between the chains) fused to a human CD34 epitope polypeptide (amino acids 30-45 of CD34; SEQ ID NO: 17 encoded by SEQ ID NO: 18) which is fused to an alpha stalk region of human CD8 (amino acids 141-182 of CD8; SEQ ID NO: 19 encoded by SEQ ID NO: 20) which is fused to the transmembrane domain of human CD8 (amino acids 183-219 of CD8; SEQ ID NO: 21 encoded by SEQ ID NO: 22) which is fused to a portion of human CD3ζ (amino acids 83-194 of CD3ζ isoform X2; SEQ ID NO: 23 encoded by SEQ ID NO: 24),
(4) a P2A polypeptide (SEQ ID NO: 25 encoded by SEQ ID NO: 26),
(5) a fusion protein containing a truncated huma MyD88 polypeptide (the amino terminal 172 amino acids of MyD88 containing the DD domain and intermediary domain; SEQ ID NO: 27 encoded by SEQ ID NO: 28) fused to a portion of a human CD40 polypeptide (the carboxy terminal 62 amino acids, i.e., amino acids 216-277 of CD40; SEQ ID NO: 29 encoded by SEQ ID NO: 30), (the entire fusion protein is termed MC).

The pB002 tricistronic SFG-based retroviral vector is an example of a vector that was used in some examples to prepare a modified Her2 CAR-T cell population, expressing a Her2-specific chimeric antigen receptor, a constitutively-active MyD88-CD40 chimeric polypeptide, and an iC9 safety switch.

Plasmid pB002 contains, in the 5′ to 3′ direction, nucleic acid encoding:
(1) an MLE linker (SEQ ID NO: 43, encoded by SEQ ID NO: 44), a mutant human FKBP12 protein (FKBP12(F36V) also known as FKBP12v36, F_{V36}, FKBP_{V}, or F_{V}; SEQ ID NO: 1 encoded by SEQ ID NO: 2) in which the phenylalanine at amino acid position 36 (or 37 if the initial methionine of the protein is counted) is substituted by a valine which is fused, through an 8-amino acid linker (SEQ ID NO: 3 encoded by SEQ ID NO: 4) to a portion of human caspase 9 polypeptide (Δcaspase9 which contains amino acids 135-416 of caspase 9; SEQ ID NO: 5 encoded by SEQ ID NO: 6; without the terminal proline of SEQ ID NO: 5, or without the terminal codon coding for proline of SEQ ID NO: 6) (the entire fusion protein is termed iC9),
(2) a T2A polypeptide (SEQ ID NO: 7 encoded by SEQ ID NO: 8),
(3) a membrane signal peptide (SEQ ID NO: 9 encoded by SEQ ID NO: 10) fused to heavy (SEQ ID NO: 45 encoded by SEQ ID NO: 46) and light chain (SEQ ID NO: 47 encoded by SEQ ID NO: 48) variable regions of anti-Her2 monoclonal antibody FRP5 (with an intervening linker (SEQ ID NO: 49 encoded by SEQ ID NO: 50) between the chains) fused to a human CD34 epitope polypeptide (amino acids 30-45 of CD34; SEQ ID NO: 17 encoded by SEQ ID NO: 18) which is fused to an alpha stalk region of human CD8 (amino acids 141-182 of CD8; SEQ ID NO: 19 encoded by SEQ ID NO: 20) which is fused to the transmembrane domain of human CD8 (amino acids 183-219 of CD8; SEQ ID NO: 21 encoded by SEQ ID NO: 22) which is fused to a portion of human CD3ζ (amino acids 83-194 of CD3ζ isoform X2; SEQ ID NO: 23 encoded by SEQ ID NO: 24),
(4) a P2A polypeptide (SEQ ID NO: 25 encoded by SEQ ID NO: 26),
(5) a fusion protein containing myristoylation domain (SEQ ID NO: 51, encoded by SEQ ID NO: 52), a truncated huma MyD88 polypeptide (the amino terminal 172 amino acids of MyD88 containing the DD domain and intermediary domain; SEQ ID NO: 27 encoded by SEQ ID NO: 28) fused to a portion of a human CD40 polypeptide (the carboxy terminal 62 amino acids, i.e., amino acids 216-277 of CD40; SEQ ID NO: 29 encoded by SEQ ID NO: 30), (the entire fusion protein is termed MC).

The pB003 tricistronic SFG-based retroviral vector is an example of a vector that was used in some examples to prepare a modified PSCA CAR-T cell population, expressing a PSCA-specific chimeric antigen receptor, a constitutively-active MyD88-CD40 chimeric polypeptide, and an iC9 safety switch.

Plasmid pB003 contains, in the 5′ to 3′ direction, nucleic acid encoding:
(1) an MLE linker (SEQ ID NO: 43, encoded by SEQ ID NO: 44), a mutant human FKBP12 protein (FKBP12(F36V) also known as FKBP12v36, F_{V36}, FKBP_{V}, or F_{V}; SEQ ID NO: 1 encoded by SEQ ID NO: 2) in which the phenylalanine at amino acid position 36 (or 37 if the initial methionine of the protein is counted) is substituted by a valine which is fused, through an 8-amino acid linker (SEQ ID NO: 3 encoded by SEQ ID NO: 4) to a portion of human caspase 9 polypeptide (Δcaspase9 which contains amino acids 135-416 of caspase 9; SEQ ID NO: 5 encoded by SEQ ID NO: 6; without the terminal proline of SEQ ID NO: 5, or without the terminal codon coding for proline of SEQ ID NO: 6) (the entire fusion protein is termed iC9),
(2) a T2A polypeptide (SEQ ID NO: 7 encoded by SEQ ID NO: 8),
(3) a membrane signal peptide (SEQ ID NO: 9 encoded by SEQ ID NO: 10) fused to light (SEQ ID NO: 53 encoded by SEQ ID NO: 54) and heavy chain (SEQ ID NO: 55 encoded by SEQ ID NO: 56) variable regions of anti-PSCA monoclonal antibody A11 (with an intervening 8-amino acid flexible glycine-serine linker, i.e., flex peptide (SEQ ID NO: 13 encoded by SEQ ID NO: 14) between the chains), fused to a human CD34 epitope polypeptide (amino acids 30-45 of CD34; SEQ ID NO: 17 encoded by SEQ ID NO: 18) which is fused to an alpha stalk region of human CD8 (amino acids 141-182 of CD8; SEQ ID NO: 19 encoded by SEQ ID NO: 20) which is fused to the transmembrane domain of human CD8 (amino acids 183-219 of CD8; SEQ ID NO: 21 encoded by SEQ ID NO: 22) which is fused to a portion of human CD3ζ (amino acids 83-194 of CD3ζ isoform X2; SEQ ID NO: 23 encoded by SEQ ID NO: 24),
(4) a P2A polypeptide (SEQ ID NO: 25 encoded by SEQ ID NO: 26),
(5) a fusion protein containing myristoylation domain (SEQ ID NO: 51, encoded by SEQ ID NO: 52), a truncated huma MyD88 polypeptide (the amino terminal 172 amino acids of MyD88 containing the DD domain and intermediary domain; SEQ ID NO: 27 encoded by SEQ ID NO: 28) fused to a portion of a human CD40 polypeptide (the carboxy terminal 62 amino acids, i.e., amino acids 216-277 of CD40; SEQ ID NO: 29 encoded by SEQ ID NO: 30), (the entire fusion protein is termed MC).

Materials and Methods

Mice. NOD.Cg-Prkdcs^{cid}II2rg^{tm1WjI}/SzJ (NSG) mice were obtained from Jackson Laboratories (Bar Harbor, Me.).

Cell lines, media and reagents. 293T (HEK 293T/17), Raji, Daudi and THP-1 cell lines were obtained from the American Type Culture Collection. Cell lines were maintained in DMEM (Invitrogen, Grand Island, N.Y.) supplemented with 10% fetal calf serum (FCS) and 2 mM glutamax (Invitrogen) at 37° C. and 5% CO₂. T cells generated from peripheral blood mononuclear cells (PBMC) were cultured in 45% RPMI 1640, 45% Click's media (Invitrogen) supplemented with 10% fetal bovine serum (FBS), 2 mM glutamax (T cell media; TCM) and 100 U/ml IL-2 (Miltenyi Biotec, Bergisch Gladbach, Germany), unless otherwise noted. Clinical grade rimiducid was diluted in ethanol to a 100 mM working solution for in vitro assays, or 0.9% saline for animal studies.

Retroviral and plasmid constructs. Initial bicistronic SFG-based retroviral vectors were generated encoding iC9 together with a first-generation anti-CD19 CAR comprising the FMC63 single chain variable fragment (scFv), the CD8a stalk and transmembrane domain and the CD3ζ chain cytoplasmic domain (iC9-CD19.ζ). In all CAR vectors, the CD34 Qbend-10 minimal epitope (10) was included in in the CD8a stalk to detect CAR expression on gene-modified T cells. A third-generation CAR was constructed, which included the MC costimulatory proteins proximal to the CD8α transmembrane region (iC9-CD19.MC.ζ). In addition, vectors were constructed with only MyD88 (M) or CD40 (C) for both the third-generation (iC9-CD19.M.ζ or iC9-CD19.C.ζ, respectively). A tricistronic iC9-enabled CD19 and CD123 (331292 scFv (11,12)) CAR construct with a constitutively expressed MC chimeric protein (iC9-CD19.ζ-MC) was constructed. iC9-expressing CD19 vectors were also synthesized encoding the CD28 and 4-1BB endodomains as previously described (13,14). Additional vectors were synthesized with enhanced 2A sequences, including GSG linkers to improve ribosomal skipping efficiency (15), as well as alternative orientations of the above transgenes. For co-culture assays and in vivo studies, tumor cell lines were modified with retroviral vectors encoding EGFPluciferase (EGFPluc).

Generation of gene-modified T cells. Retroviral supernatants were produced by transient co-transfection of 293T cells with the SFG vector plasmid, EQ-PAM3(-E) plasmid containing the sequence for MoMLV gag-pol and an RD114 envelope encoding plasmid, using GeneJuice (EMD Biosciences, Gibbstown, N.J.) transfection reagent. Activated T cells were made from peripheral blood mononuclear cells (PBMCs) obtained from the Gulf Coast Blood Bank (Houston, Tex.) and activated using anti-CD3/anti-CD28 antibodies, as previously described (5). After 3 days of activation, T cells were subsequently transduced on Retronectin-coated plates (Takara Bio, Otsu, Shiga, Japan) and expanded with 100 U/ml IL-2 and expanded for 10 to 14 days. For two transductions, the protocol was identical to above except the wells were coated with equal amounts of each retroviral supernatant.

Immunophenotyping. Gene-modified T cells were analyzed for transgene expression 10 to 14 days post-transduction by flow cytometry using CD3-PerCP.Cy5 and CD34-PE (BioLegend, San Diego, Calif.). Experiments evaluating cell selection of CAR-T cell subsets (i.e., CD4 and CD8) were tested for purity using CD4 and CD8 antibodies (BioLegend). Additional phenotypic analyses were conducted using antibodies for CD45RA and CD62L (T cell memory phenotype), and PD-1 (T cell exhaustion). All flow cytometry was performed using a Gallios flow cytometer and the data analyzed using Kaluza software (Beckman Coulter, Brea, Calif.).

Coculture Assays. Non-transduced and gene-modified T cells were cultured at a 1:1 effector to target (5×10⁵ cells each in a 24-well plate) ratio with CD19+ Raji-EGFPluc tumor cells and cultured for 7 days in the absence of exogenous IL-2. Cells were then harvested, enumerated and analyzed by flow cytometry for the frequency of T cells (CD3⁺) or tumor cells (EGFPluc⁺). In some assays non-transduced and gene-modified T cells were cultured without target cells (5×10⁵ cells each in a 24-well plate). Culture supernatants were analyzed for cytokine levels at 48 hours after the start of the coculture.

Animal Models. To evaluate anti-tumor activity of CD19-targeted CAR-T cells, NSG mice were engrafted with 5×10⁵ CD19⁺ Raji or Raji-EGFPluc tumor cells by intravenous (i.v.) tail vein injection. After 4 days, variable doses of non-transduced and gene-modified T cells were administered by i.v. (tail) injection. In some experiments, mice were rechallenged with Raji-EGFPluc T cells as above. To test CD123-specific CAR-T activity, 1×10⁶ CD123⁺ THP-1-EGFPluc were engrafted by i.v. injection, followed by infusion of 2.5×10⁶ unmodified or CAR-T cells 7 days post-tumor engraftment. iC9 titration experiments were performed by treating Raji tumor-bearing mice with 5×10⁶ iC9-CD19.ζ-MC-modified T cells followed by injection of rimiducid 7 days after T cell injection at 0.00005, 0.0005, 0.005, 0.05, 0.5 and 5 mg/kg. To evaluate cytokine-related toxicities, neutralizing antibodies against hIL-6, hIFN-γ and TNF-α or an isotype control antibody (Bio X Cell, West Lebanon, N.H.) were administered by i.p. injection with 100 ug twice weekly. Additional experiments were performed using positively selected CD4⁺ and CD8+ iC9-CD19.ζ-MC-modified T cells using CD4 or CD8 microbeads and MACS columns (Miltenyi Biotec). In vivo tumor growth and T cell proliferation was measured by bioluminescence imaging (BLI) by i.p. injection of 150 mg/kg D-luciferin (Perkin Elmer, Waltham, Mass.) and imaged using the IVIS imaging system (Perkin Elmer). Photon emission was analyzed by whole body region-of-interest (ROI) and signal measured as average radiance (photons/second/cm2/steradian).

Western Blot Analysis. Non-transduced and gene-modified T cells were harvested and lysed and lysates quantified for protein content. Protein lysates were electrophoresed on 10% sodium dodecyl sulfate-polyacrylamide gels and immunoblotted with primary antibodies to β-actin (1:1000, Thermo), caspase-9 (1:400, Thermo), and MyD88 (1:200, Santa Cruz). Secondary antibodies used were HRP-conjugated goat anti-rabbit or mouse IgG antibodies (1:500, Thermo. Membranes were developed using SuperSignal West Femto Maximum Sensitivity Substrate Kit (Thermo, 34096) and imaged using a GelLogic 6000 Pro camera and CareStream MI software (v.5.3.1.16369).

Analysis of in vitro and in vivo cytokine production. Cytokine production of IFN-γ, IL-2 and IL-6 by T cells modified with iMC or control vectors was analyzed by ELISA or cytometric bead array as recommended (eBioscience, San Diego, Calif. or Becton Dickinson, East Rutherford, N.J.). In some experiments, cytokines were analyzed using a multiplex array system (Bio-Plex MAGPIX; Bio-Rad, Hercules, Calif. or Milli-Plex; Millipore, Burlington, Mass.)).

Statistics. Data are represented as mean±SEM. Data were analyzed using Mann-Whitney statistical comparisons to determine significant differences between groups. One-way ANOVA followed by Bonferroni's multiple comparison test was used to compare multiple treatment groups. Two-way ANOVA followed by Bonferroni's test was used to assess statistical significance of differences in tumor growth between multiple treatment groups at different time points. Survival was recorded by Kaplan-Meier graphs, with significance determined by the log-rank test. Data were analyzed using GraphPad Prism v5.0 software (GraphPad, La Jolla, Calif.).

Results

Inclusion of MyD88/CD40 Endodomain within CAR Architecture Provides Costimulation but Diminishes CAR Activity In Vivo.

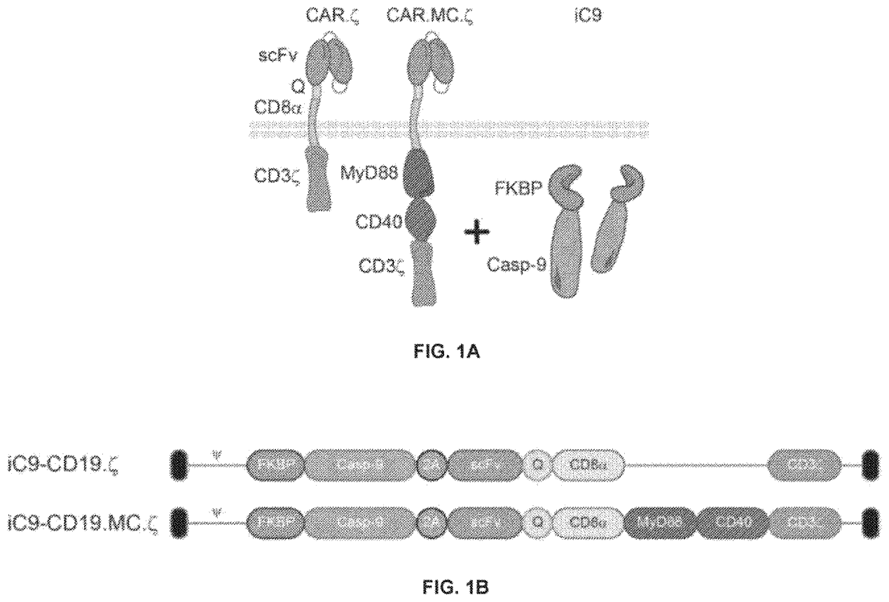

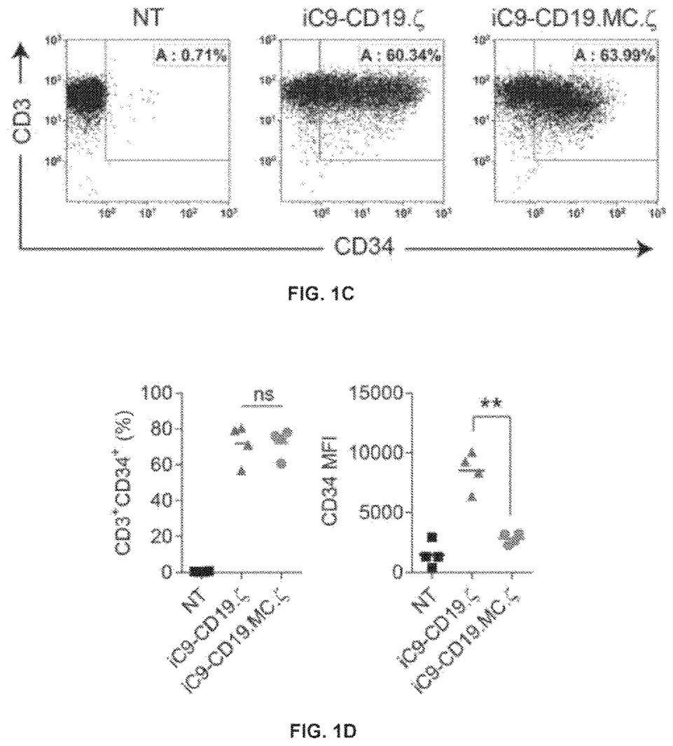

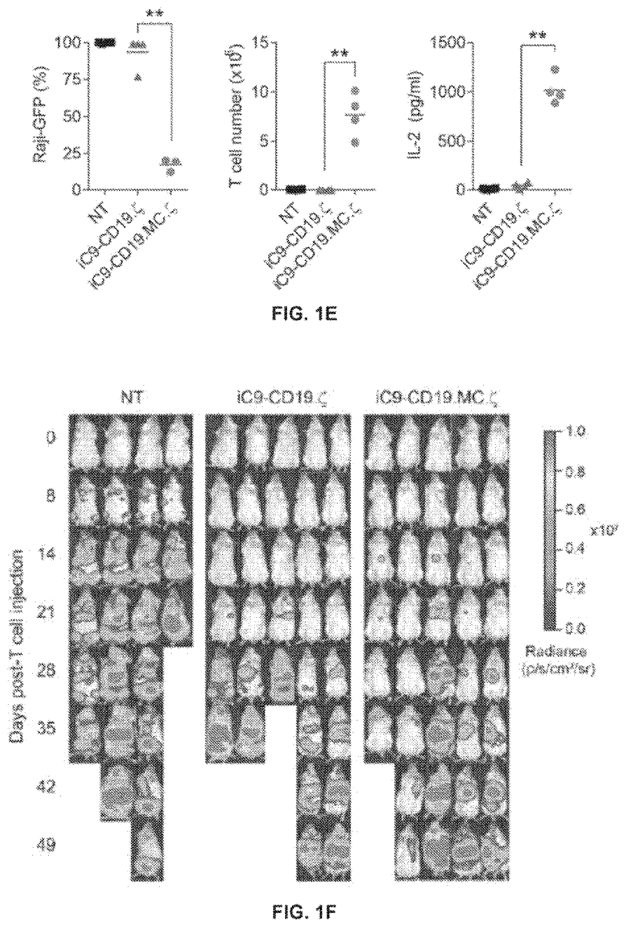

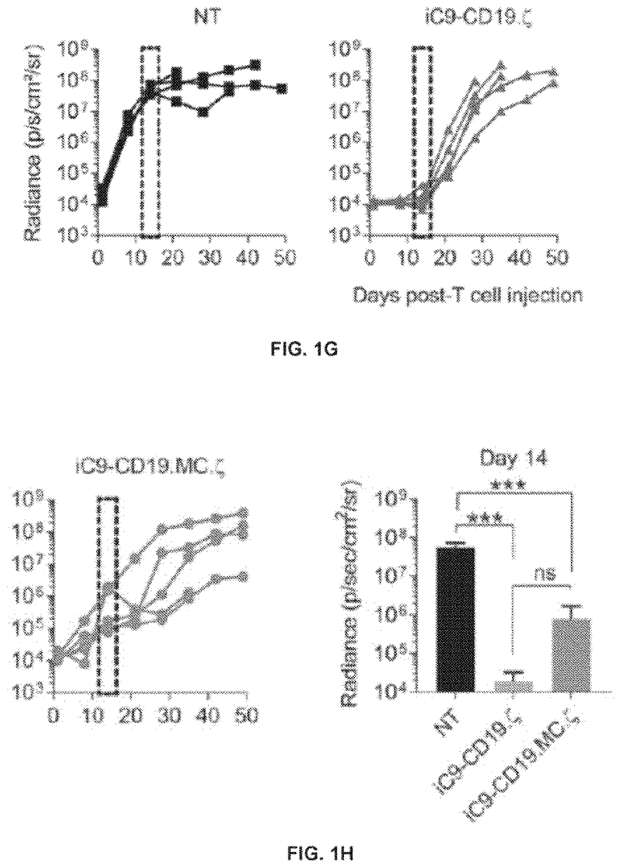

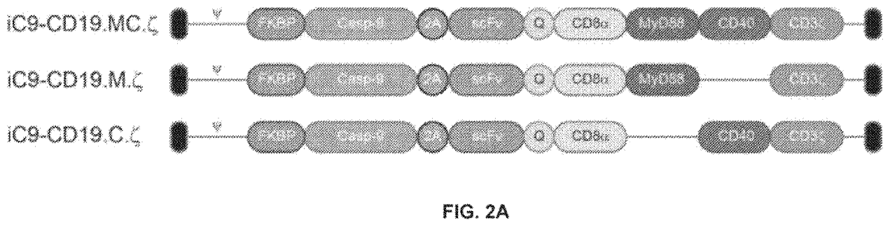

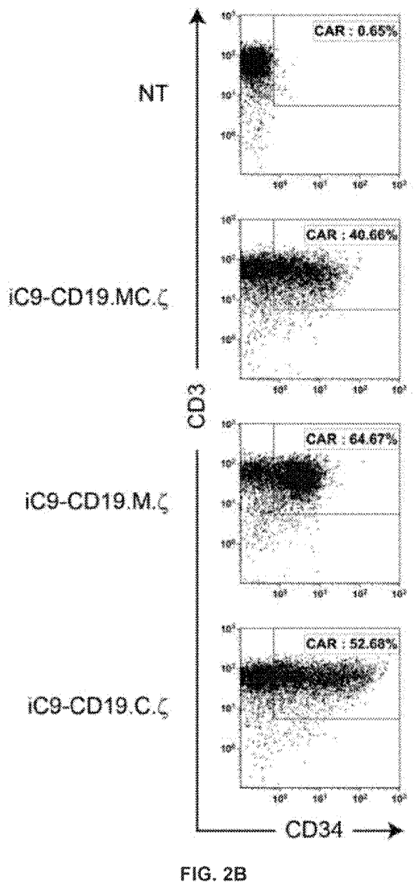

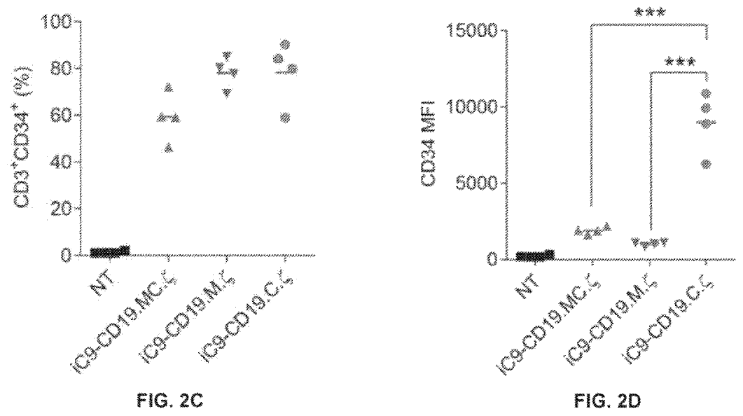

To provide CAR-T cells with MC-costimulation while retaining the ability to use the rimiducid-activated iC9 safety switch, we constructed a bicistronic retroviral vector encoding iC9 followed by a CD19-specific CAR encoding truncated MyD88 (lacking the TIR domain) and CD40 (lacking the extracellular domain) upstream of the CD3ζ signaling element and compared it to a first-generation, iC9-expressing CD19 CAR (FIGS. 1A and 1B). “CAR.z” and “CAR.ζ” both refer to a chimeric antigen receptor that comprises a CD3-ζ polypeptide, and are interchangeable with “CAR.zeta.” Transduction of primary T cells showed equivalent CAR transduction efficiencies for CD19.ζ and CD19.MC.ζ constructs (71±10% versus 72±8%, respectively), however CAR surface expression (MFI) was significantly diminished with the addition of MC (MFI 8513±1587 versus 2824±455; p<0.005) (FIGS. 1C and 1D). Construction of additional vectors expressing MC, or only MyD88 or CD40 revealed that MyD88 lowered CAR expression levels, but not transduction efficiency, suggesting that MyD88 expressed within the CAR was causing CAR instability at the membrane (FIG. 2). Despite reducing CAR cell surface levels, inclusion of the MC signaling domains enhanced CAR activity against CD19-expressing Raji tumor cells by increasing CAR-T proliferation and IL-2 cytokine production over CD19.ζ-only modified T cells (23-fold increase; p<0.0001) (FIG. 1E). We subsequently evaluated CD19-targeted CAR activity using NSG mice engrafted with CD19⁺ Raji tumors. Here, intravenous injection of 5×10⁶ iC9-CD19.ζ or iC9-CD19.MC.ζ-modified T cells showed significant anti-tumor control over non-transduced (NT) T cells (*** p≤0.0001 at day 14) but did not produce durable responses (FIGS. 1F and 1G). Importantly, the addition of MC did not improve anti-tumor activity compared to a first-generation construct. These data suggest that MyD88 is not compatible with normal expression as a costimulatory domain within the CAR architecture.

Constitutive Expression of MC Outside CAR-T Molecule Provides Robust Costimulation while Preserving CAR Expression.

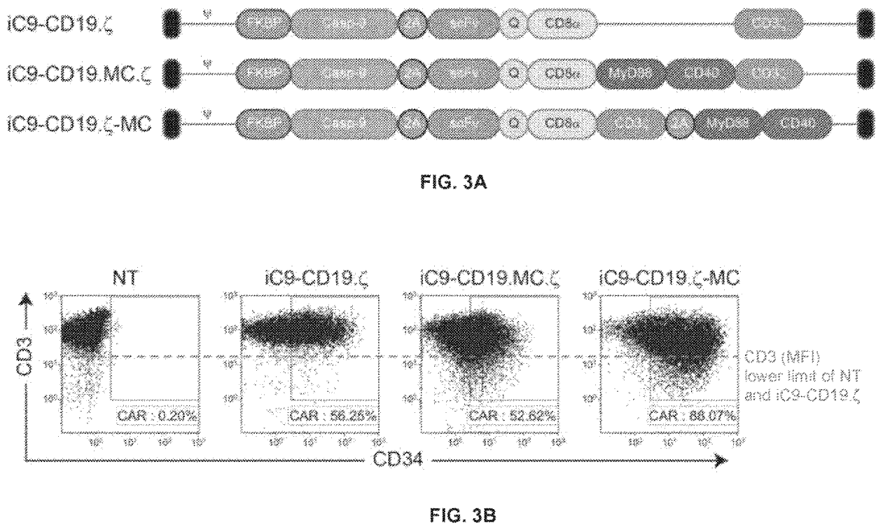

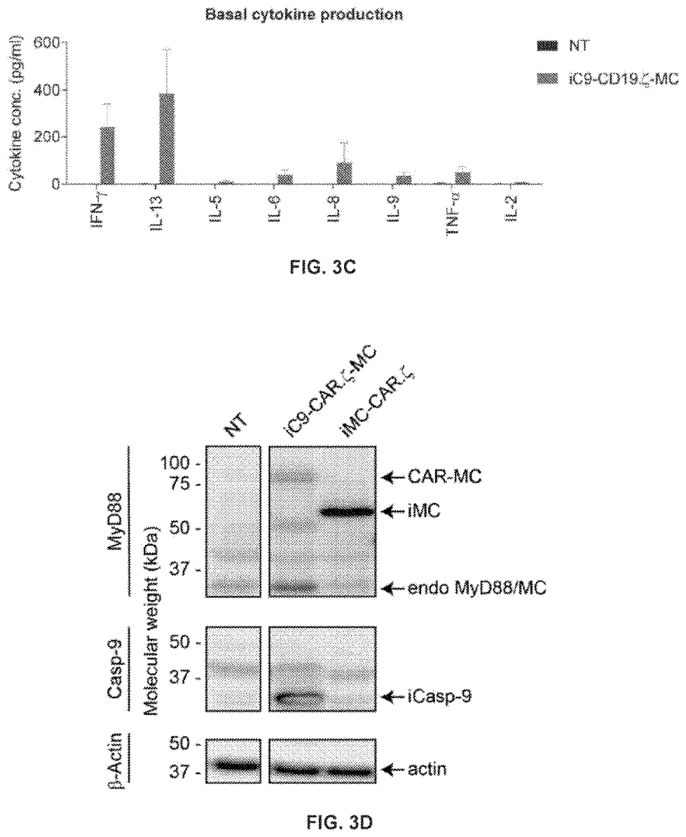

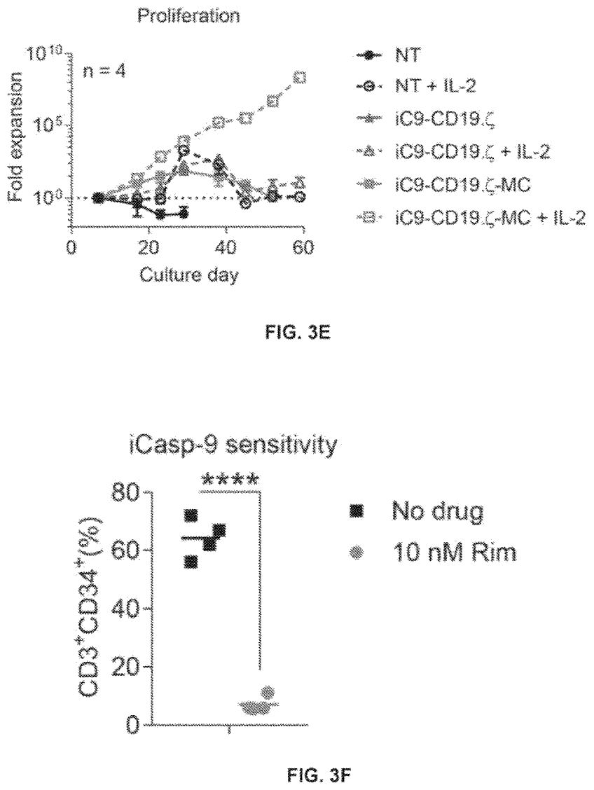

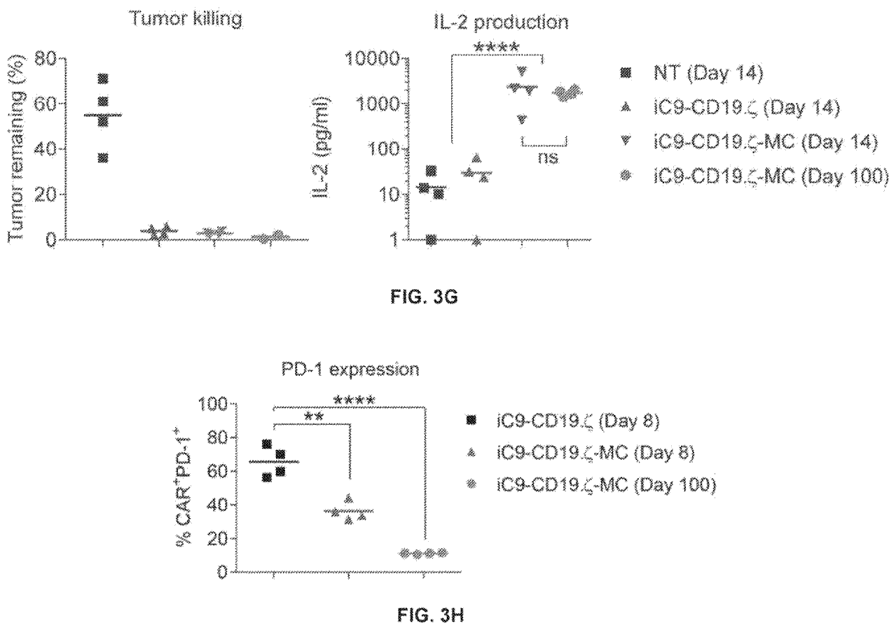

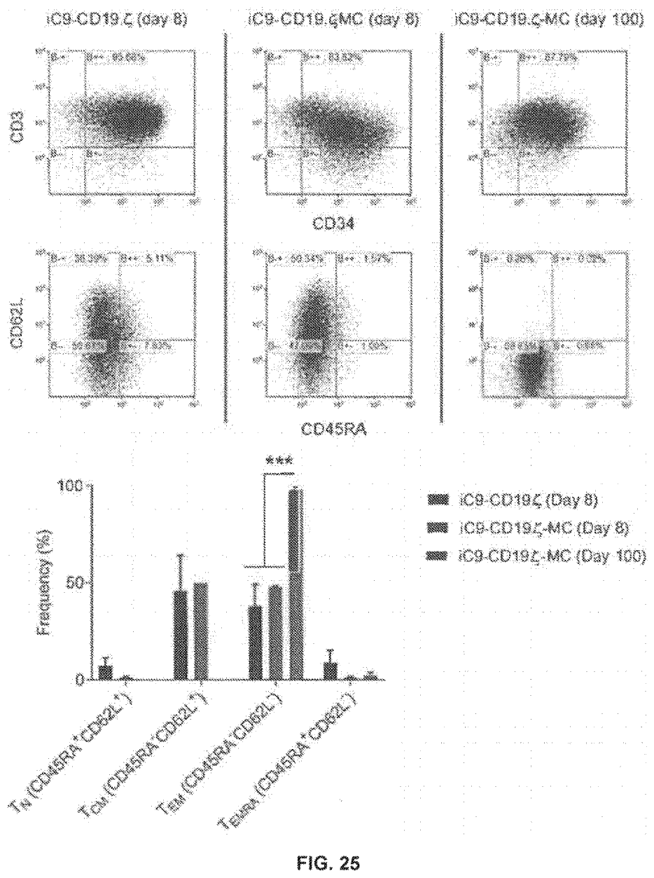

To determine if MC could be used as a constitutively expressed costimulatory module to drive T cell proliferation, we expressed MC outside of the CAR molecule using a tricistronic gene expression approach using an additional 2A sequence (FIG. 3A). Removing MC from the CAR and expressing it as a separate polypeptide (iC9-CD19.ζ-MC) improved CAR expression levels on gene-modified T cells (FIG. 3B), and downregulated endogenous T cell receptor (TCR) levels, consistent with T cell activation (FIG. 3B). Indeed, iC9-CD19.ζ-MC-modified T cells secreted pro-inflammatory cytokines, including IFN-γ, IL-5, IL-6, IL-8, IL-9 and TNF-α in the absence of antigen-stimulation, suggesting that expressing MC was providing a constitutive T cell activating signal (FIG. 3C). Importantly, iC9-CD19.ζ-MC did not trigger IL-2 secretion in the absence of CAR-T engagement. By probing MyD88 expression using Western blot analyses in non-transduced, iC9-CD19.ζ-MC modified and T cells transduced with an inducible MyD88/CD40 CD19 CAR vector (iMC-CD19.ζ), we were able to detect both a fast-migrating (˜30 kDa) and a fainter slow-migrating (˜90 kDa) fragment in iC9-CD19.ζ-MC transduced T cells, suggesting that MC was incompletely separated from the CAR.ζ molecules expressed in this context, presumably due to inefficient 2A ribosomal skipping (FIG. 3D) (15). To understand whether MC-mediated constitutive T cell activation resulted in autonomous CAR-T proliferation, we cultured non-transduced, iC9-CD19.ζ, or iC9-CD19.ζ-MC-modified T cells in the presence or absence of exogenous IL-2 (100 U/ml). In the presence of IL-2, this MC CAR tethering could induce sustained, extensive expansion (over 10⁸) of CAR-T cells after 60 days of culture, yet iC9-CD19.ζ-MC-expressing CAR-T cells failed to survive in the absence of IL-2, reducing the risk of autonomous growth (FIG. 3E). Long-term cultured (100 days) iC9-CD19.ζ-MC transduced T cells remained sensitive to iC9-induced apoptosis when exposed to rimiducid (FIG. 3F) and retained cytotoxic activity and produced IL-2 in coculture assays with CD19⁺ target cells like T cells cultured for a shorter period (14 days) (FIG. 3G). Interestingly, iC9-CD19.ζ-MC-modified T cells showed a decrease in PD-1 expression compared to a first-generation CAR suggesting that constitutive MC activity may reduce the sensitivity of iC9-CD19.ζ-MC T cells to PD-L1 expression in the tumor microenvironment. Moreover, reduced PD-1 expression may delay or prevent T cell exhaustion (FIG. 3H). Additionally, long-term culture of iC9-CD19.ζ-MC-modified T cells show that these cells exhibited a similar T cell subset distribution to that of first-generation CD19-CAR T cells (CD45RA+CD62L+ TN, CD45RA-CD62L+ TCM, CD45RA-CD62L-TEM, CD45RA+CD62L− TEMRA). However, after 100 days in culture, TEM (CD3⁺ CD45−CD62L−) cells were the predominant subtype present in iC9-CD19.ζ-MC T cell cultures (FIG. 25). Thus, iC9-CD19.ζ-MC is a constitutively active CAR construct with sustained proliferative capacity in the presence of antigen stimulation or exogenous IL-2, but is responsive to controlled elimination through the iC9 safety switch.

### Constitutive MC-CAR-T Demonstrated Robust Anti-Tumor Activity Against CD19⁺ Lymphomas in Animals.

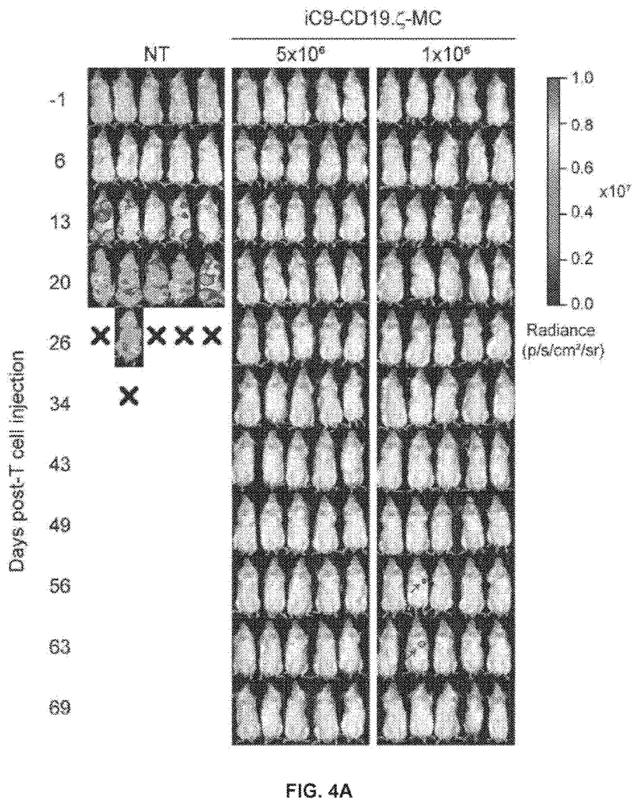

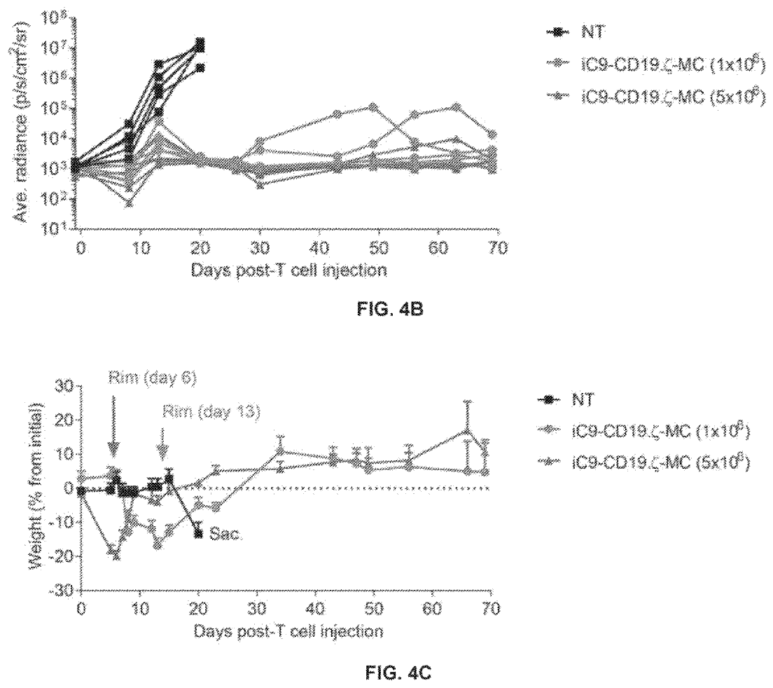

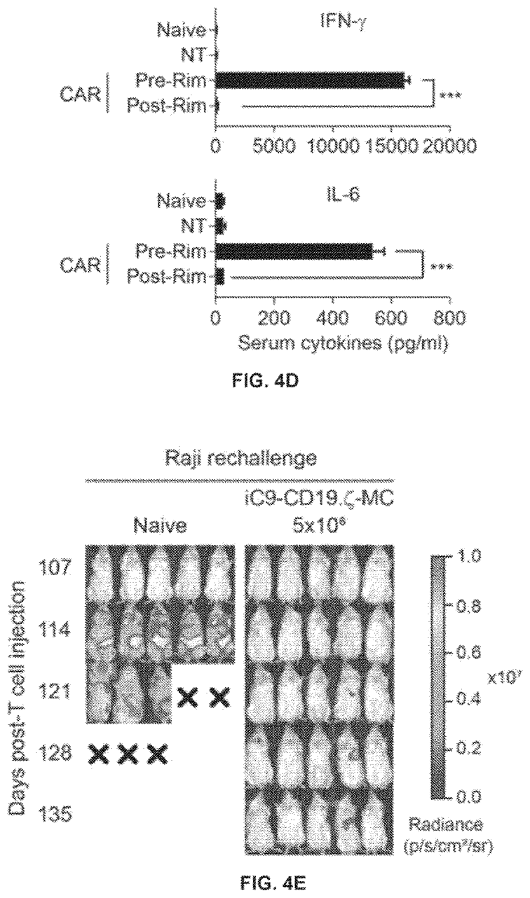

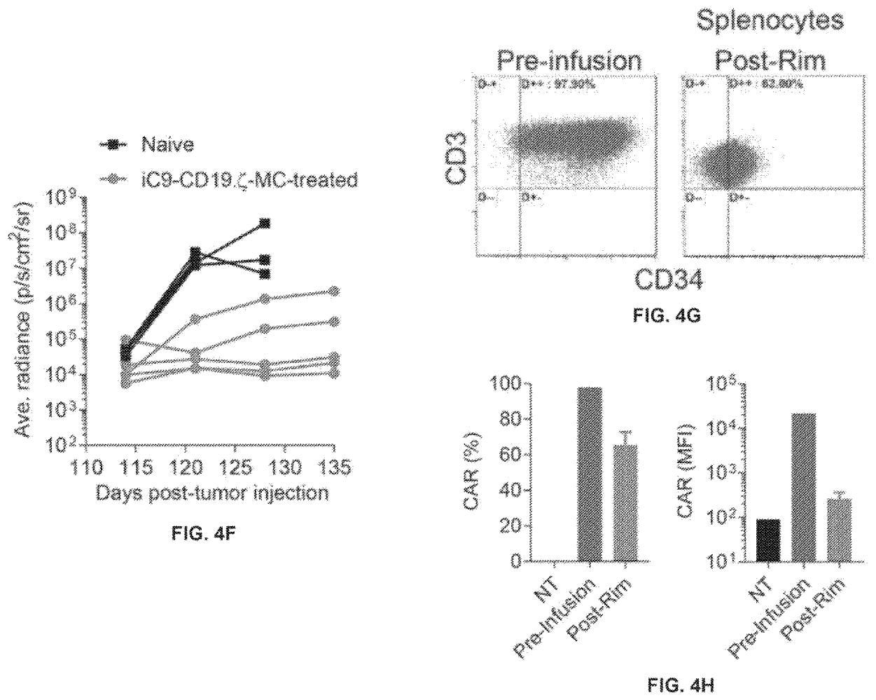

CD19-targeted CAR-T cells expressing constitutive MC were evaluated for efficacy in vivo using immune deficient NSG mice engrafted with the CD19⁺ Raji cell line, modified with the EGFPluc transgene (Raji-EGFPluc) to allow in vivo bioluminescence imaging (BLI). Raji tumor cells grew rapidly in mice treated with 5×10⁶ non-transduced (NT) T cells, requiring sacrifice by day 21 due to hind-leg paralysis (FIG. 4A). Mice treated with 1×10⁸ or 5×10⁶ iC9-CD19.ζ-MC-modified T cells showed early tumor control, which corresponded to acute weight loss in a CAR-T cell dose-dependent manner (FIGS. 4A and 4C). However, CAR-related toxicity was successfully resolved by the administration of 5 mg/kg rimiducid (i.p.) when the mice reached >10% loss in body weight (from initial measurement) (FIG. 4C).

Following rimiducid administration, therapeutic anti-tumor effects of the surviving modified CAR-T cells was observed. FIG. 4B: NSG mice (n=5 per group) were engrafted with Raji-luc tumor cells and then treated with non-transduced (NT) or iC9-CD19.ζ-MC CAR-modified T cells on day 3. Tumor growth was measured by IVIS imaging and calculated by whole-body BLI. FIG. 4C:

Mouse weight was measured to assess CAR-T-related cytokine toxicity. After ˜20% weight loss, mice were treated with rimiducid to eliminate CAR-T cells (FIG. 4C).

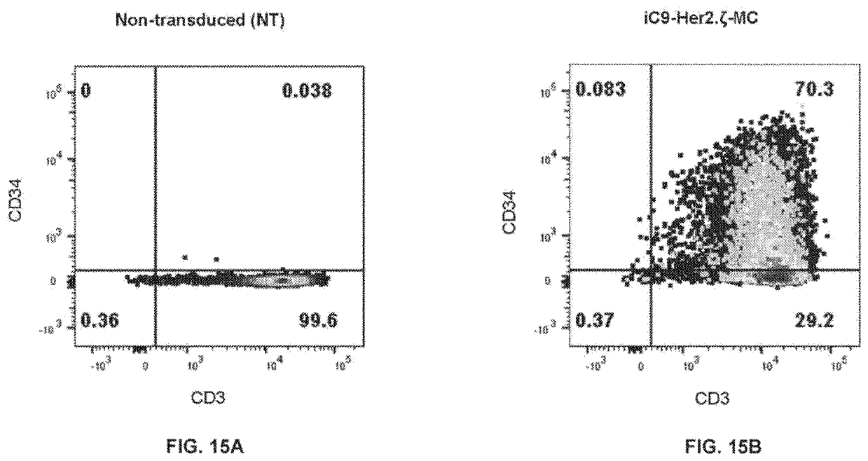

Serum samples taken before and after rimiducid treatment showed high pre-rimiducid levels of human cytokines, including IFN-γ and IL-6, which reverted to baseline levels by 24 hours post-rimiducid exposure (FIG. 4D). Long-term tumor control was not compromised by the activation of the iC9 safety switch, where all CAR-T treated mice remained tumor-free (by BLI) out to 70 days (FIGS. 4A and 4B). As observed in a previous study based on iC9 to lower CAR-T activity (16), animals were resistant to subsequent tumor challenge compared to naive mice due to residual T cells expressing reduced levels of iC9-CD19.ζ-MC (FIGS. 4E and 4F), and residual CAR-T cells could be detected in the spleens of rimiducid-treated animals (FIGS. 4G and 4H). A comparison against first (iC9-CD19.ζ) and second generation (iC9-CD19.28.ζ and iC9-CD19.BB.ζ) CAR constructs showed that antitumor activity was not impaired compared to these alternative CD19 CARs in this animal model, despite the need to deploy iC9 with rimiducid to control toxicity in animals treated with iC9-CD19.ζ-MC-modified T cells (FIGS. 15A-15D).

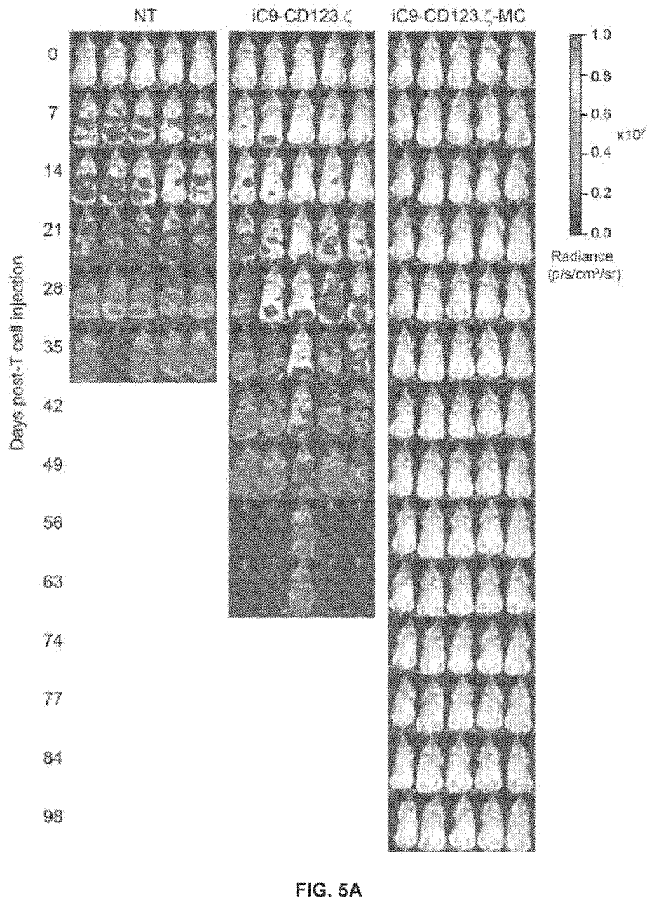

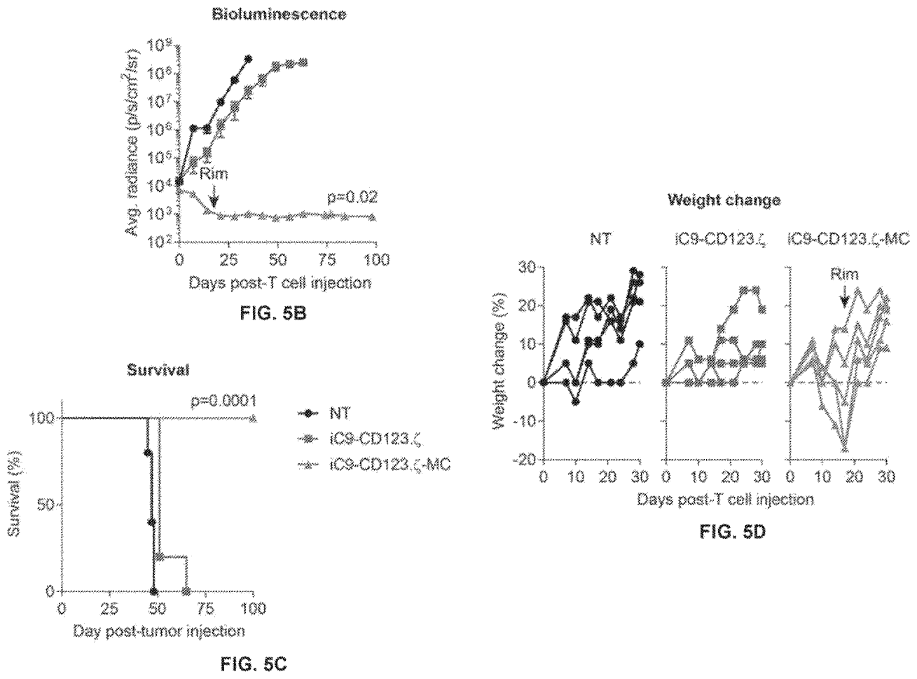

The constitutive MC CAR-T platform targeting CD123⁺ myeloid cell lines (THP-1-EGFPluc) was evaluated in vivo, and compared to non-transduced and T cells modified with an iC9-enabled, first-generation CAR (iC9-CD123.ζ) (FIG. 5A). THP-1-EGFPluc showed rapid outgrowth in mice treated with control T cells, resulting in termination by day 35, while iC9-CD123.-modified T cells showed modest antitumor activity, delaying tumor growth by 2 weeks (FIGS. 5A and 5B). However, the addition of MC to the construct provided durable antitumor responses (>day 100 post-T cell injection) (FIGS. 5A-C). As observed with iC9-CD19.ζ-MC-expressing T cells, 3/5 (60%) of the mice experienced acute toxicity in the form of cachexia by day 14 post-T cell treatment, which could be resolved by rimiducid administration without affecting tumor control (FIG. 5D). Thus, in multiple tumor models, constitutively active MC-driven CAR-T cells demonstrated robust antitumor effects, but cause cachexia in mice due to their high basal activity, necessitating iC9-mediated toxicity mitigation.

### Rimiducid Titration Allowed Partial Ablation of Constitutive CAR-T Activity and Modulates Systemic Cytokine Levels.

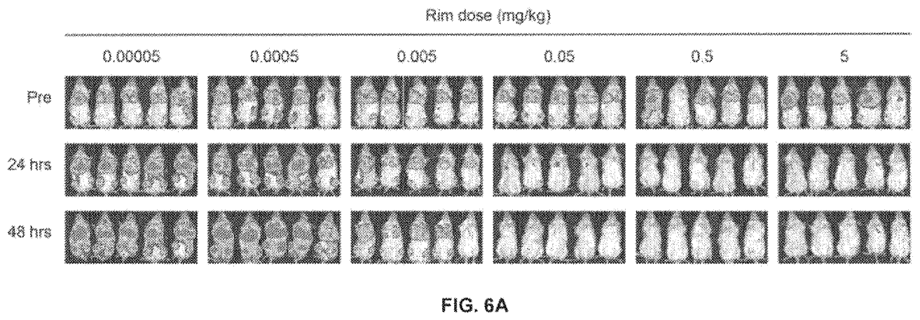

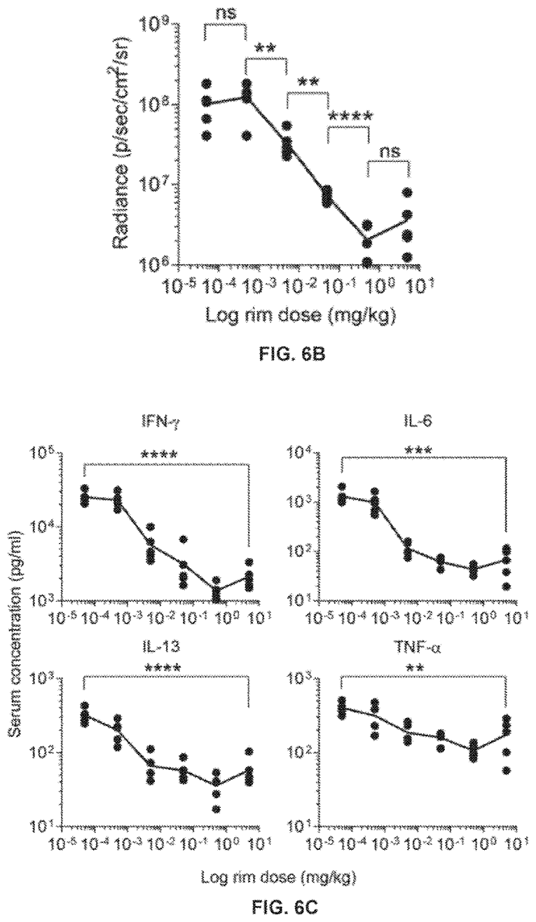

iC9-CD19.ζ-MC-modified T cells showed a high basal activation state which is linked to their antitumor activity. While administration of high dose rimiducid (5 mg/kg) allowed the persistence of low level CAR-T cells, titration of rimiducid may permit the retention of more gene-modified T cells while mitigating cytokine-related toxicities. T cells were co-transduced with iC9-CD19.ζ-MC and EGFPluc and administered into Raji-bearing mice. Following the onset of cachexia (>10% body weight loss), a log-titration of rimiducid (5-5×10⁻⁵ mg/kg) was administered as a single i.p. injection (FIG. 6A). As previously observed (16), CAR-T BLI was reduced in a rimiducid dose-dependent manner (FIG. 6B). CAR-T reduction corresponded decreased serum cytokine levels (i.e., IL-6, IFN-γ and TNF-α) (FIG. 6C). With this highly active construct, rimiducid titration could be selectively modulated to minimize excessive activity while maximizing therapeutic potency.

### MC Basal Activity is Required for CAR-T Expansion In Vivo

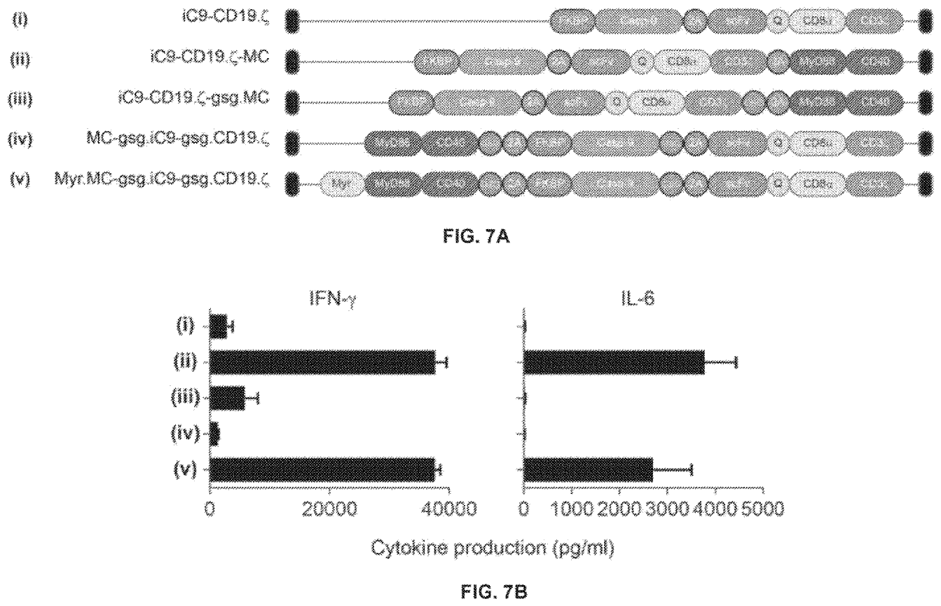

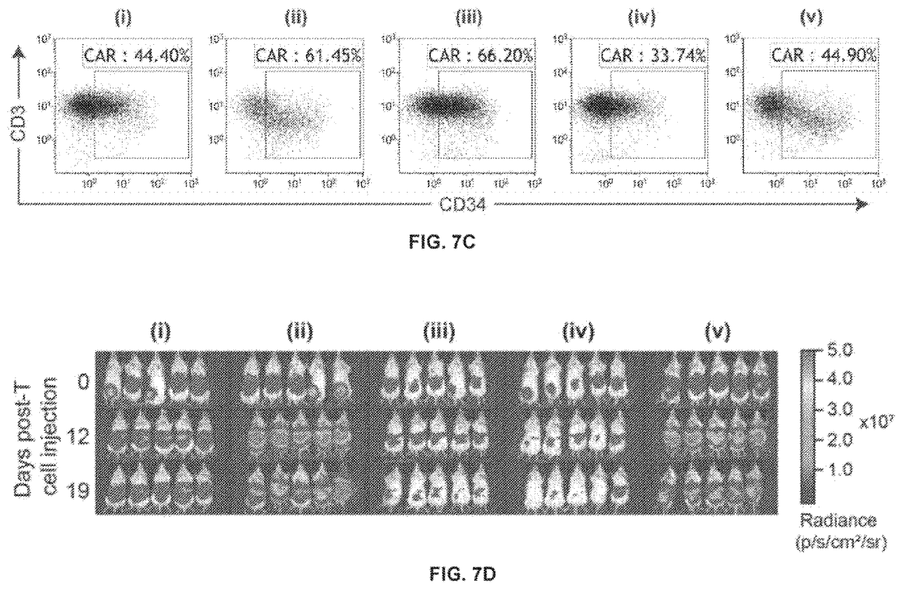

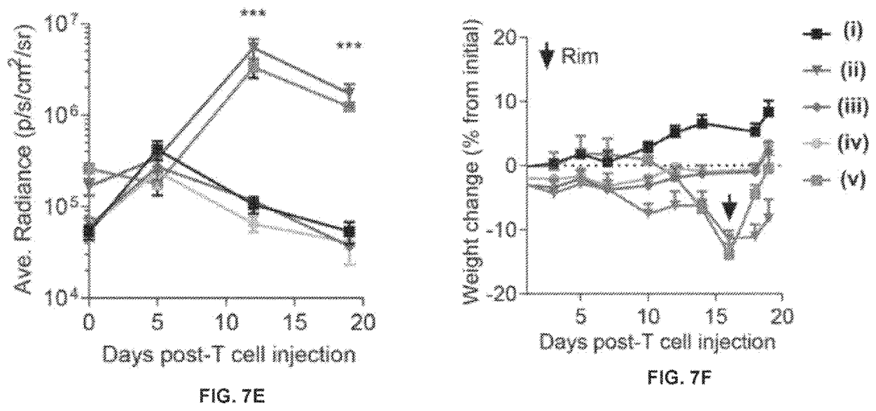

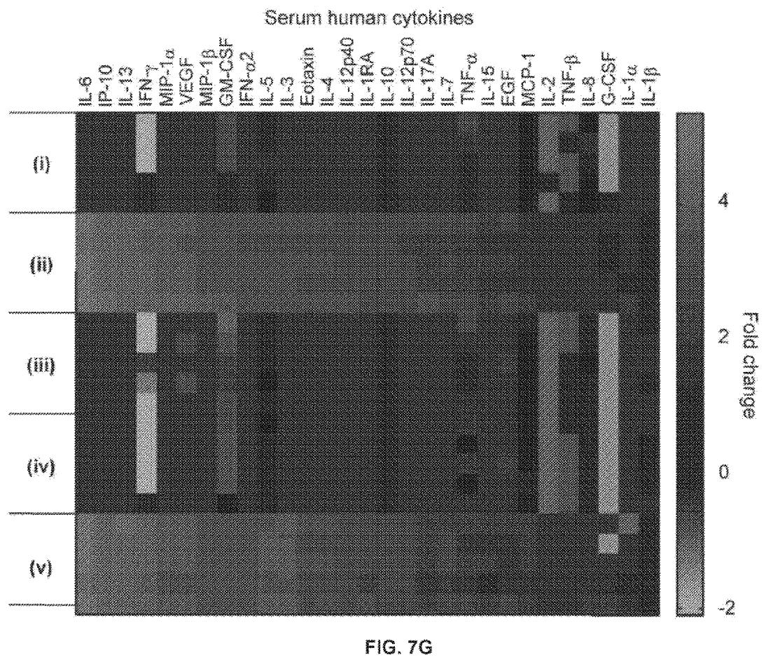

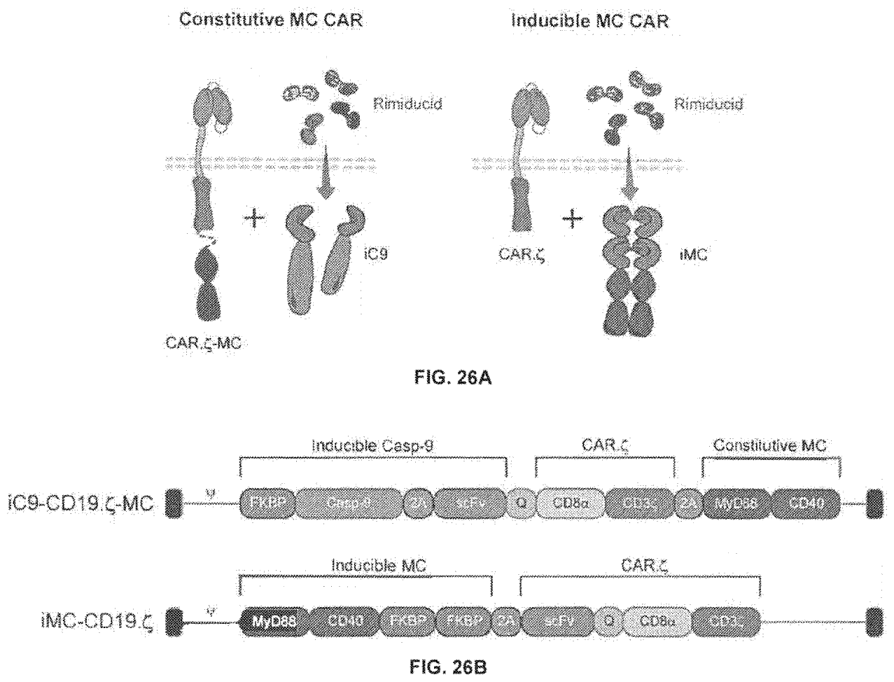

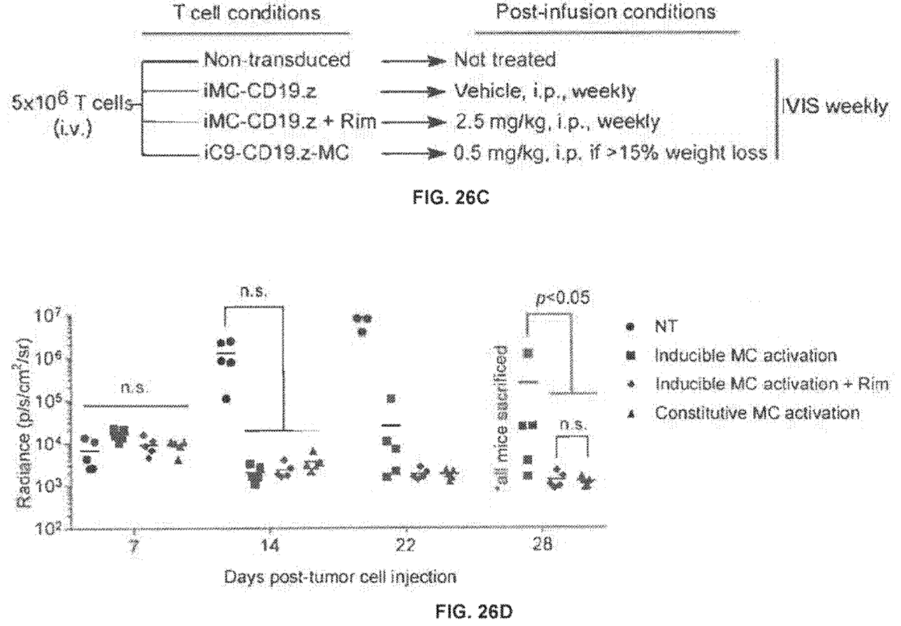

As shown in FIG. 3D, inefficient 2A cleavage appeared to result in MC association with some CAR molecules. Additional constructs using GSG-linked 2A sequences (GSG linker) (15,17) to more efficiently separate MC from the CAR were analyzed, as well as constructs where MC was positioned in the first position, 5′ of the CAR, to remove the possibility of intracellular attachment to the CD3-chain (FIG. 7A). In addition, basal signaling resulting from the juxtaposition of MC to the membrane was assayed by including a myristoylation-targeting domain to increase inner membrane association (18). Basal cytokine production from transduced T cells was assayed. Cytokine analysis showed that improved GSG-linked 2A cleavage and moving MC to the 5′ position dramatically reduced basal IFN-γ and IL-6 production, while partial CAR attachment (in iC9-CD19.ζ-MC) and membrane-associated MC (Myr-MC) revealed high levels of cytokine secretion (FIG. 7B). Interestingly, when using CAR T cells co-modified with EGFPluc to measure T cell levels in vivo, high tonic signaling was associated with rapid expansion at days 12 (˜4-fold; p<0.005) and 19 (˜8-fold; p<0.001) post-CAR-T injection (FIGS. 7C and 7E). While high basal activity enhanced CAR-T expansion, it was also associated with cachexia which required rimiducid infusion to activate iC9 (FIG. 7F). The profile of CAR-T-produced human cytokines in these animals showed that iC9-CD19.ζ-MC and MyrMC-iC9-CD19.ζ-modified T cells produced high levels of a diverse number of pro-inflammatory cytokines compared to constructs with low basal CAR-T activity (FIG. 7G). In addition, a comparison to an inducible MC system (i.e., iMC [Foster 2017; Mata 2017]), using the CD19+ Raji tumor model, indicates that high basal activity is necessary for prolonged anti-tumor efficacy (FIG. 26). Together, these data suggest that basal activation can enhance CAR-T proliferation in vivo and anti-tumor activity, but that cytokine production from rapidly proliferating T cells can cause undesired side-effects.

Selection of CAR-Modified T Cells Reduces Cytoxicity

Pre-clinical studies demonstrated that T cells transduced with SFG-iC9-CAR.ζ-MC targeting a variety of antigens (e.g., CD19, Her2 and PSCA) showed higher levels of CAR-T proliferation and killing tumor cell lines. In addition, iC9-CAR.-MC-modified T cells also produced higher levels of cytokines, including IFN-γ, IL-6 and TNF-α. In animal models, iC9-CAR.ζ-MC-modified T cells showed efficacy against both hematological and solid tumor cell lines. However, these highly active CAR-T cells also caused toxicity in mice, characterized by acute weight loss. This toxicity could be abrogated by injection of rimiducid (0.1 to 5 mg/kg, intraperitoneal (i.p.) injection) without affecting long-term tumor control. The likelihood of cachexia was reduced by enrichment of the modified cell population to obtain a higher percentage or ratio of CD8⁺ T cells before administration of the cells to the tumor-bearing mice. Enrichment for CD8⁺ CAR-T cells reduced cytokine related toxicities while preserving anti-tumor efficacy.

CD8 Selection of iC9-CD19.ζ-MC-Modified T Cells Abrogates Toxicity by Reducing Cytokine Production

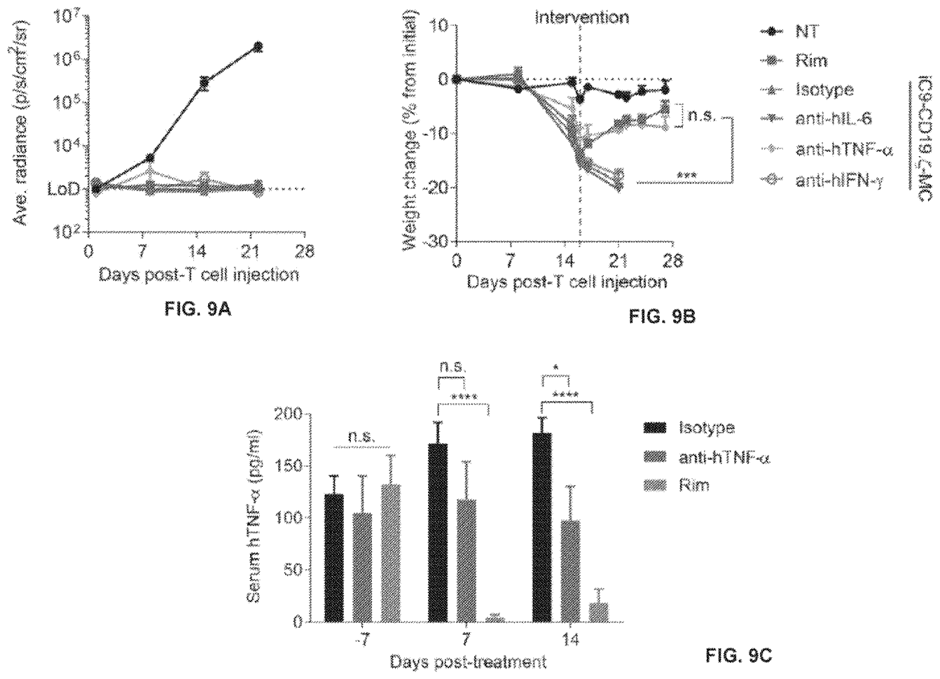

To further study cachexia associated with administration of iC9-CAR.-MC-modified T cells, the CD19-redirected construct was assayed against CD19⁺ Daudi tumors in vivo, and neutralizing antibodies targeting human IL-6, IFN-γ and TNF-α, all of which are cross-reactive with murine cytokine receptors, were administered, and followed by monitoring of mouse weight loss. Here, tumor-bearing mice were treated with 5×10⁶ iC9-CD19.ζ-MC transduced T cells and following >10% weight loss, intervention with either a single i.p. dose of rimiducid (0.5 mg/kg) or vehicle, or twice weekly injections of 100 ug per mouse anti-hIFN-γ, hIL-6 or hTNF-α was initiated (FIG. 9A). Interestingly, only anti-hTNF-α treatment was able to protect mice from further health decline to the same level of protection as activating the iC9 safety switch (FIG. 9B). 5×10⁶ iC9-CD19.ζ-MC-modified T cells were injected in to Daudi-bearing NSG mice and then treated with 0.5 mg/kg rimiducid after >10% weight loss or with i.p. injections with 100 mg twice per week with an isotype antibody (control) or with neutralizing antibodies against human TNF-α, IL-6 and IFN-γ. Weight recovery was monitored until day 28. The protection by anti-hTNF-α treatment from further weight decline was associated with only a modest, non-significant reduction in serum hTNF-α levels consistent with blockade of ligand-receptor interactions rather than mediating the clearance of antibody-bound hTNF-α (FIG. 9C). In contrast, activation of iC9 with rimiducid significantly reduced serum concentrations of hTNF-α. Like the use of iC9, control of toxicity with anti-hTNF-α did not affect antitumor activity of the CAR-T therapy (FIG. 9C). Thus, cytokine blockade provides a second effective mechanism to resolve the toxicity of this potent approach.

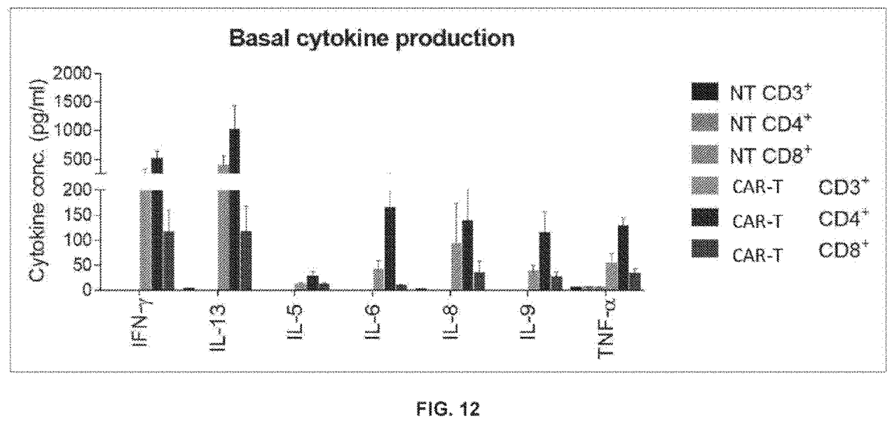

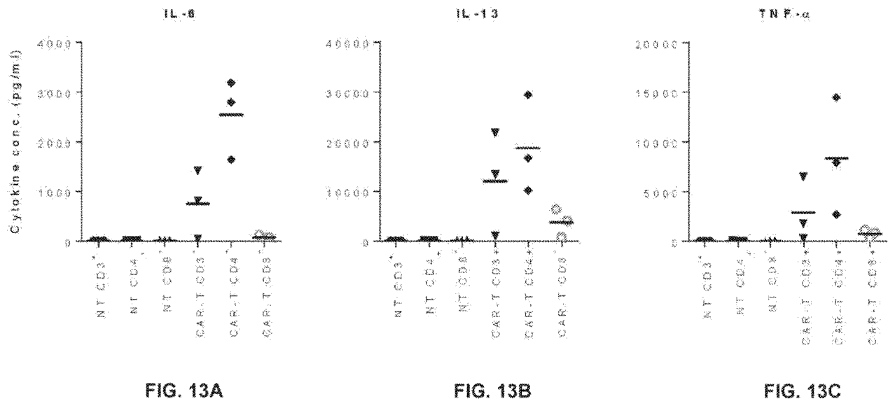

As T cell subsets can have different properties, we speculated that subset purification might provide a third avenue for controlling toxicity. CD4⁺ T cells are known for producing high levels of pro-inflammatory cytokines following activation following antigen recognition. Our studies also show that CD4⁺ T cells secreted high levels of IFN-α (IFN-γ), IL-13, IL-6, IL-8, IL-9 and TNF-α (TNF-α) (FIG. 12). Basal cytokine secretion levels were determined in the different cell populations.

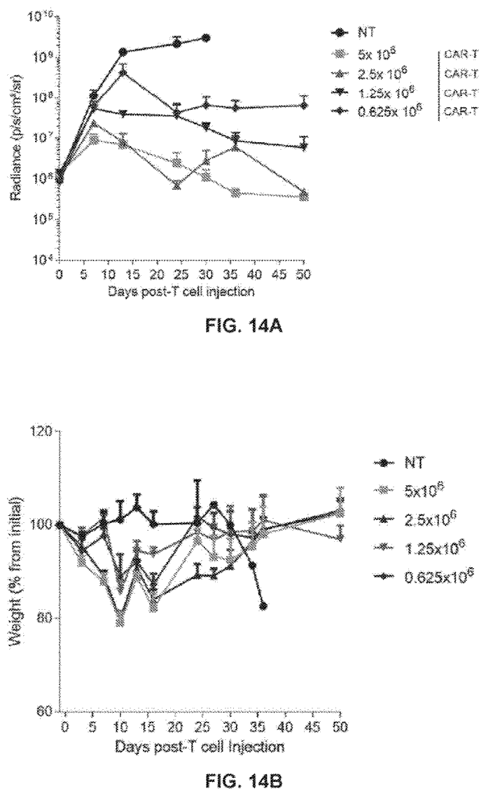

CD4⁺ T cells secreted higher levels of IFN-g (IFN-γ), IL-13, IL-6, IL-8, IL-9 and TNF-α (TNF-α) than CD8⁺ T cells or non-selected CAR-T cells (FIG. 12). In co-culture assays, CD19-specific (iC9-CD19.ζ-MC) CD4⁺ produced high levels of IL-6, IL-13 and TNF-α compared to CD8-selected iC9-CD19.ζ-MC-modified T cells (FIG. 14). CD8-selected, iC9-CD19.ζ-MC-modified T cells produced low levels of TNF-α, but retained cytotoxic activity against CD19⁺ tumor cells (FIG. 14). These data suggested that selecting CD8⁺ iC9-CAR.-MC-modified T cells may preserve anti-tumor efficacy while avoiding toxicity caused by cytokines produced by CAR-T cells.

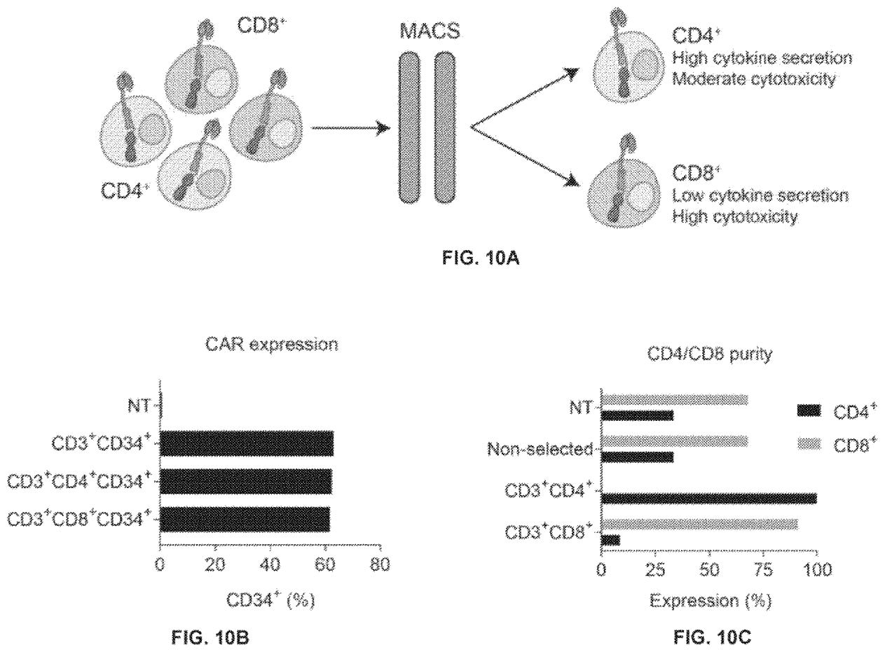

Because TNF-α, and possibly other cytokines contributed to cachexia following i.v. injection of iC9-CD19.ζ-MC-modified T cells, selection of CD8⁺ T cells (or depletion of CD4⁺ cells) was tested to determine if it could lessen toxicity while preserving antitumor activity. Here, non-transduced and CAR-modified T cells were purified into CD4⁺ and CD8⁺ T cells using magnetic bead selection (FIG. 10A).

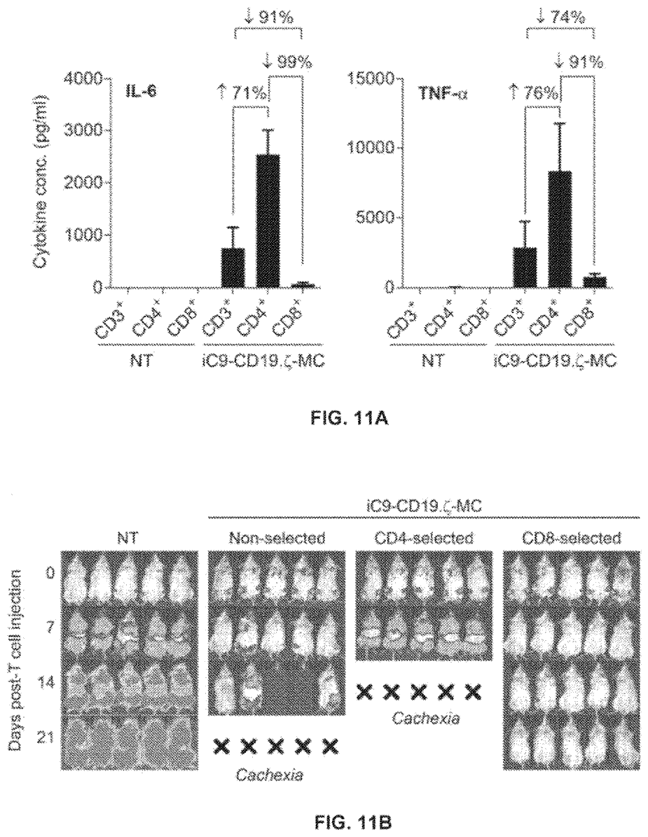

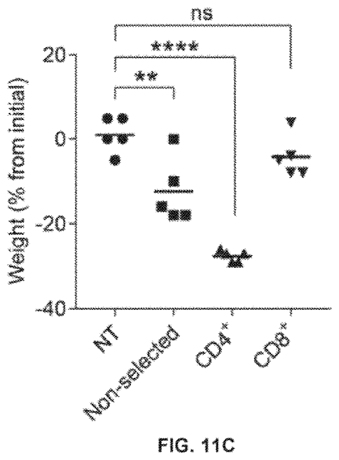

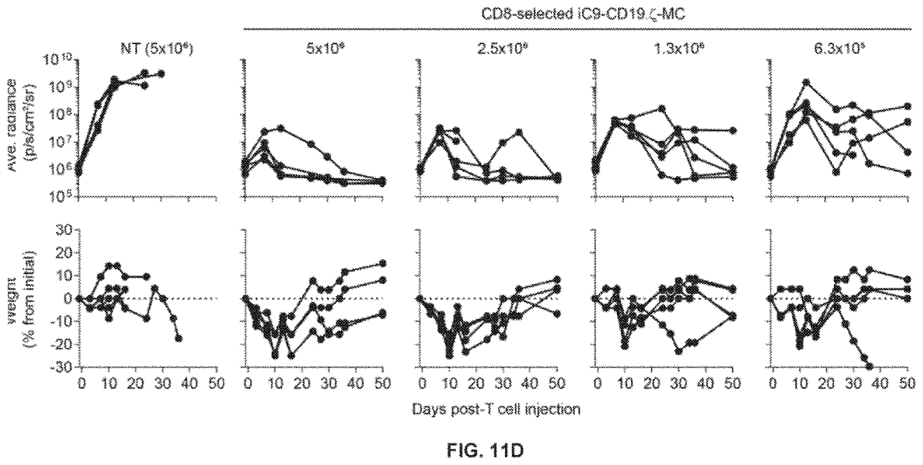

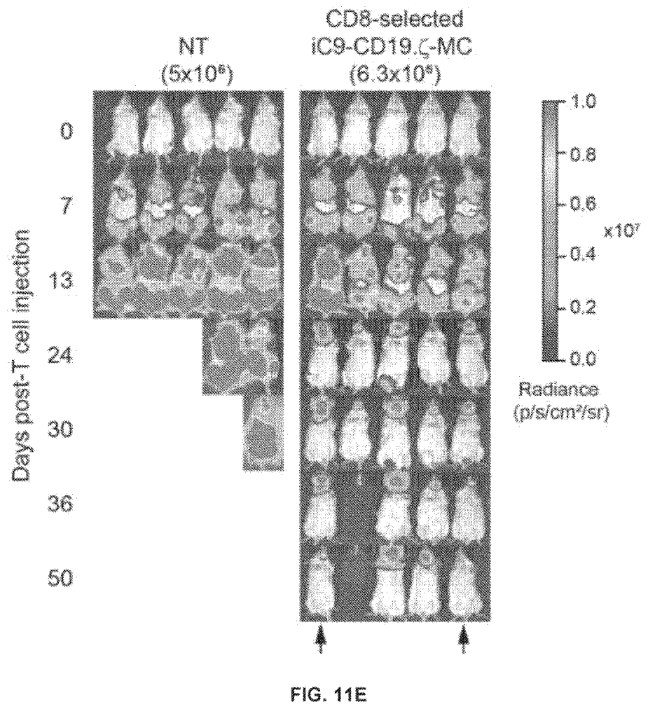

Non-selected and selected T cells were tested for purity and transduction efficiency. Whereas non-selected CAR-T cells had a CD4:CD8 ratio of 1:2, following selection they were 99% and 90% for CD4 and CD8-selected T cells, respectively (FIG. 10B). iC9-CD19.ζ-MC transduction was equivalent in both selected and non-selected gene-modified T cells (˜62% CD3⁺ CD34⁺) (FIG. 10B). Coculture assays against Raji tumor cells was performed, IL-6 and TNF-α production were measured at 48 hours. CD4-selected CAR-T cells produced 71% and 76% higher production of IL-6 and TNF-α compared to unselected CAR-T cells, whereas CD8-selected CAR-T cells produced 99% and 91% less of these molecules, respectively (FIG. 11A). To test whether this modification could reduce cachexia, non-transduced, non-selected, CD4 or CD8-enriched iC9-CD19.ζ-MC-modified T cells were administered to Raji-EGFPluc-bearing NSG mice. The results showed that non-selected and CD4-enriched CAR-T cells showed improved tumor control over NT T cells (FIG. 11B), however, these mice rapidly developed cachexia by day 7 post-CAR-T injection (FIG. 11C). In contrast, CD8-selected CAR-T cells demonstrated superior tumor control with minimal concomitant weight loss (FIG. 11B and FIG. 11C). A dose-titration was performed with CD8-enriched modified T cells using the same animal model. Here, high doses (>2.5×10⁶ cells) rapidly controlled tumor outgrowth (FIG. 11D). While these animals did show some evidence of cachexia, iC9 activation with rimiducid was not required and all animals recovered approximately 2-3 weeks post CAR-T injection (FIG. 11D). Treatment with lower doses of CD8-enriched CAR-T cells also showed tumor control, albeit with slower tumor elimination kinetics (FIG. 11D). Importantly, as few as 6.3×10⁵ CD8 cells controlled high level tumor burden with durable efficacy (FIG. 11E). These experiments suggest that CD8-enriched iC9-CD19.ζ-MC-modified T cells have potent antitumor efficacy with reduced cytokine-associated toxicity and may be helper T cell-independent.

Discussion

This Example describes an empirically discovered CAR architecture that utilizes high basal CAR signaling and costimulation (i.e., “always on” CAR) to drive T cell proliferation and anti-tumor activity against aggressive CD19+ and CD123+ lymphoma and leukemia cell lines. However, CAR-T cells using constitutively active MC produced high levels of cytokines (i.e., IFN-γ, TNF-α and IL-6) which required the use rimiducid to resolve toxicity in animal model where rimiducid could be titrated to “partially” eliminate CAR-T cells preserving long-term antitumor efficacy. In addition, recognition that CAR-T secreted cytokines were responsible for cachexia, we focused on the selection of CD8+ effector T cells which resulted in lower levels of toxicity with increased antitumor effects in a CD4+ helper-independent manner.

Initially, it was attempted to express MC in cis with CD3ζ, analogous to CARs using conventional costimulatory domains such as CD28 and 4-1BB. However, MyD88 appeared to destabilize the CAR, lowering surface expression and decreasing in vivo antitumor activity (FIG. 1). The inventors subsequently expressed MC as a constitutive protein to provide continuous costimulation to CD19-specific CAR-T cells. This resulted in the restoration of CAR surface expression on modified T cells and improved tumor activity (FIG. 3). Western blot analyses revealed additional MC species indicative of formation of fusion proteins, potentially caused by inefficient 2A skipping between CAR.ζ and the MC molecule. We hypothesize that ligation of MC to fraction of CAR molecules induces a signaling cascade that is responsible for basal activity, but also CAR potency. Indeed, the addition of a GSG linker to the 2A to increase transgene protein separation curtails basal cytokine secretion, but also abolished in vivo CAR-T proliferation (FIG. 7). Tethered to CD3ζ, MyD88/CD40 may act as a scaffold to recruit other signaling proteins (e.g., interleukin-1 receptor associated kinase (IRAK) family) as a MyDDosome complex to induce basal signaling (19-22). Alternatively, tonic signaling from scFv, amplified by MyD88/CD40, could result in constitutive stimulation (23).

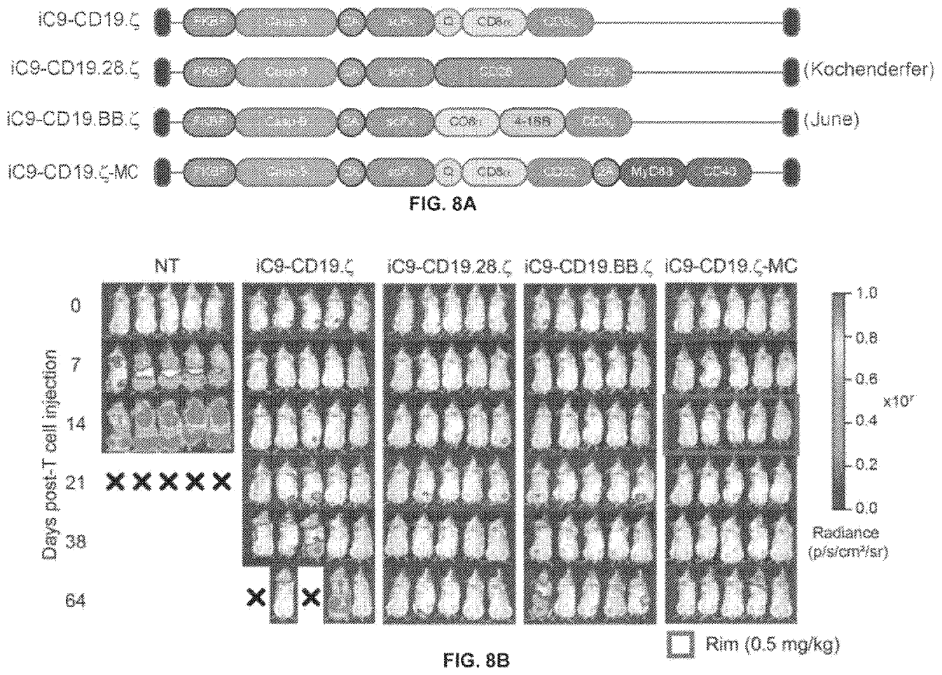

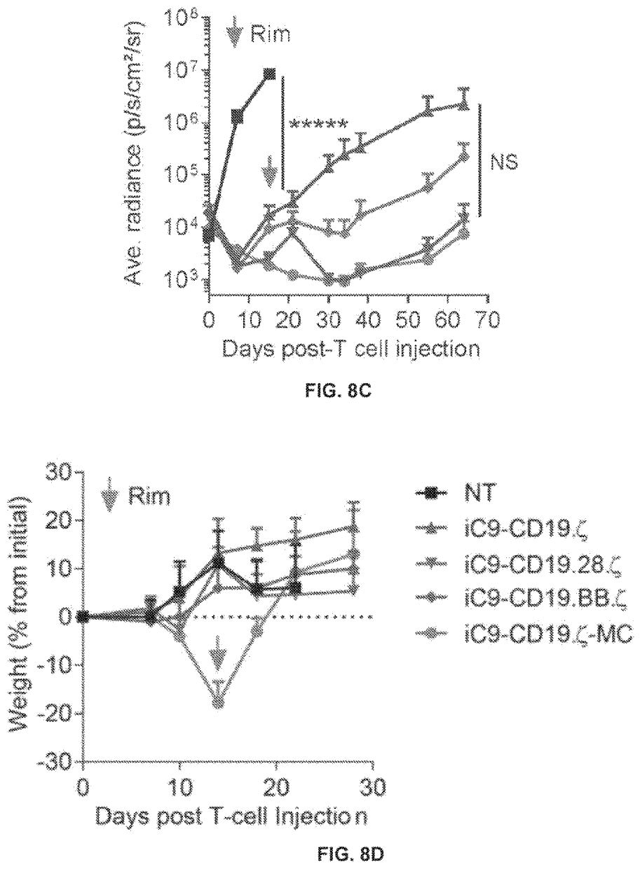

Unlike previous reports, of the deleterious effects of constitutive CAR signaling,MC costimulation did not appear to induce CAR-T exhaustion (23, 24). Indeed, MC-enabled CAR-T cells could proliferate for more than 3 months without loss of cytotoxic function, IL-2 production, and importantly, responsiveness to iC9-mediated apoptosis. Long and colleagues showed that some CAR costimulatory domains, such as 4-1BB, were protective against cellular exhaustion derived from tonic signaling (23). Others have shown, however, that 4-1BB can contribute to FAS-dependent cell death under tonic CAR conditions (25). In contrast, MC appears to phosphorylate a broad and unique set of signaling pathways. In addition to signaling through NF-κB (5,6), MC activates Akt, which has been shown to enhance survival and proliferation of CAR-T cells (26). Additional signaling nodes (e.g., AP-1, MAPK and IRF) may also contribute to enhanced function. Our (FIG. 8 and (5)) and other observations (6) suggests that MC may be a more potent driver of CAR-T activity than CD28 or 4-1BB. Whether MyD88/CD40 overcomes the limitations of conventional costimulatory molecules in T cells expressing constitutively active CARs needs further investigation.

Highly active T cell therapies are at risk for cytokine-related toxicities, which can be amplified further in patients with high tumor burden (27). In this study, constitutive MC signaling in CAR-T cells resulted in acute cachexia following infusion, which was not specific to the CAR target (i.e., CD19 or CD123), nor in the time-frame typically seen with xenogeneic graft-versus-host disease. However, toxicity could be mitigated by activation of iC9 following a single injection of rimucid. As previously demonstrated, titration of rimiducid resulted in partial elimination of MC-enabled CAR-T cells without loss of anti-tumor activity (16). Use of neutralizing blocking antibodies revealed that TNF-α decreased CAR-T-related toxicity suggesting that depletion of cell subsets that produce high level of pro-inflammatory cytokines (i.e., CD4+ T helper cells) could improve the therapeutic window for using a constitutive, MC-enabled CAR-T cell therapy. Indeed, purification of CD8+ T cells resulted in improved efficacy with minimal cytokine related toxicity and did not require the use of rimiducid to salvage animals. Interestingly, MC appeared to support the expansion of CAR-T cells in a CD4+ helper-independent manner suggesting that in a clinical application purification of CD8+ T cells might decrease cytokine release syndrome and without the inclusion of putative regulatory CAR-T cells (28). Since the animal models used did not contain human-derived myeloid cells, further investigation of iC9-CD19.ζ-MC CAR T cells using recently described preclinical models of cytokine release syndrome would yield additional insight into the utility of this strategy to mitigate potential toxicity in patients (29, 30). Overall, we identified a more efficacious CAR-T platform. Although the increased toxicity risk associated with this improved potency is expected, we also identified three approaches to mitigating that toxicity, T cell subset purification, neutralization of pro-inflammatory cytokines, and use of the iC9 safety switch.

In summary, constitutive MC costimulation provides CARs targeting CD19 or CD123 with long-term proliferative potential and high anti-tumor efficacy in animal models of lymphoma and myeloid leukemias, respectively. MC-enabled CAR-T cells exhibit substantial basal activity and are associated with cytokine-related toxicities in immune deficient mice, but this can be managed by deployment of the iC9 safety switch with rimiducid or by selecting T cell subsets with the propensity for lower cytokine secretion.

The following publications are cited in this example, or may provide supporting material.

(1) June C H, Sadelain M. N Engl J Med. 2018; 379:64-73. (2) Park J H, et al. N Engl J Med. 2018; 378:449-59. (3) Maude S L, et al. N Engl J Med. 2018; 378:439-48. (4) Neelapu S S, et al. N Engl J Med. 2017; 377:2531-44. (5) Foster A E, et al. Mol Ther. 2017; 25:2176-88. (6) Mata M, et al. Cancer Discov. 2017; 7:1306-19. (7) Narayanan P, et al. J Clin Invest. 2011; 121:1524-34. (8) Straathof K C, et al. Blood. 2005; 105:4247-54. (9) Zhou X, et al. Blood. 2015; 125:4103-13. (10) Philip B, et al. Blood. 2014; 124:1277-87. (11) Du X, et al. J Immunother. 2007; 30:607-13. (12) Mardiros A, et al. Blood. 2013; 122:3138-48. (13) Milone M C, et al. Mol Ther. 2009; 17:1453-64. (14) Kochenderfer J N, et al. Blood. 2010; 116:4099-102). (15) Chng J, et al. MAbs. 2015; 7:403-12. (16) Diaconu I, et al. Mol Ther. 2017; 25:580-92. (17) Hofacre A, et al. Hum Gene Ther. 2018; 29:437-51. (18) Hanks B A, et al. Nat Med. 2005; 11:130-7. (19) Motshwene P G, et al. J Biol Chem. 2009; 284:25404-11. (20) Lin S-C, et al. Nature. 2010; 465:885-90. (21) Wang L, et al. Proc Natl Acad Sci USA. 2017; 114:13507-12. (22) De Nardo D, et al. J Biol Chem 293: 15195 et seq., 2018. (23) Long A H, et al. Nat Med. 2015; 21:581-90. (24) Frigault M J, et al. Cancer Immunol Res. 2015; 3:356-67. (25) Gomes-Silva D, et al. Cell Rep. 2017; 21:17-26. (26) Sun J, et al. Mol Ther. 2010; 18:2006-17. (27) Neelapu S S, et al. Nat Rev Clin Oncol. 2018; 15:47-62. (28) Lee J C, et al. Cancer Res. 2011; 71:2871-81. (29) Norelli et al. Nat Med. 2018; 24:739-48. (30) Giavridis et al. Nat Med. 2018; 24:731-38

### Example 2: Modified Her2/Neu Directed CAR-T Cells

To determine if CD8-selection to producing modified cell populations of iC9-CAR.-MC-expressing T cells could be applied to other CARs targeting solid tumor antigens, animal studies were conducted using a Her2-specific CAR construct.

T cells were transduced with the SFG-iC9-Her2.ζ-MC vector, and after 5 days measured for CAR expression using the CD34 epitope. Our results show that T cells could be efficiently transduced with iC9-Her2.-MC, with >70% expression of the CAR molecule (FIG. 15). As can be seen in FIG. 15A NT do not express the CAR molecule, where FIG. 15B shows that T cells transduced with SFG-iC9-Her2.ζ-MC are 70.3% CAR positive.

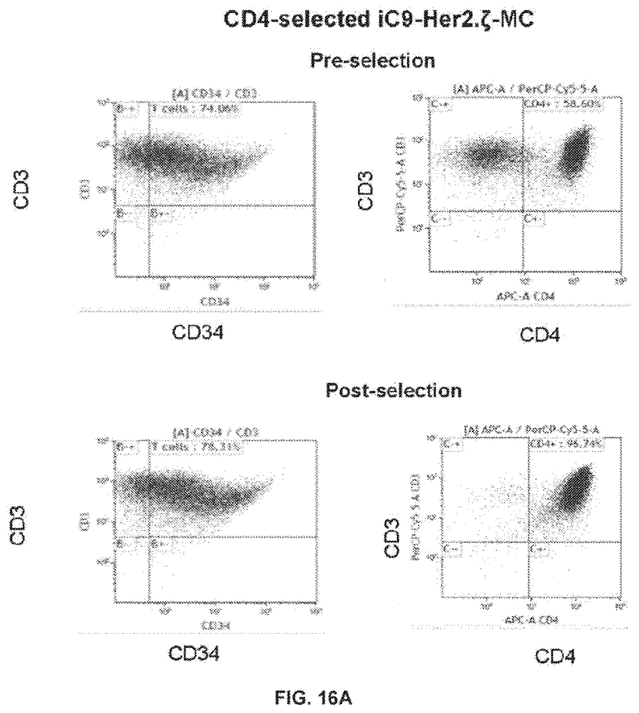

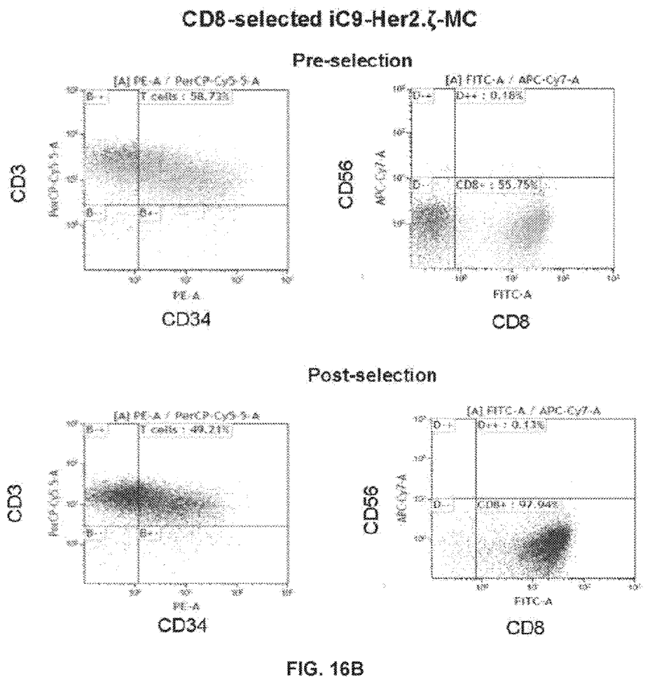

CAR-modified T cells were then selected for CD4⁺ or CD8⁺ T cell subsets to generate highly purified iC9-Her2.ζ-MC-modified T cells (FIG. 16). iC9-Her2.-MC-transduced T cells were measured for CD4⁺ and CD8⁺ T cell frequency. Subsequently, gene-modified T cells were selected for either CD4⁺ or CD8⁺ T cells using magnetic beads and MACS columns. After 4 days, CD4-selected (FIG. 16A) and CD8-selected (FIG. 16B) T cells were measured by fluorescence activated cell sorting for purity of the respective populations

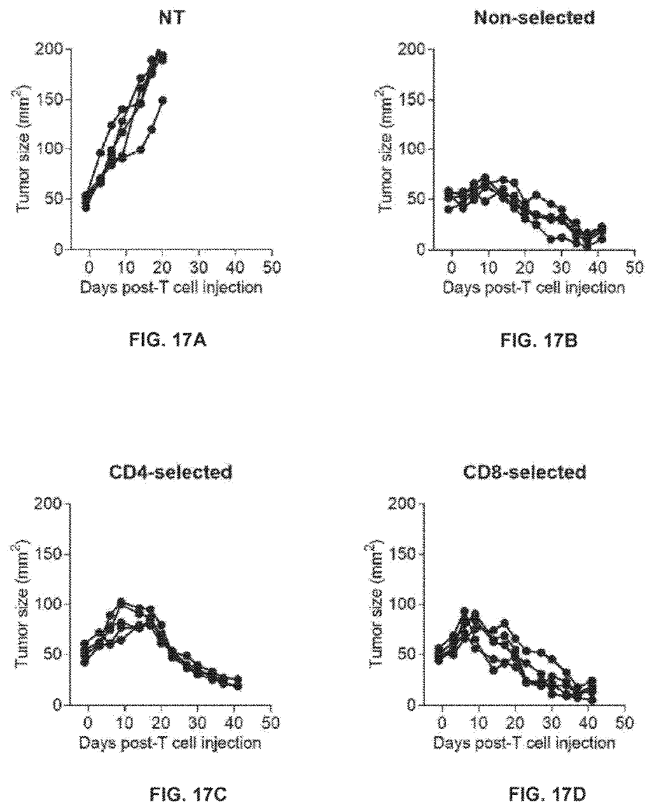

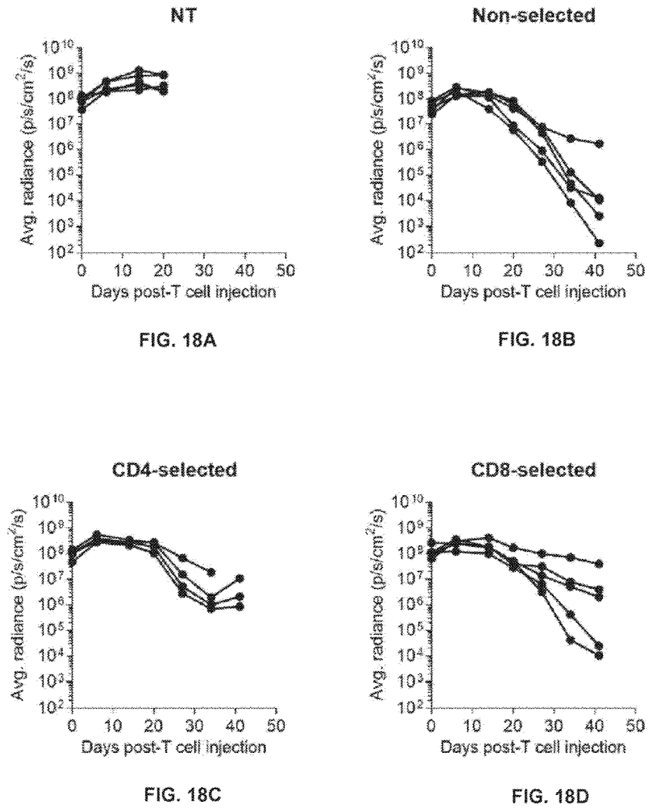

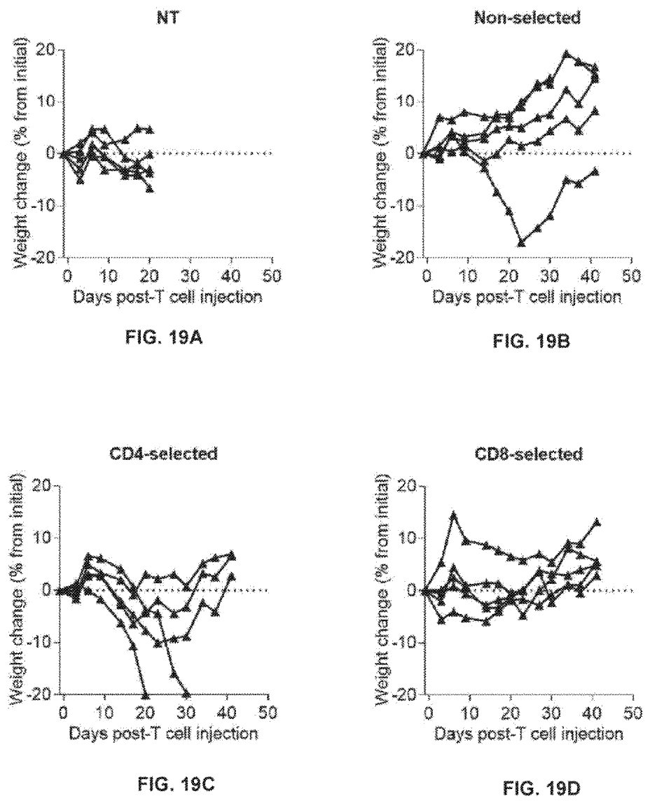

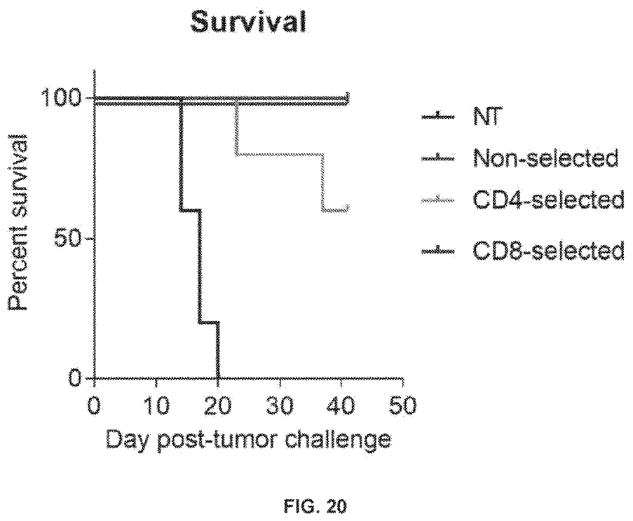

NSG mice were engrafted with Her2⁺ HPAC-EGFPluc tumor cells by subcutaneous injection. After 7 days, mice were treated with an intravenous injection of 5×10⁶ NT, non-selected, CD4-selected or CD8-selected iC9-Her2.ζ-MC-modified T cells. Tumor size was measured by calipers for 41 days post-T cell injection (FIG. 17) or by in vivo bioluminescence imaging (IVIS) by injection of the substrate D-luciferin for 41 days post-T cell injection (FIG. 18). HPAC tumor cells were efficiently controlled by all CAR-T modified cell types (FIGS. 17 and 18). However, as observed in the CD19 studies, CD4-selected iC9-Her2.ζ-MC-modified T cells showed higher rates of cachexia resulting in death in 2/5 mice (FIGS. 19 and 20).

FIG. 20 shows the survival of mice following treatment with selected modified CAR-T cells. Survival was graphed where all mice treated with NT T cells died due to tumor growth and 2 mice died in the CD4-selected group due to weight loss/cachexia.

### Example 3: Modified PSCA-Directed CAR-T Cells

To determine if CD8-selection to produce modified cell populations of iC9-CAR.-MC-expressing T cells could be applied to other CARs targeting solid tumor antigens, animal studies were conducted using a prostate stem cell antigen (PSCA)-specific CAR construct.

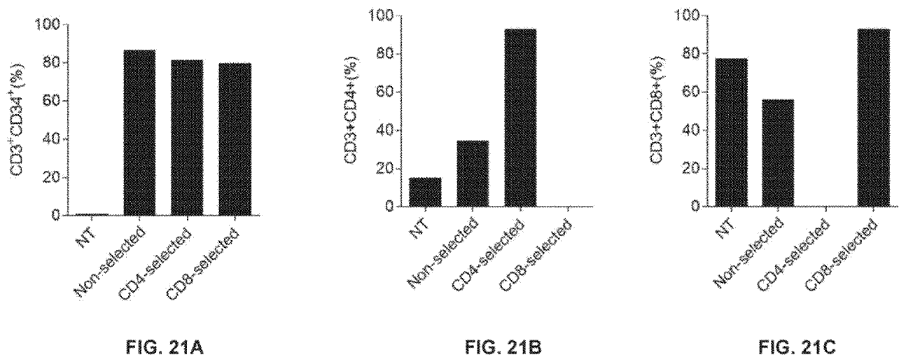

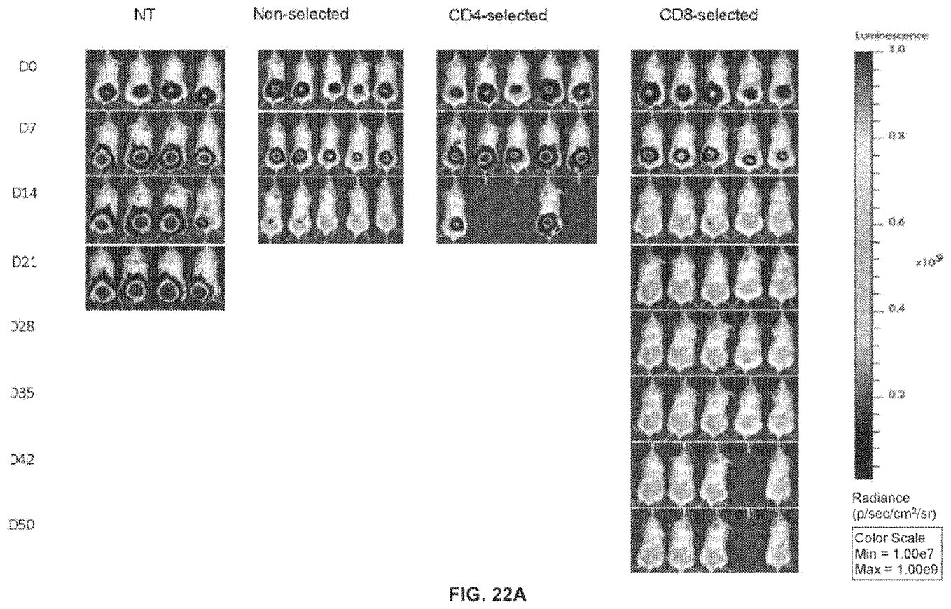

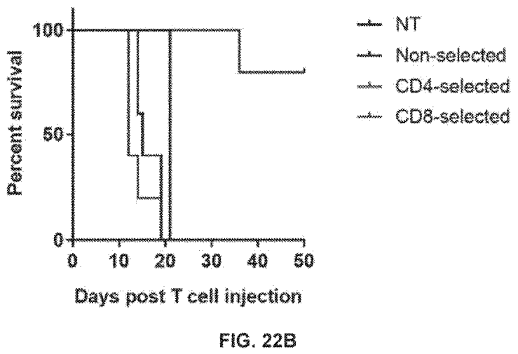

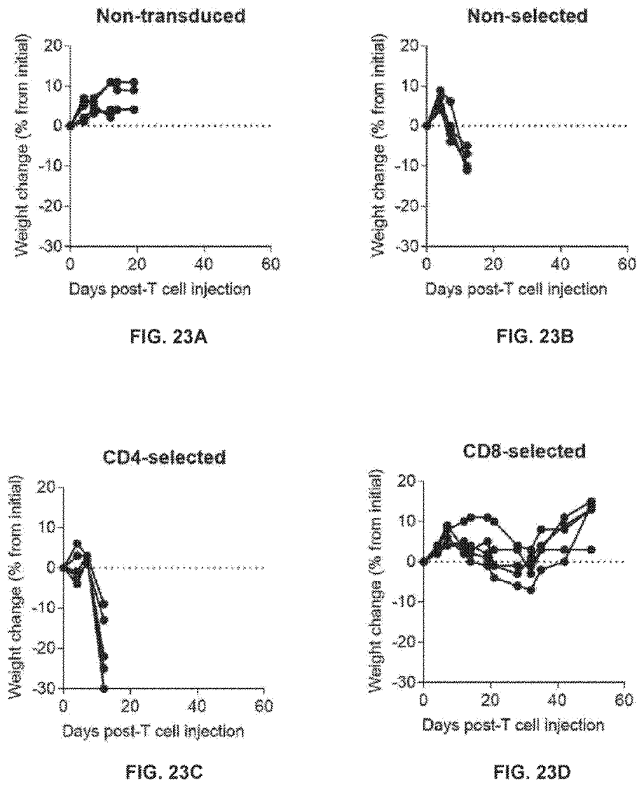

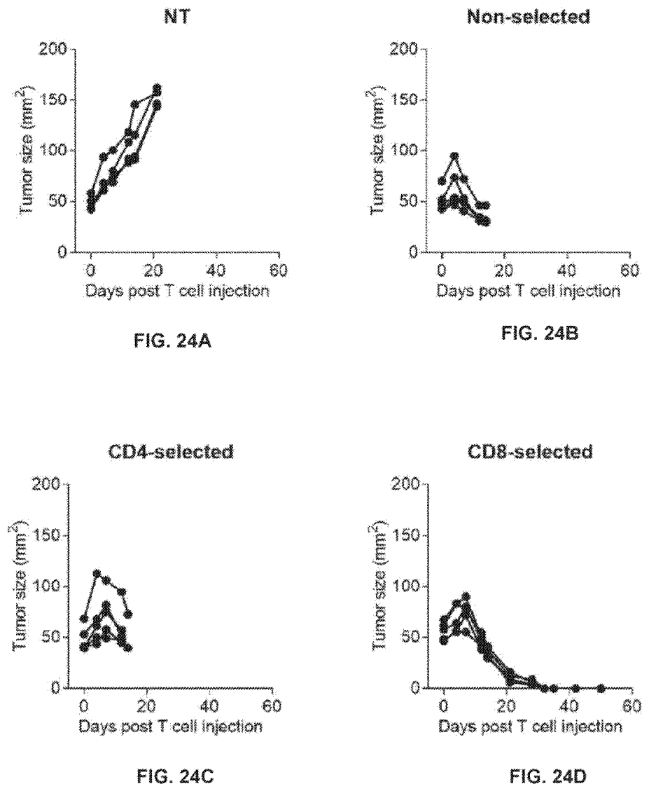

T cells could be efficiently transduced with a PSCA-directed CAR (iC9-PSCA.ζ-MC) and purified for CD4⁺ or CD8⁺ T cells (FIG. 21). Using the HPAC-EGFPluc tumor model, which also expresses high levels of PSCA, mice treated with NT failed to control tumor, whereas non-selected and CD4-selected iC9-PSCA.ζ-MC-modified T cells rapidly induced cachexia and death in NSG tumor-bearing animals (FIGS. 21-24). However, CD8-selected iC9-PSCA.ζ-MC-modified T cells can eliminate tumor while having minimal impact on weight loss and mouse health.

Cumulatively, data obtained using the CD19, Her2, and PSCA vectors suggest that the CD4⁺ T cell subset is responsible for the high cytokine production observed in iC9-CAR.ζ-MC-modified T cells, and that cytokines such as TNF-α, are responsible for the toxicity observed in NSG tumor models. Purification of CD8⁺ CAR-T cells preserves the anti-tumor effects against CD19, Her2 and PSCA positive cell lines while minimizing cytokine-related toxicities.

### Example 4: Nucleic Acid and Amino Acid Sequences

TABLE 3
Amino Acid Sequences

| SEQ | | |
|---|---|---|
| ID | | |
| NO: | PROTEIN | AMINO ACID SEQUENCE |
| 1 | Fᵥ | GVQVETISPGDGRTFPKRGQTCVVHYTGMLEDGKKVDSSRDRNKPFKFMLGKQEVIRGWEEG |
| | Human | VAQMSVGQRAKLTISPDYAYGATGHPGIIPPHATLVFDVELLKLE |
| | FKBP12v36 | |
| 3 | 8-amino acid | SGGGSGVD |
| | linker | |
| 5 | Human | GFGDVGALESLRGNADLAYILSMEPCGHCLIINNVNFCRESGLRTRTGSNIDCEKLRRRFSSLHF |
| | Δcaspase9 | MVEVKGDLTAKKMVLALLELARQDHGALDCCVVVILSHGCQASHLQFPGAVYGTDGCPVSVEKI |
| | | VNIFNGTSCPSLGGKPKLFFIQACGGEQKDHGFEVASTSPEDESPGSNPEPDATPFQEGLRTFD |
| | | QLDAISSLPTPSDIFVSYSTFPGFVSWRDPKSGSWYVETLDDIFEQWAHSEDLQSLLLRVANAV |
| | | SVKGIYKQMPGCFNFLRKKLFFKTSASRAP |
| 7 | T2A | EGRGSLLTCGDVEENPGP |
| | polypeptide | |
| 9 | Signal | MEFGLSWLFLVAILKGVQCSR |
| | peptide | |
| 11 | FMC63 VL | DIQMTQTTSSLSASLGDRVTISCRASQDISKYLNVVYQQKPDGTVKLLIYHTSRLHSGVPSRFSGS |
| | (anti-CD19) | GSGTDYSLTISNLEQEDIATYFCQQGNTLPYTFGGGTKLEIT |
| 13 | Glycine- | GGGSGGGG |
| | serine linker | |
| 15 | FMC63 VH | EVKLQESGPGLVAPSQSLSVTCTVSGVSLPDYGVSWIRQPPRKGLEWLGVIWGSETTYYNSAL |
| | (anti-CD19) | KSRLTIIKDNSKSQVFLKMNSLQTDDTAIYYCAKHYYYGGSYAMDYWGQGTSVTVSS |
| 17 | Human CD34 | ELPTQGTFSNVSTNVS |
| | epitope | |
| 19 | Human CD8 | PAPRPPTPAPTIASQPLSLRPEACRPAAGGAVHTRGLDFACD |
| | alpha stalk | |
| 21 | Human CD8 | IYIWAPLAGTCGVLLLSLVITLYCNHRNRRRVCKCPR |
| | trans- | |
| | membrane | |
| | region | |
| | between | |
| | FKBP12-1 | |
| | and FKBP12- | |
| | 2 in pM004 | |
| 23 | Portion of | RVKFSRSADAPAYQQGQNQLYNELNLGRREEYDVLDKRRGRDPEMGGKPRRKNPQEGLYNE |
| | human CD3ζ | LQKDKMAEAYSEIGMKGERRRGKGHDGLYQGLSTATKDTYDALHMQALPPR |
| | encoded by | |
| | pBP001 | |
| 25 | P2A | ATNFSLLKQAGDVEENPGP |
| | polypeptide | |
| 27 | Portion of | MAAGGPGAGSAAPVSSTSSLPLAALNMRVRRRLSLFLNVRTQVAADWTALAEEMDFEYLEIRQ |
| | huma MyD88 | LETQADPTGRLLDAWQGRPGASVGRLLDLLTKLGRDDVLLELGPSIEEDCQKYILKQQQEEAEK |
| | polypeptide | PLQVAAVDSSVPRTAELAGITTLDDPLGHMPERFDAFICYCPSDI |
| | encoded by | |
| | pBP001 | |
| 29 | Portion of | KKVAKKPTNKAPHPKQEPQEINFPDDLPGSNTAAPVQETLHGCQPVTQEDGKESRISVQERQ |
| | human CD40 | |
| | polypeptide | |
| | encoded by | |
| | pBP001 | |
| 31 | MLEMLEMLEMLE | |
| | linker | |
| | encoded 5′ of | |
| | FKBP12v36 | |
| | in pBP001 | |
| 33 | Human | MGVQVETISPGDGRTFPKRGQTCVVHYTGMLEDGKKFDSSRDRNKPFKFMLGKQEVIRGWEE |
| | FKBP12 | GVAQMSVGQRAKLTISPDYAYGATGHPGIIPPHATLVFDVELLKLE |
| | (GenBank no | |
| | AAA58476) | |
| 35 | Huma MyD88 | MAAGGPGAGSAAPVSSTSSLPLAALNMRVRRRLSLFLNVRTQVAADVVTALAEEMDFEYLEIRQ |
| | (Genbank no. | LETQADPTGRLLDAWQGRPGASVGRLLELLTKLGRDDVLLELGPSIEEDCQKYILKQQQEEAEK |
| | AAC50954) | PLQVAAVDSSVPRTAELAGITTLDDPLGHMPERFDAFICYCPSDIQFVQEMIRQLEQTNYRLKLC |
| | | VSDRDVLPGTCVWSIASELIEKRCRRMVVVVSDDYLQSKECDFQTKFALSLSPGAHQKRLIPIKY |
| | | KAMKKEFPSILRFITVCDYTNPCTKSWFWTRLAKALSLP |
| 37 | Human CD40 | MVRLPLQCVLWGCLLTAVHPEPPTACREKQYLINSQCCSLCQPGQKLVSDCTEFTETECLPCG |
| | (Genbank no. | ESEFLDTWNRETHCHQHKYCDPNLGLRVQQKGTSETDTICTCEEGWHCTSEACESCVLHRSC |
| | AAH12419) | SPGFGVKQIATGVSDTICEPCPVGFFSNVSSAFEKCHPVVTSCETKDLVVQQAGTNKTDVVCGP |
| | | QDRLRALVVIPIIFGILFAILLVLVFIKKVAKKPTNKAPHPKQEPQEINFPDDLPGSNTAAPVQETLH |
| | | GCQPVTQEDGKESRISVQERQ |
| 39 | Human CD3ζ | MKWKALFTAAILQAQLPITASSLPHPTQQSPEKKVLGPGGCTCRHNRFCNEAQSFGLLDPKLCY |
| | (GenBank no. | LLDGILFIYGVILTALFLRVKFSRSADAPAYQQGQNQLYNELNLGRREEYDVLDKRRGRDPEMG |
| | XP_016858290) | GKPRRKNPQEGLYNELQKDKMAEAYSEIGMKGERRRGKGHDGLYQGLSTATKDTYDALHMQA |
| | | LPPR |
| 41 | Homo | MDEADRRLLRRCRLRLVEELQVDQLWDALLSSELFRPHMIEDIQRAGSGSRRDQARQLIIDLET |
| | sapiens | RGSQALPLFISCLEDTGQDMLASFLRTNRQAAKLSKPTLENLTPVVLRPEIRKPEVLRPETPRPV |
| | caspase 9 | DIGSGGFGDVGALESLRGNADLAYILSMEPCGHCLIINNVNFCRESGLRTRTGSNIDCEKLRRRF |
| | (Genbank no. | SSPHFMVEVKGDLTAKKMVLALLELAQQDHGALDCCVVVILSHGCQASHLQFPGAVYGTDGCP |
| | BAA82697) | VSVEKIVNIFNGTSCPSLGGKPKLFFIQACGGEQKDHGFEVASTSPEDESPGSNPEPDATPFQE |
| | | GLRTFDQLDAISSLPTPSDIFVSYSTFPGFVSWRDPKSGSWYVETLDDIFEQWAHSEDLQSLLL |
| | | RVANAVSVKGIYKQMPGCFNFLRKKLFFKTS |
| 43 | MLE linker | MLE |
| 45 | FRP5 VH | EVQLQQSGPELKKPGETVKISCKASGYPFTNYGMNWVKQAPGQGLKWMGWINTSTGESTFAD |
| | (anti-Her2) | DFKGRFDFSLETSANTAYLQINNLKSEDMATYFCARWEVYHGYVPYWGQGTTVTVSS |
| 47 | FRP5 VL | DIQLTQSHKFLSTSVGDRVSITCKASQDVYNAVAWYQQKPGQSPKWYSASSRYTGVPSRFTG |
| | (anti-Her2) | SGSGPDFTFTISSVQAEDLAVYFCQQHFRTPFTFGSGTKLEIKAL |
| 49 | Linker | GGCGGTGGAGGCTCCGGTGGAGGCGGCTCTGGAGGAGGAGGTTCA |
| 51 | Myristoylation | MGSSKSKPKDPSQR |
| | domain | |
| 53 | A11 VL | DIQLTQSPSTLSASMGDRVTITCSASSSVRFIHWYQQKPGKAPKRLIYDTSKLASGVPSRFSGS |
| | (anti-PSCA) | GSGTDFTLTISSLQPEDFATYYCQQWGSSPFTFGQGTKVEIK |
| 55 | A11 VH | EVQLVEYGGGLVQPGGSLRLSCAASGFNIKDYYIHWVRQAPGKGLEWVAWIDPENGDTEFVPK |
| | (anti-PSCA) | FQGRATMSADTSKNTAYLQMNSLRAEDTAVYYCKTGGFWGQGTLVTVSS |
| 57 | bm2B3 VL | DIQLTQSPSSLSASVGDRVTITCSASSSVRFIHWYQQKPGKAPKRLIYDTSKLASGVPSRFSGSG |
| | (anti-PSCA) | SGTSYTLTISSLQPEDFATYYCQQWSSSPFTFGQGTKVEIK |
| 59 | bm2B3 VH | EVQLVESGGGLVQPGGSLRLSCAASGFNIKDYYIHWVRQAPGKGLEWIGWIDPENGDTEFVPK |
| | (anti-PSCA) | FQGKATMSADTSKNTAYLQMNSLRAEDTAVYYCKTGGFWGQGTLVTVSS |
| 61 | ΔCaspase 9 | VDGFGDVGALESLRGNADLAYILSMEPCGHCLIINNVNFCRESGLRTRTGSNIDCEKLRRRFSS |
| | D330E | LHFMVEVKGDLTAKKMVLALLELARQDHGALDCCVVVILSHGCQASHLQFPGAVYGTDGC |
| | | PVSVEKIVNIFNGTSCPSLGGKPKLFFIQACGGEQKDHGFEVASTSPEDESPGSNPEPDA |
| | | TPFQEGLRTFDQLeAISSLPTPSDIFVSYSTFPGFVSWRDPKSGSWYVETLDDIFEQWAH |
| | | SEDLQSLLLRVANAVSVKGIYKQMPGCFNFLRKKLFFKTSASRA |
| 63 | ΔCasp9 (res. | GFGDVGALESLRGNADLAYILSMEPCGHCLIINNVNFCRESGLRTRTGSNIDCEKLRRRFSSLHF |
| | 135-416) | MVEVKGDLTAKKMVLALLELARQDHGALDCCVVVILSHGCQASHLQFPGAVYGTDGCPVSVEKI |
| | D330A | VNIFNGTSCPSLGGKPKLFFIQACGGEQKDHGFEVASTSPEDESPGSNPEPDATPFQEGLRTFD |
| | N405Q | QLAAISSLPTPSDIFVSYSTFPGFVSWRDPKSGSWYVETLDDIFEQWAHSEDLQSLLLRVANAV |
| | | SVKGIYKQMPGCFQFLRKKLFFKTS |
| 65 | ΔCasp9 (res. | GFGDVGALESLRGNADLAYILSMEPCGHCLIINNVNFCRESGLRTRTGSNIDCEKLRRRFSSLHF |
| | 135-416) | MVEVKGDLTAKKMVLALLELARQDHGALDCCVVVILSHGCQASHLQFPGAVYGTDGCPVSVEKI |
| | N405Q | VNIFNGTSCPSLGGKPKLFFIQACGGEQKDHGFEVASTSPEDESPGSNPEPDATPFQEGLRTFD |
| | | QLDAISSLPTPSDIFVSYSTFPGFVSWRDPKSGSWYVETLDDIFEQWAHSEDLQSLLLRVANAV |
| | | SVKGIYKQMPGCFQFLRKKLFFKTS |
| 67 | ΔCasp9 (res. | GFGDVGALESLRGNADLAYILSMEPCGHCLIINNVNFCRESGLRTRTGSNIDCEKLRRRFSSLHF |
| | 135-416) | MVEVKGDLTAKKMVLALLELARQDHGALDCCVVVILSHGCQASHLQFPGAVYGTDGCPVSVEKI |
| | D330A | VNIFNGTSCPSLGGKPKLFFIQACGGEQKDHGFEVASTSPEDESPGSNPEPDATPFQEGLRTFD |
| | | QLAAISSLPTPSDIFVSYSTFPGFVSWRDPKSGSWYVETLDDIFEQWAHSEDLQSLLLRVANAV |
| | | SVKGIYKQMPGCFNFLRKKLFFKTS |
| 69 | ΔCD19 | MPPPRLLFFLLFLTPMEVRPEEPLVVKVEEGDNAVLQCLKGTSDGPTQQLTWSRESPLKPFLKL |
| | marker | SLGLPGLGIHMRPLAIWLFIFNVSQQMGGFYLCQPGPPSEKAWQPGWTVNVEGSGELFRWNV |
| | polypeptide | SDLGGLGCGLKNRSSEGPSSPSGKLMSPKLYVWAKDRPEIWEGEPPCLPPRDSLNQSLSQDL |
| | | TMAPGSTLWLSCGVPPDSVSRGPLSWTHVHPKGPKSLLSLELKDDRPARDMWVMETGLLLPR |
| | | ATAQDAGKYYCHRGNLTMSFHLEITARPVLWHWLLRTGGWKVSAVTLAYLIFCLCSLVGILHLQ |
| | | RALVLRRKRKRMTDPTRRF |
| 71 | OX40 | VAAILGLGLVLGLLGPLAILLALYLLRRDQRLPPDAHKPPGGGSFRTPIQEEQADAHSTLAKI |
| | cytoplasmic | |
| | Signaling | |
| | region | |
| 73 | 4-1BB | SVVKRGRKKLLYIFKQPFMRPVQTTQEEDGCSCRFPEEEEGGCEL |
| | cytoplasmic | |
| | Signaling | |
| | region | |
| 75 | CD28 | FWVLVVVGGVLACYSLLVTVAFIIFWVRSKRSRLLHSDYMNMTPRRPGPTRKHYQPYAPPRDF |
| | cytoplasmic | AAYRS |
| | Signaling | |
| | region | |
| 77 | Fv | GVQVETISPGDGRTFPKRGQTCVVHYTGMLEDGKKVDSSRDRNKPFKFMLGKQEVIRGWEEG |
| | | VAQMSVGQRAKLTISPDYAYGATGHPGIIPPHATLVFDVELLKLE |
| 79 | Fv′ | GVQVETISPGDGRTFPKRGQTCVVHYTGMLEDGKKVDSSRDRNKPFKFMLGKQEVIRGWEEG |
| | | VAQMSVGQRAKLTISPDYAYGATGHPGIIPPHATLVFDVELLKL |
| 81 | FKBP12 | GVQVETISPGDGRTFPKRGQTCVVHYTGMLEDGKKFDSSRDRNKPFKFMLGKQEVIRGWEEG |
| | Wild type | VAQMSVGQRAKLTISPDYAYGATGHPGIIPPHATLVFDVELLKLE |
| 83 | MyD88 | MAAGGPGAGSAAPVSSTSSLPLAALNMRVRRRLSLFLNVRTQVAADWTALAEEMDFEYLEIRQ |
| | Full length | LETQADPTGRLLDAWQGRPGASVGRLLELLTKLGRDDVLLELGPSIEEDCQKYILKQQQEEAEK |
| | | PLQVAAVDSSVPRTAELAGITTLDDPLGHMPERFDAFICYCPSDIQFVQEMIRQLEQTNYRLKLC |
| | | VSDRDVLPGTCVWSIASELIEKRCRRMVVVVSDDYLQSKECDFQTKFALSLSPGAHQKRLIPIKY |
| | | KAMKKEFPSILRFITVCDYTNPCTKSWFWTRLAKALSLP |
| 85 | ICOS | TKKKYSSSVHDPNGEYMFMRAVNTAKKSRLTDVTL |
| | Signaling | |
| | domain | |
| 87 | CD27 | QRRKYRSNKGESPVEPAEPCRYSCPREEEGSTIPIQEDYRKPEPACSP |
| 89 | RANK | CYRKKGKALTANLWHWINEACGRLSGDKESSGDSCVSTHTANFGQQGACEGVLLLTLEEKTFP |
| | | EDMCYPDQGGVCQGTCVGGGPYAQGEDARMLSLVSKTEIEEDSFRQMPTEDEYMDRPSQPT |
| | | DQLLFLTEPGSKSTPPFSEPLEVGENDSLSQCFTGTQSTVGSESCNCTEPLCRTDWTPMSSEN |
| | | YLQKEVDSGHCPHWAASPSPNWADVCTGCRNPPGEDCEPLVGSPKRGPLPQCAYGMGLPPE |
| | | EEASRTEARDQPEDGADGRLPSSARAGAGSGSSPGGQSPASGNVTGNSNSTFISSGQVMNFK |
| | | GDIIVVYVSQTSQEGAAAAAEPMGRPVQEETLARRDSFAGNGPRFPDPCGGPEGLREPEKASR |
| | | PVQEQGGAKA |

TABLE 4
Nucleic Acid Sequences

| SEQ | | |
|---|---|---|
| ID | ENCODED | |
| NO: | PROTEIN | NUCLEIC ACID SEQUENCE |
| 2 | Fv | ATGGGAGTGCAGGTGGAGACTATTAGCCCCGGAGATGGCAGAACATTCCCCAAAAGAGGAC |
| | Human | AGACTTGCGTCGTGCATTATACTGGAATGCTGGAAGACGGCAAGAAGGTGGACAGCAGCCG |
| | FKBP12v36 | GGACCGAAACAAGCCCTTCAAGTTCATGCTGGGGAAGCAGGAAGTGATCCGGGGCTGGGA |
| | | GGAAGGAGTCGCACAGATGTCAGTGGGACAGAGGGCCAAACTGACTATTAGCCCAGACTAC |
| | | GCTTATGGAGCAACCGGCCACCCCGGGATCATTCCCCCTCATGCTACACTGGTCTTCGATGT |
| | | GGAGCTGCTGAAGCTGGAA |
| 4 | 8-amino acid | AGCGGAGGAGGATCCGGA |
| | linker | |
| 6 | Human | GTGGACGGGTTTGGAGATGTGGGAGCCCTGGAATCCCTGCGGGGCAATGCCGATCTGGCT |
| | Δcaspase9 | TACATCCTGTCTATGGAGCCTTGCGGCCACTGTCTGATCATTAACAATGTGAACTTCTGCAGA |
| | | GAGAGCGGGCTGCGGACCAGAACAGGATCCAATATTGACTGTGAAAAGCTGCGGAGAAGGT |
| | | TCTCTAGTCTGCACTTTATGGTCGAGGTGAAAGGCGATCTGACCGCTAAGAAAATGGTGCTG |
| | | GCCCTGCTGGAACTGGCTCGGCAGGACCATGGGGCACTGGATTGCTGCGTGGTCGTGATC |
| | | CTGAGTCACGGCTGCCAGGCTTCACATCTGCAGTTCCCTGGGGCAGTCTATGGAACTGACG |
| | | GCTGTCCAGTCAGCGTGGAGAAGATCGTGAACATCTTCAACGGCACCTCTTGCCCAAGTCT |
| | | GGGCGGGAAGCCCAAACTGTTCTTTATTCAGGCCTGTGGAGGCGAGCAGAAAGATCACGGC |
| | | TTCGAAGTGGCTAGCACCTCCCCCGAGGACGAATCACCTGGAAGCAACCCTGAGCCAGATG |
| | | CAACCCCCTTCCAGGAAGGCCTGAGGACATTTGACCAGCTGGATGCCATCTCAAGCCTGCC |
| | | CACACCTTCTGACATTTTCGTCTCTTACAGTACTTTCCCTGGATTTGTGAGCTGGCGCGATCC |
| | | AAAGTCAGGCAGCTGGTACGTGGAGACACTGGACGATATCTTTGAGCAGTGGGCCCATTCT |
| | | GAAGACCTGCAGAGTCTGCTGCTGCGAGTGGCCAATGCTGTCTCTGTGAAGGGGATCTACA |
| | | AACAGATGCCAGGATGCTTCAACTTTCTGAGAAAGAAACTGTTCTTTAAGACCTCCGCATCTA |
| | | GGGCC |
| 8 | T2A | GAAGGCCGAGGGAGCCTGCTGACATGTGGCGATGTGGAGGAAAACCCAGGACCA |
| | polypeptide | |
| 10 | Signal | ATGGAGTTTGGACTTTCTTGGTTGTTTTTGGTGGCAATTCTGAAGGGTGTCCAGTGTAGCAG |
| | peptide | G |
| 12 | FMC63 VL | GACATCCAGATGACACAGACTACATCCTCCCTGTCTGCCTCTCTGGGAGACAGAGTCACCAT |
| | (anti-CD19) | CAGTTGCAGGGCAAGTCAGGACATTAGTAAATATTTAAATTGGTATCAGCAGAAACCAGATG |
| | | GAACTGTTAAACTCCTGATCTACCATACATCAAGATTACACTCAGGAGTCCCATCAAGGTTCA |
| | | GTGGCAGTGGGTCTGGAACAGATTATTCTCTCACCATTAGCAACCTGGAGCAAGAAGATATT |
| | | GCCACTTACTTTTGCCAACAGGGTAATACGCTTCCGTACACGTTCGGAGGGGGGACTAAGTT |
| | | GGAAATAACA |
| 14 | Glycine- | GGCGGAGGAAGCGGAGGTGGGGGC |
| | serine linker | |
| 16 | FMC63 VH | GAGGTGAAACTGCAGGAGTCAGGACCTGGCCTGGTGGCGCCCTCACAGAGCCTGTCCGTC |
| | (anti-CD19) | ACATGCACTGTCTCAGGGGTCTCATTACCCGACTATGGTGTAAGCTGGATTCGCCAGCCTCC |
| | | ACGAAAGGGTCTGGAGTGGCTGGGAGTAATATGGGGTAGTGAAACCACATACTATAATTCAG |
| | | CTCTCAAATCCAGACTGACCATCATCAAGGACAACTCCAAGAGCCAAGTTTTCTTAAAAATGA |
| | | ACAGTCTGCAAACTGATGACACAGCCATTTACTACTGTGCCAAACATTATTACTACGGTGGTA |
| | | GCTATGCTATGGACTACTGGGGTCAAGGAACCTCAGTCACCGTCTCCTCA |
| 18 | Human CD34 | GAACTTCCTACTCAGGGGACTTTCTCAAACGTTAGCACAAACGTAAGT |
| | epitope | |
| 20 | Human CD8 | CCCGCCCCAAGACCCCCCACACCTGCGCCGACCATTGCTTCTCAACCCCTGAGTTTGAGAC |
| | alpha stalk | CCGAGGCCTGCCGGCCAGCTGCCGGCGGGGCCGTGCATACAAGAGGACTCGATTTCGCTT |
| | | GCGAC |
| 22 | Human CD8 | ATCTATATCTGGGCACCTCTCGCTGGCACCTGTGGAGTCCTTCTGCTCAGCCTGGTTATTAC |
| | trans- | TCTGTACTGTAATCACCGGAATCGCCGCCGCGTTTGTAAGTGTCCCAGG |
| | membrane | |
| | region | |
| | between | |
| | FKBP12-1 | |
| | and FKBP12- | |
| | 2 in pM004 | |
| 24 | Portion of | AGAGTGAAGTTCAGCAGGAGCGCAGACGCCCCCGCGTACCAGCAGGGCCAGAACCAGCTC |
| | human CD3ζ | TATAACGAGCTCAATCTAGGACGAAGAGAGGAGTACGATGTTTTGGACAAGAGACGTGGCC |
| | encoded by | GGGACCCTGAGATGGGGGGAAAGCCGAGAAGGAAGAACCCTCAGGAAGGCCTGTACAATG |
| | pBP001 | AACTGCAGAAAGATAAGATGGCGGAGGCCTACAGTGAGATTGGGATGAAAGGCGAGCGCC |
| | | GGAGGGGCAAGGGGCACGATGGCCTTTACCAGGGTCTCAGTACAGCCACCAAGGACACCT |
| | | ACGACGCCCTTCACATGCAAGCTCTTCCACCTCGT |
| 26 | P2A | GCAACGAATTTTTCCCTGCTGAAACAGGCAGGGGACGTAGAGGAAAATCCTGGTCCT |
| | polypeptide | |
| 28 | truncated | atggctgcaggaggtcccggcgcggggtctgcggccccggtctcctccacatcctcccttcccctggctgctctcaaca |
| | MyD88 | tgcgagtgcggcgccgcctgtctctgttcttgaacgtgcggacacaggtggcggccgactggaccgcgctggcggagga |
| | polypeptide | gatggactttgagtacttggagatccggcaactggagacacaagcggaccccactggcaggctgctggacgcctggcag |
| | encoded by | ggacgccctggcgcctctgtaggccgactgctcgatctgcttaccaagctgggccgcgacgacgtgctgctggagctgg |
| | pBP001 | gacccagcattgaggaggattgccaaaagtatatcttgaagcagcagcaggaggaggctgagaagcctttacaggtggc |
| | | cgctgtagacagcagtgtcccacggacagcagagctggcgggcatcaccacacttgatgaccccctggggcatatgcct |
| | | gagcgtttcgatgccttcatctgctattgccccagcgacatc |
| 30 | Portion of | aaaaaggtggccaagaagccaaccaataaggccccccaccccaagcaggagccccaggagatcaattttcccgacgatc |
| | human CD40 | ttcctggctccaacactgctgctccagtgcaggagactttacatggatgccaaccggtcacccaggaggatggcaaaga |
| | polypeptide | gagtcgcatctcagtgcaggagagacag |
| | encoded by | |
| | pBP001 | |
| 32 | MLEMLE | ATGCTCGAGATGCTGGAG |
| | linker | |
| | encoded 5′ of | |
| | FKBP12v36 | |
| | in pBP001 | |
| 34 | Human | ATGGGAGTGCAGGTGGAAACCATCTCCCCAGGAGACGGGCGCACCTTCCCCAAGCGCGGC |
| | FKBP12 | CAGACCTGCGTGGTGCACTACACCGGGATGCTTGAAGATGGAAAGAAATTTGATTCCTCCCG |
| | (Genbank no | GGACAGAAACAAGCCCTTTAAGTTTATGCTAGGCAAGCAGGAGGTGATCCGAGGCTGGGAA |
| | AH002818) | GAAGGGGTTGCCCAGATGAGTGTGGGTCAGAGAGCCAAACTGACTATATCTCCAGATTATG |
| | | CCTATGGTGCCACTGGGCACCCAGGCATCATCCCACCACATGCCACTCTCGTCTTCGATGT |
| | | GGAGCTTCTAAAACTGGAATGA |
| 36 | Huma | ATGGCTGCAGGAGGTCCCGGCGCGGGGTCTGCGGCCCCGGTCTCCTCCACATCCTCCCTT |
| | MyD88- | CCCCTGGCTGCTCTCAACATGCGAGTGCGGCGCCGCCTGTCTCTGTTCTTGAACGTGCGGA |
| | encoding | CACAGGTGGCGGCCGACTGGACCGCGCTGGCGGAGGAGATGGACTTTGAGTACTTGGAGA |
| | DNA | TCCGGCAACTGGAGACACAAGCGGACCCCACTGGCAGGCTGCTGGACGCCTGGCAGGGAC |
| | (Genbank no. | GCCCTGGCGCCTCTGTAGGCCGACTGCTCGAGCTGCTTACCAAGCTGGGCCGCGACGACG |
| | U84408) | TGCTGCTGGAGCTGGGACCCAGCATTGAGGAGGATTGCCAAAAGTATATCTTGAAGCAGCA |
| | | GCAGGAGGAGGCTGAGAAGCCTTTACAGGTGGCCGCTGTAGACAGCAGTGTCCCACGGAC |
| | | AGCAGAGCTGGCGGGCATCACCACACTTGATGACCCCCTGGGGCATATGCCTGAGCGTTTC |
| | | GATGCCTTCATCTGCTATTGCCCCAGCGACATCCAGTTTGTGCAGGAGATGATCCGGCAACT |
| | | GGAACAGACAAACTATCGACTGAAGTTGTGTGTGTCTGACCGCGATGTCCTGCCTGGCACCT |
| | | GTGTCTGGTCTATTGCTAGTGAGCTCATCGAAAAGAGGTGCCGCCGGATGGTGGTGGTTGT |
| | | CTCTGATGATTACCTGCAGAGCAAGGAATGTGACTTCCAGACCAAATTTGCACTCAGCCTCT |
| | | CTCCAGGTGCCCATCAGAAGCGACTGATCCCCATCAAGTACAAGGCAATGAAGAAAGAGTTC |
| | | CCCAGCATCCTGAGGTTCATCACTGTCTGCGACTACACCAACCCCTGCACCAAATCTTGGTT |
| | | CTGGACTCGCCTTGCCAAGGCCTTGTCCCTGCCCTGA |
| 38 | Human CD40 | ATGGTTCGTCTGCCTCTGCAGTGCGTCCTCTGGGGCTGCTTGCTGACCGCTGTCCATCCAG |
| | (Genbank no. | AACCACCCACTGCATGCAGAGAAAAACAGTACCTAATAAACAGTCAGTGCTGTTCTTTGTGC |
| | BC012419) | CAGCCAGGACAGAAACTGGTGAGTGACTGCACAGAGTTCACTGAAACGGAATGCCTTCCTT |
| | | GCGGTGAAAGCGAATTCCTAGACACCTGGAACAGAGAGACACACTGCCACCAGCACAAATA |
| | | CTGCGACCCCAACCTAGGGCTTCGGGTCCAGCAGAAGGGCACCTCAGAAACAGACACCATC |
| | | TGCACCTGTGAAGAAGGCTGGCACTGTACGAGTGAGGCCTGTGAGAGCTGTGTCCTGCACC |
| | | GCTCATGCTCGCCCGGCTTTGGGGTCAAGCAGATTGCTACAGGGGTTTCTGATACCATCTG |
| | | CGAGCCCTGCCCAGTCGGCTTCTTCTCCAATGTGTCATCTGCTTTCGAAAAATGTCACCCTT |
| | | GGACAAGCTGTGAGACCAAAGACCTGGTTGTGCAACAGGCAGGCACAAACAAGACTGATGT |
| | | TGTCTGTGGTCCCCAGGATCGGCTGAGAGCCCTGGTGGTGATCCCCATCATCTTCGGGATC |
| | | CTGTTTGCCATCCTCTTGGTGCTGGTCTTTATCAAAAAGGTGGCCAAGAAGCCAACCAATAA |
| | | GGCCCCCCACCCCAAGCAGGAACCCCAGGAGATCAATTTTCCCGACGATCTTCCTGGCTCC |
| | | AACACTGCTGCTCCAGTGCAGGAGACTTTACATGGATGCCAACCGGTCACCCAGGAGGATG |
| | | GCAAAGAGAGTCGCATCTCAGTGCAGGAGAGACAGTGA |
| 40 | Human CD3ζ | ATGAAGTGGAAGGCGCTTTTCACCGCGGCCATCCTGCAGGCACAGTTGCCGATTACAGCCT |
| | (GenBank no. | CCAGCCTCCCCCACCCAACTCAGCAGAGCCCTGAGAAGAAAGTCCTGGGTCCCGGAGGCT |
| | XM_017002801) | GCACCTGCAGACACAACAGATTCTGCAATGAGGCACAGAGCTTTGGCCTGCTGGATCCCAA |
| | | ACTCTGCTACCTGCTGGATGGAATCCTCTTCATCTATGGTGTCATTCTCACTGCCTTGTTCCT |
| | | GAGAGTGAAGTTCAGCAGGAGCGCAGACGCCCCCGCGTACCAGCAGGGCCAGAACCAGCT |
| | | CTATAACGAGCTCAATCTAGGACGAAGAGAGGAGTACGATGTTTTGGACAAGAGACGTGGC |
| | | CGGGACCCTGAGATGGGGGGAAAGCCGAGAAGGAAGAACCCTCAGGAAGGCCTGTACAAT |
| | | GAACTGCAGAAAGATAAGATGGCGGAGGCCTACAGTGAGATTGGGATGAAAGGCGAGCGC |
| | | CGGAGGGGCAAGGGGCACGATGGCCTTTACCAGGGTCTCAGTACAGCCACCAAGGACACC |
| | | TACGACGCCCTTCACATGCAGGCCCTGCCCCCTCGCTAA |
| 42 | Homo | CODING FOR Homo sapiens caspase 9 (Genbank no. BAA82697) SEQ ID NO: 41 |
| | sapiens | in amino acid sequence table |
| | caspase 9 | |
| | (Genbank no. | |
| | BAA82697) | |
| 44 | MLE linker | ATGCTCGAG |
| 46 | FRP5 VH | GAAGTCCAATTGCAACAGTCAGGCCCCGAATTGAAAAAGCCCGGCGAAACAGTGAAGATAT |
| | (anti-Her2) | CTTGTAAAGCCTCCGGTTACCCTTTTACGAACTATGGAATGAACTGGGTCAAACAAGCCCCT |
| | | GGACAGGGATTGAAGTGGATGGGATGGATCAATACATCAACAGGCGAGTCTACCTTCGCAG |
| | | ATGATTTCAAAGGTCGCTTTGACTTCTCACTGGAGACCAGTGCAAATACCGCCTACCTTCAG |
| | | ATTAACAATCTTAAAAGCGAGGATATGGCAACCTACTTTTGCGCAAGATGGGAAGTTTATCAC |
| | | GGGTACGTGCCATACTGGGGACAAGGAACGACAGTGACAGTTAGTAGC |
| 48 | FRP5 VL | GACATCCAATTGACACAATCACACAAATTTCTCTCAACTTCTGTAGGAGACAGAGTGAGCATA |
| | (anti-Her2) | ACCTGCAAAGCATCCCAGGACGTGTACAATGCTGTGGCTTGGTACCAACAGAAGCCTGGAC |
| | | AATCCCCAAAATTGCTGATTTATTCTGCCTCTAGTAGGTACACTGGGGTACCTTCTCGGTTTA |
| | | CGGGCTCTGGGTCCGGACCAGATTTCACGTTCACAATCAGTTCCGTTCAAGCTGAAGACCTC |
| | | GCTGTTTATTTTTGCCAGCAGCACTTCCGAACCCCTTTTACTTTTGGCTCAGGCACTAAGTTG |
| | | GAAATCAAGGCTTTG |
| 50 | Linker | |
| 52 | Myristoylation | atggggagtagcaagagcaagcctaaggaccccagccagcgc |
| | domain | |
| 54 | A11 VL | GACATCCAACTGACGCAAAGCCCATCTACACTCAGCGCTAGCATGGGGGACAGGGTCACAA |
| | (anti-PSCA) | TCACGTGCTCTGCCTCAAGTTCCGTTAGGTTTATCCATTGGTATCAGCAGAAACCTGGAAAG |
| | | GCCCCAAAAAGACTGATCTATGATACCAGCAAGCTGGCTTCCGGAGTGCCCTCAAGGTTCTC |
| | | AGGATCTGGCAGTGGGACCGATTTCACCCTGACAATTAGCAGCCTTCAGCCAGAGGATTTC |
| | | GCAACCTATTACTGTCAGCAATGGGGGTCCAGCCCATTCACTTTCGGCCAAGGAACAAAGGT |
| | | GGAGATAAAA |
| 56 | A11 VH | GAGGTGCAGCTCGTGGAGTATGGCGGGGGCCTGGTGCAGCCTGGGGGTAGTCTGAGGCTC |
| | (anti-PSCA) | TCCTGCGCTGCCTCTGGCTTTAACATTAAAGACTACTACATACATTGGGTGCGGCAGGCCCC |
| | | AGGCAAAGGGCTCGAATGGGTGGCCTGGATTGACCCTGAGAATGGTGACACTGAGTTTGTC |
| | | CCCAAGTTTCAGGGCAGAGCCACCATGAGCGCTGACACAAGCAAAAACACTGCTTATCTCCA |
| | | AATGAATAGCCTGCGAGCTGAAGATACAGCAGTCTATTACTGCAAGACGGGAGGATTCTGG |
| | | GGCCAGGGAACTCTGGTGACAGTTAGTTCC |
| 58 | bm2B3 VL | GACATCCAGCTGACACAAAGTCCCAGTAGCCTGTCAGCCAGTGTCGGCGATAGGGTGACAA |
| | (anti-PSCA) | TTACATGCTCCGCAAGTAGTAGCGTCAGATTCATACACTGGTACCAGCAGAAGCCTGGGAAG |
| | | GCCCCAAAGAGGCTTATCTACGATACCAGTAAACTCGCCTCTGGAGTTCCTAGCCGGTTTTC |
| | | TGGATCTGGCAGCGGAACTAGCTACACCCTCACAATCTCCAGTCTGCAACCAGAGGACTTTG |
| | | CAACCTACTACTGCCAGCAATGGAGCAGCTCCCCTTTCACCTTTGGGCAGGGTACTAAGGTG |
| | | GAGATCAAG |
| 60 | bm2B3 VH | GAGGTGCAGCTTGTAGAGAGCGGGGGAGGCCTCGTACAGCCAGGGGGCTCTCTGCGCCTG |
| | (anti-PSCA) | TCATGTGCAGCTTCAGGATTCAATATAAAGGACTATTACATTCACTGGGTACGGCAAGCTCC |
| | | CGGTAAGGGCCTGGAATGGATCGGTTGGATCGACCCTGAAAACGGAGATACAGAATTTGTG |
| | | CCCAAGTTCCAGGGAAAGGCTACCATGTCTGCCGATACTTCTAAGAATACAGCATACCTTCA |
| | | GATGAATTCTCTCCGCGCCGAGGACACAGCCGTGTATTATTGTAAAACGGGAGGGTTCTGG |
| | | GGTCAGGGTACCCTTGTGACTGTGTCTTCC |
| 62 | Caspase-9 | GTCGACGGATTTGGTGATGTCGGTGCTCTTGAGAGTTTGAGGGGAAATGCAGATTTGGCTTA |
| | D330E | CATCCTGAGCATGGAGCCCTGTGGCCACTGCCTCATTATCAACAATGTGAACTTCTGCCGTG |
| | | AGTCCGGGCTCCGCACCCGCACTGGCTCCAACATCGACTGTGAGAAGTTGCGGCGTCGCTT |
| | | CTCCTCGCTGCATTTCATGGTGGAGGTGAAGGGCGACCTGACTGCCAAGAAAATGGTGCTG |
| | | GCTTTGCTGGAGCTGGCGCGGCAGGACCACGGTGCTCTGGACTGCTGCGTGGTGGTCATT |
| | | CTCTCTCACGGCTGTCAGGCCAGCCACCTGCAGTTCCCAGGGGCTGTCTACGGCACAGATG |
| | | GATGCCCTGTGTCGGTCGAGAAGATTGTGAACATCTTCAATGGGACCAGCTGCCCCAGCCT |
| | | GGGAGGGAAGCCCAAGCTCTTTTTCATCCAGGCCTGTGGTGGGGAGCAGAAAGACCATGG |
| | | GTTTGAGGTGGCCTCCACTTCCCCTGAAGACGAGTCCCCTGGCAGTAACCCCGAGCCAGAT |
| | | GCCACCCCGTTCCAGGAAGGTTTGAGGACCTTCGACCAGCTGGcCGCCATATCTAGTTTGCC |
| | | CACACCCAGTGACATCTTTGTGTCCTACTCTACTTTCCCAGGTTTTGTTTCCTGGAGGGACCC |
| | | CAAGAGTGGCTCCTGGTACGTTGAGACCCTGGACGACATCTTTGAGCAGTGGGCTCACTCT |
| | | GAAGACCTGCAGTCCCTCCTGCTTAGGGTCGCTAATGCTGTTTCGGTGAAAGGGATTTATAA |
| | | ACAGATGCCTGGTTGCTTTAATTTCCTCCGGAAAAAACTTTTCTTTAAAACATCAGCTAGCAG |
| | | AGCC |
| 64 | ΔCasp9 (res. | GGATTTGGTGATGTCGGTGCTCTTGAGAGTTTGAGGGGAAATGCAGATTTGGCTTACATCCT |
| | 135-416) | GAGCATGGAGCCCTGTGGCCACTGCCTCATTATCAACAATGTGAACTTCTGCCGTGAGTCCG |
| | D330A | GGCTCCGCACCCGCACTGGCTCCAACATCGACTGTGAGAAGTTGCGGCGTCGCTTCTCCTC |
| | N405Q | GCTGCATTTCATGGTGGAGGTGAAGGGCGACCTGACTGCCAAGAAAATGGTGCTGGCTTTG |
| | | CTGGAGCTGGCGCgGCAGGACCACGGTGCTCTGGACTGCTGCGTGGTGGTCATTCTCTCTC |
| | | ACGGCTGTCAGGCCAGCCACCTGCAGTTCCCAGGGGCTGTCTACGGCACAGATGGATGCC |
| | | CTGTGTCGGTCGAGAAGATTGTGAACATCTTCAATGGGACCAGCTGCCCCAGCCTGGGAGG |
| | | GAAGCCCAAGCTCTTTTTCATCCAGGCCTGTGGTGGGGAGCAGAAAGACCATGGGTTTGAG |
| | | GTGGCCTCCACTTCCCCTGAAGACGAGTCCCCTGGCAGTAACCCCGAGCCAGATGCCACCC |
| | | CGTTCCAGGAAGGTTTGAGGACCTTCGACCAGCTGGCCGCCATATCTAGTTTGCCCACACC |
| | | CAGTGACATCTTTGTGTCCTACTCTACTTTCCCAGGTTTTGTTTCCTGGAGGGACCCCAAGA |
| | | GTGGCTCCTGGTACGTTGAGACCCTGGACGACATCTTTGAGCAGTGGGCTCACTCTGAAGA |
| | | CCTGCAGTCCCTCCTGCTTAGGGTCGCTAATGCTGTTTCGGTGAAAGGGATTTATAAACAGA |
| | | TGCCTGGTTGCTTTCAGTTCCTCCGGAAAAAACTTTTCTTTAAAACATCA |
| 66 | ΔCasp9 (res. | GGATTTGGTGATGTCGGTGCTCTTGAGAGTTTGAGGGGAAATGCAGATTTGGCTTACATCCT |
| | 135-416) | GAGCATGGAGCCCTGTGGCCACTGCCTCATTATCAACAATGTGAACTTCTGCCGTGAGTCCG |
| | N405Q | GGCTCCGCACCCGCACTGGCTCCAACATCGACTGTGAGAAGTTGCGGCGTCGCTTCTCCTC |
| | | GCTGCATTTCATGGTGGAGGTGAAGGGCGACCTGACTGCCAAGAAAATGGTGCTGGCTTTG |
| | | CTGGAGCTGGCGCgGCAGGACCACGGTGCTCTGGACTGCTGCGTGGTGGTCATTCTCTCTC |
| | | ACGGCTGTCAGGCCAGCCACCTGCAGTTCCCAGGGGCTGTCTACGGCACAGATGGATGCC |
| | | CTGTGTCGGTCGAGAAGATTGTGAACATCTTCAATGGGACCAGCTGCCCCAGCCTGGGAGG |
| | | GAAGCCCAAGCTCTTTTTCATCCAGGCCTGTGGTGGGGAGCAGAAAGACCATGGGTTTGAG |
| | | GTGGCCTCCACTTCCCCTGAAGACGAGTCCCCTGGCAGTAACCCCGAGCCAGATGCCACCC |
| | | CGTTCCAGGAAGGTTTGAGGACCTTCGACCAGCTGGACGCCATATCTAGTTTGCCCACACC |
| | | CAGTGACATCTTTGTGTCCTACTCTACTTTCCCAGGTTTTGTTTCCTGGAGGGACCCCAAGA |
| | | GTGGCTCCTGGTACGTTGAGACCCTGGACGACATCTTTGAGCAGTGGGCTCACTCTGAAGA |
| | | CCTGCAGTCCCTCCTGCTTAGGGTCGCTAATGCTGTTTCGGTGAAAGGGATTTATAAACAGA |
| | | TGCCTGGTTGCTTTCAGTTCCTCCGGAAAAAACTTTTCTTTAAAACATCA |
| 68 | ΔCasp9 (res. | GGATTTGGTGATGTCGGTGCTCTTGAGAGTTTGAGGGGAAATGCAGATTTGGCTTACATCCT |
| | 135-416) | GAGCATGGAGCCCTGTGGCCACTGCCTCATTATCAACAATGTGAACTTCTGCCGTGAGTCCG |
| | D330A | GGCTCCGCACCCGCACTGGCTCCAACATCGACTGTGAGAAGTTGCGGCGTCGCTTCTCCTC |
| | | GCTGCATTTCATGGTGGAGGTGAAGGGCGACCTGACTGCCAAGAAAATGGTGCTGGCTTTG |
| | | CTGGAGCTGGCGCgGCAGGACCACGGTGCTCTGGACTGCTGCGTGGTGGTCATTCTCTCTC |
| | | ACGGCTGTCAGGCCAGCCACCTGCAGTTCCCAGGGGCTGTCTACGGCACAGATGGATGCC |
| | | CTGTGTCGGTCGAGAAGATTGTGAACATCTTCAATGGGACCAGCTGCCCCAGCCTGGGAGG |
| | | GAAGCCCAAGCTCTTTTTCATCCAGGCCTGTGGTGGGGAGCAGAAAGACCATGGGTTTGAG |
| | | GTGGCCTCCACTTCCCCTGAAGACGAGTCCCCTGGCAGTAACCCCGAGCCAGATGCCACCC |
| | | CGTTCCAGGAAGGTTTGAGGACCTTCGACCAGCTGGCCGCCATATCTAGTTTGCCCACACC |
| | | CAGTGACATCTTTGTGTCCTACTCTACTTTCCCAGGTTTTGTTTCCTGGAGGGACCCCAAGA |
| | | GTGGCTCCTGGTACGTTGAGACCCTGGACGACATCTTTGAGCAGTGGGCTCACTCTGAAGA |
| | | CCTGCAGTCCCTCCTGCTTAGGGTCGCTAATGCTGTTTCGGTGAAAGGGATTTATAAACAGA |
| | | TGCCTGGTTGCTTTAATTTCCTCCGGAAAAAACTTTTCTTTAAAACATCA |
| 70 | ΔCD19 | ATGCCCCCTCCTAGACTGCTGTTTTTCCTGCTCTTTCTCACCCCAATGGAAGTTAGACCTGAG |
| | marker | GAACCACTGGTCGTTAAAGTGGAAGAAGGTGATAATGCTGTCCTCCAATGCCTTAAAGGGAC |
| | polypeptide | CAGCGACGGACCAACGCAGCAACTGACTTGGAGCCGGGAGTCCCCTCTCAAGCCGTTTCTC |
| | | AAGCTGTCACTTGGCCTGCCAGGTCTTGGTATTCACATGCGCCCCCTTGCCATTTGGCTCTT |
| | | CATATTCAATGTGTCTCAACAAATGGGTGGATTCTACCTTTGCCAGCCCGGCCCCCCTTCTG |
| | | AGAAAGCTTGGCAGCCTGGATGGACCGTCAATGTTGAAGGCTCCGGTGAGCTGTTTAGATG |
| | | GAATGTGAGCGACCTTGGCGGACTCGGTTGCGGACTGAAAAATAGGAGCTCTGAAGGACCC |
| | | TCTTCTCCCTCCGGTAAGTTGATGTCACCTAAGCTGTACGTGTGGGCCAAGGACCGCCCCG |
| | | AAATCTGGGAGGGCGAGCCTCCATGCCTGCCGCCTCGCGATTCACTGAACCAGTCTCTGTC |
| | | CCAGGATCTCACTATGGCGCCCGGATCTACTCTTTGGCTGTCTTGCGGCGTTCCCCCAGATA |
| | | GCGTGTCAAGAGGACCTCTGAGCTGGACCCACGTACACCCTAAGGGCCCTAAGAGCTTGTT |
| | | GAGCCTGGAACTGAAGGACGACAGACCCGCACGCGATATGTGGGTAATGGAGACCGGCCT |
| | | TCTGCTCCCTCGCGCTACCGCACAGGATGCAGGGAAATACTACTGTCATAGAGGGAATCTG |
| | | ACTATGAGCTTTCATCTCGAAATTACAGCACGGCCCGTTCTTTGGCATTGGCTCCTCCGGAC |
| | | TGGAGGCTGGAAGGTGTCTGCCGTAACACTCGCTTACTTGATTTTTTGCCTGTGTAGCCTGG |
| | | TTGGGATCCTGCATCTTCAGCGAGCCCTTGTATTGCGCCGAAAAAGAAAACGAATGACTGAC |
| | | CCTACACGACGATTCTGA |
| 72 | OX40 | GTTGCCGCCATCCTGGGCCTGGGCCTGGTGCTGGGGCTGCTGGGCCCCCTGGCCATCCTG |
| | cytoplasmic | CTGGCCCTGTACCTGCTCCGGGACCAGAGGCTGCCCCCCGATGCCCACAAGCCCCCTGGG |
| | Signaling | GGAGGCAGTTTCCGGACCCCCATCCAAGAGGAGCAGGCCGACGCCCACTCCACCCTGGCC |
| | region | AAGATC |
| 74 | 4-1BB | AGTGTAGTTAAAAGAGGAAGAAAAAAGTTGCTGTATATATTTAAACAACCATTTATGAGACCA |
| | cytoplasmic | GTGCAAACCACCCAAGAAGAAGACGGATGTTCATGCAGATTCCCAGAAGAAGAAGAAGGAG |
| | Signaling | GATGTGAATTG |
| | region | |
| 76 | 4-1BB | TTCTGGGTACTGGTTGTAGTCGGTGGCGTACTTGCTTGTTATTCTCTTCTTGTTACCGTAGCC |
| | cytoplasmic | TTCATTATATTCTGGGTCCGATCAAAGCGCTCAAGACTCCTCCATTCCGATTATATGAACATG |
| | Signaling | ACACCTCGCCGACCTGGTCCTACACGCAAACATTATCAACCCTACGCACCCCCCCGAGACTT |
| | region | CGCTGCTTATCGATCC |
| 78 | Fv | ggagtgcaggtggagactatctccccaggagacgggcgcaccttccccaagcgcggccagacctgcgtggtgcactaca |
| | | ccgggatgcttgaagatggaaagaaagttgattcctcccgggacagaaacaagccctttaagtttatgctaggcaagca |
| | | ggaggtgatccgaggctgggaagaaggggttgcccagatgagtgtgggtcagagagccaaactgactatatctccagat |
| | | tatgcctatggtgccactgggcacccaggcatcatcccaccacatgccactctcgtcttcgatgtggagcttctaaaac |
| | | tggaa |
| 80 | Fv′ | GGcGTcCAaGTcGAaACcATtagtCCcGGcGAtGGcaGaACaTTtCCtAAaaGgGGaCAaACaTGtGTc |
| | | GTcCAtTAtACaGGcATGtTgGAgGAcGGcAAaAAgGTgGAcagtagtaGaGAtcGcAAtAAaCCtTTcAAa |
| | | TTcATGtTgGGaAAaCAaGAaGTcATtaGgGGaTGGGAgGAgGGcGTgGCtCAaATGtccGTcGGcCA |
| | | acGcGCtAAgCTcACcATcagcCCcGAcTAcGCaTAcGGcGCtACcGGaCAtCCcGGaATtATtCCcCCt |
| | | CAcGCtACctTgGTgTTtGAcGTcGAaCTgtTgAAgCTc |
| 82 | FKBP12 | GGcGTGCAaGTGGAaACTATaAGCCCgGGAGAcGGCcGcACATTtCCCAAgAGAGGcCAGACcT |
| | Wild type | GCGTgGTGCAcTATACaGGAATGCTGGAgGACGGgAAGAAaTTCGAtAGCtcCCGGGAtCGAAAt |
| | | AAGCCtTTCAAaTTCATGCTGGGcAAGCAaGAAGTcATCaGaGGCTGGGAaGAAGGcGTCGCcC |
| | | AGATGTCcGTGGGtCAGcGcGCCAAgCTGACaATTAGtCCAGAtTACGCcTATGGcGCAACaGGC |
| | | CAtCCCGGcATCATcCCCCCaCATGCcACACTcGTCTTtGATGTcGAGCTcCTGAAaCTGGAg |
| 84 | MyD88 | atggctgcaggaggtcccggcgcggggtctgcggccccggtctcctccacatcctcccttcccctggctgctctcaaca |
| | Full length | tgcgagtgcggcgccgcctgtctctgttcttgaacgtgcggacacaggtggcggccgactggaccgcgctggcggagga |
| | | gatggactttgagtacttggagatccggcaactggagacacaagcggaccccactggcaggctgctggacgcctggcag |
| | | ggacgccctggcgcctctgtaggccgactgctcgagctgcttaccaagctgggccgcgacgacgtgctgctggagctgg |
| | | gacccagcattgaggaggattgccaaaagtatatcttgaagcagcagcaggaggaggctgagaagcctttacaggtggc |
| | | cgctgtagacagcagtgtcccacggacagcagagctggcgggcatcaccacacttgatgaccccctggggcatatgcct |
| | | gagcgtttcgatgccttcatctgctattgccccagcgacatccagtttgtgcaggagatgatccggcaactggaacaga |
| | | caaactatcgactgaagttgtgtgtgtctgaccgcgatgtcctgcctggcacctgtgtctggtctattgctagtgagct |
| | | catcgaaaagaggtgccgccggatggtggtggttgtctctgatgattacctgcagagcaaggaatgtgacttccagacc |
| | | aaatttgcactcagcctctctccaggtgcccatcagaagcgactgatccccatcaagtacaaggcaatgaagaaagagt |
| | | tccccagcatcctgaggttcatcactgtctgcgactacaccaacccctgcaccaaatcttggttctggactcgccttgc |
| | | caaggccttgtccctgccc |
| 86 | ICOS | ACAAAAAAGAAGTATTCATCCAGTGTGCACGACCCTAACGGTGAATACATGTTCATGAGAGC |
| | signaling | AGTGAACACAGCCAAAAAATCTAGACTCACAGATGTGACCCTA |
| | domain | |

### Example 5: Representative Embodiments

Provided hereafter are examples of certain embodiments of the technology.

A1. A modified cell population, comprising modified T cells, wherein:
the modified T cells comprise a polynucleotide that encodes a chimeric antigen receptor, wherein the chimeric antigen receptor comprises:
(i) a transmembrane region;
(ii) a T cell activation molecule; and
(iii) an antigen recognition moiety
   wherein the ratio of CD8⁺ to CD4⁺ T cells in the modified cell population is 3:2 or greater.
   A2. The modified cell population of embodiment A1, wherein the chimeric antigen receptor comprises
(i) a transmembrane region;
(ii) a costimulatory polypeptide cytoplasmic signaling region, a truncated MyD88 polypeptide region lacking the TIR domain, a truncated MyD88 polypeptide region lacking the TIR domain and a costimulatory polypeptide cytoplasmic signaling region, or a truncated MyD88 polypeptide region lacking the TIR domain and a CD40 cytoplasmic polypeptide region lacking the CD40 extracellular domain;
(iii) a T cell activation molecule; and
(iv) an antigen recognition moiety.
   A2.1. The modified cell population of any one of claims A1 to A2, wherein the costimulatory polypeptide cytoplasmic signaling region is selected from the group consisting of CD27, CD28, 4-1BB, OX40, ICOS, RANK, TRANCE, and DAP10.
   A2.2. The modified cell population of any one of embodiments A1 to A2.1, wherein the chimeric antigen receptor comprises two costimulatory polypeptide cytoplasmic signaling regions selected from the group consisting of CD27, CD28, 4-1BB, OX40, ICOS, RANK, TRANCE, and DAP10.
   A3. A modified cell population, comprising a polynucleotide that encodes a chimeric antigen receptor, wherein:
the chimeric antigen receptor comprises (i) a transmembrane region; (ii) a MyD88 polypeptide or a truncated MyD88 polypeptide lacking a TIR domain; (iii) a CD40 cytoplasmic polypeptide region lacking a CD40 extracellular domain; (iv) a T cell activation molecule; and (v) an antigen recognition moiety; and
at least 80% of the modified cells are CD8⁺ T cells.
   A4. A modified cell population, comprising modified T cells, wherein:
the modified T cells comprise a polynucleotide that encodes a chimeric antigen receptor, wherein the chimeric antigen receptor comprises (i) a transmembrane region; (ii) a MyD88 polypeptide or a truncated MyD88 polypeptide lacking a TIR domain; (iii) a CD40 cytoplasmic polypeptide region lacking a CD40 extracellular domain; (iv) a T cell activation molecule; and (v) an antigen recognition moiety; and the ratio of CD8⁺ to CD4⁺ T cells is 4:1 or greater.
   A5. The modified cell population of any one of embodiments A1 to A4, wherein the modified T cells comprise a second polynucleotide that encodes an inducible chimeric pro-apoptotic polypeptide.
   A6. The modified cell population of any one of embodiments A1 to A5, wherein the modified cells or modified T cells comprise
a first polynucleotide that encodes a chimeric antigen receptor, wherein the chimeric antigen receptor comprises (i) a transmembrane region; (ii) a MyD88 polypeptide or a truncated MyD88 polypeptide lacking a TIR domain; (iii) a CD40 cytoplasmic polypeptide region lacking a CD40 extracellular domain; (iv) a T cell activation molecule; and (v) an antigen recognition moiety; and
a second polynucleotide that encodes a chimeric Caspase-9 polypeptide comprising a multimeric ligand binding region and a Caspase-9 polypeptide.
   A7. The modified cell population of any one of embodiments A1 to A5, wherein the modified cells or modified T cells comprise a nucleic acid, wherein the nucleic acid comprises
a first polynucleotide that encodes a chimeric antigen receptor, wherein the chimeric antigen receptor comprises (i) a transmembrane region; (ii) a MyD88 polypeptide or a truncated MyD88 polypeptide lacking a TIR domain; (iii) a CD40 cytoplasmic polypeptide region lacking a CD40 extracellular domain; (iv) a T cell activation molecule; and (v) an antigen recognition moiety; and
a second polynucleotide that encodes a chimeric Caspase-9 polypeptide comprising a multimeric ligand binding region and a Caspase-9 polypeptide.
   A8. A modified cell population, comprising a polynucleotide that encodes a chimeric antigen receptor, wherein:
the chimeric antigen receptor comprises (i) a transmembrane region; (ii) a costimulatory polypeptide cytoplasmic signaling region selected from the group consisting of CD27, CD28, ICOS, 4-1BB, and OX40; (iii) a T cell activation molecule; and (iv) an antigen recognition moiety; and
at least 80% of the modified cells are CD8⁺ T cells.
   A9. A modified cell population, comprising modified T cells, wherein:
the modified T cells comprise a polynucleotide that encodes a chimeric antigen receptor, wherein the chimeric antigen receptor comprises (i) a transmembrane region; (ii) a costimulatory polypeptide cytoplasmic signaling region selected from the group consisting of CD27, CD28, ICOS, 4-1BB, and OX40; (iii) a T cell activation molecule; and (iv) an antigen recognition moiety and
the ratio of CD8⁺ to CD4⁺ T cells is 4:1 or greater.
   A10. The modified cell population of any one of embodiments A1 to A9, wherein the modified cells or modified T cells comprise
a first polynucleotide that encodes a chimeric antigen receptor, wherein the chimeric antigen receptor comprises (i) a transmembrane region; (ii) a costimulatory polypeptide cytoplasmic signaling region selected from the group consisting of CD27, CD28, ICOS, 4-1BB, and OX40; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A11. The modified cell population of embodiment A1 to A10, wherein the modified cells or modified T cells comprise a nucleic acid, wherein the nucleic acid comprises
a first polynucleotide that encodes a chimeric antigen receptor, wherein the chimeric antigen receptor comprises (i) a transmembrane region; (ii) a costimulatory polypeptide cytoplasmic signaling region selected from the group consisting of CD27, CD28, ICOS, 4-1BB, and OX40; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A12. A modified cell population, comprising a polynucleotide that encodes a chimeric antigen receptor, wherein:
the chimeric antigen receptor comprises (i) a transmembrane region; (ii) two costimulatory polypeptide cytoplasmic signaling regions selected from the group consisting of CD27, CD28, ICOS, 4-1BB, and OX40; (iii) a T cell activation molecule; and (iv) an antigen recognition moiety; and
at least 80% of the modified cells are CD8⁺ T cells.
   A13. A modified cell population, comprising modified T cells, wherein:
the modified T cells comprise a polynucleotide that encodes a chimeric antigen receptor, wherein the chimeric antigen receptor comprises (i) a transmembrane region; (ii) two costimulatory polypeptide cytoplasmic signaling regions selected from the group consisting of CD27, CD28, ICOS, 4-1BB, and OX40; (iii) a T cell activation molecule; and (iv) an antigen recognition moiety; and
the ratio of CD8⁺ to CD4⁺ T cells is 4:1 or greater.
   A14. The modified cell population of any one of embodiments A1 to A13, wherein:
the modified cells or modified T cells comprise a first polynucleotide that encodes a chimeric antigen receptor, wherein the chimeric antigen receptor comprises (i) a transmembrane region; (ii) two costimulatory polypeptide cytoplasmic signaling regions selected from the group consisting of CD27, CD28, ICOS, 4-1BB, and OX40; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A15. The modified cell population of embodiment A14, wherein the modified cells or modified T cells comprise a nucleic acid, wherein the nucleic acid comprises:
a first polynucleotide that encodes a chimeric antigen receptor, wherein the chimeric antigen receptor comprises (i) a transmembrane region; (ii) two costimulatory polypeptide cytoplasmic signaling regions selected from the group consisting of CD27, CD28, ICOS, 4-1BB, and OX40; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A16. A modified cell population, comprising a polynucleotide that encodes a chimeric antigen receptor, wherein:
the chimeric antigen receptor comprises (i) a transmembrane region; (ii) a MyD88 polypeptide or truncated MyD88 polypeptide lacking a TIR domain; (iii) a T cell activation molecule; and (iv) an antigen recognition moiety; and
at least 80% of the modified cells are CD8⁺ T cells.
   A17. A modified cell population, comprising modified T cells, wherein:
the modified T cells comprise a polynucleotide that encodes a chimeric antigen receptor, wherein the chimeric antigen receptor comprises (i) a transmembrane region; (ii) a MyD88 polypeptide or truncated MyD88 polypeptide lacking a TIR domain; (iii) a T cell activation molecule; and (iv) an antigen recognition moiety; and
the ratio of CD8⁺ to CD4⁺ T cells is 4:1 or greater.
   A18. The modified cell population of any one of embodiments A1 to A17, wherein the modified cells or modified T cells comprise
a first polynucleotide that encodes a chimeric antigen receptor, wherein the chimeric antigen receptor comprises (i) a transmembrane region; (ii) a MyD88 polypeptide or truncated MyD88 polypeptide lacking a TIR domain; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A19. The modified cell population of embodiment A18, wherein the modified cells or modified T cells comprise a nucleic acid, wherein the nucleic acid comprises:
a first polynucleotide that encodes a chimeric antigen receptor, wherein the chimeric antigen receptor comprises (i) a transmembrane region; (ii) a MyD88 polypeptide or truncated MyD88 polypeptide lacking a TIR domain; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A20. A modified cell population, comprising a polynucleotide that encodes a chimeric antigen receptor, wherein:
the chimeric antigen receptor comprises (i) a transmembrane region; (ii) a MyD88 polypeptide or truncated MyD88 polypeptide lacking a TIR domain and a costimulatory polypeptide cytoplasmic signaling regions selected from the group consisting of CD27, CD28, ICOS, 4-1BB, and OX40; (iii) a T cell activation molecule; and (iv) an antigen recognition moiety; and
at least 80% of the modified cells are CD8⁺ T cells.
   A21. A modified cell population, comprising modified T cells, wherein:
the modified T cells comprise a polynucleotide that encodes a chimeric antigen receptor, wherein the chimeric antigen receptor comprises (i) a transmembrane region; (ii) a MyD88 polypeptide or truncated MyD88 polypeptide lacking a TIR domain and a costimulatory polypeptide cytoplasmic signaling regions selected from the group consisting of CD27, CD28, ICOS, 4-1BB, and OX40; (iii) a T cell activation molecule; and (iv) an antigen recognition moiety; and
the ratio of CD8⁺ to CD4⁺ T cells is 4:1 or greater.
   A22. The modified cell population of any one of embodiments A1 to A22, wherein:
the modified cells or modified T cells comprise a first polynucleotide that encodes a chimeric antigen receptor, wherein the chimeric antigen receptor comprises (i) a transmembrane region; (ii) a MyD88 polypeptide or truncated MyD88 polypeptide lacking a TIR domain and a costimulatory polypeptide cytoplasmic signaling regions selected from the group consisting of CD27, CD28, ICOS, 4-1BB, and OX40; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A23. The modified cell population of embodiment A22, wherein the modified cells or modified T cells comprise a nucleic acid, wherein the nucleic acid comprises
a first polynucleotide that encodes a chimeric antigen receptor, wherein the chimeric antigen receptor comprises (i) a transmembrane region; (ii) a MyD88 polypeptide or truncated MyD88 polypeptide lacking a TIR domain and a costimulatory polypeptide cytoplasmic signaling regions selected from the group consisting of CD27, CD28, ICOS, 4-1BB, and OX40; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A24. A modified cell population, comprising a polynucleotide that encodes a chimeric antigen receptor, wherein:
the chimeric antigen receptor comprises (i) a transmembrane region; (ii) a CD40 polypeptide lacking an extracellular domain; (iii) a T cell activation molecule; and (iv) an antigen recognition moiety; and
at least 80% of the modified cells are CD8⁺ T cells.
   A25. A modified cell population, comprising modified T cells, wherein:
the modified T cells comprise a polynucleotide that encodes a chimeric antigen receptor, wherein the chimeric antigen receptor comprises (i) a transmembrane region; (ii) a CD40 polypeptide lacking an extracellular domain; (iii) a T cell activation molecule; and (iv) an antigen recognition moiety; and
the ratio of CD8⁺ to CD4⁺ T cells is 4:1 or greater.
   A26. The modified cell population of any one of embodiments A1 to A25, wherein the modified cells or modified T cells comprise
a first polynucleotide that encodes a chimeric antigen receptor, wherein the chimeric antigen receptor comprises (i) a transmembrane region; (ii) a CD40 polypeptide lacking an extracellular domain; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A27. The modified cell population of embodiment A26, wherein the modified cells or modified T cells comprise a nucleic acid, wherein the nucleic acid comprises
a first polynucleotide that encodes a chimeric antigen receptor, wherein the chimeric antigen receptor comprises (i) a transmembrane region; (ii) a CD40 polypeptide lacking an extracellular domain; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A28. A modified cell population, comprising a polynucleotide that encodes a chimeric antigen receptor, wherein:
the chimeric antigen receptor comprises (i) a transmembrane region; (ii) a CD40 polypeptide lacking an extracellular domain and a costimulatory polypeptide cytoplasmic signaling regions selected from the group consisting of CD27, CD28, ICOS, 4-1BB, and OX40; (iii) a T cell activation molecule; and (iv) an antigen recognition moiety; and
at least 80% of the modified cells are CD8⁺ T cells.
   A29. A modified cell population, comprising modified T cells, wherein:
the modified T cells comprise a polynucleotide that encodes a chimeric antigen receptor, wherein the chimeric antigen receptor comprises (i) a transmembrane region; (ii) a CD40 polypeptide lacking an extracellular domain and a costimulatory polypeptide cytoplasmic signaling regions selected from the group consisting of CD27, CD28, ICOS, 4-1BB, and OX40; (iii) a T cell activation molecule; and (iv) an antigen recognition moiety; and
the ratio of CD8⁺ to CD4⁺ T cells is 4:1 or greater.
   A30. The modified cell population of any one of embodiments A1 to A29, wherein the modified cells or modified T cells comprise
a first polynucleotide that encodes a chimeric antigen receptor, wherein the chimeric antigen receptor comprises (i) a transmembrane region; (ii) a CD40 polypeptide lacking an extracellular domain and a costimulatory polypeptide cytoplasmic signaling regions selected from the group consisting of CD27, CD28, ICOS, 4-1BB, and OX40; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A31. The modified cell population of embodiment A30, wherein the modified cells or modified T cells comprise a nucleic acid, wherein the nucleic acid comprises
a first polynucleotide that encodes a chimeric antigen receptor, wherein the chimeric antigen receptor comprises (i) a transmembrane region; (ii) a CD40 polypeptide lacking an extracellular domain and a costimulatory polypeptide cytoplasmic signaling regions selected from the group consisting of CD27, CD28, ICOS, 4-1BB, and OX40; (iii) a T cell activation molecule; and (iv) an antigen recognition moiety; and
a second polynucleotide that encodes a chimeric Caspase-9 polypeptide comprising a multimeric ligand binding region and a Caspase-9 polypeptide.
   A32. The modified cell population of any one of embodiments A1-A31, wherein the chimeric antigen receptor is a polypeptide which comprises regions (i)-(v) in order, from the amino terminus to the carboxy terminus of the polypeptide, of (v), (i), (iv), (ii), (iii).
   A33. The modified cell population of any one of embodiments A1-A31, wherein the chimeric antigen receptor is a polypeptide which comprises regions (i)-(v) in order, from the amino terminus to the carboxy terminus of the polypeptide, of (v), (i), (iv), (iii), (ii).
   A34. The modified cell population of any one of embodiments A1-A31, wherein the chimeric antigen receptor is a polypeptide which comprises regions (i)-(v) in order, from the amino terminus to the carboxy terminus of the polypeptide, of (v), (i), (ii), (iii), (iv).
   A35. The modified cell population of any one of embodiments A1-A31, wherein the chimeric antigen receptor is a polypeptide which comprises regions (i)-(v) in order, from the amino terminus to the carboxy terminus of the polypeptide, of (v), (i), (iii), (ii), (iv).
   A36. The modified cell population of embodiment A32, wherein the polynucleotide that encodes the chimeric antigen receptor encodes a linker polypeptide between regions (iv) and (ii)
   A37. The modified cell population of embodiment A33, wherein the polynucleotide that encodes the chimeric antigen receptor encodes a linker polypeptide between regions (iv) and (iii).
   A38. The modified cell population of embodiment A34, wherein the polynucleotide that encodes the chimeric antigen receptor encodes a linker polypeptide between regions (iii) and (iv).
   A39. The modified cell population of embodiment A35, wherein the polynucleotide that encodes the chimeric antigen receptor encodes a linker polypeptide between regions (ii) and (iv).
   A40. The modified cell population of any one of embodiments A36-A39, wherein the linker is a non-cleavable linker.
   A41. The modified cell population of any one of embodiments A36-A39, wherein the linker is a cleavable linker.
   A42. The modified cell population of embodiment A41, wherein the linker is cleaved by an enzyme endogenous to the modified cells in the population.
   A43. The modified cell population of embodiment A41, wherein the linker is cleaved by an enzyme exogenous to the modified cells in the population.
   A44. The modified cell population of any one of embodiments A36 to A39, wherein the linker polypeptide comprises a peptide bond skipping sequence.
   A45. The modified cell population of any one of embodiments A36 to A39, wherein the linker polypeptide comprises a 2A polypeptide.
   A46. The modified cell population of any one of embodiments A1-A45, wherein the antigen recognition moiety binds to an antigen on a target cell.
   B1. The modified cell population of embodiment A1, wherein the modified T cells comprise a second polynucleotide that encodes a chimeric signaling polypeptide, wherein the chimeric signaling polypeptide comprises:
(i) a costimulatory polypeptide cytoplasmic signaling region;
(ii) a truncated MyD88 polypeptide region lacking the TIR domain;
(iii) a truncated MyD88 polypeptide region lacking the TIR domain and a costimulatory polypeptide cytoplasmic signaling region; or
(iv) a truncated MyD88 polypeptide region lacking the TIR domain and a CD40 cytoplasmic polypeptide region lacking the CD40 extracellular domain.
   B2. The modified cell population of embodiment B1, wherein the chimeric signaling polypeptide comprises a membrane targeting region.
   B3. The modified cell population of embodiment B1, wherein the chimeric signaling polypeptide does not include a membrane targeting region.
   B4. The modified cell population of embodiment B1, wherein the modified T cells comprise a nucleic acid comprising a promoter operably linked to
(i) a first polynucleotide encoding the chimeric antigen receptor; and
(ii) a second polynucleotide encoding a chimeric signaling polypeptide, wherein the chimeric signaling polypeptide comprises
   a. a costimulatory polypeptide cytoplasmic signaling region;
   b. a truncated MyD88 polypeptide region lacking the TIR domain;
   c. a truncated MyD88 polypeptide region lacking the TIR domain and a costimulatory polypeptide cytoplasmic signaling region; or
   d. a truncated MyD88 polypeptide region lacking the TIR domain and a CD40 cytoplasmic polypeptide region lacking the CD40 extracellular domain.
      B5. The modified cell population of embodiment B4, wherein the nucleic acid comprises, in 5′ to 3′ order, the first polynucleotide and the second polynucleotide.
      B6. The modified cell population of any one of embodiments B4 or B5, wherein the first polynucleotide encodes, in 5′ to 3′ order, an antigen recognition moiety, a transmembrane region, and a T cell activation molecule, and the second polynucleotide is 3′ of the polynucleotide sequence encoding the T cell activation molecule.
      B7. The modified cell population of any one of embodiments B4 to B6, wherein the nucleic acid comprises a third polynucleotide that encodes a linker polypeptide between the first and the second polynucleotides.
      B8. The modified cell population of embodiment B7, wherein the linker polypeptide comprises a 2A polypeptide.
      B9. The modified cell population of any one of embodiments B7 to B8, wherein the nucleic acid comprises a fourth polynucleotide encoding an inducible chimeric pro-apoptotic polypeptide.
      B10. The modified cell population of any one of embodiments B1 to B9, wherein 80% or more of the modified cells are CD8⁺ T cells.
      B10.1. The modified cell population of any one of embodiments B1 to B10, wherein the chimeric signaling polypeptide comprises two costimulatory polypeptide cytoplasmic signaling regions selected from the group consisting of CD27, CD28, 4-1BB, OX40, ICOS, RANK, TRANCE, and DAP10.
      B11. A modified cell population, comprising a nucleic acid, wherein:
the nucleic acid comprises: a promoter operably linked to a first polynucleotide encoding a cytoplasmic chimeric stimulating molecule, wherein the cytoplasmic chimeric stimulating molecule comprises (i) a MyD88 polypeptide or a truncated MyD88 polypeptide lacking the TIR domain; and (ii) a CD40 cytoplasmic polypeptide region lacking the CD40 extracellular domain; and a second polynucleotide encoding a chimeric antigen receptor; and
at least 80% of the modified cells are CD8⁺ cells.
   B12. A modified cell population, comprising modified T cells, wherein:
the modified T cells comprise a nucleic acid, wherein the nucleic acid comprises:
   a promoter operably linked to a first polynucleotide encoding a cytoplasmic chimeric stimulating molecule, wherein the cytoplasmic chimeric stimulating molecule comprises (i) a MyD88 polypeptide or a truncated MyD88 polypeptide lacking the TIR domain; and (ii) a CD40 cytoplasmic polypeptide region lacking the CD40 extracellular domain; and a second polynucleotide encoding a chimeric antigen receptor; and
the ratio of CD8⁺ to CD4⁺ T cells is 4:1 or greater.
   B13. The modified cell population of any one of embodiments B1-B12, wherein the chimeric antigen receptor comprises an antigen recognition moiety, a transmembrane region, and a T cell activation molecule.
   B14. The modified cell population of any one of embodiments B1-B13, wherein the nucleic acid comprises a polynucleotide that encodes a linker polypeptide between the first and second polynucleotides.
   B15. The modified cell population of any one of embodiments B1-B14, wherein the modified cells or modified T cells comprise a polynucleotide that encodes a chimeric Caspase-9 polypeptide comprising a multimeric ligand binding region and a Caspase-9 polypeptide.
   B16. The modified cell population of any one of embodiments B1-B14, wherein the nucleic acid comprises a polynucleotide that encodes a chimeric Caspase-9 polypeptide comprising a multimeric ligand binding region and a Caspase-9 polypeptide.
   B17. The modified cell population of any one of embodiments B14-B16, wherein the linker is a non-cleavable linker.
   B18. The modified cell population of any one of embodiments B14-B16, wherein the linker is a cleavable linker.
   B19. The modified cell population of embodiment B18, wherein the linker is cleaved by an enzyme endogenous to the modified cells in the population.
   B20. The modified cell population of embodiment B18, wherein the linker is cleaved by an enzyme exogenous to the modified cells in the population.
   B21. The modified cell population of any one of embodiments B14 to B16, wherein the linker polypeptide comprises a peptide bond skipping sequence.
   B22. The modified cell population of any one of embodiments B14 to B16, wherein the linker polypeptide comprises a 2A polypeptide.
   B23. The modified cell population of any one of embodiments B1 to B22, wherein the chimeric signaling polypeptide or the cytoplasmic chimeric stimulating molecule comprises a membrane targeting region.
   B24. The modified cell population of any one of embodiments B1 to B22, wherein the chimeric signaling polypeptide or the cytoplasmic chimeric stimulating molecule does not include a membrane targeting region.
   B25. The modified cell population of any one of embodiments B1-B24, wherein the antigen recognition moiety binds to an antigen on a target cell.
   B26. A modified cell population, comprising a nucleic acid, wherein:
the nucleic acid comprises a promoter operably linked to a first polynucleotide encoding a costimulatory polypeptide cytoplasmic signaling region selected from the group consisting of CD27, CD28, ICOS, 4-1BB, and OX40; and a second polynucleotide encoding a chimeric antigen receptor; and
at least 80% of the modified cells are CD8⁺ T cells.
   B26. A modified cell population, comprising modified T cells, wherein:
the modified T cells comprise a nucleic acid, wherein the nucleic acid comprises:
   a promoter operably linked to a first polynucleotide encoding a costimulatory polypeptide cytoplasmic signaling region selected from the group consisting of CD27, CD28, ICOS, 4-1BB, and OX40; and a second polynucleotide encoding a chimeric antigen receptor; and
the ratio of CD8⁺ to CD4⁺ T cells is 4:1 or greater.
   B2.1. A modified cell population, comprising a nucleic acid, wherein:
the nucleic acid comprises a promoter operably linked to a first polynucleotide encoding two costimulatory polypeptide cytoplasmic signaling regions selected from the group consisting of CD27, CD28, ICOS, 4-1BB, and OX40; and a second polynucleotide encoding a chimeric antigen receptor; and
at least 80% of the modified cells are CD8⁺ T cells.
   B27. A modified cell population, comprising modified T cells, wherein:
the modified T cells comprise a nucleic acid, wherein the nucleic acid comprises a promoter operably linked to a first polynucleotide encoding two costimulatory polypeptide cytoplasmic signaling regions selected from the group consisting of CD27, CD28, ICOS, 4-1BB, and OX40; and a second polynucleotide encoding a chimeric antigen receptor; and
the ratio of CD8⁺ to CD4⁺ T cells is 4:1 or greater.
   B28. A modified cell population, comprising a nucleic acid, wherein:
the nucleic acid comprises a promoter operably linked to a first polynucleotide encoding a MyD88 polypeptide or truncated MyD88 polypeptide lacking a TIR domain; and
   a second polynucleotide encoding a chimeric antigen receptor; and
at least 80% of the modified cells are CD8⁺ T cells.
   B29. A modified cell population, comprising modified T cells, wherein:
the modified T cells comprise a nucleic acid, wherein the nucleic acid comprises a promoter operably linked to a first polynucleotide encoding a MyD88 polypeptide or truncated MyD88 polypeptide lacking a TIR domain; and a second polynucleotide encoding a chimeric antigen receptor; and
the ratio of CD8⁺ to CD4⁺ T cells is 4:1 or greater.
   B30. A modified cell population, comprising a nucleic acid, wherein:
the nucleic acid comprises a promoter operably linked to a first polynucleotide encoding a MyD88 polypeptide or truncated MyD88 polypeptide lacking a TIR domain and a costimulatory polypeptide cytoplasmic signaling regions selected from the group consisting of CD27, CD28, ICOS, 4-1BB, and OX40; and a second polynucleotide encoding a chimeric antigen receptor; and
at least 80% of the modified cells are CD8⁺ T cells.
   B31. A modified cell population, comprising modified T cells, wherein:
the modified T cells comprise a nucleic acid, wherein the nucleic acid comprises a promoter operably linked to a first polynucleotide encoding a MyD88 polypeptide or truncated MyD88 polypeptide lacking a TIR domain and a costimulatory polypeptide cytoplasmic signaling regions selected from the group consisting of CD27, CD28, ICOS, 4-1BB, and OX40; and a second polynucleotide encoding a chimeric antigen receptor; and
the ratio of CD8⁺ to CD4⁺ T cells is 4:1 or greater.
   B32. A modified cell population, comprising a nucleic acid, wherein:
the nucleic acid comprises a promoter operably linked to a first polynucleotide encoding a CD40 polypeptide lacking an extracellular domain; and a second polynucleotide encoding a chimeric antigen receptor; and
at least 80% of the modified cells are CD8⁺ T cells.
   B33. A modified cell population, comprising modified T cells, wherein:
the modified T cells comprise a nucleic acid, wherein the nucleic acid comprises a promoter operably linked to a first polynucleotide encoding a CD40 polypeptide lacking an extracellular domain; and a second polynucleotide encoding a chimeric antigen receptor; and
the ratio of CD8⁺ to CD4⁺ T cells is 4:1 or greater.
   B34. A modified cell population, comprising a nucleic acid, wherein:
the nucleic acid comprises a promoter operably linked to a first polynucleotide encoding a CD40 polypeptide lacking an extracellular domain and a costimulatory polypeptide cytoplasmic signaling regions selected from the group consisting of CD27, CD28, ICOS, 4-1BB, and OX40; and a second polynucleotide encoding a chimeric antigen receptor; and
at least 80% of the modified cells are CD8⁺ T cells.
   B35. A modified cell population, comprising modified T cells, wherein:
the modified T cells comprise a nucleic acid, wherein the nucleic acid comprises a promoter operably linked to a first polynucleotide encoding a CD40 polypeptide lacking an extracellular domain and a costimulatory polypeptide cytoplasmic signaling regions selected from the group consisting of CD27, CD28, ICOS, 4-1BB, and OX40; and a second polynucleotide encoding a chimeric antigen receptor; and
the ratio of CD8⁺ to CD4⁺ T cells is 4:1 or greater.
   B36. The modified cell population of any one of embodiments B26-B35, wherein the chimeric antigen receptor comprises an antigen recognition moiety, a transmembrane region, and a T cell activation molecule.
   B37. The modified cell population of any one of embodiments B26-B36, wherein the nucleic acid comprises a polynucleotide that encodes a linker polypeptide between the first and second polynucleotides.
   B38. The modified cell population of any one of embodiments B26-B37, wherein the modified cells or modified T cells comprise a polynucleotide that encodes a chimeric Caspase-9 polypeptide comprising a multimeric ligand binding region and a Caspase-9 polypeptide.
   B39. The modified cell population of any one of embodiments B26-B38, wherein the nucleic acid comprises a polynucleotide that encodes a chimeric Caspase-9 polypeptide comprising a multimeric ligand binding region and a Caspase-9 polypeptide.
   B40. The modified cell population of any one of embodiments B37-B39, wherein the linker is a non-cleavable linker.
   B41. The modified cell population of any one of embodiments B37-B39, wherein the linker is a cleavable linker.
   B42. The modified cell population of embodiment B41, wherein the linker is cleaved by an enzyme endogenous to the modified cells in the population.
   B43. The modified cell population of embodiment B41, wherein the linker is cleaved by an enzyme exogenous to the modified cells in the population.
   B44. The modified cell population of any one of embodiments B37 to B39, wherein the linker polypeptide comprises a peptide bond skipping sequence.
   B45. The modified cell population of any one of embodiments B37 to B39, wherein the linker polypeptide comprises a 2A polypeptide.
   B46. The modified cell population of any one of embodiments B26 to B45, wherein the chimeric signaling polypeptide or the cytoplasmic chimeric stimulating molecule comprises a membrane targeting region.
   B47. The modified cell population of any one of embodiments B26 to B45, wherein the chimeric signaling polypeptide or the cytoplasmic chimeric stimulating molecule does not include a membrane targeting region.
   B48. The modified cell population of any one of embodiments B26-B47, wherein the antigen recognition moiety binds to an antigen on a target cell.
   C1. The modified cell population of any one of embodiments A1-B48, wherein the chimeric antigen receptor comprises a stalk polypeptide.
   C2. The modified cell population of any one of embodiments A1-C1, wherein the T cell activation molecule is an ITAM-containing, Signal 1 conferring molecule.
   C3. The modified cell population of any one of embodiments A1-C1, wherein the T cell activation molecule is a CD3 ζ polypeptide.
   C4. The modified cell population of any one of embodiments A1-C1, wherein the T cell activation molecule is an Fc epsilon receptor gamma (FcεR1γ) subunit polypeptide.
   C5. The modified cell population of any one of embodiments A1-C4, wherein the linker polypeptide separates the translation products of the first and second polynucleotides during or after translation.
   C5.1. The modified cell population of embodiment C5, wherein the linker polypeptide is cleaved during or after translation of the first and second polynucleotides.
   C5.2. The modified cell population of any one of embodiments A1-C5.1, wherein the chimeric antigen receptor comprises a membrane targeting region linked to the MyD88 or CD40 polypeptides.
   C5.3. The modified cell population of any one of embodiments A1-C5.1, wherein the polynucleotide that encodes the MyD88 and CD40 polypeptides encodes a membrane targeting region linked to the MyD88 or CD40 polypeptides.
   C5.4. The modified cell population of any one of embodiments C5.2 or C5.3, wherein the membrane targeting region is a myristoylation region.
   C5.5. The modified cell population of any one of embodiments A1-C5.4, wherein the chimeric antigen receptor comprises a membrane targeting region linked to one of the costimulatory molecule cytoplasmic signaling regions.
   C5.6. The modified cell population of any one of embodiments A1-C5.5, wherein the polynucleotide that encodes the costimulatory cytoplasmic signaling region encodes a membrane targeting region.
   C6. The modified cell population of any one of embodiments A1-C5.6, wherein the linker polypeptide is not cleaved during translation of the polynucleotide that encodes the chimeric antigen receptor, and the modified cell expresses a chimeric antigen receptor linked to the MyD88 and CD40 polypeptides.
   C6.1. The modified cell population of any one of embodiments A1 to C6, wherein the linker polypeptide is not cleaved during translation of the polynucleotide that encodes the chimeric antigen receptor.
   C6.2. The modified cell population of any one of embodiments A1-C6, wherein the linker polypeptide is cleaved during or after translation of the polynucleotide that encodes the chimeric antigen receptor.
   C7. The modified cell population of any one of embodiments A1-C6, wherein the linker polypeptide is a 2A polypeptide.
   C8. The modified cell population of any one of embodiments A1-C7, wherein the transmembrane region is a CD8 transmembrane region.
   C9. The modified cell population of any one of embodiments A1-C8, wherein the MyD88 polypeptide has the amino acid sequence of SEQ ID NO: 35 or SEQ ID NO: 83, or a functional fragment thereof.
   C10. The modified cell population of any one of embodiments A1-08, wherein the truncated MyD88 polypeptide has the amino acid sequence of SEQ ID NO: 27, or a functional fragment thereof.
   C11. The modified cell population of any one of embodiments A1-C10, wherein the truncated MyD88 polypeptide comprises the amino acid sequence of SEQ ID NO: 35 or SEQ ID NO: 83, or lacking the TIR domain, or a functional fragment thereof.
   C11.1. The modified cell population of any one of embodiments A1-C10, wherein the truncated MyD88 polypeptide does not comprise contiguous amino acid residues 156 to the C-terminus of the full length MyD88 polypeptide.
   C11.2. The modified cell population of any one of embodiments A1-C10, wherein the truncated MyD88 polypeptide does not comprise contiguous amino acid residues 152 to the C-terminus of the full length MyD88 polypeptide.
   C11.3. The modified cell population of any one of embodiments A1-C10, wherein the truncated MyD88 polypeptide does not comprise contiguous amino acid residues 173 to the C-terminus of the full length MyD88 polypeptide.
   C11.4. The modified cell population of any one of embodiments A1-C8, wherein the full length MyD88 polypeptide comprises the amino acid sequence of SEQ ID NO: 35 or SEQ ID NO: 83.
   C11.5. The modified cell population of any one of embodiments A1-C10, wherein the truncated MyD88 polypeptide consists of the amino acid sequence of SEQ ID NO: 35 or SEQ ID NO: 83, or a functional fragment thereof.
   C12. The modified cell population of any one of embodiments A1-C11.5, wherein the cytoplasmic CD40 polypeptide comprises the amino acid sequence of SEQ ID NO: 29, or a functional fragment thereof.
   C13. The modified cell population of any one of embodiments A1-C11.5, wherein the cytoplasmic CD40 polypeptide consists of the amino acid sequence of SEQ ID NO: 29, or a functional fragment thereof.
   C14. The modified cell population of any one of embodiments A1-C13, wherein the CD3 polypeptide comprises an amino acid sequence of SEQ ID NO: 23, or a functional fragment thereof.
   C15. The modified cell population of any one of embodiments A1-C14, wherein the transmembrane region polypeptide comprises an amino acid sequence of SEQ ID NO: 21, or a functional fragment thereof.
   C16. The modified cell population of any one of embodiments A1-C15, wherein the antigen recognition moiety binds to an antigen on a tumor cell.
   C17. The modified cell population of any one of embodiments A1-C16, wherein the antigen recognition moiety binds to an antigen on a cell involved in a hyperproliferative disease.
   C18. The modified cell population of any one of embodiments A1-C17, wherein the antigen recognition moiety binds to an antigen selected from the group consisting of PSMA, PSCA, MUC1, CD19, ROR1, Mesothelin, GD2, CD123, MUC16, and Her2/Neu.
   C19. The modified cell population of any one of embodiments A1-C18, wherein the antigen recognition moiety binds to Her2/Neu.
   C20. The modified cell population of any one of embodiments A1-C18, wherein the antigen recognition moiety binds to CD19.
   C21. The modified cell population of any one of embodiments A1-C18, wherein the antigen recognition moiety binds to a viral or bacterial antigen.
   C22. The modified cell population of any one of embodiments A1-C21, wherein the antigen recognition moiety is a single chain variable fragment.
   C23. The modified cell population of any one of embodiments A4-C22, wherein the multimeric ligand binding region binds to dimeric FK506, or a dimeric FK506-like analog.
   C23.1. The modified cell population of any one of embodiments A4-C22, wherein the multimeric ligand binding region binds to rimiducid or to AP20187.
   C23.2. The modified cell population of any one of embodiments A4-C23.1, wherein the multimeric ligand binding region comprises an FKBP12 variant polypeptide.
   C23.3. The modified cell population of embodiment C23.2, wherein the FKBP12 variant polypeptide binds with higher affinity to the multimeric ligand than the wild type FKBP12 polypeptide.
   C23.4. The modified cell population of any one of embodiments C23.2 or C23.3, wherein the FKBP12 variant polypeptide comprises an amino acid substitution at position 36 that binds with higher affinity to the multimeric ligand than the wild type FKBP12 polypeptide.
   C23.5. The modified cell population of embodiment C23.4, wherein the amino acid substitution at position 36 is selected from the group consisting of valine, isoleucine, leucine, and alanine.
   C23.6. The modified cell population of embodiment C23.5, wherein the multimeric ligand binding region is an FKB12v36 region.
   C24. The modified cell population of any one of embodiments A1 to C23.6, wherein the ratio of CD8+ to CD4+ T cells is 9:1 or greater
   C25. The modified cell population of any one of embodiments A1 to C23.6, wherein at least 90% of the modified cells are CD8+ T cells.
   C26. The modified cell population of any one of embodiments A1 to C23.6, wherein at least 95% of the modified cells are CD8+ T cells
   C27. The modified cell population of any one of embodiments A4-C26, wherein the inducible Caspase-9 polypeptide comprises the amino acid sequence of SEQ ID NO: 5.
   C27.1. The modified cell population of any one of embodiments A4-C26, wherein the Caspase-9 polypeptide is a modified Caspase-9 polypeptide comprising an amino acid substitution selected from the group consisting of D330A, D330E, and N405Q.
   C28. The modified cell population of any one of embodiments A1-C27.1, wherein the polynucleotide that encodes the chimeric antigen receptor, or the nucleic acid is contained within a viral vector.
   C29. The modified cell population of embodiment C28, wherein the viral vector is a retroviral vector.
   C30. The modified cell population of embodiment C29, wherein the retroviral vector is a murine leukemia virus vector.
   C31. The modified cell population of embodiment C29, wherein the retroviral vector is an SFG vector.
   C32. The modified cell population of embodiment C26, wherein the viral vector is an adenoviral vector.
   C33. The modified cell population of embodiment C26, wherein the viral vector is a lentiviral vector.
   C34. The modified cell population of embodiment C26, wherein the viral vector is selected from the group consisting of adeno-associated virus (AAV), Herpes virus, and Vaccinia virus.
   C35. The modified cell population of any one of embodiments A1-C34, wherein the polynucleotide that encodes the chimeric antigen receptor or the nucleic acid is prepared or in a vector designed for electroporation, sonoporation, or biolistics, or is attached to or incorporated in chemical lipids, polymers, inorganic nanoparticles, or polyplexes.
   C36. The modified cell population of any one of embodiments A1-C34, wherein the polynucleotide that encodes the chimeric antigen receptor or the nucleic acid is contained within a plasmid.

### C37. Reserved.

C38. The modified cell population of any one of embodiments A1-C37, wherein the cells are obtained or prepared from bone marrow.
C39. The modified cell population of any one of embodiments A1-C37, wherein the cells are obtained or prepared from umbilical cord blood.
C40. The modified cell population of any one of embodiments A1-C37, wherein the cells are obtained or prepared from peripheral blood.
C41. The modified cell population of any one of embodiments A1-C37, wherein the cells are obtained or prepared from peripheral blood mononuclear cells.
C42. The modified cell population of any one of embodiments A1-C41, wherein the modified cells are human cells.
C43. The method of any one of embodiments A1-C41, wherein the modified cells are autologous T cells.
C44. The method of any one of embodiments A1-C41, wherein the modified cells are allogeneic T cells.
C45. The modified cell population of any one of embodiments A1-C44, wherein the cells are transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

### C46-C48.

D1. A method for stimulating a cell mediated immune response to a target cell or tissue in a subject, comprising administering a modified cell population of any one of embodiments A1-048 to the subject.
D1.1. A method for treating a subject having a disease or condition associated with an elevated expression of a target antigen, comprising administering to the subject an effective amount of a modified cell population of any one of embodiments A1 to C48.
D1.2. A method for reducing the size of a tumor in a subject, comprising administering a modified cell population of any one of embodiments A1 to C48 to the subject, wherein the antigen recognition moiety binds to an antigen on the tumor.
D2. The method of any one of embodiments D1 to D1.2, wherein the target cell is a tumor cell.
D3. The method of any one of embodiments D1 to D2, wherein the number or concentration of target cells in the subject is reduced following administration of the modified cell population.
D4. The method of any one of embodiments D1-D3, comprising measuring the number or concentration of target cells in a first sample obtained from the subject before administering the modified cell population, measuring the number concentration of target cells in a second sample obtained from the subject after administration of the modified cell population, and determining an increase or decrease of the number or concentration of target cells in the second sample compared to the number or concentration of target cells in the first sample.
D4. The method of embodiment D4, wherein the concentration of target cells in the second sample is decreased compared to the concentration of target cells in the first sample.
D5. The method of embodiment D4, wherein the concentration of target cells in the second sample is increased compared to the concentration of target cells in the first sample.
D6. The method of any one of embodiments D1-D5, wherein an additional dose of modified cells is administered to the subject.
D7. A method for providing anti-tumor immunity to a subject, comprising administering to the subject an effective amount of a modified cell population of any one of embodiments A1-C48.
D8. A method for treating a subject having a disease or condition associated with an elevated expression of a target antigen, comprising administering to the subject an effective amount of a modified cell population of any one of embodiments A1-C48.
D9. The method of embodiment D8, wherein the target antigen is a tumor antigen.
D10. A method for reducing the size of a tumor in a subject, comprising administering a modified cell population of any one of embodiments A1-C48 to the subject, wherein the antigen recognition moiety binds to an antigen on the tumor.
D11. The method of any one of embodiments D1-D10, wherein the subject has been diagnosed as having a tumor.
D12. The method of any one of embodiments D1-D11, wherein the subject has cancer.
D13. The method of any one of embodiments D1-D12, wherein the subject has a solid tumor.
D14. The method of any one of embodiments D1-D13 wherein the modified cell population is administered intravenously.
D15. The method of any one of embodiments D1-D14, wherein the modified cell population is delivered to a tumor bed.
D16. The method of embodiment D12, wherein the cancer is present in the blood or bone marrow of the subject.
D17. The method of any one of embodiments D1-D16, wherein the subject has a blood or bone marrow disease.
D18. The method of any one of embodiments D1-D17, wherein the subject has been diagnosed with any condition that can be alleviated by stem cell transplantation.
D19. The method of any one of embodiments D1-D18, wherein the subject has been diagnosed with sickle cell anemia or metachromatic leukodystrophy.
D20. The method of any one of embodiments D1-D18, wherein the patient has been diagnosed with a condition selected from the group consisting of a primary immune deficiency condition, hemophagocytosis lymphohistiocytosis (HLH) or other hemophagocytic condition, an inherited marrow failure condition, a hemoglobinopathy, a metabolic condition, and an osteoclast condition.
D21. The method of any one of embodiments D1-D18, wherein the disease or condition is selected from the group consisting of Severe Combined Immune Deficiency (SCID), Combined Immune Deficiency (CID), Congenital T cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/I L-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.
D22. The method of any one of embodiments D1-D21, comprising administering an additional dose of the modified cell to the subject, wherein the disease or condition symptoms remain or are detected following a reduction in symptoms.
D23. The method of any one of embodiments D1-D22, comprising
identifying the presence, absence or stage of a condition or disease in a subject; and
transmitting an indication to administer modified cell population of any one of embodiments A1-C48, maintain a subsequent dosage of the modified cell population, or adjust a subsequent dosage of the modified cell population administered to the patient based on the presence, absence or stage of the condition or disease identified in the subject.
   D24. The method of any one of embodiments D1-D23, wherein the condition is leukemia.
   D25. The method of any one of embodiments D1-D23, wherein the subject has been diagnosed with an infection of viral etiology selected from the group consisting HIV, influenza, Herpes, viral hepatitis, Epstein Bar, polio, viral encephalitis, measles, chicken pox, Cytomegalovirus (CMV), adenovirus (ADV), HHV-6 (human herpesvirus 6, I), and Papilloma virus, or has been diagnosed with an infection of bacterial etiology selected from the group consisting of pneumonia, tuberculosis, and syphilis, or has been diagnosed with an infection of parasitic etiology selected from the group consisting of malaria, trypanosomiasis, leishmaniasis, trichomoniasis, and amoebiasis.
   D26. The method of any one of embodiments D1-D25, wherein the subject has been administered a modified cell population of any one of embodiments A1 to C48, wherein the modified cell population comprises a polynucleotide that encodes an inducible chimeric pro-apoptotic polypeptide comprising a multimeric ligand binding region, comprising administering a multimeric ligand that binds to the multimeric ligand binding region to the subject following administration of the modified cell population to the subject.
   D27. The method of embodiment D26, wherein after administration of the multimeric ligand, the number of modified cells comprising the inducible chimeric pro-apoptotic polypeptide is reduced.
   D28. The method of any one of embodiments D26 or D27, wherein the number of modified cells comprising the inducible chimeric pro-apoptotic polypeptide is reduced by 90%.
   D28.1. The method of any one of embodiments D26 or D27, wherein the number of modified cells comprising the inducible chimeric pro-apoptotic polypeptide is reduced by 70%.
   D28.2. The method of any one of embodiments D26 or D27, wherein the number of modified cells comprising the inducible chimeric pro-apoptotic polypeptide is reduced by 50%.
   D28.3. The method of any one of embodiments D26 or D27, wherein the number of modified cells comprising the inducible chimeric pro-apoptotic polypeptide is reduced by 30%.
   D28.4. The method of any one of embodiments D26 or D27, wherein the number of modified cells comprising the inducible chimeric pro-apoptotic polypeptide is reduced by 20%.
   D28.5. The method of any one of embodiments D26 to D28.4, wherein the inducible chimeric pro-apoptotic polypeptide is an inducible chimeric Caspase-9 polypeptide.
   D29. The method of any one of embodiments D26-D28.4, comprising determining that the subject is experiencing a negative symptom following administration of the modified cell population to the subject, and administering the ligand to reduce or alleviate the negative symptom.
   D30. The method of any one of embodiments D26-D29, comprising the steps of
detecting cytokine toxicity the subject;
administering a sufficient dose of a multimeric ligand that binds to the multimeric ligand binding region to reduce the level of cytokine toxicity in the subject.
   D31. The method of embodiment D30, wherein cytokine toxicity is detected by observing physical symptoms in the subject.
   D32. The method of embodiment D31, wherein cytokine toxicity is detected by measuring weight loss in the subject.
   D33. The method of any one of embodiments D26-D33, wherein the subject is diagnosed with cachexia following administration of the modified cell population.
   D34. The method of any one of embodiments D26-D33, wherein the level of at least one cytokine associated with cytokine-related toxicity is elevated in a sample obtained from the subject following administration of the modified cell population, and before administration of the multimeric ligand.
   D35. The method of embodiment D34, wherein the level of the at least one cytokine is decreased in a sample obtained from the subject following administration of the multimeric ligand, compared to the level of the at least one cytokine in the sample obtained from the subject before administration of the multimeric ligand.
   D36. The method of any one of embodiments D26-D35, wherein the multimeric ligand is rimiducid or AP20187.
   D37. The method of any one of embodiments D1-D36, comprising the step of enriching the modified cell population to obtain a cell population enriched for CD8⁺ T cells prior to administering the modified cell population to the subject.
   D38. The method of embodiment D37, comprising enriching the modified cell population to obtain a cell population comprising at least 80% CD8⁺ T cells prior to administering the modified cell population to the subject.
   D39. The method of any one of embodiments D1-D36, comprising the step of purifying CD8⁺ T cells prior to administering the modified cell population to the subject.
   D40. The method of any one of embodiments D37 to D39, wherein the CD8⁺ T cells are enriched using magnetic activated cell sorting.
   D41. The method of embodiment D39, wherein the CD8⁺ T cells are purified using magnetic activated cell sorting.
   E1. A method for preparing a modified cell population of any one of embodiments A1-C48, comprising contacting a cell population with nucleic acid that comprises the polynucleotide that encodes the chimeric antigen receptor with a cell population under conditions in which the nucleic acid is incorporated into the cell, whereby the cell expresses the chimeric antigen receptor from the incorporated nucleic acid.
   E2. A method for preparing a modified cell population of any one of embodiments B1-048, comprising contacting a cell population with the nucleic acid that comprises the polynucleotide that encodes the chimeric antigen receptor with a cell population under conditions in which the nucleic acid is incorporated into the cell, whereby the cell expresses the chimeric antigen receptor from the incorporated nucleic acid.
   E3. A method for preparing a modified cell population of any one of embodiments A1 to C48, comprising contacting T cells with a nucleic acid that comprises a polynucleotide that encodes the chimeric antigen receptor with a cell population under conditions in which the nucleic acid is incorporated into the cells, and enriching the T cells to obtain a modified cell population wherein the ratio of CD8⁺ to CD4⁺ T cells in the cell population is 3:2 or greater.
   E3. The method of any one of embodiments E1 to E2, wherein the cells of the cell population are transfected or transduced with the nucleic acid.
   E4. The method of any one of embodiments E1 to E3, wherein the nucleic acid is contained in a viral vector.
   E5. The method of any one of embodiments E1 to E3, wherein the nucleic acid is contained in a plasmid vector.
   E6. A method for preparing a modified cell population of any one of embodiments A1-C48, comprising enriching a population of modified T cells to obtain a ratio of CD8⁺ to CD4⁺ T cells of 3:2 or greater, wherein the modified T cells comprise a polynucleotide that encodes a chimeric antigen receptor, wherein the chimeric antigen receptor comprises:
(i) a transmembrane region;
(ii) a T cell activation molecule; and
(iii) an antigen recognition moiety.
   E7. The method of embodiment E6, wherein the chimeric antigen receptor comprises
(i) a transmembrane region;
(ii) a costimulatory polypeptide cytoplasmic signaling region, a truncated MyD88 polypeptide region lacking the TIR domain, a truncated MyD88 polypeptide region lacking the TIR domain and a costimulatory polypeptide cytoplasmic signaling region, or a truncated MyD88 polypeptide region lacking the TIR domain and a CD40 cytoplasmic polypeptide region lacking the CD40 extracellular domain;
(iii) a T cell activation molecule; and
(iv) an antigen recognition moiety.
   E8. The method of any one of embodiments E6 or E7, wherein the modified T cells comprise a second polynucleotide that encodes an inducible chimeric pro-apoptotic polypeptide.
   E9. The method of embodiment E6, wherein the modified T cells comprise a second polynucleotide that encodes a chimeric signaling polypeptide, wherein the chimeric signaling polypeptide comprises:
(i) a costimulatory polypeptide cytoplasmic signaling region;
(ii) a truncated MyD88 polypeptide region lacking the TIR domain;
(iii) a truncated MyD88 polypeptide region lacking the TIR domain and a costimulatory polypeptide cytoplasmic signaling region; or
(iv) a truncated MyD88 polypeptide region lacking the TIR domain and a CD40 cytoplasmic polypeptide region lacking the CD40 extracellular domain.
   E10. The method of embodiment E9, wherein the chimeric signaling polypeptide comprises a membrane targeting region.
   E11. The method of embodiment E9, wherein the chimeric signaling polypeptide does not include a membrane targeting region.
   E12. The method of embodiment E6, wherein the modified T cells comprise a nucleic acid comprising a promoter operably linked to
(i) a first polynucleotide encoding the chimeric antigen receptor; and
(ii) a second polynucleotide encoding a chimeric signaling polypeptide, wherein the chimeric signaling polypeptide comprises
   a. a costimulatory polypeptide cytoplasmic signaling region;
   b. a truncated MyD88 polypeptide region lacking the TIR domain;
   c. a truncated MyD88 polypeptide region lacking the TIR domain and a costimulatory polypeptide cytoplasmic signaling region; or
   d. a truncated MyD88 polypeptide region lacking the TIR domain and a CD40 cytoplasmic polypeptide region lacking the CD40 extracellular domain.
      E13. The method of embodiment E12, wherein the nucleic acid comprises, in 5′ to 3′ order, the first polynucleotide and the second polynucleotide.
      E14. The method of any one of embodiments E12 or E13, wherein the first polynucleotide encodes, in 5′ to 3′ order, an antigen recognition moiety, a transmembrane region, and a T cell activation molecule, and the second polynucleotide is 3′ of the polynucleotide sequence encoding the T cell activation molecule.
      E15. The method of any one of embodiments E12 to E14, wherein the nucleic acid comprises a third polynucleotide that encodes a linker polypeptide between the first and the second polynucleotides.
      E16. The method of embodiment E15, wherein the linker polypeptide comprises a 2A polypeptide.
      E17. The method of any one of embodiments E15 or E16, wherein the nucleic acid comprises a fourth polynucleotide encoding an inducible chimeric pro-apoptotic polypeptide.
      E18. The method of any one of embodiments E7 to E17, wherein the costimulatory polypeptide cytoplasmic signaling region is selected from the group consisting of CD27, CD28, 4-1BB, OX40, ICOS, RANK, TRANCE, and DAP10.
      E19. The method of any one of embodiments E7 to E8, wherein the chimeric antigen receptor comprises two costimulatory polypeptide cytoplasmic signaling regions selected from the group consisting of CD27, CD28, 4-1BB, OX40, ICOS, RANK, TRANCE, and DAP10.
      E20. The method of any one of embodiments E9 to E17, wherein the chimeric signaling polypeptide comprises two costimulatory polypeptide cytoplasmic signaling regions selected from the group consisting of CD27, CD28, 4-1BB, OX40, ICOS, RANK, TRANCE, and DAP10.
      E21. The method of any one of embodiments E1 to E20, wherein the modified cell population is subjected to magnetic activated cell sorting (MACS).
      E22. The method of any one of embodiments E1 to E21, wherein the modified cell population is selected to comprise CD4⁺ and CD8⁺ T cell fractions.
      E23. The method of any one of embodiments E1 to E22, wherein the modified cell population is tested to determine the percentage of CD8⁺ T cells.
      E24. The method of embodiment E23, comprising the step of administering the modified cell population to a subject.
      F1. A method for preparing a CD8⁺ T cell enriched modified cell population, comprising enriching a modified cell population to obtain a modified cell population that comprises at least 80% CD8⁺ T cells, wherein the modified cells comprise a polynucleotide that encodes a chimeric antigen receptor, wherein:
the chimeric antigen receptor comprises (i) a transmembrane region; (ii) a MyD88 polypeptide or a truncated MyD88 polypeptide lacking a TIR domain; (iii) a CD40 cytoplasmic polypeptide region lacking a CD40 extracellular domain; (iv) a T cell activation molecule; and (v) an antigen recognition moiety.
   F1.1. A method for preparing a CD8⁺ T cell enriched modified cell population of any one of embodiments A1 to C48, comprising enriching a modified cell population to obtain a modified cell population that comprises at least 80% CD8⁺ T cells,
   F2. A method for preparing a CD8⁺ T cell enriched modified cell population, comprising enriching a modified cell population to obtain a modified cell population wherein the ratio of CD8⁺ to CD4⁺ T cells is 4:1 or greater, wherein the modified cell population comprises modified T cells that comprise a polynucleotide that encodes a chimeric antigen receptor, wherein the chimeric antigen receptor comprises (i) a transmembrane region; (ii) a MyD88 polypeptide or a truncated MyD88 polypeptide lacking a TIR domain; (iii) a CD40 cytoplasmic polypeptide region lacking a CD40 extracellular domain; (iv) a T cell activation molecule; and (v) an antigen recognition moiety.
   F3. The method of any one of embodiments F1 to F2, wherein the modified cells or modified T cells comprise
a first polynucleotide that encodes a chimeric antigen receptor, wherein the chimeric antigen receptor comprises (i) a transmembrane region; (ii) a MyD88 polypeptide or a truncated MyD88 polypeptide lacking a TIR domain; (iii) a CD40 cytoplasmic polypeptide region lacking a CD40 extracellular domain; (iv) a T cell activation molecule; and (v) an antigen recognition moiety; and
a second polynucleotide that encodes a chimeric Caspase-9 polypeptide comprising a multimeric ligand binding region and a Caspase-9 polypeptide.
   F4. The method of embodiment F3, wherein the modified cells or modified T cells comprise a nucleic acid, wherein:
the nucleic acid comprises a first polynucleotide that encodes a chimeric antigen receptor, wherein the chimeric antigen receptor comprises (i) a transmembrane region; (ii) a MyD88 polypeptide or a truncated MyD88 polypeptide lacking a TIR domain; (iii) a CD40 cytoplasmic polypeptide region lacking a CD40 extracellular domain; (iv) a T cell activation molecule; and (v) an antigen recognition moiety; and
a second polynucleotide that encodes a chimeric Caspase-9 polypeptide comprising a multimeric ligand binding region and a Caspase-9 polypeptide.
   F5. The method of any one of embodiments F1-F4, wherein the chimeric antigen receptor is a polypeptide which comprises regions (i)-(v) in order, from the amino terminus to the carboxy terminus of the polypeptide, of (v), (i), (iv), (ii), (iii).
   F6. The method of any one of embodiments F1-F4, wherein the chimeric antigen receptor is a polypeptide which comprises regions (i)-(v) in order, from the amino terminus to the carboxy terminus of the polypeptide, of (v), (i), (iv), (iii), (ii).
   F7. The method of any one of embodiments F1-F4, wherein the chimeric antigen receptor is a polypeptide which comprises regions (i)-(v) in order, from the amino terminus to the carboxy terminus of the polypeptide, of (v), (i), (ii), (iii), (iv).
   F8. The method of any one of embodiments F1-F4, wherein the chimeric antigen receptor is a polypeptide which comprises regions (i)-(v) in order, from the amino terminus to the carboxy terminus of the polypeptide, of (v), (i), (iii), (ii), (iv).
   F9. The method of embodiment F5, wherein the polynucleotide that encodes the chimeric antigen receptor encodes a linker polypeptide between regions (iv) and (ii).
   F10. The method of embodiment F6, wherein the polynucleotide that encodes the chimeric antigen receptor encodes a linker polypeptide between regions (iv) and (iii).
   F11. The method of embodiment F7, wherein the polynucleotide that encodes the chimeric antigen receptor encodes a linker polypeptide between regions (iii) and (iv).
   F12. The method of embodiment F8, wherein the polynucleotide that encodes the chimeric antigen receptor encodes a linker polypeptide between regions (ii) and (iv).
   F13. The method of any one of embodiments F9-F12, wherein the linker is a non-cleavable linker.
   F14. The method of any one of embodiments F9-F12, wherein the linker is a cleavable linker.
   F15. The method of embodiment F14, wherein the linker is cleaved by an enzyme endogenous to the modified cells in the population.
   F16. The method of embodiment F14, wherein the linker is cleaved by an enzyme exogenous to the modified cells in the population.
   F17. The method of any one of embodiments F1-F16, wherein the antigen recognition moiety binds to an antigen on a target cell.
   G1. A method for preparing a CD8⁺ T cell enriched modified cell population, comprising enriching a modified cell population to obtain a modified cell population that comprises at least 80% CD8⁺ T cells, wherein the modified cells comprise a nucleic acid, wherein:
the nucleic acid comprises: a promoter operably linked to a first polynucleotide encoding a cytoplasmic chimeric stimulating molecule, wherein the cytoplasmic chimeric stimulating molecule comprises (i) a MyD88 polypeptide or a truncated MyD88 polypeptide lacking the TIR domain; and (ii) a CD40 cytoplasmic polypeptide region lacking the CD40 extracellular domain; and a second polynucleotide encoding a chimeric antigen receptor.
   G1.1. A method for preparing a CD8⁺ T cell enriched modified cell population of any one of embodiments A1 to C48.
   G2. A method for preparing a CD8⁺ T cell enriched modified cell population, comprising enriching a modified cell population to obtain a modified cell population wherein the ratio of CD8⁺ to CD4⁺ T cells is 4:1 or greater, wherein the modified cell population comprises modified T cells that comprise a nucleic acid, wherein the nucleic acid comprises:
   a promoter operably linked to a first polynucleotide encoding a cytoplasmic chimeric stimulating molecule, wherein the cytoplasmic chimeric stimulating molecule comprises (i) a MyD88 polypeptide or a truncated MyD88 polypeptide lacking the TIR domain; and (ii) a CD40 cytoplasmic polypeptide region lacking the CD40 extracellular domain; and a second polynucleotide encoding a chimeric antigen receptor.
   G3. The method of any one of embodiments G1-G2, wherein the chimeric antigen receptor comprises an antigen recognition moiety, a transmembrane region, and a T cell activation molecule.
   G4. The method of any one of embodiments G1-G3, wherein the nucleic acid comprises a polynucleotide that encodes a linker polypeptide between the first and second polynucleotides.
   G5. The method of any one of embodiments G1-G4, wherein the modified cells or modified T cells comprise a polynucleotide that encodes a chimeric Caspase-9 polypeptide comprising a multimeric ligand binding region and a Caspase-9 polypeptide.
   G6. The method of any one of embodiments G1-G4, wherein the nucleic acid comprises a polynucleotide that encodes a chimeric Caspase-9 polypeptide comprising a multimeric ligand binding region and a Caspase-9 polypeptide.
   G7. The method of any one of embodiments G4-G6, wherein the linker is a non-cleavable linker.
   G8. The method of any one of embodiments G4-G6, wherein the linker is a cleavable linker.
   G9. The method of embodiment G8, wherein the linker is cleaved by an enzyme endogenous to the modified cells in the population.
   G10. The method of embodiment G8, wherein the linker is cleaved by an enzyme exogenous to the modified cells in the population.
   G11. The method of any one of embodiments G1-G10, wherein the antigen recognition moiety binds to an antigen on a target cell.
   G12. The method of any one of embodiments E1-G11, comprising the step of purifying CD8⁺ T cells.
   G13. The method of any one of embodiments E1-G11, wherein the CD8⁺ T cells are enriched using magnetic activated cell sorting.
   G14. The method of embodiment G12, wherein the CD8⁺ T cells are purified using magnetic activated cell sorting.
   H1. The method of any one of embodiments E1-F17, or G1-G14, wherein the chimeric antigen receptor comprises a stalk polypeptide.
   H2. The method of any one of embodiments E1-F17, G1-G14, or H1, wherein the T cell activation molecule is an ITAM-containing, Signal 1 conferring molecule.
   H3. The method of any one of embodiments E1-F17, G1-G14, or H1, wherein the T cell activation molecule is a CD3 polypeptide.
   H4. The method of any one of embodiments E1-F17, G1-G14, or H1, wherein the T cell activation molecule is an Fc epsilon receptor gamma (FcεR1γ) subunit polypeptide.
   H5. The method of any one of embodiments G4-G14, wherein the linker polypeptide separates the translation products of the first and second polynucleotides during or after translation.
   H5.1. The method of embodiment H5, wherein the linker polypeptide is cleaved during or after translation of the first and second polynucleotides.
   H6. The method of any one of embodiments F9-F17 or G4-G14, wherein the linker polypeptide is not cleaved during translation of the polynucleotide that encodes the chimeric antigen receptor, and the modified cell expresses a chimeric antigen receptor linked to the MyD88 and CD40 polypeptides.
   H6.1. The method of any one of embodiments F9-F17 or G4-G14, wherein the linker polypeptide is cleaved during or after translation of the polynucleotide that encodes the chimeric antigen receptor.
   H7. The method of any one of embodiments F9-F17, G4-G14, or H1-H6, wherein the linker polypeptide is a 2A polypeptide.
   H8. The method of any one of embodiments E1-F17. G1-G14, or H1-H7, wherein the transmembrane region is a CD8 transmembrane region.
   H9. The method of any one of embodiments E1-F17, G1-G14, or H1-H8, wherein the MyD88 polypeptide has the amino acid sequence of SEQ ID NO: 35 or SEQ ID NO: 83, or a functional fragment thereof.
   H10. The method of any one of embodiments E1-F17, G1-G14, or H1-H8, wherein the truncated MyD88 polypeptide has the amino acid sequence of SEQ ID NO: 27, or a functional fragment thereof.
   H11. The method of any one of embodiments E1-F17, G1-G14, or H1-H10, wherein the truncated MyD88 polypeptide comprises the amino acid sequence of SEQ ID NO: 35 or SEQ ID NO: 83, lacking the TIR domain, or a functional fragment thereof.
   H11.1. The method of any one of embodiments E1-F17, G1-G14, or H1-H10, wherein the truncated MyD88 polypeptide does not comprise contiguous amino acid residues 156 to the C-terminus of the full length MyD88 polypeptide.
   H11.2. The method of any one of embodiments E1-F17, G1-G14, or H1-H10, wherein the truncated MyD88 polypeptide does not comprise contiguous amino acid residues 152 to the C-terminus of the full length MyD88 polypeptide.
   H11.3. The method of any one of embodiments E1-F17, G1-G14, or H1-H10, wherein the truncated MyD88 polypeptide does not comprise contiguous amino acid residues 173 to the C-terminus of the full length MyD88 polypeptide.
   H11.4. The method of any one of embodiments E1-F17, G1-G14, or H1-H8, wherein the full length MyD88 polypeptide comprises the amino acid sequence of SEQ ID NO: 35 or SEQ ID NO: 83.
   H11.5. The method of any one of embodiments E1-F17, G1-G14, or H1-H10, wherein the truncated MyD88 polypeptide consists of the amino acid sequence of SEQ ID NO: 27, or a functional fragment thereof.
   H12. The method of any one of embodiments E1-F17, G1-G14, or H1-H11, wherein the cytoplasmic CD40 polypeptide comprises the amino acid sequence of SEQ ID NO: 29, or a functional fragment thereof.
   H13. The method of any one of embodiments E1-F17, G1-G14, or H1-H11, wherein the cytoplasmic CD40 polypeptide consists of the amino acid sequence of SEQ ID NO: 29, or a functional fragment thereof.
   H14. The method of any one of embodiments E1-F17, G1-G14, or H1-H13, wherein the CD3 ζ polypeptide comprises an amino acid sequence of SEQ ID NO:23, or a functional fragment thereof.
   H15. The method of any one of embodiments E1-F17, G1-G14, or H1-H14, wherein the transmembrane region polypeptide comprises an amino acid sequence of SEQ ID NO: 21, or a functional fragment thereof.
   H16. The method of any one of embodiments E1-F17, G1-G14, or H1-H15, wherein the target cell is a tumor cell.
   H17. The method of any one of embodiments E1-F17, G1-G14, or H1-H16, wherein the target cell is a cell involved in a hyperproliferative disease.
   H18. The method of any one of embodiments E1-F17, G1-G14, or H1-H17, wherein the antigen recognition moiety binds to an antigen selected from the group consisting of PSMA, PSCA, MUC1, CD19, ROR1, Mesothelin, GD2, CD123, MUC16, and Her2/Neu.
   H19. The method of any one of embodiments E1-F17, G1-G14, or H1-H18, wherein the antigen recognition moiety binds to Her2/Neu.
   H20. The method of any one of embodiments E1-F17, G1-G14, or H1-H18, wherein the antigen recognition moiety binds to CD19.
   H21. The method of any one of embodiments E1-F17, G1-G14, or H1-H18, wherein the antigen recognition moiety binds to a viral or bacterial antigen.
   H22. The method of any one of embodiments E1-F17, G1-G14, or H1-H21, wherein the antigen recognition moiety is a single chain variable fragment.
   H23. The method of any one of embodiments F4-F17, G5-G14, or H1-H22, wherein the multimeric ligand binding region binds to dimeric FK506, or a dimeric FK506-like analog.
   H23.1. The method of any one of embodiments F4-F17, G5-G14, or H1-H22, wherein the multimeric ligand binding region binds to rimiducid or to AP20187.
   H23.2. The method of any one of embodiments F4-F17, G5-G14, or H1-H23.1, wherein the multimeric ligand binding region comprises FKBP12 variant polypeptide.
   H23.3. The method of embodiment H23.2, wherein the FKBP12 variant polypeptide binds with higher affinity to the multimeric ligand than the wild type FKBP12 polypeptide.
   H23.4. The method of any one of embodiments H23.2 or H23.3, wherein the FKBP12 variant polypeptide comprises an amino acid substitution at position 36 that binds with higher affinity to the multimeric ligand than the wild type FKBP12 polypeptide.
   H23.5. The method of embodiment H23.4, wherein the amino acid substitution at position 36 is selected from the group consisting of valine, isoleucine, leucine, and alanine.
   H23.6. The method of embodiment H23.5, wherein the multimeric ligand binding region is an FKB12v36 region.
   H24. The method of any one of embodiments F1 to H23.6, wherein the ratio of CD8⁺ to CD4+ T cells is 9:1 or greater
   H25. The method of any one of embodiments F1 to H23.6, wherein at least 90% of the modified cells are CD8+ T cells.
   H26. The method of any one of embodiments F1 to H23.6, wherein at least 95% of the modified cells are CD8⁺ T cells
   H27. The method of any one of embodiments F4-F17, G5-G14, or H1-H26, wherein the inducible Caspase-9 polypeptide comprises the amino acid sequence of SEQ ID NO: 5.
   H27.1. The method of any one of embodiments F4-F17, G5-G14, or H1-H26, wherein the Caspase-9 polypeptide is a modified Caspase-9 polypeptide comprising an amino acid substitution selected from the group consisting of the caspase variants D330A, D330E, and N405Q.
   H28. The method of any one of embodiments E1-F17, G1-G14, or H1-H27.1, wherein the polynucleotide that encodes the chimeric antigen receptor, or the nucleic acid is contained within a viral vector.
   H29. The method of embodiment H28, wherein the viral vector is a retroviral vector.
   H30. The method of embodiment H29, wherein the retroviral vector is a murine leukemia virus vector.
   H31. The method of embodiment H29, wherein the retroviral vector is an SFG vector.
   H32. The method of embodiment H26, wherein the viral vector is an adenoviral vector.
   H33. The method of embodiment H26, wherein the viral vector is a lentiviral vector.
   H34. The method of embodiment H26, wherein the viral vector is selected from the group consisting of adeno-associated virus (AAV), Herpes virus, and Vaccinia virus.
   H35. The method of any one of embodiments E1-F17, G1-G14, or H1-H34, wherein the polynucleotide that encodes the chimeric antigen receptor or the nucleic acid is prepared or in a vector designed for electroporation, sonoporation, or biolistics, or is attached to or incorporated in chemical lipids, polymers, inorganic nanoparticles, or polyplexes.
   H36. The method of any one of embodiments E1-F25, wherein the polynucleotide that encodes the chimeric antigen receptor or the nucleic acid is contained within a plasmid.

### H37. Reserved.

H38. The modified cell of any one of embodiments E1-H37, wherein the cells are obtained or prepared from bone marrow.
H39. The modified cell of any one of embodiments E1-H37, wherein the cells are obtained or prepared from umbilical cord blood.
H40. The modified cell of any one of embodiments E1-H37, wherein the cells are obtained or prepared from peripheral blood.
H41. The modified cell of any one of embodiments E1-H37, wherein the cells are obtained or prepared from peripheral blood mononuclear cells.
H42. The modified cell of any one of embodiments E1-H41, wherein the modified cells are human cells.
H43. The method of any one of embodiments E1-H41, wherein the modified cells are autologous T cells.
H44. The method of any one of embodiments E1-H41, wherein the modified cells are allogeneic T cells.
H45. The method of any one of embodiments E1-H44, wherein the cells are transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

### H46-H48. Reserved.

11. The method of any one of embodiments J1-H48, comprising the step of administering the CD8+ T cell enriched modified cell population to a subject.
12. The method of embodiment II, wherein the antigen recognition moiety binds to an antigen on the tumor.

### I3-I10. Reserved.

I11. The method of any one of embodiments I1-I10, wherein the subject has been diagnosed as having a tumor.
I12. The method of any one of embodiments I1-I11, wherein the subject has cancer.
I13. The method of any one of embodiments I1-I12, wherein the subject has a solid tumor.
I14. The method of any one of embodiments I1-I13, wherein the modified cell population is administered intravenously.
I15. The method of any one of embodiments I1-I14, wherein the modified cell population is delivered to a tumor bed.
I16. The method of embodiment I12, wherein the cancer is present in the blood or bone marrow of the subject.
I17. The method of any one of embodiments I1-I16, wherein the subject has a blood or bone marrow disease.
I18. The method of any one of embodiments I1-I17, wherein the subject has been diagnosed with any condition that can be alleviated by stem cell transplantation.
I19. The method of any one of embodiments I1-I18, wherein the subject has been diagnosed with sickle cell anemia or metachromatic leukodystrophy.
I20. The method of any one of embodiments I1-I18, wherein the patient has been diagnosed with a condition selected from the group consisting of a primary immune deficiency condition, hemophagocytosis lymphohistiocytosis (HLH) or other hemophagocytic condition, an inherited marrow failure condition, a hemoglobinopathy, a metabolic condition, and an osteoclast condition.
I21. The method of any one of embodiments I1-I18, wherein the disease or condition is selected from the group consisting of Severe Combined Immune Deficiency (SCID), Combined Immune Deficiency (CID), Congenital T cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/I L-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.
I22. The method of any one of embodiments I1-I21, comprising administering an additional dose of the modified cell to the subject, wherein the disease or condition symptoms remain or are detected following a reduction in symptoms.
I23. The method of any one of embodiments I1-I22, comprising identifying the presence, absence or stage of a condition or disease in a subject; and transmitting an indication to administer modified cell population of any one of embodiments E1-E45, maintain a subsequent dosage of the modified cell population, or adjust a subsequent dosage of the modified cell population administered to the patient based on the presence, absence or stage of the condition or disease identified in the subject.
I24. The method of any one of embodiments I23, wherein the condition is leukemia.
I25. The method of any one of embodiments I1-I22, wherein the subject has been diagnosed with an infection of viral etiology selected from the group consisting HIV, influenza, Herpes, viral hepatitis, Epstein Bar, polio, viral encephalitis, measles, chicken pox, Cytomegalovirus (CMV), adenovirus (ADV), HHV-6 (human herpesvirus 6, I), and Papilloma virus, or has been diagnosed with an infection of bacterial etiology selected from the group consisting of pneumonia, tuberculosis, and syphilis, or has been diagnosed with an infection of parasitic etiology selected from the group consisting of malaria, trypanosomiasis, leishmaniasis, trichomoniasis, and amoebiasis.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow.

What is claimed is:

1. A modified cell population, comprising modified T cells, wherein:
the modified T cells comprise a polynucleotide that encodes a chimeric antigen receptor, wherein the chimeric antigen receptor comprises:
(i) a transmembrane region;
(ii) a T cell activation molecule; and
(iii) an antigen recognition moiety
wherein the ratio of CD8⁺ to CD4⁺ T cells in the modified cell population is 3:2 or greater.

2. The modified cell population of claim 1, wherein the chimeric antigen receptor comprises
(i) a transmembrane region;
(ii) a costimulatory polypeptide cytoplasmic signaling region, a truncated MyD88 polypeptide region lacking the TIR domain, a truncated MyD88 polypeptide region lacking the TIR domain and a costimulatory polypeptide cytoplasmic signaling region, or a truncated MyD88 polypeptide region lacking the TIR domain and a CD40 cytoplasmic polypeptide region lacking the CD40 extracellular domain;
(iii) a T cell activation molecule; and
(iv) an antigen recognition moiety.

3. The modified cell population of any one of claims 1 to 2, wherein the modified T cells comprise a second polynucleotide that encodes an inducible chimeric pro-apoptotic polypeptide.

4. The modified cell population of claim 1, wherein the modified T cells comprise a second polynucleotide that encodes a chimeric signaling polypeptide, wherein the chimeric signaling polypeptide comprises:
(i) a costimulatory polypeptide cytoplasmic signaling region;
(ii) a truncated MyD88 polypeptide region lacking the TIR domain;
(iii) a truncated MyD88 polypeptide region lacking the TIR domain and a costimulatory polypeptide cytoplasmic signaling region; or
(iv) a truncated MyD88 polypeptide region lacking the TIR domain and a CD40 cytoplasmic polypeptide region lacking the CD40 extracellular domain.

5. The modified cell population of claim 4, wherein the chimeric signaling polypeptide comprises a membrane targeting region.

6. The modified cell population of claim 4, wherein costimulatory polypeptide cytoplasmic signaling region is a signaling region that activates the signaling pathways activated by MyD88, CD40 and/or MyD88-CD40 fusion chimeric polypeptide.

7. The modified cell population of claim 1, wherein the modified T cells comprise a nucleic acid comprising a promoter operably linked to
(i) a first polynucleotide encoding the chimeric antigen receptor; and
(ii) a second polynucleotide encoding a chimeric signaling polypeptide, wherein the chimeric signaling polypeptide comprises
a. a costimulatory polypeptide cytoplasmic signaling region;
b. a truncated MyD88 polypeptide region lacking the TIR domain;
c. a truncated MyD88 polypeptide region lacking the TIR domain and a costimulatory polypeptide cytoplasmic signaling region; or
d. a truncated MyD88 polypeptide region lacking the TIR domain and a CD40 cytoplasmic polypeptide region lacking the CD40 extracellular domain.

8. The modified cell population of claim 7, wherein the nucleic acid comprises, in 5′ to 3′ order, the first polynucleotide and the second polynucleotide.

9. The modified cell population of any one of claim 7 or 8, wherein the first polynucleotide encodes, in 5′ to 3′ order, an antigen recognition moiety, a transmembrane region, and a T cell activation molecule, and the second polynucleotide is 3′ of the polynucleotide sequence encoding the T cell activation molecule.

10. The modified cell population of any one of claims 7 to 9, wherein the nucleic acid comprises a third polynucleotide that encodes a linker polypeptide between the first and the second polynucleotides.

11. The modified cell population of claim 10, wherein the linker polypeptide comprises a 2A polypeptide.

12. The modified cell population of any one of claims 10 to 11, wherein the nucleic acid comprises a fourth polynucleotide encoding an inducible chimeric pro-apoptotic polypeptide.

13. The modified cell population of any one of claims 2 to 12, wherein the costimulatory polypeptide cytoplasmic signaling region is selected from the group consisting of CD27, CD28, 4-1BB, OX40, ICOS, RANK, TRANCE, and DAP10, or a signaling region that activates the signaling pathways activated by MyD88, CD40, CD27, CD28, 4-1BB, OX40, ICOS, RANK, TRANCE, and DAP10.

14. The modified cell population of any one of claims 2 to 3, wherein the chimeric antigen receptor comprises two costimulatory polypeptide cytoplasmic signaling regions selected from the group consisting of CD27, CD28, 4-1BB, OX40, ICOS, RANK, TRANCE, and DAP10, or a signaling region that activates the signaling pathways activated by CD27, CD28, 4-1BB, OX40, ICOS, RANK, TRANCE, and DAP10, or a signaling region that activates the signaling pathways activated by MyD88, CD40, CD27, CD28, 4-1BB, OX40, ICOS, RANK, TRANCE, and DAP10.

15. The modified cell population of any one of claims 4 to 12, wherein the chimeric signaling polypeptide comprises two costimulatory polypeptide cytoplasmic signaling regions selected from the group consisting of CD27, CD28, 4-1BB, OX40, ICOS, RANK, TRANCE, and DAP10, or a signaling region that activates the signaling pathways activated by MyD88, CD40, CD27, CD28, 4-1BB, OX40, ICOS, RANK, TRANCE, and DAP10.

16. The modified cell population of any one of claims 1 to 15, wherein 80% or more of the modified cells are CD8⁺ T cells.

17. A method for stimulating a cell mediated immune response to a target cell or tissue in a subject, comprising administering a modified cell population of any one of claims 1 to 16.

18. A method for treating a subject having a disease or condition associated with an elevated expression of a target antigen, comprising administering to the subject an effective amount of a modified cell population of any one of claims 1 to 16.

19. A method for reducing the size of a tumor in a subject, comprising administering a modified cell population of any one of claims 1 to 16 to the subject, wherein the antigen recognition moiety binds to an antigen on the tumor.

20. A method for preparing a modified cell population of any one of claims 1 to 16, comprising contacting T cells with a nucleic acid that comprises a polynucleotide that encodes the chimeric antigen receptor with a cell population under conditions in which the nucleic acid is incorporated into the cells, and enriching the T cells to obtain a modified cell population wherein the ratio of CD8⁺ to CD4⁺ T cells in the cell population is 3:2 or greater.

21. The method of claim 20, comprising the step of administering the modified cell population to a subject.

22. The method of claims 17 to 19, further comprising administering a cytokine neutralizing agent.

23. The method of claim 23 wherein the neutrailizing agent is an antibody.

24. The method of claim 23, wherein the neutrailizing agent is an anti-TNFα antibody.
